(19) United States
(12) Patent Application Publication
(10) Pub. No.: US 2010/0204159 A1
(43) Pub. Date: Aug. 12, 2010

(54) ORGANIC COMPOUNDS AND THEIR USES

(76) Inventors: Trixl Brandl, Basel (CH); Sylvain Cottens, Witterswil (CH); Claus Ehrhardt, Lorrach (DE); Jiping Fu, Arlington, MA (US); Subramanian Karur, Medford, MA (US); David Thomas Parker, Lexington, MA (US); Michael A. Patane, Reading, MA (US); Prakash Raman, Acton, MA (US); Stefan Andreas Randl, Basel (CH); Pascal Rigollier, Mulhouse (FR); Mohindra Seepersaud, Belmont, MA (US); Oliver Simic, Basel (CH)

Correspondence Address: NOVARTIS INSTITUTES FOR BIOMEDICAL RESEARCH, INC., 220 MASSACHUSETTS AVENUE, CAMBRIDGE, MA, 02139, US

(21) Appl. No.: 12/440,139

(22) PCT Filed: Sep. 11, 2007
(86) PCT No.: PCT/US07/19801

§ 371 Date: Apr. 15, 2010

Related U.S. Application Data
(60) Provisional application No. 60/825,509, filed on Sep. 13, 2006.

(51) Int. Cl. A61K 38/06; C07D 417/14; A61P 31/14; A61K 38/05; C07K 5/083

(52) U.S. Cl. 514/18; 540/455; 514/19; 530/331

(57) ABSTRACT

The present application describes organic compounds that are useful for the treatment, prevention and/or amelioration of human diseases.

### BACKGROUND

Hepatitis C virus (HCV) is a (+)-sense single-stranded RNA virus that has been implicated as the major causative agent in non-A, non-B hepatitis (NANBH), particularly in blood-associated NANBH (BB-NANBH). NANBH is to be distinguished from other types of viral-induced liver disease, such as hepatitis A virus (HAV), hepatitis B virus (HBV), delta hepatitis virus (HDV), cytomegalovirus (CMV) and Epstein-Barr virus (EBV), as well as from other forms of liver disease such as alcoholism and primary biliar cirrhosis.

Recently, an HCV protease necessary for polypeptide processing and viral replication has been identified, cloned and expressed. (See, e.g., U.S. Pat. No. 5,712,145). This approximately 3000 amino acid polyprotein contains, from the amino terminus to the carboxy terminus, a nucleocapsid protein (C), envelope proteins (E1 and E2) and several non-structural proteins (NS1, 2, 3,4a, 5a and 5b). NS3 is an approximately 68 kda protein, encoded by approximately 1893 nucleotides of the HCV genome, and has two distinct domains: (a) a serine protease domain consisting of approximately 200 of the N-terminal amino acids; and (b) an RNA-dependent ATPase domain at the C-terminus of the protein. The NS3 protease is considered a member of the chymotrypsin family because of similarities in protein sequence, overall three-dimensional structure and mechanism of catalysis. The HCV NS3 serine protease is responsible for proteolysis of the polypeptide (polyprotein) at the NS3/NS4a, NS4a/NS4b, NS4b/NS5a and NS5a/NS5b junctions and is thus responsible for generating four viral proteins during viral replication. This has made the HCV NS3 serine protease an attractive target for antiviral chemotherapy.

It has been determined that the NS4a protein, an approximately 6 kda polypeptide, is a co-factor for the serine protease activity of NS3. Autocleavage of the NS3/NS4a junction by the NS3/NS4a serine protease occurs intramolecularly (i.e., cis) while the other cleavage sites are processed intermolecularly (i.e., trans).

HCV has been implicated in cirrhosis of the liver and in induction of hepatocellular carcinoma. The prognosis for patients suffering from HCV infection is currently poor. HCV infection is more difficult to treat than other forms of hepatitis due to the lack of immunity or remission associated with HCV infection. Current data indicates a less than 50% survival rate at four years post cirrhosis diagnosis. Patients diagnosed with localized resectable hepatocellular carcinoma have a five-year survival rate of 10-30%, whereas those with localized unresectable hepatocellular carcinoma have a five-year survival rate of less than 1%.

Current therapies for hepatitis C include interferon-α (INF_{α}) and combination therapy with ribavirin and interferon. See, e.g., Beremguer et al. (1998) Proc. Assoc. Am. Physicians 110(2):98-112. These therapies suffer from a low sustained response rate and frequent side effects. See, e.g., Hoofnagle et al. (1997) N. Engl. J. Med. 336:347. Currently, no vaccine is available for HCV infection.

### SUMMARY OF THE INVENTION

There remains a need for new treatments and therapies for HCV infection, as well as HCV-associated disorders. There is also a need for compounds useful in the treatment or prevention or amelioration of one or more symptoms of HCV, as well as a need for methods of treatment or prevention or amelioration of one or more symptoms of HCV. Furthermore, there is a need for methods for modulating the activity of HCV-serine proteases, particularly the HCV NS3/NS4a serine protease, using the compounds provided herein.

In one aspect, the invention provides compounds of the Formula I:

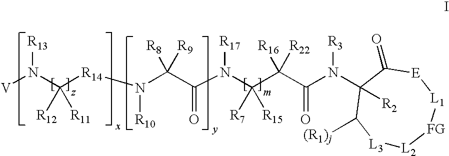

and pharmaceutically acceptable salts, enantiomers, stereoisomers, rotamers, tautomers, diastereomers, or racemates thereof;

wherein
the macrocycle:

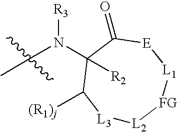

comprises between 10 to 25 ring atoms;

m, x and z are each independently selected from 0 or 1;

j, p and y are independently selected at each occurrence from the group consisting of 0, 1 and 2;

R₁ and R₂ are independently selected from hydrogen or from the group consisting of alkyl, alkenyl, alkynyl, cycloalkyl, cyano, alkoxy, and cycloalkyloxy, each of which is unsubstituted or substituted with 1-6 moieties which can be the same or different and are independently selected from the group consisting of hydroxy, oxo, alkyl, aryl, alkoxy, aryloxy, thio, alkylthio, arylthio, amino, alkylamino, arylamino, alkylsulfonyl, arylsulfonyl, alkylsulfonamido, arylsulfonamido, carboxy, carbalkoxy, amido, carboxamido, alkoxycarbonylamino, alkoxycarbonyloxy, alkylureido, arylureido, halogen, cyano, or nitro; wherein each of said alkyl, alkoxy, and aryl can be unsubstituted or optionally independently substituted with one or more moieties which can be the same or different and are independently selected from alkyl, alkenyl, alkynyl, cycloalkyl, cycloalkyl-alkyl, heterocyclyl, heterocyclylalkyl, aryl, alkylaryl, aralkyl, arylheteroaryl, heteroaryl, heterocyclylamino, alkylheteroaryl and heteroaralkyl;

R₃ is selected from the group consisting of H and C₁₋₄-alkyl;

E is a divalent residue selected from the group consisting of NR₂₃, C(O)NR₂₃, NR₂₃S(O)ₚ, and NR₂₃S(O)ₚNR₂₃;

L₁ and L₂ are divalent residues independently selected from the group consisting of alkylene, (CH₂)ᵢ-FG-(CH₂)ₖ, alkenylene, alkynylene, arylene, heteroarylene, and cycloalkylene, each of which is substituted with 0 to 4 independently selected X₁ or X₂ groups;

i and k are independently selected integers of from 0 to 7;

L₃ is absent or a divalent ethylene or acetylene residue, wherein the divalent ethylene is substituted by 0-2 substituents selected from alkyl, aryl, heteroaryl, mono- or di-alkylamino-C₀-C₆alkyl, hydroxyl alkyl or alkoxyalkyl;

FG is absent or a divalent residue selected from the group consisting of O, S(O)ₚ, NR₂₃, C(O), C(O)NR₂₃, NR₂₃C(O), OC(O)NR₂₃, NR₂₃C(O)O, NR₂₃C(O)NR₂₃, S(O)ₚNR₂₃, NR₂₃S(O)ₚ, and NR₂₃S(O)ₚNR₂₃;

R₂₃ is independently selected at each occurrence from hydrogen or the group consisting of alkyl, alkenyl, alkynyl, cycloalkyl, cycloalkylalkyl, aryl, heteroaryl, heteroaralkyl and aralkyl, each of which is substituted with 0-2 substituents independently selected from halogen, alkyl, and alkoxy;

R₇, R₈, R₉, R₁₀, R₁₁, R₁₂, R₁₃ R₁₆, R₁₅, R₁₇, R₂₂, and V are each, independently, selected from the group consisting of H, alkyl, alkenyl, alkynyl, aryl, alkyl-aryl, heteroalkyl, heterocyclyl, heteroaryl, aryl-heteroaryl, alkyl-heteroaryl, cycloalkyl, alkyloxy, alkyl-aryloxy, aryloxy, heteroaryloxy, heterocyclyloxy, cycloalkyloxy, amino, alkylamino, arylamino, alkyl-arylamino, arylamino, heteroarylamino, cycloalkylamino, carboxyalkylamino, aralkyloxy and heterocyclylamino; each of which may be further independently substituted one or more times with X₁ and X₂;

X₁ is alkyl, alkenyl, alkynyl, cycloalkyl, spirocycloalkyl, cycloalkyl-alkyl, heterocyclyl, heterocyclylalkyl, aryl, alkylaryl, aralkyl, arylheteroaryl, heteroaryl, heterocyclylamino, alkylheteroaryl, or heteroaralkyl; wherein X₁ can be independently substituted with one or more of X₂ moieties which can be the same or different and are independently selected;

X₂ is hydroxy, oxo, alkyl, aryl, heteroaryl, alkoxy, aryloxy, heteroaryloxy, thio, alkylthio, arylthio, heteroarylthio, amino, alkylamino, arylamino, heteroarylamino, alkylsulfonyl, arylsulfonyl, heteroarylsulfonyl, alkylsulfonamido* arylsulfonamido, heteroarylsulfonamido, arylaminosulfonyl, heteroarylaminosulfonyl, mono and dialkylamino sulfonyl, carboxy, carbalkoxy, amido, carboxamido, alkoxycarbonylamino, alkoxycarbonyloxy, carbamoyl, ureido, alkylureido, arylureido, halogen, cyano, or nitro; wherein each of said alkyl, alkoxy, and aryl can be unsubstituted or optionally independently substituted with one or more moieties which can be the same or different and are independently selected from alkyl, alkenyl, alkynyl, cycloalkyl, cycloalkyl-alkyl, heterocyclyl, heterocyclylalkyl, aryl, alkylaryl, aralkyl, arylheteroaryl, heteroaryl, heterocyclylamino, alkylheteroaryl and heteroaralkyl;

R₁₄ is C(O) or SOₚ

V is selected from the group consisting of -Q¹-Q², wherein Q¹ is absent, C(O), S(O)ₚ, N(H), N(C₁₋₄-alkyl), C═N(CN), C═N(SO₂CH₃), or C═N—COH, and Q² is H, C₁₋₄-alkyl, C═N—COH—C₁₋₄-alkyl, O—C₁₋₄-alkyl, NH₂, N(H)—C₁₋₄-alkyl, N(C₁₋₄-alkyl)₂, SO₂-aryl, SO₂—C₁₋₄-alkyl, C₃₋₆-cycloalkyl-C₀₋₄-alkyl, aryl, heteroaryl and heterocycle, each of which may be independently substituted one or more times with a halogen atom, C₁₋₄-alkyl, C₂₋₄-alkenyl, C₂₋₄-alkynyl, C₁₋₄-alkoxy, C₂₋₄-alkenyloxy, C₂₋₄-alkynyloxy, C₁₋₄-alkyl substituted by one or more halogen atoms, C₃₋₆-cycloalkyl, carboxylate, carboxamido, mono- and di-alkylamino, or mono- and di-alkylcarboxamido;

or R₂₂ and R₁₆ may together form a 3, 4, 5, 6 or 7-membered ring and may contain one or more heteroatoms, wherein the ring may be further substituted one or more times;

or R₇ and R₁₅ may together form a 3, 4, 5, 6 or 7-membered ring and may contain one or more heteroatoms, wherein the ring may be further substituted one or more times;

or R₁₅ and R₁₇ may together form a 3, 4, 5, 6 or 7-membered ring and may contain one or more heteroatoms, wherein the ring may be further substituted one or more times;

or R₁₅ and R₁₆ may together form a 4, 5, 6 or 7-membered ring and may contain one or more heteroatoms, wherein the ring may be further substituted one or more times;

or R₁₅ and R₁₆may together form an arylene or heteroarylene ring and R₇ and R₂₂ are absent, wherein the ring may be further substituted one or more times;

or R₁ and R₂ may together form a 3, 4, 5, 6 or 7-membered ring that is saturated or partially unsaturated and may contain one or more heteroatoms, wherein the ring may be further substituted one or more times;

or R₁₇ and R₁₆ may together form a 4, 5, 6, 7 or 8-membered ring of the formula:

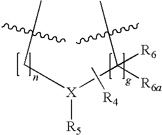

wherein

n and g are each, independently, 0, 1 or 2;

X is O, S, N, NR₅, CR₅ or CR₅R₅ₐ;

R₄ is selected from the group consisting of H, C₁₋₆-alkyl, C₃₋₇-cycloalkyl, aryl, heterocycle and heteroaryl, each of which may be independently substituted one or more times with a halogen atom or C₁₋₄-alkyl;

R₅ is selected from the group consisting of H, hydroxyl, oxo, C₁₋₈-alkyl, C₂₋₈-alkenyl, C₂₋₈-alkynyl, C₃₋₈-cycloalkyl-C₀₋₄-alkyl, aryl-C₀₋₄-alkyl, heterocycle-C₀₋₄-alkyl, heteroaryl-C₀₋₄-alkyl, C₃₋₈-cycloalkyloxy, aryloxy, NR₂₃COR₂₃, CONR₂₃R₂₃, NR₂₃CONHR₂₃, OCONR₂₃R₂₃, NR₂₃COOR₂₃, OCOR₂₃, COOR₂₃, aryl-C(O)O, aryl-C(O)NR₂₃, heteroaryloxy, heteroaryl-C(O)O, heteroaryl-C(O)NR₂₃, each of which may be independently substituted one or more times with a halogen atom, aryl, heteroaryl, trihalomethyl, C₁₋₄-alkyl, or C₁₋₄-alkoxy;

R₅ₐ is selected from the group consisting of H, hydroxyl, C₁₋₈-alkyl, C₂₋₈-alkenyl, C₂₋₈-alkynyl, C₃₋₈-cycloalkyl-C₀₋₄-alkyl, aryl-C₀₋₄-alkyl and heteroaryl-C₀₋₄-alkyl,

or R₄ and R₅ may together form a fused dimethyl cyclopropyl ring, a fused cyclopentane ring, a fused phenyl ring or a fused pyridyl ring, each of which may be substituted with a halogen atom, aryl, heteroaryl, trihalomethyl, C₁₋₄-alkoxy or C₁₋₄-alkyl;

or R₅ and R₅ₐ may together form a spirocyclic ring having between 3 and 7 ring atoms which is optionally substituted by 0-4 substitutents selected from cyano, halogen, hydroxyl, amino, thiol, C₁₋₈-alkyl, C₂₋₈-alkenyl, C₂₋₈-alkynyl, C₁₋₈-alkoxide, C₁₋₈-haloalkyl, C₂₋₈-haloalkenyl, C₂₋₈-haloalkynyl, C₁₋₈-haloalkoxide, C₁₋₈-alkylthio, C₁₋₈-alkylsulfonyl, C₁₋₈-alkylsulfoxide, C₁₋₈-alkanoyl, C₁₋₈-alkoxycarbonyl, C₃₋₇-cycloalkyl-C₀₋₄-alkyl, aryl-C₀₋₄-alkyl, heterocyclyl-C₀₋₄-alkyl, heteroaryl-C₀₋₄-alkyl, COOH, C(O)NH₂, mono- and di-C₁₋₄-alkyl-carboxamide, SO₃H, SO₂NH₂, and mono-and di-C₁₋₄-alkylsulfonamide, or two substitutents taken together form a fused or spirocyclic 3 to 7 membered ring having 0, 1 or 2 ring heteroatoms selected from N, O and S, which fused or spirocyclic ring has 0 to 2 independently selected substituents selected from halogen, C₁₋₄alkyl, C₁₋₄alkoxy, C₁₋₄alkanoyl, mono- and di-C₁₋₄-alkylamino, mono- and di-C₁₋₄-alkyl-carboxamide, C₁₋₄alkoxycarbonyl, and phenyl; and

R₆ and R₆ₐ are independently selected at each occurrence from the group consisting of H, C₁₋₄-alkyl and (CH₂)₀₋₄—C₃₋₆-cycloalkyl; or R₆ and R₆ₐ may together form a spirocyclic ring having between 3 and 7 ring atoms which is optionally substituted by 0-4 substitutents selected from cyano, halogen, hydroxyl, amino, thiol, C₁₋₈alkyl, C₂₋₈-alkenyl, C₂₋₈-alkynyl, C₁₋₈-alkoxide, C₁₋₈-haloalkyl, C₂₋₈-haloalkenyl, C₂₋₈-haloalkynyl, C₁₋₈-haloalkoxide, C₁₋₈-alkylthio, C₁₋₈-alkylsulfonyl, C₁₋₈-alkylsulfoxide, C₁₋₈-alkanoyl, C₁₋₈-alkoxycarbonyl, C₃₋₇-cycloalkyl-C₀₋₄-alkyl, aryl-C₀₋₄-alkyl, heterocyclyl-C₀₋₄-alkyl, heteroaryl-C₀₋₄-alkyl, COOH, C(O)NH₂, mono- and di-C₁₋₄-alkyl-carboxamide, SO₃H, SO₂NH₂, and mono-and di-C₁₋₄-alkylsulfonamide, or two substitutents taken together form a fused or spirocyclic 3 to 7membered ring having 0, 1 or 2 ring heteroatoms selected from N, O and S, which fused or spirocyclic ring has 0 to 2 independently selected substituents selected from halogen, C₁₋₄alkyl, C₁₋₄alkoxy, C₁₋₄alkanoyl, mono- and di-C₁₋₄-alkylamino, mono- and di-C₁₋₄-alkyl-carboxamide, C₁₋₄-alkoxycarbonyl, and phenyl.

In certain embodiments, compounds of Formula I include those compounds in which

E is a divalent residue selected from the group consisting of NR₂₃, C(O)NR₂₃ and NR₂₃S(O)ₚNR₂₃;

L₁ and L₂ are divalent residues independently selected from the group consisting of alkylene, (CH₂)ᵢ-FG-(CH₂)ₖ, alkenylene, alkynylene, arylene, heteroarylene, and cycloalkylene, each of which is substituted with 0 to 4 independently selected X₁ or X₂ groups;

i and k are independently selected integers of from 0 to 7;

L₃ is absent or a divalent ethylene or acetylene residue, wherein the divalent ethylene is substituted by 0-2 substituents selected from alkyl, aryl, heteroaryl, mono- or di-alkylamino-C₀-C₆alkyl, hydroxyl alkyl or alkoxyalkyl; and

FG is absent or a divalent residue selected from the group consisting of O, S(O)ₚ, NR₂₃, C(O), C(O)NR₂₃, NR₂₃C(O), OC(O)NR₂₃, NR₂₃C(O)O, NR₂₃C(O)NR₂₃, S(O)ₚNR₂₃, NR₂₃S(O)ₚ, and NR₂₃S(O)ₚNR₂₃. Said compounds are referred to as compounds of Formula Ia.

In certain other embodiments, compounds of Formula I include those compounds in which

E is a divalent residue selected from the group consisting of NR₂₃, C(O)NR₂₃, NR₂₃S(O)ₚ, NR₂₃S(O)ₚNR₂₃;

L₁ and L₂ are divalent residue independently selected from the group consisting of alkylene, (CH₂)ᵢ-FG-(CH₂)ₖ, arylene, heteroarylene, and cycloalkylene, each of which is substituted with 0 to 4 independently selected X₁ or X₂ groups;

i and k are independently selected integers of from 0 to 7;

L₃ is absent or a divalent ethylene or acetylene residue, wherein the divalent ethylene is substituted by 0-2 substituents selected from alkyl, aryl, heteroaryl, mono- or di-alkylamino-C₀-C₆alkyl, hydroxyl alkyl or alkoxyalkyl; and

FG is a divalent residue selected from the group consisting of O, S(O)ₚ, NR₂₃, C(O), C(O)NR₂₃, NR₂₃C(O), OC(O)NR₂₃, NR₂₃C(O)O, NR₂₃C(O)NR₂₃, S(O)ₚNR₂₃, NR₂₃S(O)ₚ, and NR₂₃S(O)ₚNR₂₃. Said compounds are referred to as compounds of Formula Ib.

In certain compounds of Formula I or any subformulae thereof, E is NHSO₂NR₂₃ and R₂₃ is hydrogen or a residue selected from C₁₋₄alkyl or C₃₋₇-cycloalkylC₀₋₄alkyl, each of which is substituted with 0-2 residues independently selected from the group consisting of halogen, hydroxy, amino, C₁₋₄alkyl, C₁₋₄alkoxy, and mono- and di-C₁₋₄alkylamino.

In yet other embodiments, compounds of Formula I include those compounds in which

E is a divalent residue selected from the group consisting of NR₂₃, C(O)NR₂₃, NR₂₃S(O)ₚ, NR₂₃S(O)ₚNR₂₃;

L₁ is a divalent residue selected from the group consisting of arylene, heteroarylene, and cycloalkylene, which is substituted with 0 to 4 independently selected X₁ or X₂ groups;

L₂ is a divalent residue selected from the group consisting of alkylene, (CH₂)ᵢ-FG-(CH₂)ₖ, arylene, heteroarylene, and cycloalkylene, which is substituted with 0 to 4 independently selected X₁ or X₂ groups;

i and k are independently selected integers of from 0 to 7;

L₃ is absent or a divalent ethylene or acetylene residue, wherein the divalent ethylene is substituted by 0-2 substituents selected from alkyl, aryl, heteroaryl, mono- or di-alkylamino-C₀-C₆alkyl, hydroxyl alkyl or alkoxyalkyl; and

FG is absent or a divalent residue selected from the group consisting of O, S(O)ₚ, NR₂₃, C(O), C(O)NR₂₃, NR₂₃C(O), OC(O)NR₂₃, NR₂₃C(O)O, NR₂₃C(O)NR₂₃, S(O)ₚNR₂₃, NR₂₃S(O)ₚ, and NR₂₃S(O)ₚNR₂₃. Said compounds are referred to as compounds of Formula Ic.

In one embodiment, the invention provides a method of treating an HCV-associated disorder comprising administering to a subject in need thereof a pharmaceutically acceptable amount of a compound of the invention, such that the HCV-associated disorder is treated.

In another embodiment, the invention provides a method of treating an HIV infection comprising administering to a subject in need thereof a pharmaceutically acceptable amount of a compound of the invention.

In still another embodiment, the invention provides a method of treating, inhibiting or preventing the activity of HCV in a subject in need thereof, comprising administering to the subject a pharmaceutically acceptable amount of a compound of the invention. In one embodiment, the compounds of the invention inhibit the activity of the NS2 protease, the NS3 protease, the NS3 helicase, the NS5a protein, and/or the NS5b polymerase. In another embodiment, the interaction between the NS3 protease and NS4A cofactor is disrupted. In yet another embodiment, the compounds of the invention prevent or alter the severing of one or more of the NS4A-NS4B, NS4B-NS5A and NS5A-NS5B junctions of the HCV. In another embodiment, the invention provides a method of inhibiting the activity of a serine protease, comprising the step of contacting said serine protease with a compound of the invention. In another embodiment, the invention provides a method of treating, inhibiting or preventing the activity of HCV in a subject in need thereof, comprising administering to the subject a pharmaceutically acceptable amount of a compound of the invention, wherein the compound interacts with any target in the HCV life cycle. In one embodiment, the target of the HCV life cycle is selected from the group consisting of NS2 protease, NS3 protease, NS3 helicase, NS5a protein and NS5b polymerase.

In another embodiment, the invention provides a method of decreasing the HCV RNA load in a subject in need thereof comprising administering to the subject a pharmaceutically acceptable amount of a compound of the invention.

In another embodiment, the compounds of the invention exhibit HCV protease activity. In one embodiment, the compounds are an HCV NS3-4A protease inhibitor.

In another embodiment, the invention provides a method of treating an HCV-associated disorder in a subject, comprising administering to a subject in need thereof a pharmaceutically acceptable amount of a compound of the invention, and a pharmaceutically acceptable carrier, such that the HCV-associated disorder is treated.

In still another embodiment, the invention provides a method of treating an HCV-associated disorder comprising administering to a subject in need thereof a pharmaceutically effective amount of a compound of the invention, in combination with a pharmaceutically effective amount of an additional HCV-modulating compound, such as interferon or derivatized interferon, or a cytochrome P450 monooxygenase inhibitor, such that the HCV-associated disorder is treated. In one embodiment, the additional HCV-modulating compound is selected from the group consisting of Sch 503034, ITMN-191 and VX-950.

In another embodiment, the invention provides a method of inhibiting hepatitis C virus replication in a cell, comprising contacting said cell with a compound of the invention.

In yet another embodiment, the invention provides a packaged HCV-associated disorder treatment, comprising an HCV-modulating compound of the invention, packaged with instructions for using an effective amount of the HCV-modulating compound to treat an HCV-associated disorder.

In certain embodiments, the HCV-associated disorder is selected from the group consisting of HCV infection, liver cirrhosis, chronic liver disease, hepatocellular carcinoma, cryoglobulinaemia, non-Hodgkin's lymphoma, and a suppressed innate intracellular immune response.

In another embodiment, the invention provides a method of treating HCV infection, liver cirrhosis, chronic liver disease, hepatocellular carcinoma, cryoglobulinaemia, non-Hodgkin's lymphoma, and/or a suppressed innate intracellular immune response in subject in need thereof comprising administering to the subject a pharrnaceutically acceptable amount of a compound of the invention.

In one embodiment, the HCV to be treated is selected of any HCV genotype. In another embodiment, the HCV is selected from HCV genotype 1, 2 and/or 3.

### DETAILED DESCRIPTION OF THE INVENTION

This invention is directed to compounds, e.g., peptide compounds, and intermediates thereto, as well as pharmaceutical compositions containing the compounds for use in treatment of HCV infection. This invention is also directed to the compounds of the invention or compositions thereof as protease inhibitors, particularly as serine protease inhibitors, and more particularly as HCV NS3 protease inhibitors. The compounds are particularly useful in interfering with the life cycle of the hepatitis C virus and in treating or preventing an HCV infection or physiological conditions associated therewith. The present invention is also directed to methods of combination therapy for inhibiting HCV replication in cells, or for treating or preventing an HCV infection in patients using the compounds of the invention or pharmaceutical compositions, or kits thereof.

In one aspect, the compounds of the invention are compounds of any one of Formulae I, Ia, Ib, and/or Ic, in which R₁ and R₂ taken in combination form a 3, 4, 5, or 6-membered saturated carbdcyclic ring which is substituted with 0-2 substituents independently selected from halogen, alkyl, alkenyl, and alkoxy. In other aspects, compounds of the invention are compounds of any one of Formulae I, Ia, Ib, and/or Ic, in which R₁ and R₂ taken in combination form a cyclopropyl ring. In certain compounds of any one of Formulae I, Ia, Ib, and/or Ic include those compounds in which R₁ and R₂ are taken in combination to form a cyclopropyl ring substituted with 0-2 substituents independently selected from halogen, alkyl, alkenyl, and alkoxy or substituted with 0 to 2 C₁-C₄alkyl residues.

In another aspect, the compounds of the invention are compounds of any one of Formulae I, Ia, Ib, and/or Ic, in which R₁ is H or C₁₋₄ alkyl; and R₂ is H, C₁-C₄alkyl, C₁-C₄fluoroalkyl, C₂-C₄alkenyl, or C₃-C₇cycloalkylC₀₋₂alkyl.

Certain other compounds of Formulae I, Ia, Ib, and/or Ic comprise a macrocycle having between 10 and 25 ring atoms, between 11 and 24 ring atoms, between 12 and 22 ring atoms or between 14 and 20 ring atoms. Certain compounds of Formulae I, Ia, Ib, and/or Ic comprise a macrocycle having 10, 11, 12, 13, 14, 15, 16, 17, 18, 19, 20, 21, 22, 23, 24, or 25 ring atoms. In certain instances, compounds of Formulae I, Ia, Ib, and/or Ic comprise a macrocycle having 14, 15, 16, 17, 18, 19, or 20 ring atoms.

Certain other compounds of Formulae I, Ib, and/or Ic comprise a macrocycle selected from the group consisting of macrocycles of the formulae:

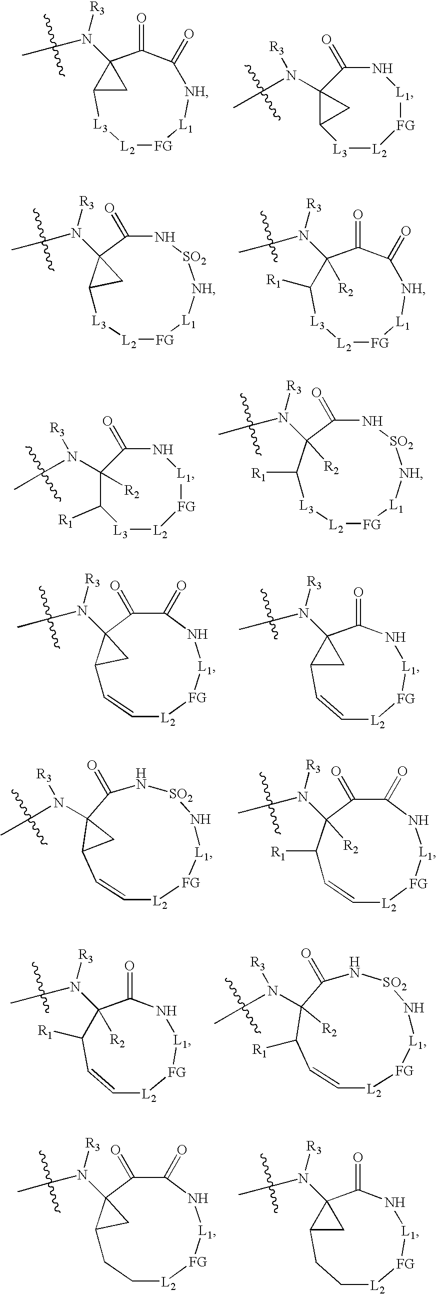

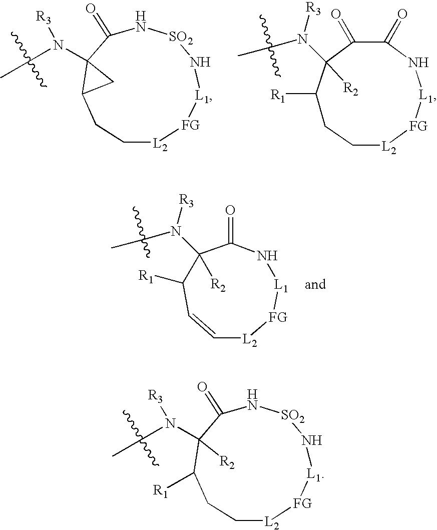

Certain other compounds of Formulae I, Ia, Ib, and/or Ic comprise a macrocycle selected from the group consisting of macrocycles of the formulae:

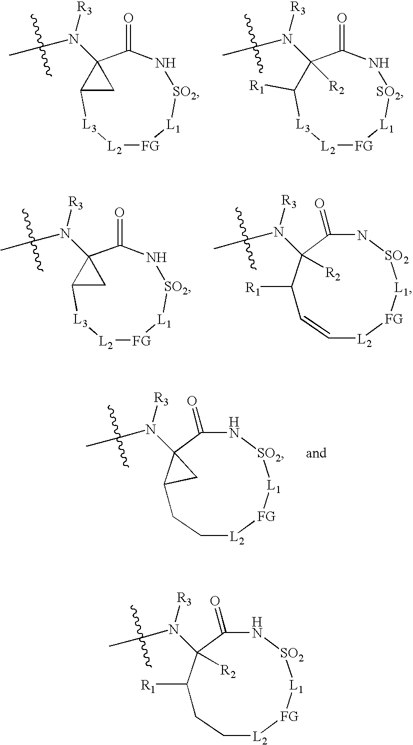

In certain compounds of Formulae I, Ia, and/or Ib, L₁ is C₁-C₆alkylene, C₃-C₇cycloalkylene, arylene or heteroarylene, each of which is substituted by 0-4 residues independently selected from C₁-C₄alkyl, C₁-C₄alkoxy, hydroxyl, amino, mono- and di-C₁-C₄alkylamino, halogen, cyano, C₁-C₄fluoroalkyl, C₁-C₄fluoroalkoxy, COOH, carboxamide (CONH₂), mono- and di-C₁-C₄alkylcarboxamide, aryl, heteroaryl and 5 or 6 membered saturated heterocycles;

L₂ is selected from C₁-C₆alkylene and C₂-C₆alkenylene, each of which is substituted by 0-4 residues independently selected from C₁-C₄alkyl, C₁-C₄alkoxy, hydroxyl, amino, mono- and di-C₁-C₄alkylamino, halogen, cyano, C₁-C₄fluoroalkyl, C₁-C₄fluoroalkoxy, COOH, carboxamide (CONH₂), mono- and di-C₁-C₄alkylcarboxamide, aryl, heteroaryl and 5 or 6 membered saturated heterocycles; and

L₃ is absent or a divalent ethylene residue which is substituted by 0 to 2 independently selected methyl or ethyl residues.

In yet other compounds of Formulae I, Ia, and/or Ib, L₁ is a divalent residue selected from C₂-C₄alkylene, 1,2-phenylene, 1,3-phenylene, 2,4-pyridylene, 2,3-pyridylene, 3,4-pyridylene or 1,7-indolylene, 2,7-indolylene, each of which is substituted with 0-3 residues selected from C₁-C₄alkyl, C₁-C₄alkoxy, hydroxyl, amino, mono- and di-C₁-C₄alkylamino, halogen, cyano, C₁-C₂fluoroalkyl, C₁-C₂fluoroalkoxy, COOH, carboxamide (CONH₂), and mono- and di-C₁-C₄alkylcarboxamide.

In certain compounds of Formulae I, Ia, Ib, and/or Ic, L₁ is C₃-C₇cycloalkylene, arylene or heteroarylene which is substituted by 0-4 residues independently selected from C₁-C₄alkyl, C₁-C₄alkoxy, hydroxyl, amino, mono- and di-C₁-C₄alkylamino, halogen, cyano, C₁-C₄fluoroalkyl, C₁-C₄fluoroalkoxy, COOH, carboxamide (CONH₂), mono- and di-C₁-C₄alkylcarboxamide, aryl, heteroaryl and 5 or 6 membered saturated heterocycles;

L₂ is selected from C₁-C₆alkylene and C₂-C₆alkenylene, each of which is substituted by 0-4 residues independently selected from C₁-C₄alkyl, C₁-C₄alkoxy, hydroxyl, amino, mono- and di-C₁-G₄alkylamino, halogen, cyano, C₁-C₄fluoroalkyl, C₁-C₄fluoroalkoxy, COOH, carboxamide (CONH₂), mono- and di-C₁-C₄alkylcarboxamide, aryl, heteroaryl and 5 or 6 membered saturated heterocycles; and

L₃ is absent or a divalent ethylene residue which is substituted by 0 to 2 independently selected methyl or ethyl residues.

In yet other compounds of Formulae I, Ia, Ib and/or Ic, L₁ is a divalent residue selected from 1,2-phenylene, 1,3-phenylene, 2,4-pyridylene, 2,3-pyridylene, 3,4-pyridylene or 1,7-indolylene, 2,7-indolylene, each of which is substituted with 0-3 residues selected from C₁-C₄alkyl, C₁-C₄alkoxy, hydroxyl, amino, mono- and dir C₁-C₄alkylamino, halogen, cyano, C₁-C₂fluoroalkyl, C₁-C₂fluoroalkoxy, COOH, carboxamide (CONH₂), and mono- and di-C₁-C₄alkylcarboxamide.

Certain compounds of Formulae I, Ia, Ib and/or Ic include compounds of Formula II:

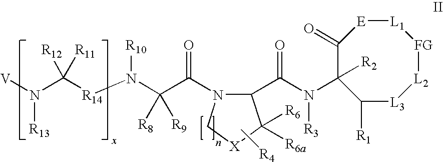

and pharmaceutically acceptable salts, enantiomers, stereoisomers, rotamers, tautomers, diastereomers, or racemates thereof.

Certain compounds of Formulae I, Ia, Ib and/or Ic include compounds of Formula IIa:

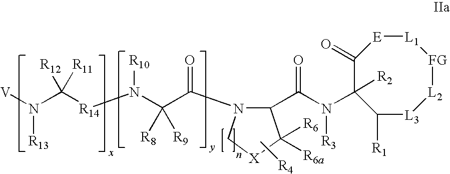

and pharmaceutically acceptable salts, enantiomers, stereoisomers, rotamers, tautomers, diastereomers, or racemates thereof.

Certain compounds of Formula IIa include compounds in which y is 0. Yet other compounds of Formula IIa include compounds in which x and y are 0. Still further compounds of Formula IIa include compounds in which x and y are 0 and V is a C₁-C₄alkanoyl.

Certain compounds of Formula II or Formula IIa include those compounds in which:

x is 0 or 1;

n is 0 or 1;

R₁₄ is C(O) or S(O)ₚ

R₁ is selected from the group consisting of H and C₁₋₄-alkyl;

R₂ is selected from the group consisting of C₁₋₄-alkyl, C(O)C₁₋₄-alkyl, C(O)OC₁₋₄-alkyl, and (CH₂)₀₋₄—C₃₋₆-cycloalkyl;

or R₁ and R₂ together form a cyclopropane ring;

R₃ is selected from the group consisting of H and C₁₋₄-alkyl;

X is O, NR₅ or CR₅R₅ₐ;

R₄ is selected from the group consisting of H, C₁₋₄-alkyl, C₃₋₆-cycloalkyl, aryl, heterocycle and heteroaryl, each of which may be independently substituted one or more times with a halogen atom or C₁₋₄-alkyl;

R₅ is selected from the group consisting of H, hydroxyl, oxo, C₁₋₈-alkyl, C₂₋₈-alkenyl, C₂₋₈-alkynyl, C₃₋₈-cycloalkyl-C₀₋₄-alkyl, aryl-C₀₋₄-alkyl, heterocycle-C₀₋₄-alkyl and heteroaryl-C₀₋₄-alkyl, each of which may be independently substituted one or more times with a halogen atom, aryl, heteroaryl, trihalomethyl, C₁₋₄-alkoxy or C₁₋₄-alkyl;

R₅ₐ is selected from the group consisting of H, hydroxyl, C₁₋₈-alkyl, C₂₋₈-alkenyl, C₂₋₈-alkynyl, C₃₋₈-cycloalkyl-C₀₋₄-alkyl, aryl-C₀₋₄-alkyl and heteroaryl-C₀₋₄-alkyl,

or R₄ and R₅ may together form a fused dimethyl cyclopropyl ring, a fused cyclopentane ring, a fused phenyl ring or a fused pyridyl ring, each of which may be substituted with a halogen atom, aryl, heteroaryl, trihalomethyl, C₁₋₄-alkoxy or C₁₋₄-alkyl;

or R₅ and R₅ₐ may together form a spirocarbocyclic saturated ring having between 3 and 6 carbon ring atoms which is optionally substituted by 0-2 substitutents selected from halogen, C₁₋₆-alkyl, C₂₋₆-alkenyl, C₂₋₆-alkynyl, C₁₋₆-alkoxide, C₃₋₇-cycloalkyl-C₀₋₄-alkyl, phenyl-C₀₋₄-alkyl, naphthyl-C₀₋₄-alkyl, heteroaryl-C₀₋₄-alkyl, or two substitutents taken together form a fused or spirocyclic 3 to 7 membered carbocyclic ring, each of which is substituted with 0-3 independently selected halogen atoms or C₁₋₄-alkyl groups;

R₈, R₁₀ and R₁₁ are each, independently, selected from the group consisting of H and C₁₋₄-alkyl;

R₆, R₆ₐ, and R₁₃ is H;

R₉ and R₁₂ are each, independently, selected from the group consisting of H, C₁₋₄-alkyl and C₃₋₆-cycloalkyl; and

V is selected from the group consisting of -Q¹-Q², wherein Q¹ is absent, C(O), N(H), N(C₁₋₄-alkyl), C═N(CN), C═N(SO₂CH₃), or C═N—COH, and Q² is H, C₁₋₄-alkyl, C═N—COH—C₁₋₄-alkyl, O—C₁₋₄-alkyl, NH₂, N(H)—C₁₋₄-alkyl, N(C₁₋₄-alkyl)₂, SO₂-aryl, SO₂—C₁₋₄-alkyl, C₃₋₆-cycloalkyl-C₀₋₄-alkyl, aryl, heteroaryl and heterocycle, each of which may be independently substituted one or more times with a halogen atom, C₁₋₄-alkyl, C₁₋₄alkoxy, C₂-C₄alkenyloxy, C₂-C₄alkynyloxy, C₁₋₄-alkyl substituted by one or more halogen atoms, or C₃₋₆-cycloalkyl;

or when x is 0, R₁₀ and V can form a cyclopropyl ring that may be further substituted by an amide group.

In yet other compounds of Formula II or Formula IIa, R₁₄ is C(O).

Certain compounds of Formula II or Formula IIa include compounds in which X is CR₅R₅ₐ, R₅ₐ is H, and R₅ is selected from the group consisting of piperidine, phenyl, —O-pyridinyl and CH₂-pyridinyl, wherein the phenyl and pyridinyl groups may be independently substituted one or more times with a halogen atom or C₁₋₄-alkyl. Certain other compounds of Formula I, Ia, Ib, Ic and/or II include compounds in which X is CR₅R₅ₐ, R₅ₐ is H and R₅ is selected from the group consisting of 7-methoxy-2-phenyl-quinolin-4-yloxy, 2-(2-isopropylamino-thiazol-4-yl)-7-methoxy-quinolin-4-yloxy and 4-fluoro-1,3-dihydro-isoindole-2-carbonyloxy.

In certain compounds of Formula II or Formula IIa, X is CR⁵R^{5a}, R^{5a} is hydrogen, and R⁵ is selected from the group consisting of piperidine, phenyl, pyridinyl, pyridinyloxy and pyridinylmethyl, wherein the phenyl and pyridinyl groups may be independently substituted one or more times with a halogen atom or C₁₋₄-alkyl.

Certain other compounds of Formula II or Formula IIa include compounds in which X is CR₅R₅ₐ, R₄ is H, R^{5a} is hydrogen, and R⁵ is selected from the group consisting of

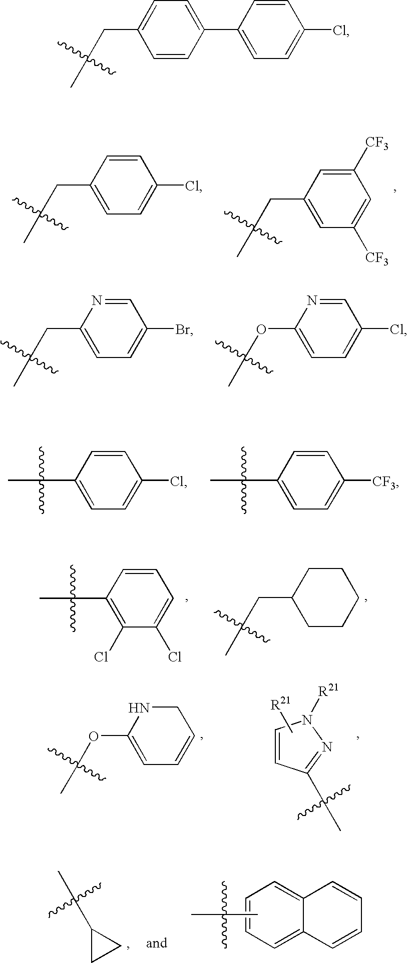

wherein R²¹ is independently selected from the group consisting of C₁₋₄-alkyl and aryl.

Certain other compounds of Formula II or Formula IIa include compounds in which X is CR⁵R^{5a}, R⁴ is hydrogen, and R⁵ and R^{5a} taken in combination form a 3 to 6 member spirocyclic carbocycle substituted with 0-2 substituents selected from halogen, C₁₋₆-alkyl, C₂₋₆-alkenyl, C₂₋₆-alkynyl, C₁₋₆-alkoxy, C₃₋₇-cycloalkyl-C₀₋₄-alkyl, phenyl-C₀₋₄-alkyl, naphthyl-C₀₋₄-alkyl, heteroaryl-C₀₋₄-alkyl, or two substituents taken together form a fused or spirocyclic 3 to 7 membered carbocyclic ring, each of which is substituted with 0-3 independently selected halogen atoms or C₁₋₄-alkyl groups.

Certain other compounds of Formula II or Formula IIa include compounds in which X is CR₅R₅ₐ, R₄ is H, and R₅ and R₅ₐ taken in combination form a 3 to 6 member spirocyclic carbocycle substituted with 0-2 substitutents selected from halogen, C₁₋₆-alkyl, C₂₋₆-alkenyl, C₂₋₆-alkynyl, C₁₋₆-alkoxide, C₃₋₇-cycloalkyl-C₀₋₄-alkyl, phenyl-C₀₋₄-alkyl, naphthyl-C₀₋₄-alkyl, heteroaryl-C₀₋₄-alkyl, or two substitutents taken together form a fused or spirocyclic 3 to 7 membered carbocyclic ring, each of which is substituted with 0-3 independently selected halogen atoms or C₁₋₄-alkyl groups.

In certain compounds of Formula II or Formula IIa, the divalent residue:

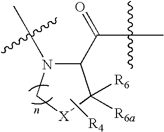

is selected from the group consisting of:

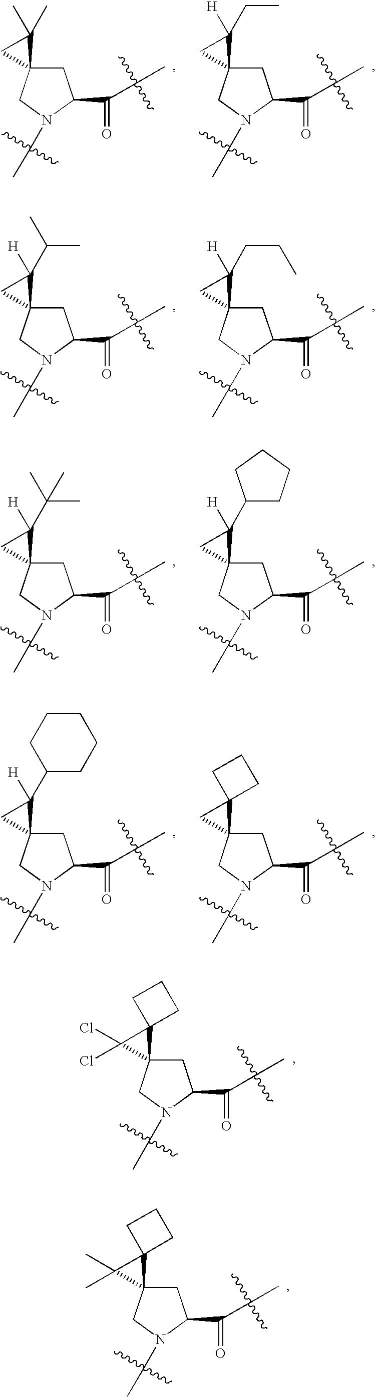

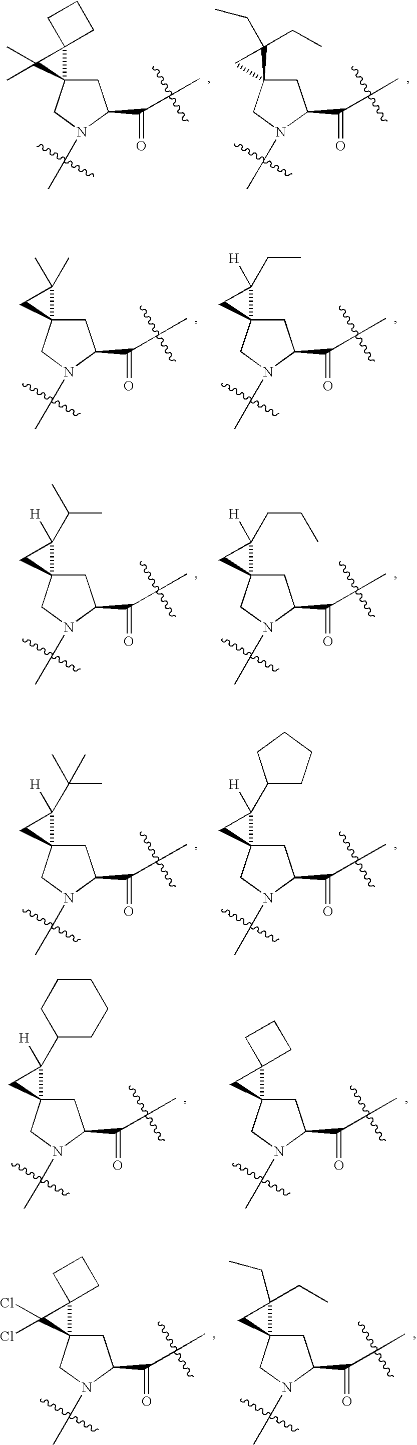

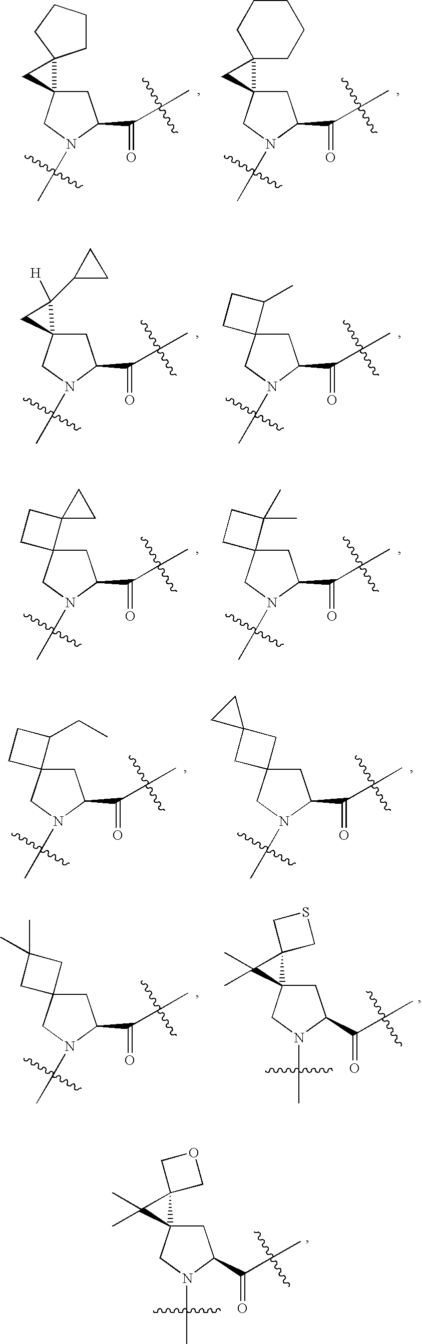

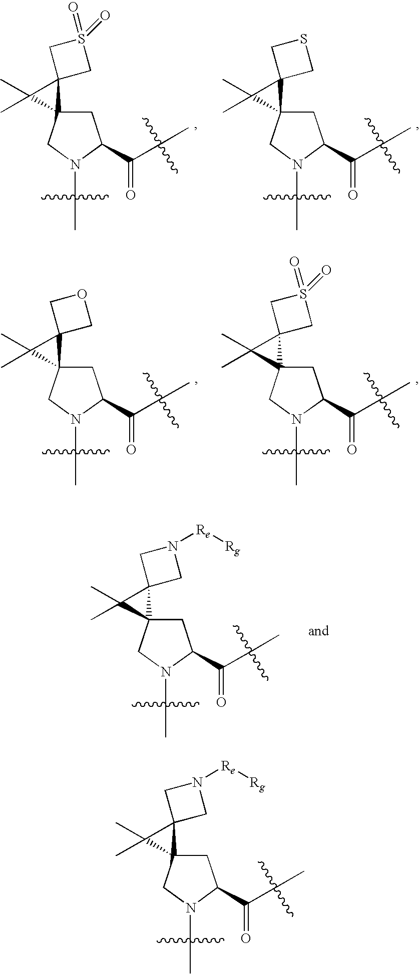

wherein Rₑ is absent, C(O), or S(O)₂; and R_{g} is selected hydrogen or selected from the group consisting of C₁₋₆alkyl, arylC₀₋₄alkyl, heteroarylC₀₋₄alkyl, heterocyclylC₀₋₄alkyl, and C₃₋₇cycloalkylC₀₋₄alkyl, each of which is substituted with 0 to 4 independently selected substituents selected from the group consisting of cyano, halogen, hydroxyl, amino, thiol, C₁₋₈-alkyl, C₂₋₈-alkenyl, C₂₋₈-alkynyl, C₁₋₈-alkoxy-C₀₋₄alkyl, C₁₋₈-haloalkyl, C₂₋₈-haloalkenyl, C₂₋₈-haloalkynyl, C₁₋₈-haloalkoxy, C₁₋₈-alkylthio, C₁₋₈-alkylsulfonyl, C₁₋₈-alkylsulfoxy, C₁₋₈-alkanoyl, C₁₋₈-alkoxycarbonyl, C₃₋₇-cycloalkyl-C₀₋₄-alkyl, aryl-C₀₋₄-alkyl, heteroaryl-C₀₋₄-alkyl, COOH, C(O)NH₂, mono- and di-C₁₋₄-alkyl-carboxamide, mono- and di-C₁₋₄-alkyl-amino-C₀₋₄alkyl, SO₃H, SO₂NH₂, and mono-and di-C₁₋₄-alkylsulfonamide.

Certain compounds of Formula II or Formula IIa, include those compounds in which the

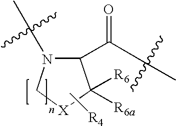

ring is a divalent residue derived from a proline residue selected from the group consisting of:

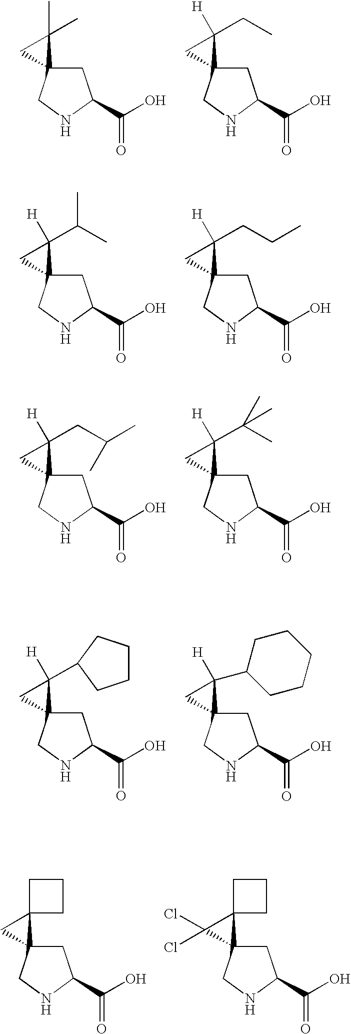

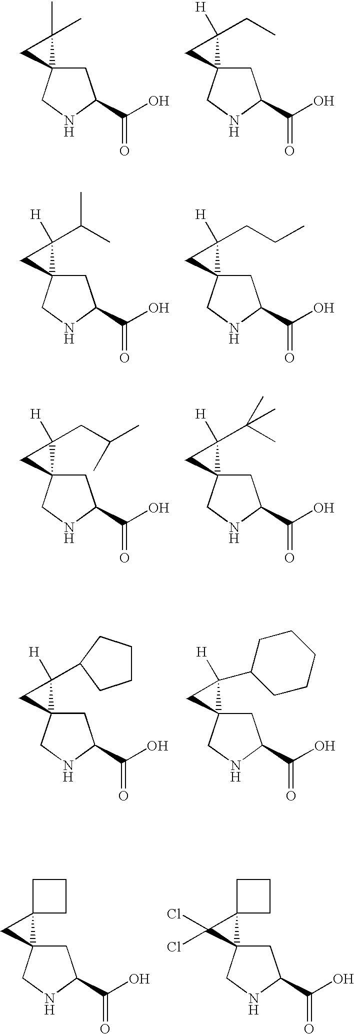

Yet other compounds of Formula II or Formula IIa include compounds in which V is C(O)—N(H)-t-butyl or C(O)—R₂₀, wherein R₂₀ is selected from the group consisting of C₃₋₆-cycloalkyl, phenyl, pyrazine, benzooxazole, 4,4-dimethyl-4,5-dihydro-oxazole, benzoimidazole, pyrimidine, thiazole, benzothiazole, benzothiazole 1,1-dioxide and quinazoline, each of which may be further independently substituted with a halogen atom, CF₃, C₁₋₄-alkyl, C₁₋₄alkoxy, C₂-C₄alkenyloxy, C₂-C₄alkynyloxy, or C₃₋₆-cycloalkyl.

In certain other compounds of Formula II or Formula IIa, V is selected from the group consisting of C₃₋₆-cycloalkyl, phenyl, pyrazine, benzooxazole, 4,4-dimethyl-4,5-dihydro-oxazole, benzoimidazole, pyrimidine, thiazole, benzothiazole, benzothiazole 1,1-dioxide and quinazoline, each of which may be further independently substituted with a halogen atom, CF₃, C₁₋₄-alkyl, C₁₋₄alkoxy, C₂-C₄alkenyloxy, C₂-C₄alkynyloxy, or C₃₋₆-cycloalkyl.

Certain compounds of Formulae I, Ia, Ib and/or Ic include compounds of Formula III:

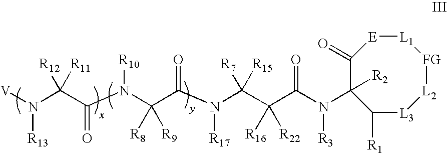

and pharmaceutically acceptable salts, enantiomers, stereoisomers, rotamers, tautomers, diastereomers, or racemates thereof.

Certain compounds of Formula III include compounds in which:

R₃ is selected from the group consisting of H, C₁₋₄-alkyl, and C₃₋₆-cycloalkylC₀-C₄alkyl;

R₈, R₁₁, R₁₅ and R₂₂ are selected from the group consisting of H, alkyl-aryl, C₁₋₄-alkyl, O—C₁₋₄-alkyl, N(H)—C₁₋₄-alkyl, and C₃₋₆-cycloalkylC₀-C₄alkyl;

R₁₀ and R₁₇ are each, independently, selected from the group consisting of H, C₁₋₄-alkyl and (CH₂)₀₋₄—C₃₋₆-cycloalkyl; or

R₁₅ and R₁₆ may together form a 3, 4, 5, 6 or 7-membered ring that may comprise between 0 to 3 additional heteroatoms, wherein the ring may be further substituted with 0-5 substitutents; or

R₁₆ and R₁₇ may together form a 3, 4, 5, 6 or 7-membered ring that may comprise between 0 to 3 additional heteroatoms, wherein the ring may be further substituted with 0-5 substitutents; and

V is selected from the group consisting of -Q¹-Q², wherein Q¹ is absent, C(O), N(H), N(C₁₋₄-alkyl), C═N(CN), C═N(SO₂CH₃), or C═N—COH, and Q² is H, C₁₋₄-alkyl, C═N—COH—C₁₋₄-alkyl, O—C₁₋₄-alkyl, NH₂, N(H)—C₁₋₄-alkyl, N(C₁₋₄-alkyl)₂, SO₂-aryl, SO₂—C₁₋₄-alkyl, C₃₋₆-cycloalkyl-C₀₋₄-alkyl, aryl, heteroaryl and heterocycle, each of which may be independently substituted one or more times with a halogen atom, C₁₋₄-alkyl, C₁₋₄alkoxy, C₂-C₄alkenyloxy, C₂-C₄alkynyloxy, C₁₋₄-alkyl substituted by one or more halogen atoms, or C₃₋₆-cycloalkyl;

Certain other compounds of Formula III provided herein include compounds in which

R₃ is selected from the group consisting of H, C₁₋₄-alkyl, and C₃₋₇cycloalkyl-C₀₋₄-alkyl;

R₁₃ is H;

R₈, R₁₀ and R₁₁ are each, independently, selected from the group consisting of H and C₁₋₄-alkyl;

R₉ and R₁₂ are each, independently, selected from the group consisting of H, C₁₋₄-alkyl and (CH₂)₀₋₄—C₃₋₆-cycloalkyl; and

V is selected from the group consisting of -Q¹-Q², wherein Q¹ is absent, C(O), N(H), N(C₁₋₄-alkyl), C═N(CN), C═N(SO₂CH₃), or C═N—COH, and Q² is H, C₁₋₄-alkyl, C═N—COH—C₁₋₄-alkyl, O—C₁₋₄-alkyl, NH₂, N(H)—C₁₋₄-alkyl, N(C₁₋₄-alkyl)₂, SO₂-aryl, SO₂—C₁₋₄-alkyl, C₃₋₆-cycloalkyl-C₀₋₄-alkyl, aryl, heteroaryl and heterocycle, each of which may be independently substituted one or more times with a halogen atom, C₁₋₄-alkyl, C₁₋₄alkoxy, C₂-C₄alkenyloxy, C₂-C₄alkynyloxy, C₁₋₄-alkyl substituted by one or more halogen atoms, or C₃₋₆-cycloalkyl;

Certain compounds of Formula III include compounds represented by Formula IV:

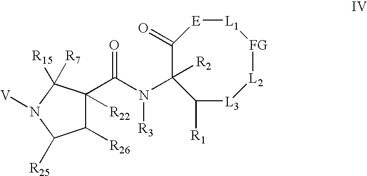

and pharrnaceutically acceptable salts, enantiomers, stereoisomers, rotamers, tautomers, diastereomers, or racemates thereof;
wherein

R₂₅ and R₂₆ are each, independently, selected from the group consisting of H, C₁₋₄-alkyl, O—C₁₋₄-alkyl, N(R₂₄)₂, C₃₋₆cycloalkylC₀-C₄alkyl, substituted or unsubstituted aryl and substituted or unsubstituted heterocycle, wherein each R₂₄ is independently selected from the group consisting of H, halogen, hydroxy, COOH, amino, carboxamide, substituted or unsubstituted-C₁₋₄-alkyl, substituted or unsubstituted C₃₋₆cycloalkylC₀-C₄alkyl, substituted or unsubstituted-C₁₋₄-alkoxy, substituted or unsubstituted C₃₋₆cycloalkylC₀-C₄alkyl-oxy-, substituted or unsubstituted arylC₀-C₄alkyl, substituted or unsubstituted heterocycleC₀-C₄alkyl, substituted or unsubstituted arylC₀-C₄alkyl-oxy and substituted or unsubstituted heterocycleC₀-C₄alkyl-oxy;

or R₂₂ or R₂₆ may together form a 3-membered ring that is substituted or unsubstituted.

In another embodiment of Formula IV, R₂₅ is H and R₂₆ is amine, substituted or unsubstiuted phenyl, or substituted or unsubstiuted benzyl.

Certain other compounds of Formula III include compounds represented by Formula V:

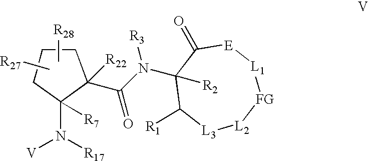

and pharmaceutically acceptable salts, enantiomers, stereoisomers, rotamers, tautomers, diastereomers, or racemates thereof;
wherein

R₂₇ and R₂₈ are each, independently, selected from the group consisting of H, C₁₋₄-alkyl, O—C₁₋₄-alkyl, N(R₂₄)₂, C₃₋₆CycloalkylC₀-C₄alkyl, substituted or unsubstituted aryl, substituted or unsubstituted O-aryl and substituted or unsubstituted heterocycle, wherein R₂₄ is independently selected at each occurrence from the group consisting of H, halogen, hydroxy, COOH, amino, carboxamide, substituted or unsubstituted-C₁₋₄-alkyl, substituted or unsubstituted C₃₋₆cycloalkylC₀-C₄alkyl, substituted or unsubstituted-C₁₋₄-alkoxy, substituted or unsubstituted C₃₋₆cycloalkylC₀-C₄alkyl-oxy-, substituted or unsubstituted arylC₀-C₄alkyl, substituted or unsubstituted heterocycleC₀-C₄alkyl, substituted or unsubstituted arylC₀-C₄alkyl-oxy and substituted or unsubstituted heterocycleC₀-C₄alkyl-oxy.

In one embodiment of Formula V, R₂₈ is quinoline, C₁₋₄-alkyl, O—C₁₋₄-alkyl, or O-quinoline, wherein the quinoline and O-quinoline substituents may be independently substituted one or more times with halogen, amino, O—C₁₋₄-alkyl, substituted or unsubstituted-C₁₋₄-alkyl, substituted or unsubstituted-(CH₂)₀₋₄—C₃₋₆-cycloalkyl, substituted or unsubstituted aryl, substituted or unsubstituted O-aryl, and substituted or unsubstituted heterocycle.

Yet other compounds of Formula III include compounds represented by Formula VI:

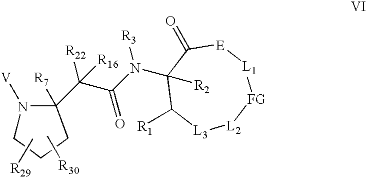

and pharrnaceutically acceptable salts, enantiomers, stereoisomers, rotamers, tautomers, diastereomers, or racemates thereof;
wherein

R₂₉ and R₃₀ are selected from the group consisting of H, C₁₋₄-alkyl, O—C₁₋₄-alkyl, N(R₂₄)₂, C₃₋₆CycloalkylC₀-C₄alkyl, substituted or unsubstituted aryl, substituted or unsubstituted aryl-oxy and substituted or unsubstituted heterocycle, wherein each R₂₄ is independently selected at each occurrence from the group consisting of H, halogen, hydroxy, COOH, amino, carboxamide, substituted or unsubstituted-C₁₋₄-alkyl, substituted or unsubstituted C₃₋₆cycloalkylC₀-C₄alkyl, substituted or unsubstituted-C₁₋₄-alkoxy, substituted or unsubstituted C₃₋₆cycloalkylC₀-C₄alkyl-oxy-, substituted or unsubstituted arylC₀-C₄alkyl, substituted or unsubstituted heterocycleC₀-C₄alkyl, substituted or unsubstituted arylC₀-C₄alkyl-oxy and substituted or unsubstituted heterocycleC₀-C₄alkyl-oxy.

In one embodiment of Formula VI, R₂₉ is selected from the group consisting of O-phenyl and O-benzyl.

Still other compounds of Formula III include compounds represented by Formula VII:

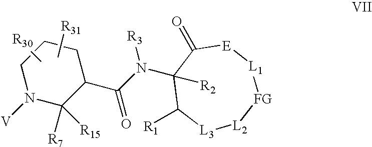

and pharmaceutically acceptable salts, enantiomers, stereoisomers, rotamers, tautomers, diastereomers, or racemates thereof;
wherein

R₃₀ and R₃₁ are selected from the group consisting of H, C₁₋₄-alkyl, O—C₁₋₄-alkyl, N(R₂₄)₂, (CH₂)₀₋₄-C₃₋₆-cycloalkyl, substituted or unsubstituted aryl, substituted or unsubstituted O-aryl and substituted or unsubstituted heterocycle, wherein each R₂₄ is independently selected from the group consisting of H, halogen, hydroxy, COOH, amino, carboxamide, substituted or unsubstituted-C₁₋₄-alkyl, substituted or unsubstituted C₃₋₆cycloalkylC₀-C₄alkyl, substituted or unsubstituted-C₁₋₄-alkoxy, substituted or unsubstituted C₃₋₆cycloalkylC₀-C₄alkyl-oxy-, substituted or unsubstituted arylC₀-C₄alkyl, substituted or unsubstituted heterocycleC₀-C₄alkyl, substituted or unsubstituted arylC₀-C₄alkyl-oxy and substituted or unsubstituted heterocycleC₀-C₄alkyl-oxy;

or R₃₀ and R₃₁ may together form a 3, 4, 5, 6 or 7-membered ring that is aromatic or non-aromatic and may contain one or more heteroatoms selected from N, O or S, wherein the ring may be further substituted one or more times.

In another embodiment, Formula VII is represented by a compound of the Formula VIIa:

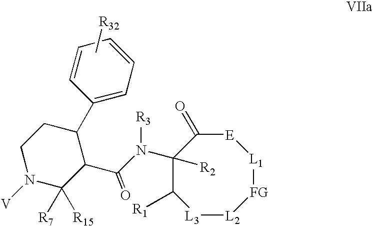

and pharmaceutically acceptable salts, enantiomers, stereoisomers, rotamers, tautomers, diastereomers, or racemates thereof;
wherein

R₃₂ is -Q¹-Q², wherein Q¹ is absent, C(O), S(O)ₚ, N(H), N(C₁₋₄-alkyl), C═N(CN), C═N(SO₂CH₃), or C═N—COH, and Q² is H, C₁₋₄-alkyl, C═N—COH—C₁₋₄-alkyl, O—C₁₋₄-alkyl, NH₂, N(H)—C₁₋₄-alkyl, N(C₁₋₄-alkyl)₂, SO₂-aryl, SO₂—C₁₋₄-alkyl, C₃₋₆-cycloalkyl-C₀₋₄-alkyl, aryl, heteroaryl and heterocycle, each of which may be independently substituted one or more times with a halogen atom, C₁₋₄-alkyl, C₁₋₄-alkyl substituted by one or more halogen atoms, or C₃₋₆-cycloalkyl.

In another embodiment, Formula VII is represented by a compound of the Formula VIIb:

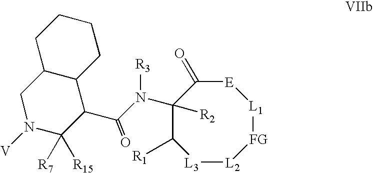

and pharmaceutically acceptable salts, enantiomers, stereoisomers, rotamers, tautomers, diastereomers, or racemates thereof.

In another embodiment, Formula VII is represented by a compound of the Formula VIIc:

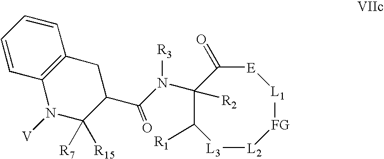

and pharmaceutically acceptable salts, enantiomers, stereoisomers, rotamers, tautomers, diastereomers, or racemates thereof:

Certain compounds of Formula III include compounds represented by Formula VIII:

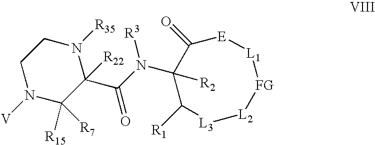

and pharmaceutically acceptable salts, enantiomers, stereoisomers, rotamers, tautomers, diastereomers, or racemates thereof;
wherein

R₃₅ is H, halogen, hydroxy, COOH, amino, carboxamide, substituted or unsubstituted-C₁₋₄-alkyl, substituted or unsubstituted C₃₋₆cycloalkylC₀-C₄alkyl, substituted or unsubstituted-C₁₋₄-alkoxy, substituted or unsubstituted C₃₋₆cycloalkylC₀-C₄alkyl-oxy-, substituted or unsubstituted arylC₀-C₄alkyl, substituted or unsubstituted heterocycleC₀-C₄alkyl, substituted or unsubstituted arylC₀-C₄alkyl-oxy and substituted or unsubstituted heterocycleC₀-C₄alkyl-oxy.

In one embodiment of Formula VIII, R₃₅ is phenyl, optionally substituted with chloro.

Certain compounds of Formulae I, Ia, Ib and/or Ic include compounds of Formula IX:

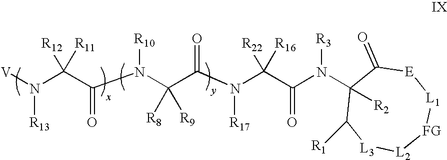

and pharmaceutically acceptable salts, enantiomers, stereoisomers, rotamers, tautomers, diastereomers, or racemates thereof.

Certain compounds of Formula IX include those compounds in which:

y is 0 or 1;

R₃ is selected from the group consisting of H and C₁₋₄-alkyl;

R₁₇ are each, independently selected at each occurrence from the group consisting of H, C₁₋₄-alkyl, C₁₋₆-cycloalkyl, (CH₂)₀₋₄—C₃₋₆-cycloalkyl, aryl, alkyl-aryl and heterocycle, each of which may be independently substituted one or more times;

R₈, R₁₀ and R₁₁ are each, independently, selected from the group consisting of H and C₁₋₄-alkyl;

R₉ is selected from the group consisting of H, C₁₋₄-alkyl and C₁₋₆-cycloalkyl;

R₁₂ is selected from the group consisting of H, C₁₋₄-alkyl, C₁₋₆-cycloalkyl and aryl; and

V is selected from the group consisting of -Q¹-Q², wherein Q¹ is absent, C(O), N(H), N(C₁₋₄-alkyl), C═N(CN), C═N(SO₂CH₃), or C═N—COH, and Q² is H, C₁₋₄-alkyl, C═N—COH—C₁₋₄-alkyl, O—C₁₋₄-alkyl, NH₂, N(H)—C₁₋₄-alkyl, N(C₁₋₄-alkyl)₂, SO₂-aryl, SO₂—C₁₋₄-alkyl, C₃₋₆-cycloalkyl-C₀₋₄-alkyl, aryl, heteroaryl and heterocycle, each of which may be independently substituted one or more times with a halogen atom, C₁₋₄-alkyl, C₁₋₄alkoxy, C₂-C₄alkenyloxy, C₂-C₄alkynyloxy, C₁₋₄-alkyl substituted by one or more halogen atoms, or C₃₋₆-cycloalkyl;

or R₁₁ and V form the following 5-membered ring which may be further substituted:

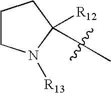

Certain other compounds of Formula IX include those compounds in which R₁₇ is selected from the group consisting of H, cyclopropylC₀-C₂alkyl, cyclopentylC₀-C₂alkyl, phenylC₁-C₂alkyl, and naphthylC₁-C₂alkyl.

Certain other compounds of Formulae I, Ia, Ib, Ic, II, III, IV, V, VI, VII, VIII, and/or IX include those compounds in which V is selected from the group consisting of C(O)R₂₄, C(O)C(O)OR₂₄, C(O)N(H)R₂₄, C(O)C(O)N(H)R₂₄ and C(O)OR₂₄, wherein each R₂₄ is independently selected from the group consisting of H, halogen, substituted or unsubstituted-C₁₋₄-alkyl, substituted or unsubstituted C₃₋₆-cycloalkylC₀-C₄alkyl, substituted or unsubstituted arylC₀-C₄alkyl and substituted or unsubstituted heterocycleC₀-C₄alkyl, and any combination thereof. Vet other compounds of Formulae I, Ia, Ib, Ic, II, III, IV, V, VI, VII, VIII, and/or IX include compounds in which V is C(O)—R₂₀, wherein R₂₀ is selected from the group consisting of tert-butyl, C₃₋₆-cycloalkyl, phenyl, pyrazine, benzooxazole, 4,4-dimethyl-4,5-dihydro-oxazole, benzoimidazole, pyrimidine, thiazole, benzothiazole, benzothiazole 1,1-dioxide and quinazoline, each of which may be further independently substituted with 0-5 substitutents selected from a halogen atom, C₁₋₄-alkyl, C₁₋₄alkoxy, C₂-C₄alkenyloxy, C₂-C₄alkynyloxy, C₁₋₄-alkyl substituted by one or more halogen atoms, or C₃₋₆-cycloalkyl.

In another embodiment of Formula I or Formula III, V is selected from the group consisting of C(O)—N(H)-t-butyl.

In yet another embodiment of Formula I or Formula III, V is C(O)—R²⁰, wherein R²⁰ is selected from the group consisting of C₃₋₆-cycloalkyl, phenyl, pyrazine, benzoxazole, 4,4-dimethyl-4,5-dihydro-oxazole, benzoimidazole, pyrimidine, benzothiazole 1,1-dioxide and quinazoline, each of which may be further independently substituted with a halogen atom, CF₃, C₁₋₄-alkyl or C₃₋₆-cycloalkyl.

In still another embodiment of Formula I or Formula III, V is R²⁰ or C(O)—R²⁰, wherein R²⁰ is selected from the group consisting of

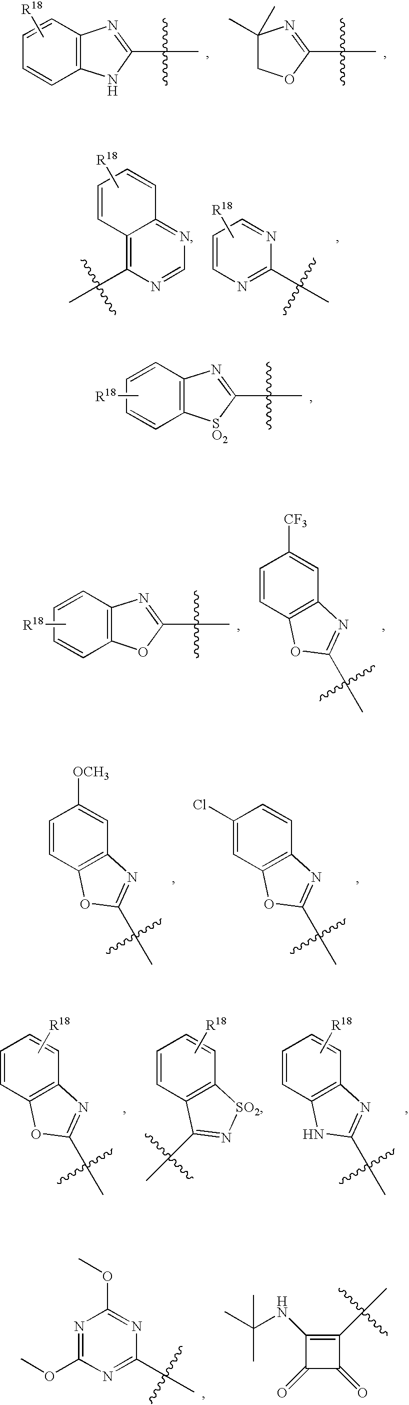

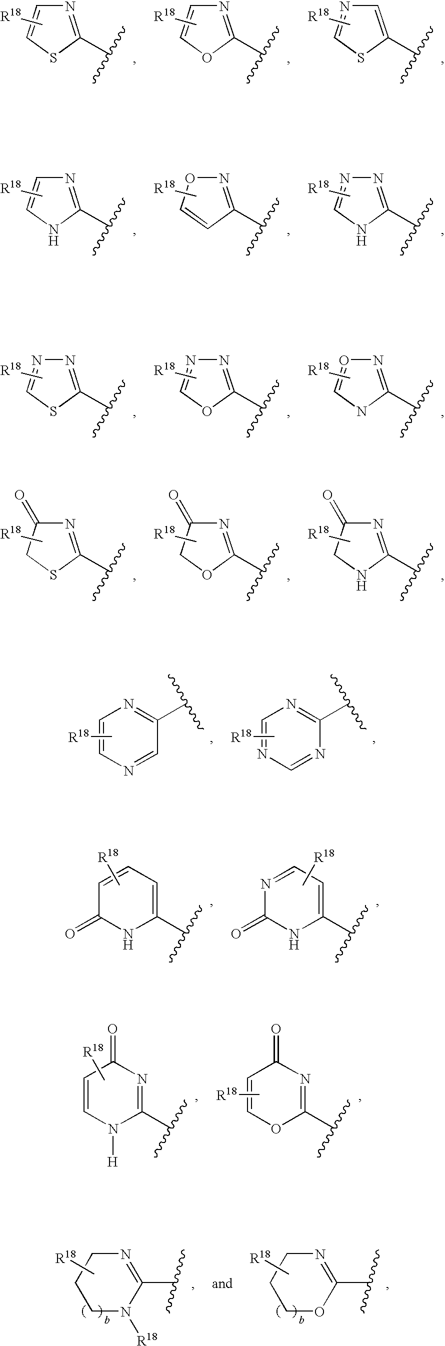

wherein b is 0, 1, or 2; and R₁₈ is selected from the group consisting of hydrogen, a halogen atom, aryl, trihalomethyl, and C₁₋₄-alkyl.

In another embodiment of Formula I or Formula III, V is selected from the group consisting of C₃₋₆-cycloalkyl, phenyl, pyrazine, benzooxazole, 4,4-dimethyl-4,5-dihydro-oxazole, benzoimidazole, pyrimidine, benzothiazole 1,1-dioxide and quinazoline, each of which may be further independently substituted with a halogen atom, CF₃, C₁₋₄-alkyl or C₃₋₆-cycloalkyl.

In certain embodiments, compounds of Formula I or Formula III comprise a V group selected from residues having the formula —C(O)—R²⁰, wherein R²⁰ is a residue of the formula (i):

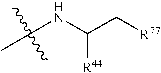

wherein R⁴⁴ is selected from the group consisting of: tert-butyl, isopropyl, cyclohexyl, spirocyclohexyl

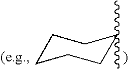

and 1-methylcyclohexyl;
and R⁷⁷ is selected from the group consisting of:

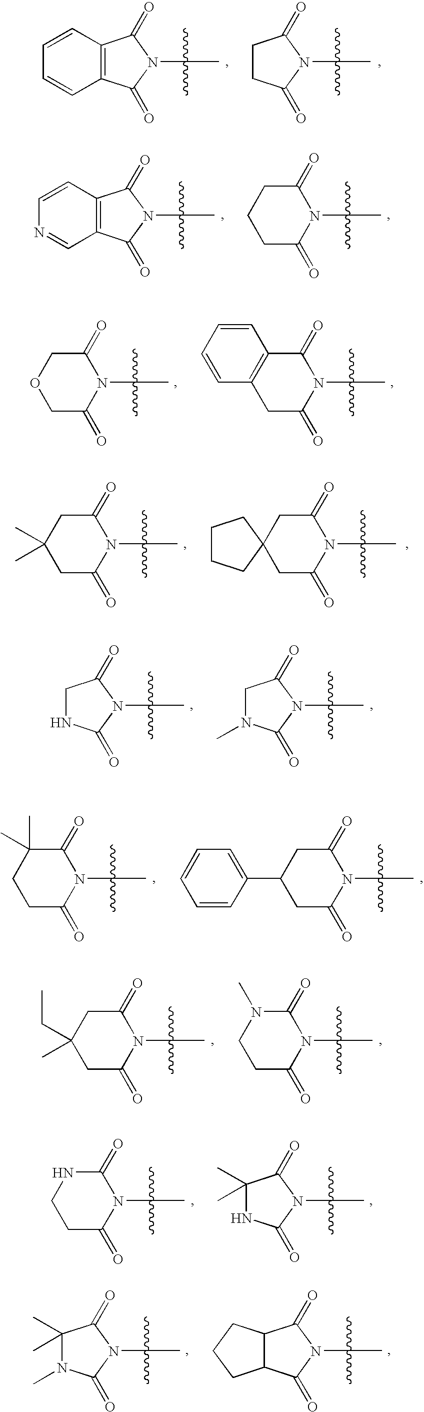

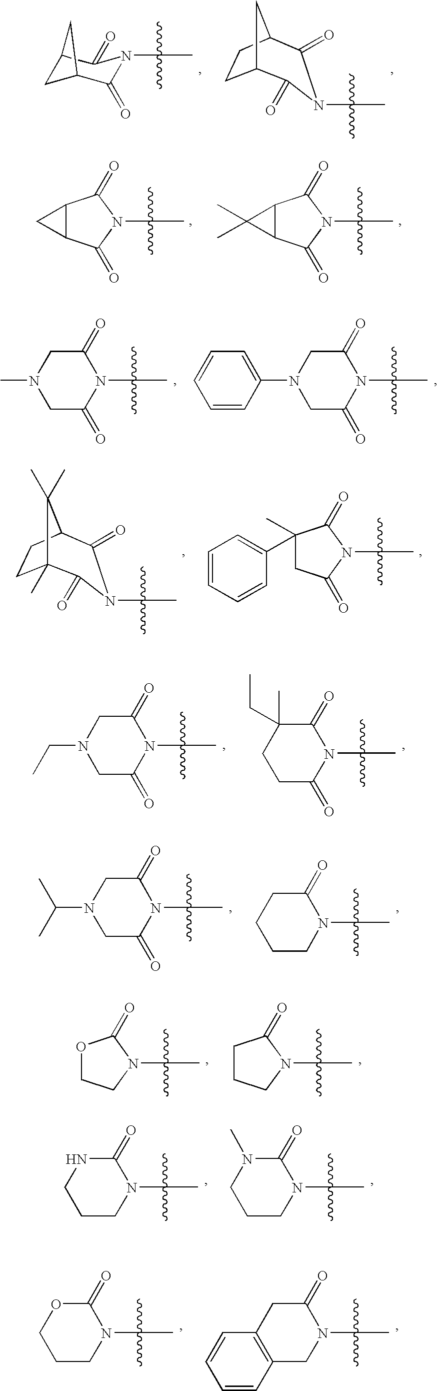

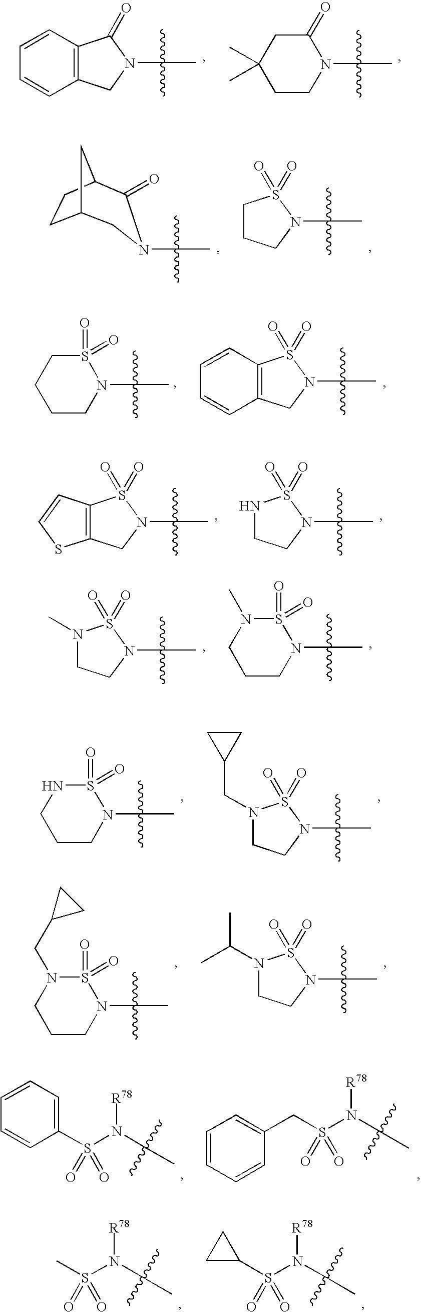

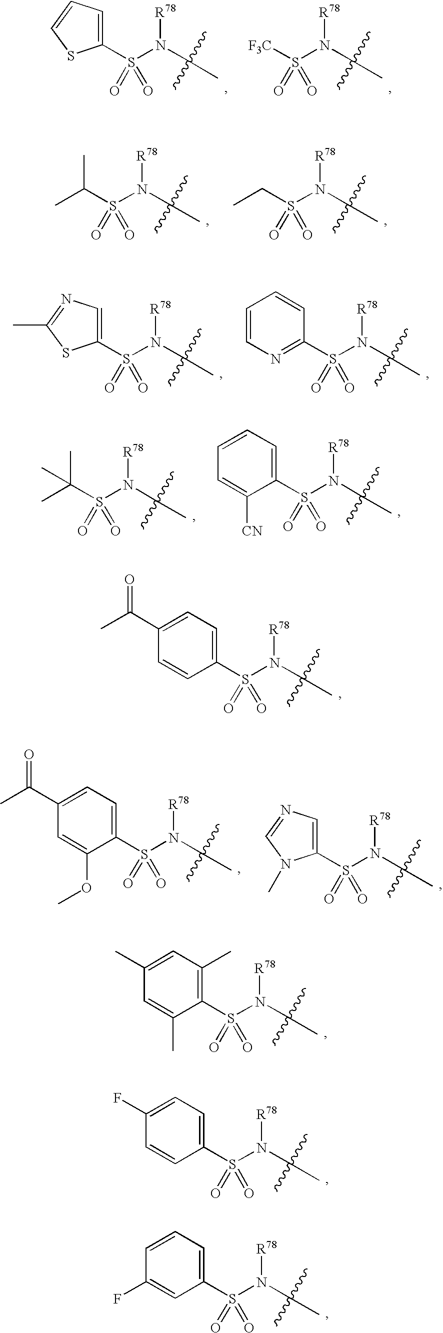

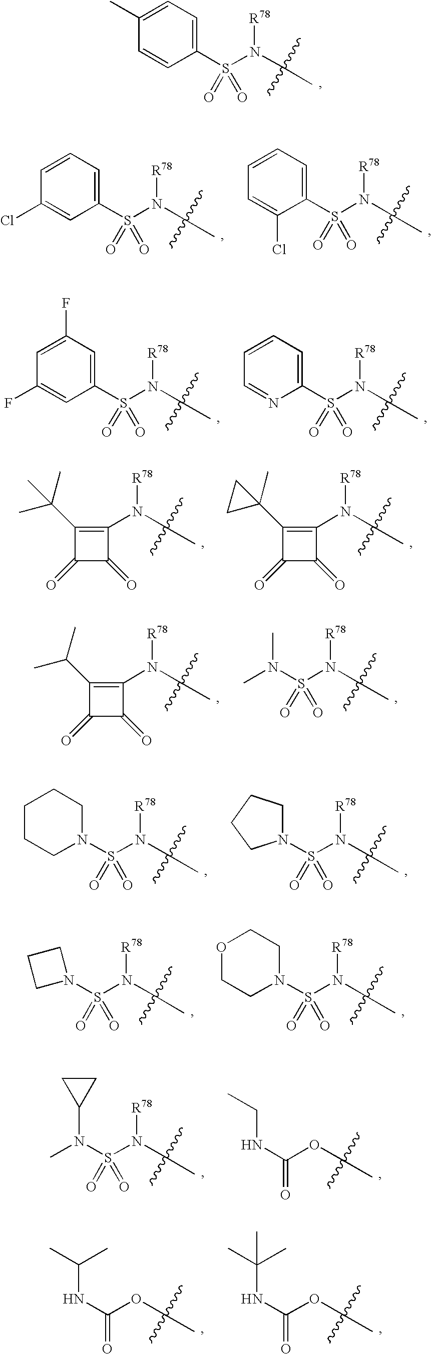

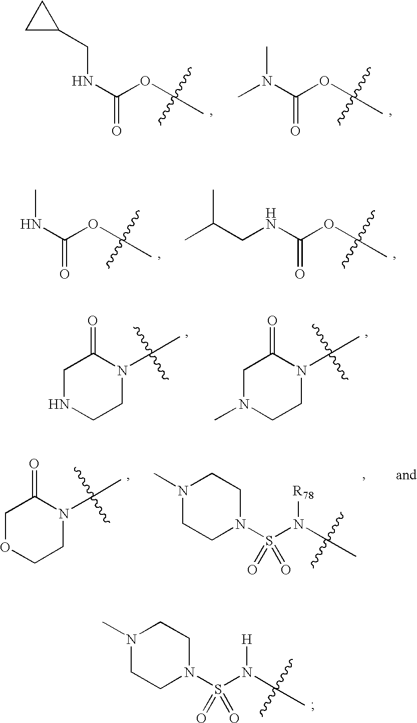

Where R⁷⁸ is selected from methyl, ethyl, isopropyl, tert-butyl and phenyl.

In yet other embodiments, compounds of Formula I or Formula III comprise a V group selected from residues having the formula —C(O)—V′, wherein V′ is a residue of the formula (ii):

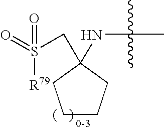

wherein R⁷⁹ is selected form the group consisting of methyl, ethyl, isopropyl, tert-butyl, sec-butyl, 4-methyl-butyl, 1,1-dimethylpropyl, 1,1-dimethylbutyl, phenyl, benzyl, cyclopentyl, cyclohexyl, furylmethyl, and pyridyl (e.g., 2-pyridyl, 3-pyridyl, or 4-pyridyl).

In certain other embodiments, compounds of Formula I or Formula III comprise a V group selected from residues having the formula —C(O)—V′, wherein V′ is a residue of the formula (iii):

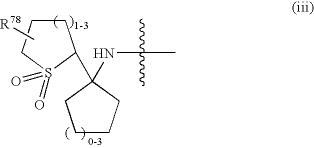

wherein R⁷⁸ represents 0-3 groups independently selected from C₁₋₆alkyl and C₃₋₆cycloalkyl.

In certain embodiments, compounds of Formula I or Formula III comprise a V group selected from residues having the formula —C(O)—V′, wherein V′ is a residue of the formula (iv):

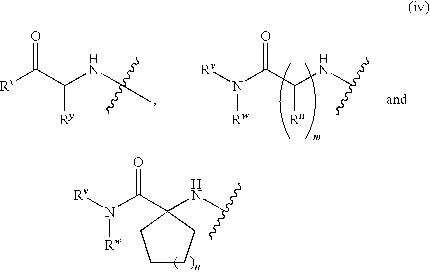

wherein m is 1 or 2; n is 0, 1, 2, or 3;

R^{u}, R^{v}, and R^{w}, are independently selected at each occurrence from the group consisting of hydrogen, C₁₋₆alkyl, arylC₀₋₄alkyl, heteroarylC₀₋₄alkyl, heterocyclylC₀₋₄alkyl, and cycloalkylC₀₋₄alkyl; or

R^{v} and R^{w}, taken in combination, form a ring having between 3 and 7 ring atoms and having 0, 1, 2 ring heteroatoms which is substituted with 0-2 alkyl groups and 0-1 spirocyclic groups.

R^{x} and R^{y} are each independently selected from the group consisting of phenyl, C₁₋₆alkyl, and C₃₋₆cycloalkyl, or R^{x} and R^{y} are each independently selected from the group consisting of phenyl, cyclopropyl, isopropyl, tert-butyl, and cyclohexyl.

In certain embodiments, compounds of Formula I, Ia, Ib, Ic, II, IIa, III, IV, V, VI, VII, VIIa, VIIb, VIIc, VIII, and/or IX comprise a V group selected from residues having the formula —C(O)—V′, wherein V′ is a residue selected from the group consisting of tert-butoxy, 2,2-dimethylpropoxy, sec-butoxy, 1,2-dimethylpropoxy, 3-pentoxy, isopropoxy, C₁₋₉alkoxy, 2,2,2-trichloroethoxy,

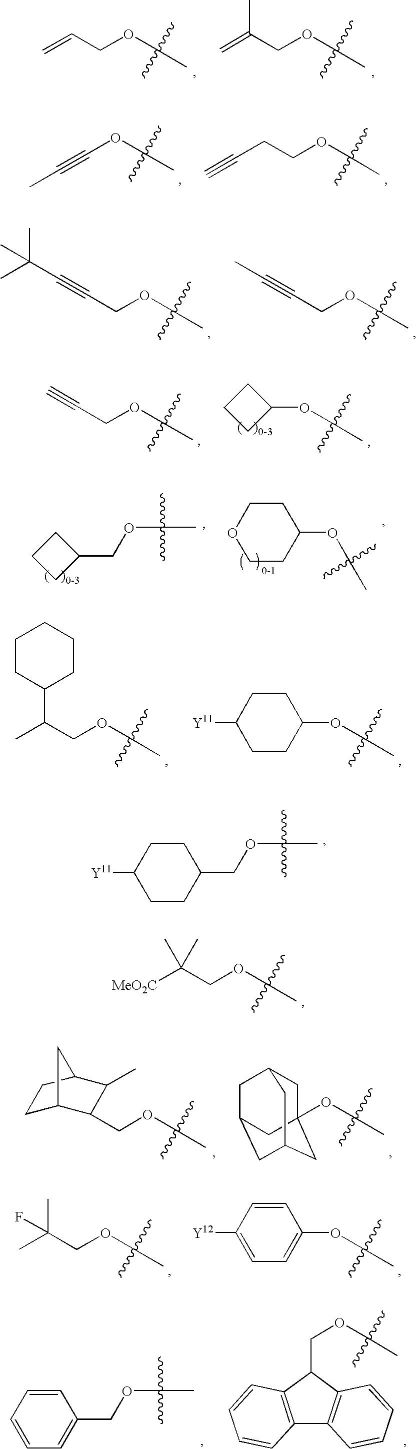

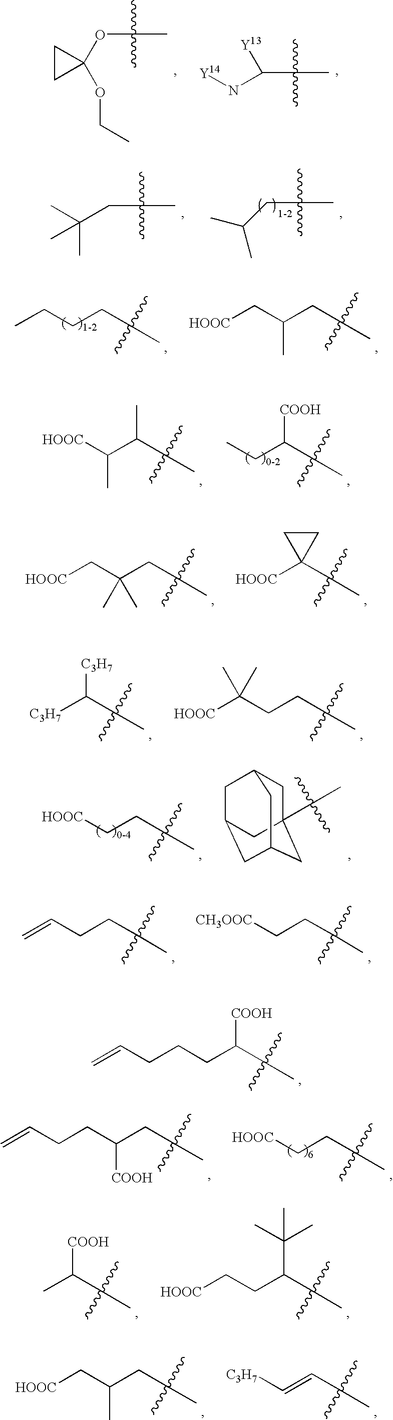

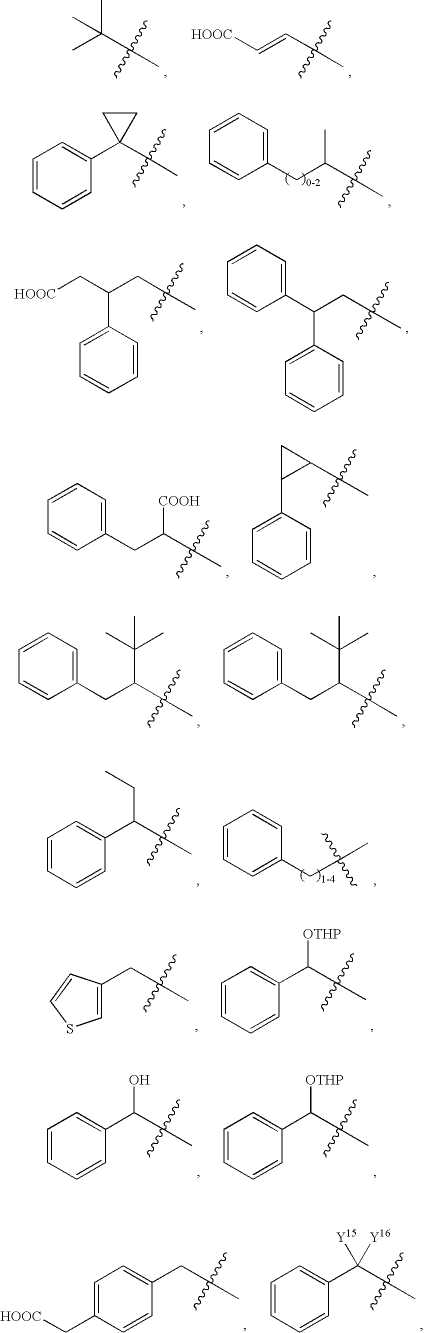

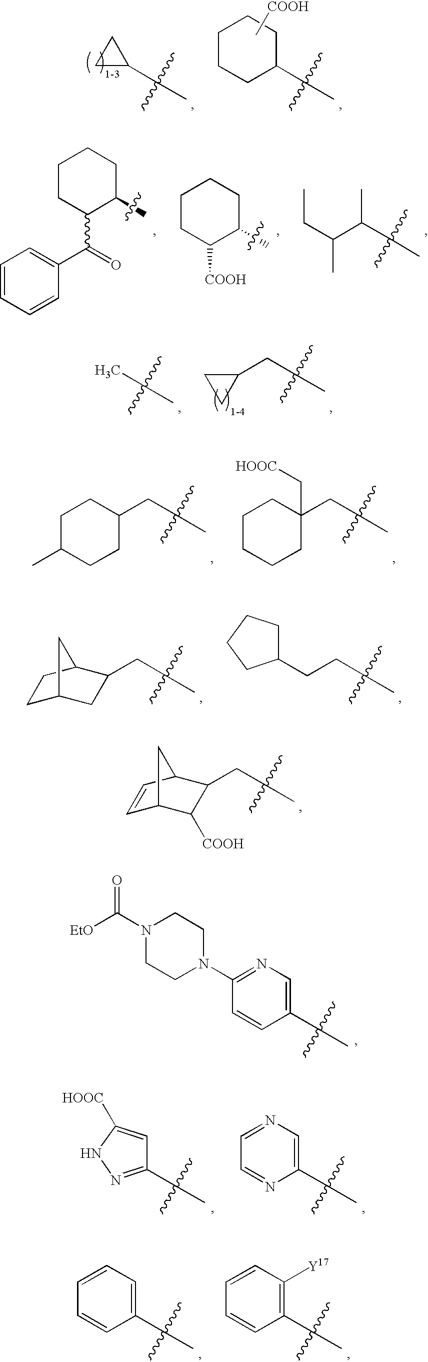

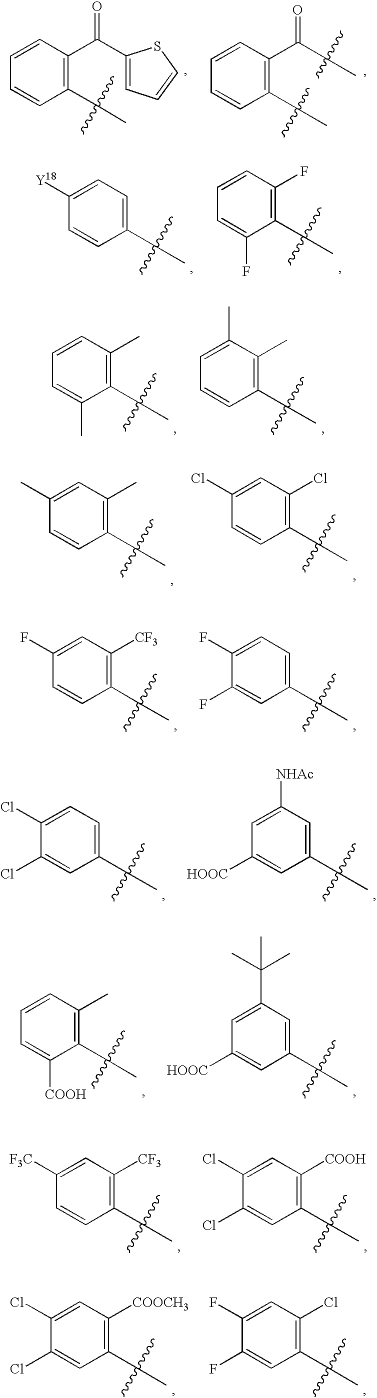

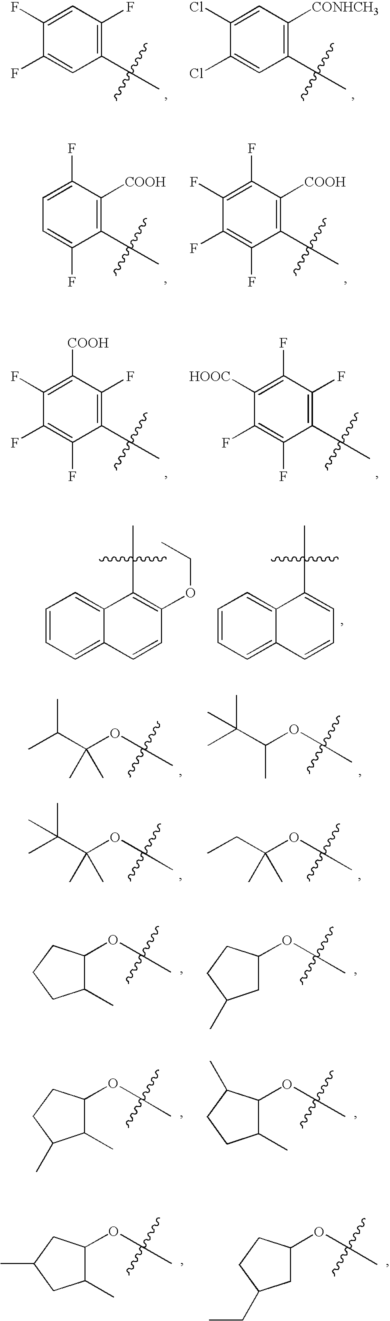

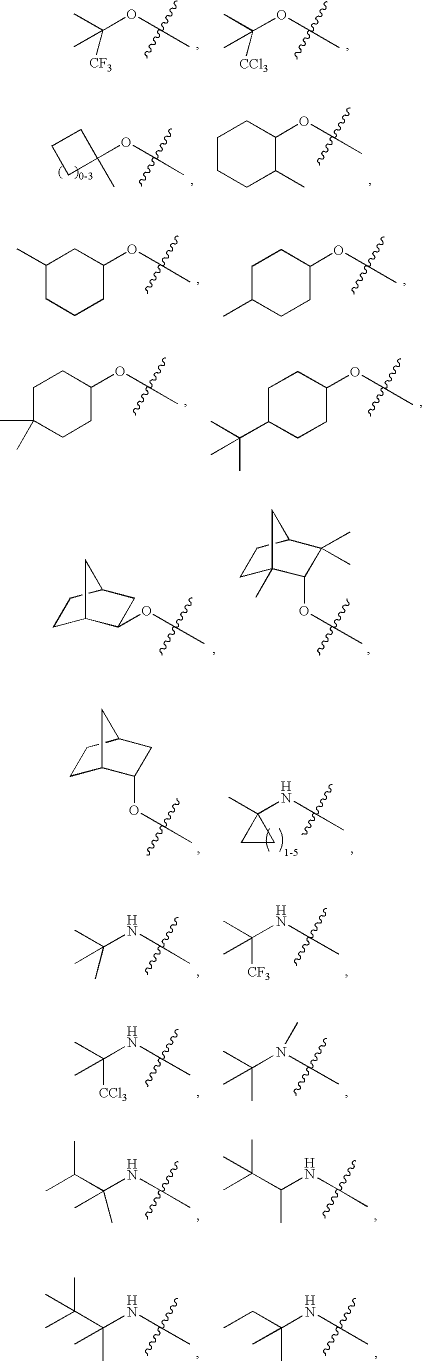

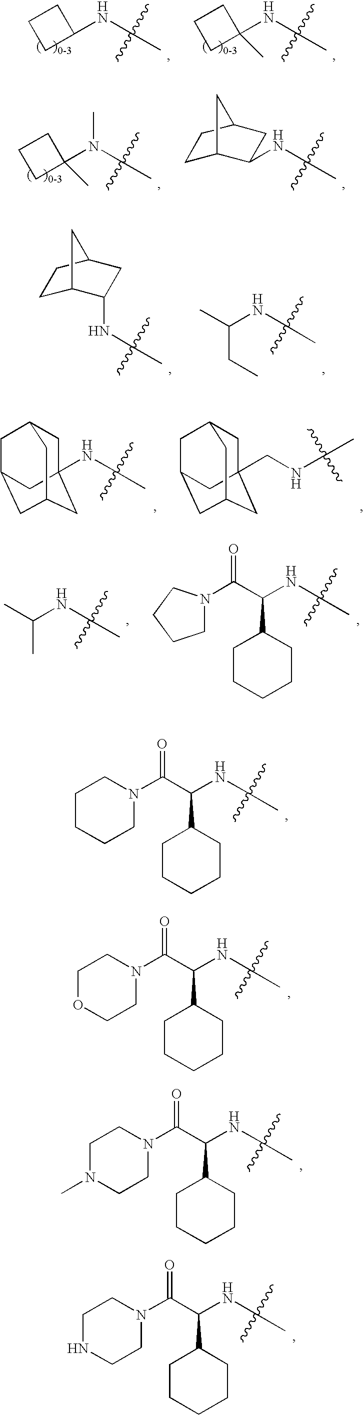

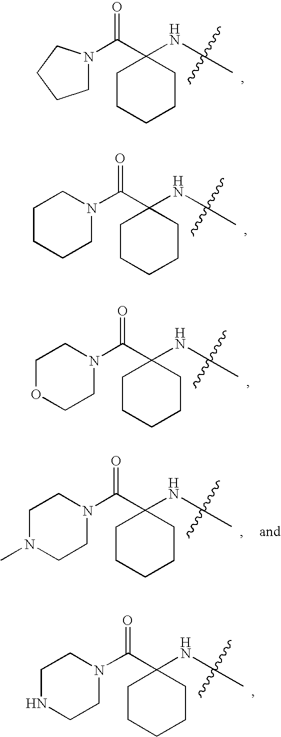

wherein

Y¹¹ is selected from the group consisting of hydrogen, —C(O)OH, —C(O)OEt, —OMe, -Ph, —OPh, —NHMe, —NHAc, —NHPh, —CH(Me)₂, 1-triazolyl, 1-imidazolyl, and —NHCH₂COOH;

Y¹² is selected from the group consisting of hydrogen, —C(O)OH, —C(O)OMe, —OMe, F, Cl, and Br;

Y¹³ is selected from the group consisting of the following moieties:

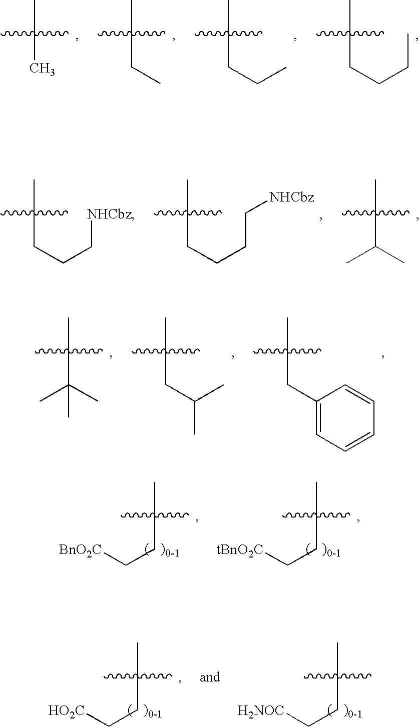

Y¹⁴ is S(O)₂Me, —C(O)Me, -Boc, -iBoc, Cbz, or -Alloc:

Y¹⁵ and Y¹⁶ can be the same or different and are independently selected from the group consisting of alkyl, aryl, heteroalkyl, and heteroaryl;

Y¹⁷ is —CF₃, —NO₂, —C(O)NH₂, —OH, —C(O)OCH₃, —OCH₃, —OC₆H₅, —C₆H₅, —C(O)C₆H₅, —NH₂, or —C(O)OH; and

Y¹⁸ is —C(O)OCH₃, —NO₂, —N(CH₃)₂, F, —OCH₃, —C(H)₂C(O)OH, —C(O)OH, —S(O)₂NH₂, or —N(H)C(O)CH₃

Certain compounds of Formulae I, Ia, Ib, and/or Ic, include compounds in which X is CR₅R₅ₐ, R₅ₐ is H, and R₅ is selected from the group consisting of piperidine, phenyl, —O-pyridinyl and CH₂-pyridinyl, wherein the phenyl and pyridinyl groups may be independently substituted one or more times with a halogen atom or C₁₋₄-alkyl. Certain other compounds of Formula I, Ia, Ib, Ic and/or II include compounds in which X is CR₅R₅ₐ, R₅ₐ is H and R₅ is selected from the group consisting of 7-methoxy-2-phenyl-quinolin-4-yloxy, 2-(2-isopropylamino-thiazol-4-yl)-7-methoxy-quinolin-4-yloxy and 4-fluoro-1,3-dihydro-isoindole-2-carbonyloxy.

In certain compounds of Formulae I, Ia, Ib, and/or Ic, X is CR⁵R^{5a}, R^{5a} is hydrogen, and R⁵ is selected from the group consisting of piperidine, phenyl, pyridinyl, pyridinyloxy and pyridinylmethyl, wherein the phenyl and pyridinyl groups may be independently substituted one or more times with a halogen atom or C₁₋₄-alkyl.

In yet another embodiment of Formula I, R₅ is 5-chloro-pyridin-2-yl.

Certain other compounds of Formulae I, Ia, Ib, and/or Ic, include compounds in which X is CR₅R₅ₐ, R₄ is H, R^{5a} is hydrogen, and R⁵ is selected from the group consisting of

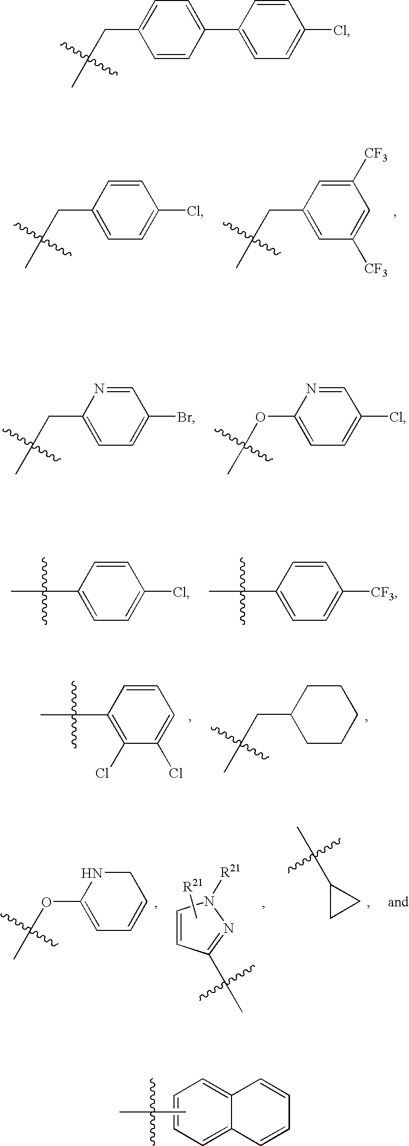

wherein R²¹ is independently selected from the group consisting of C₁₋₄-alkyl and aryl.

Certain other compounds of Formulae I, Ia, Ib, and/or Ic include compounds in which X is CR⁵R^{5a}, R⁴ is hydrogen, and R⁵ and R^{5a} taken in combination form a 3 to 6 member spirocyclic carbocycle substituted with 0-2 substituents selected from halogen, C₁₋₆-alkyl, C₂₋₆-alkenyl, C₂₋₆-alkynyl, C₁₋₆-alkoxy, C₃₋₇-cycloalkyl-C₀₋₄-alkyl, phenyl-C₀₋₄-alkyl, naphthyl-C₀₋₄-alkyl, heteroaryl-C₀₋₄-alkyl, or two substituents taken together form a fused or spirocyclic 3 to 7 membered carbocyclic ring, each of which is substituted with 0-3 independently selected halogen atoms or C₁₋₄-alkyl groups.

Certain other compounds of Formulae I, Ia, Ib, and/or Ic include compounds in which X is CR₅R_{5B}, R₄ is H, and R₅ and R₅ₐ taken in combination form a 3 to 6 member spirocyclic carbocycle substituted with 0-2 substituents selected from halogen, C₁₋₆-alkyl, C₂₋₆-alkenyl, C₂₋₆-alkynyl, C₁₋₆-alkoxide, C₃₋₇-cycloalkyl-C₀₋₄-alkyl, phenyl-C₀₋₄-alkyl, naphthyl-C₀₋₄-alkyl, heteroaryl-C₀₋₄-alkyl, or two substituents taken together form a fused or spirocyclic 3 to 7 membered carbocyclic ring, each of which is substituted with 0-3 independently selected halogen atoms or C₁₋₄-alkyl groups.

In still other embodiments, CR₅R₅ₐ, taken in combination, form a spirocyclic 3 to 6 member carbocyclic ring. Certain spirocyclic rings include groups of the formula:

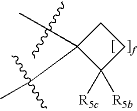

wherein
f is 0, 1, 2, 3, 4 or 5;
R_{5b} and R_{5c} are independently selected from hydrogen halogen, C₁₋₆-alkyl, C₂₋₆-alkenyl, C₂₋₆-alkynyl, C₁₋₆-alkoxide, C₃₋₇-cycloalkyl-C₀₋₄-alkyl, phenyl-C₀₋₄-alkyl, naphthyl-C₀₋₄-alkyl, heteroaryl-C₀₋₄-alkyl, or two substituents taken together form a fused or spirocyclic 3 to 7 membered carbocyclic ring, each of which is substituted with 0-3 independently selected halogen atoms or C₁₋₄-alkyl groups.

In yet another embodiment of Formulae I, Ia, Ib, and/or Ic, R₂ is selected from the group consisting of propyl and (CH₂)₂-cyclobutyl.

In still another embodiment of Formulae I, Ia, Ib, and/or Ic, R₁₁ is H and R₁₂ is C₃₋₆-cycloalkyl.

In one embodiment of Formulae I, Ia, Ib, and/or Ic, R₁₂ is cyclohexyl.

In another embodiment of formula I, V is selected from the group consisting of C(O)—N(H)-t-butyl.

In yet another embodiment of Formulae I, Ia, Ib, and/or Ic, V is R₂₀ or C(O)—R₂₀, wherein R₂₀ is selected from the group consisting of C₃₋₆-cycloalkyl, phenyl, pyrazine, benzooxazole, 4,4-dimethyl-4,5-dihydro-oxazole, benzoimidazole, pyrimidine, benzothiazole 1,1-dioxide and quinazoline, each of which may be further independently substituted with a halogen atom, CF₃, C₁₋₄-alkyl or C₃₋₆-cycloalkyl.

In still another embodiment of Formulae I, Ia, Ib, and/or Ic, V is R₂₀ or C(O)—R₂₀, wherein R₂₀ is selected from the group consisting of

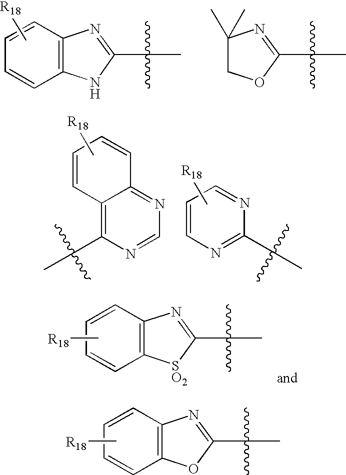

wherein R₁₈ is selected from the group consisting of hydrogen, a halogen atom, aryl, C₁₋₄-alkyl, C₁₋₄alkoxy, C₂-C₄alkenyloxy, C₂-C₄alkynyloxy, C₁₋₄-alkyl substituted by one or more halogen atoms, or C₃₋₆-cycloalkyl.

In one embodiment of Formulae I, Ia, Ib, and/or Ic, V is R₂₀ or C(O)—R₂₀, wherein R₂₀ is selected from the group consisting of

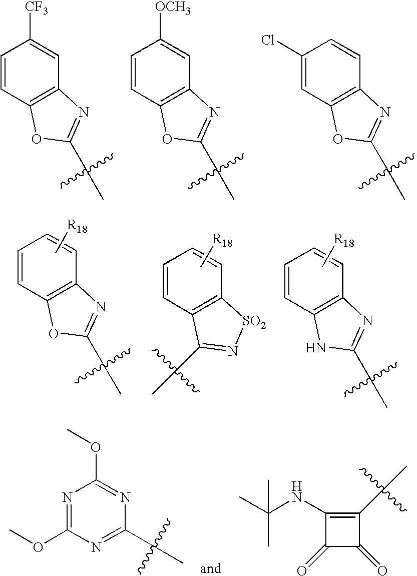

wherein R₁₈ is selected from the group consisting of hydrogen, a halogen atom, aryl, C₁₋₄-alkyl, C₁₋₄alkoxy, C₂-C₄alkenyloxy, C₂-C₄alkynyloxy, C₁₋₄-alkyl substituted by one or more halogen atoms, or C₃₋₆-cycloalkyl.

In another embodiment of Formula I, Ia, Ib, and/or Ic, V is selected from the group consisting of C₃₋₆-cycloalkyl, phenyl, pyrazine, benzooxazole, 4,4-dimethyl-4,5-dihydro-oxazole, benzoimidazole, pyrimidine, thiazole, benzothiazole, benzothiazole 1,1-dioxide and quinazoline, each of which may be further independently substituted with a halogen atom, C₁₋₄-alkyl, C₁₋₄alkoxy, C₂-C₄alkenyloxy, C₂-C₄alkynyloxy, C₁₋₄-alkyl substituted by one or more halogen atoms, or C₃₋₆-cycloalkyl.

In one embodiment, any of the C₃₋₆-cycloalkyl groups of Formulae I, Ia, Ib, Ic, or any subformula thereof, may be independently substituted one or more times with a halogen atom, aryl, heteroaryl, trihalomethyl, C₁₋₄-alkoxy or C₁₋₄-alkyl. .

In one embodiment of Formulae I, Ia, Ib, and/or Ic, or any subformulae thereof, any of the heterocycle groups are independently selected from the group consisting of acridinyl, carbazolyl, cinnolinyl, quinoxalinyl, pyrrazolyl, indolyl, benzotriazolyl, furanyl, thienyl, benzothienyl, benzofuranyl, quinolinyl, isoquinolinyl, oxazolyl, isoxazolyl, indolyl, pyrazinyl, pyridazinyl, pyridinyl, pyrimidinyl, pyrrolyl, tetrahydroquinoline, benzoimidazolyl, benzofuranyl, benzofurazanyl, benzopyrazolyl, benzotriazolyl, benzothiophenyl, benzoxazolyl, carbazolyl, carbolinyl, cinnolinyl, furanyl, imidazolyl, indolinyl, indolyl, indolazinyl, indazolyl, isobenzofuranyl, isoindolyl, isoquinolyl, isothiazolyl, isoxazolyl, naphthpyridinyl, oxadiazolyl, oxazolyl, oxazoline, isoxazoline, oxetanyl, pyranyl, pyrazinyl, pyrazolyl, pyridazinyl, pyridopyridinyl, pyridazinyl, pyridyl, pyrimidyl, pyrrolyl, quinazolinyl, quinolyl, quinoxalinyl, tetrahydropyranyl, tetrazolyl, tetrazolopyridyl, thiadiazolyl, thiazolyl, thienyl, triazolyl, azetidinyl, 1,4-dioxanyl, hexahydroazepinyl, piperazinyl, piperidinyl, pyridin-2-onyl, pyrrolidinyl, morpholinyl, thiomorpholinyl, dihydrobenzoimidazolyl, dihydrobenzofuranyl, dihydrobenzothiophenyl, dihydrobenzoxazolyl, dihydrofuranyl, dihydroimidazolyl, dihydroindolyl, dihydroisooxazolyl, dihydroisothiazolyl, dihydrooxadiazolyl, dihydrooxazolyl, dihydropyrazinyl, dihydropyrazolyl, dihydropyridinyl, dihydropyrimidinyl, dihydropyrrolyl, dihydroquinolinyl, dihydrotetrazolyl, dihydrothiadiazolyl, dihydrothiazolyl, dihydrothienyl, dihydrotriazolyl, dihydroazetidinyl, methylenedioxybenzoyl, tetrahydrofuranyl, and tetrahydrothienyl, and N-oxides thereof, each of which may be independently further substituted one or more times with a halogen atom, C₁₋₄-alkyl, C₁₋₄-alkyl substituted by one or more halogen atoms, or C₃₋₆-cycloalkyl.

Preferred embodiments of the compounds of the invention (including pharmaceutically acceptable salts thereof, as well as enantiomers, stereoisomers, rotamers, tautomers, diastereomers, or racemates thereof) are shown below in Table A and Table B, and are also considered to be “compounds of the invention.”

TABLE A

| Structure | Compound No. |
|---|---|
| 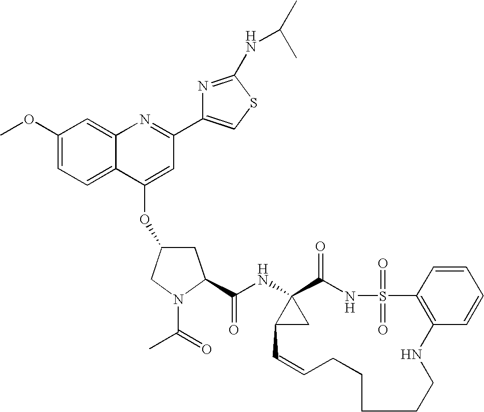 | A-1 |
| 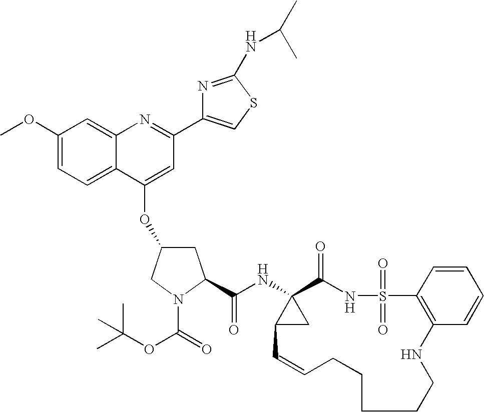 | A-2 |
| 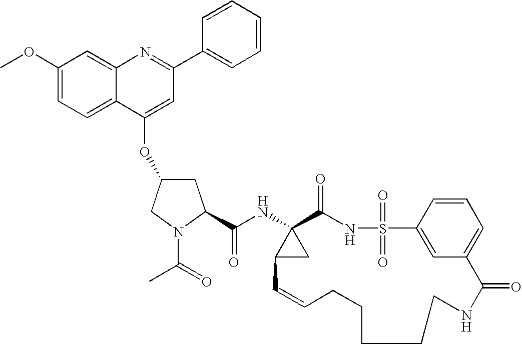 | A-3 |
| 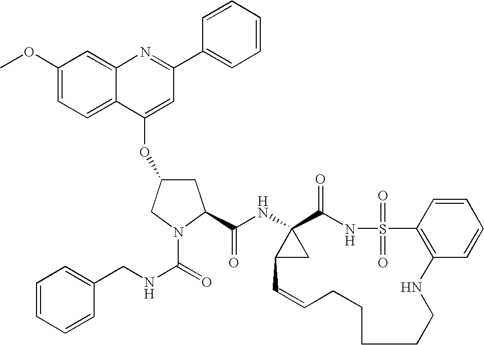 | A-4 |
| 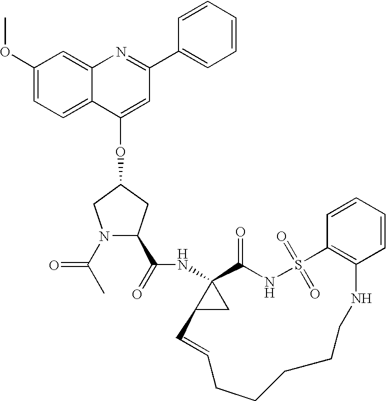 | A-5 |
| 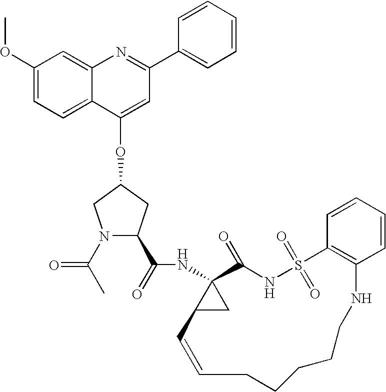 | A-6 |
| 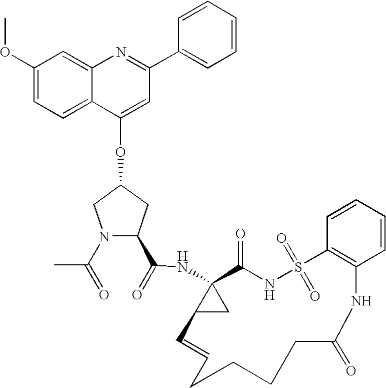 | A-7 |
| 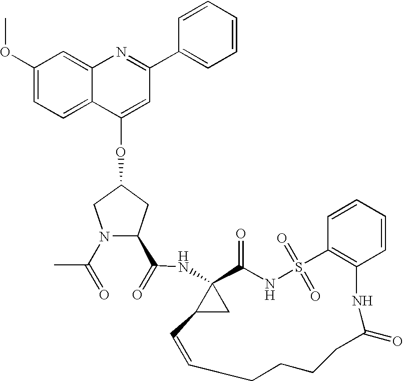 | A-8 |
| 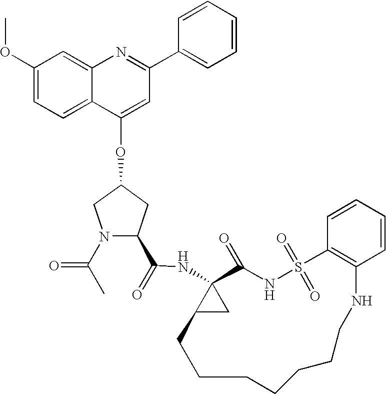 | A-9 |
| 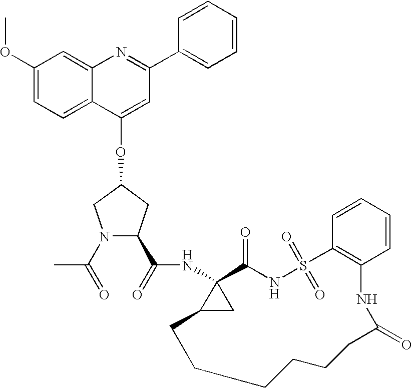 | A-10 |
| 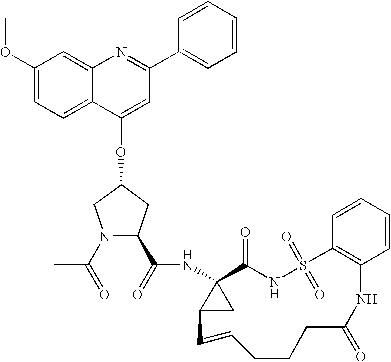 | A-11 |
| 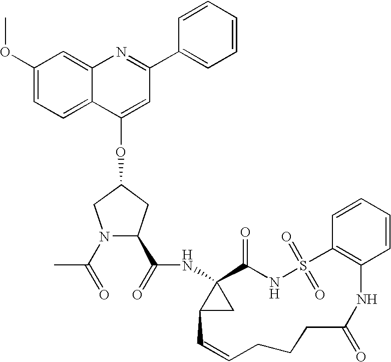 | A-12 |
| 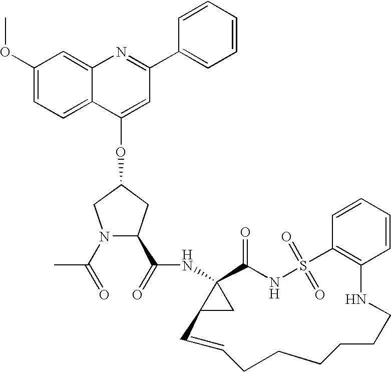 | A-13 |
| 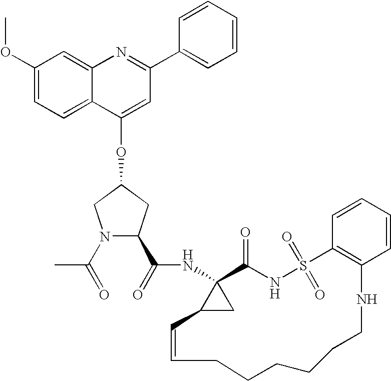 | A-14 |
| 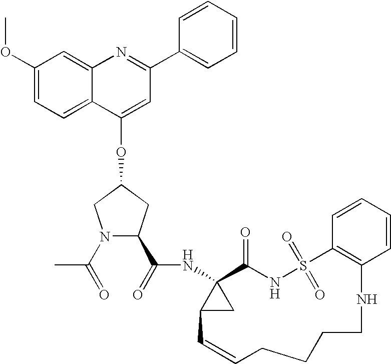 | A-15 |
| 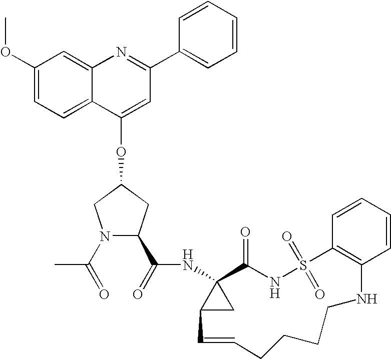 | A-16 |
| 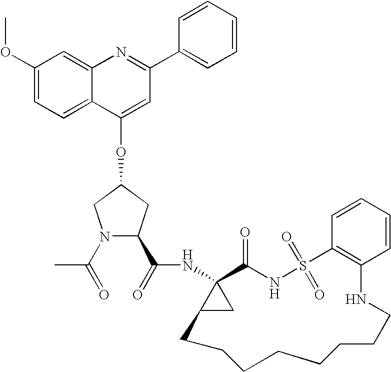 | A-17 |
| 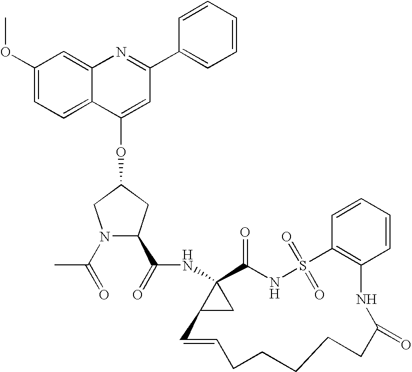 | A-18 |
| 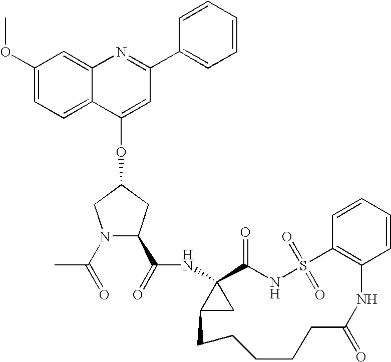 | A-19 |
| 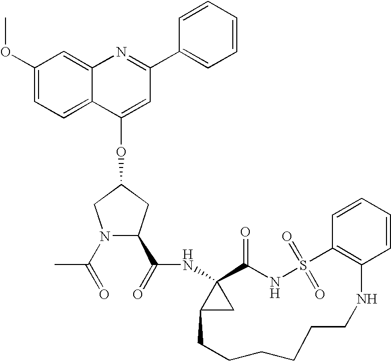 | A-20 |
| 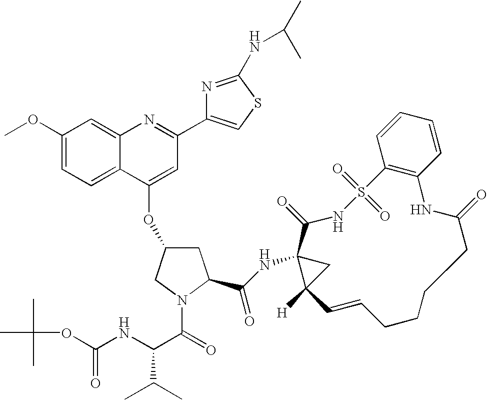 | A-21 |
| 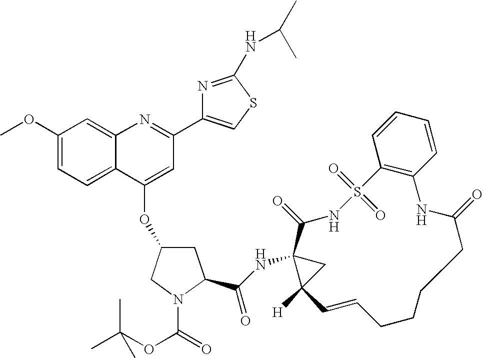 | A-22 |
| 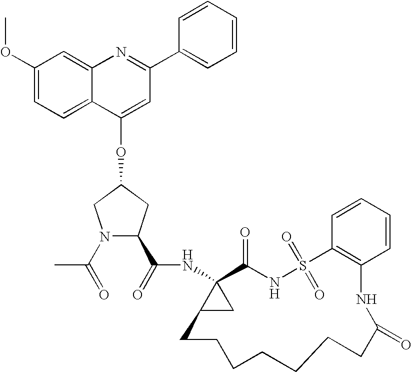 | A-23 |
| 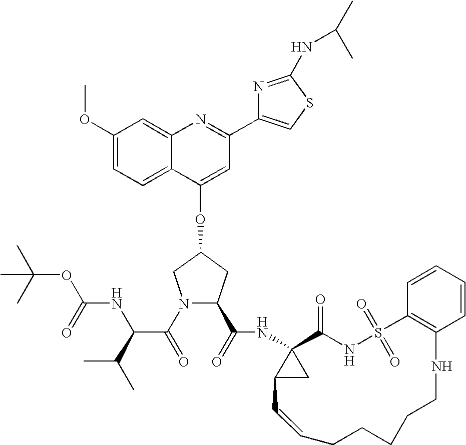 | A-24 |
| 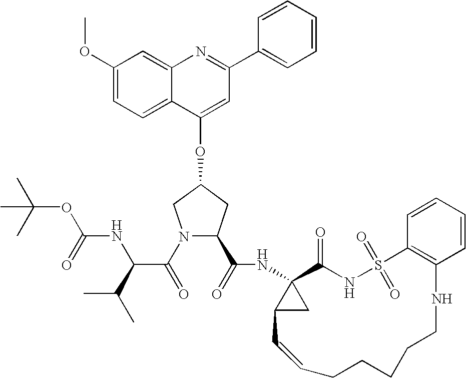 | A-25 |
| 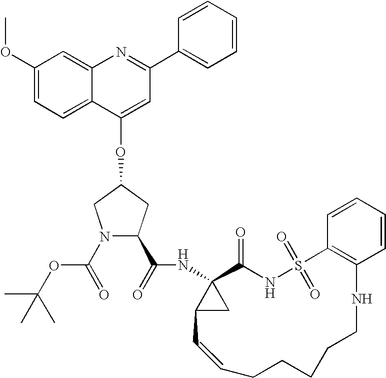 | A-26 |
| 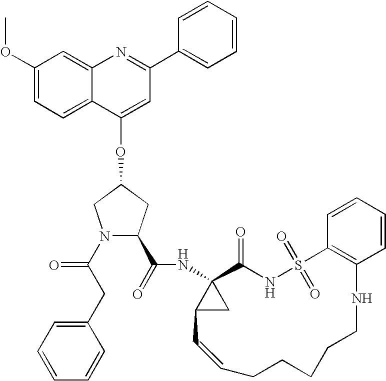 | A-27 |
| 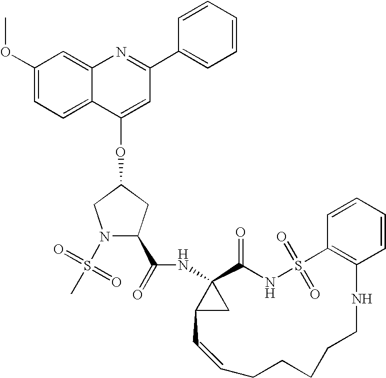 | A-28 |
| 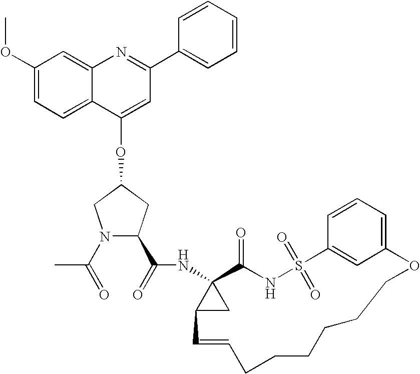 | A-29 |
| 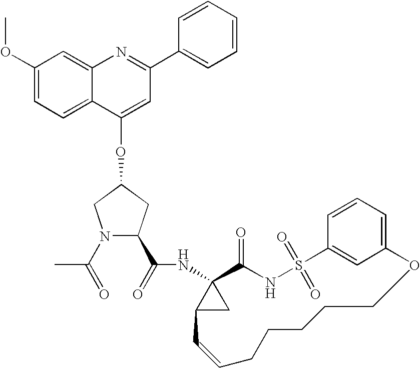 | A-30 |
| 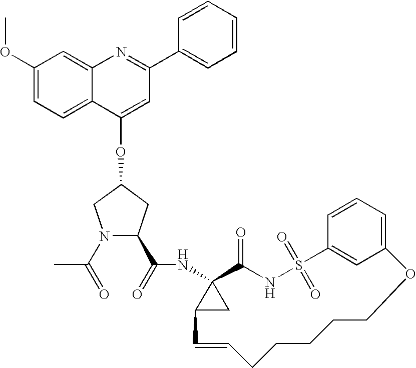 | A-31 |
| 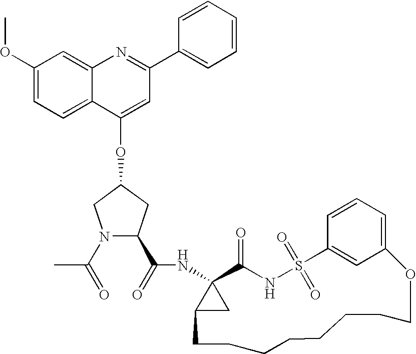 | A-32 |
| 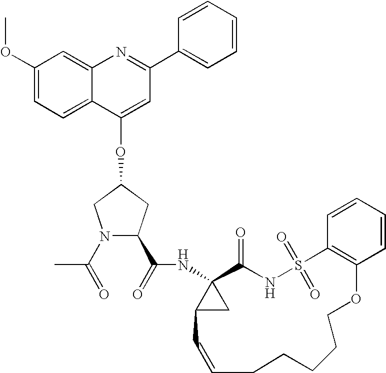 | A-33 |
| 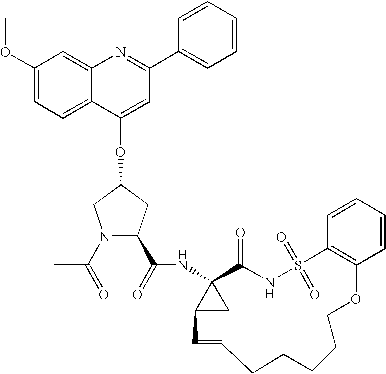 | A-34 |
| 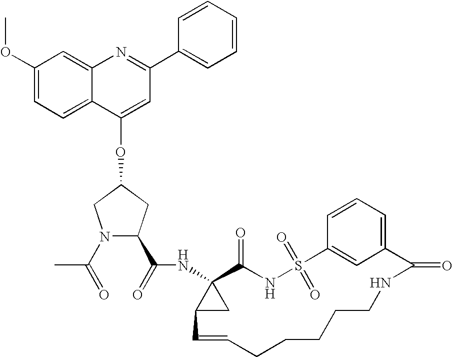 | A-35 |
| 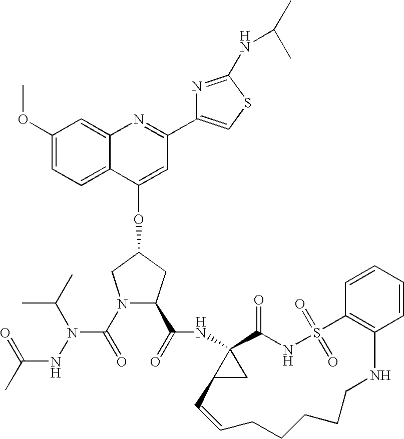 | A-36 |
| 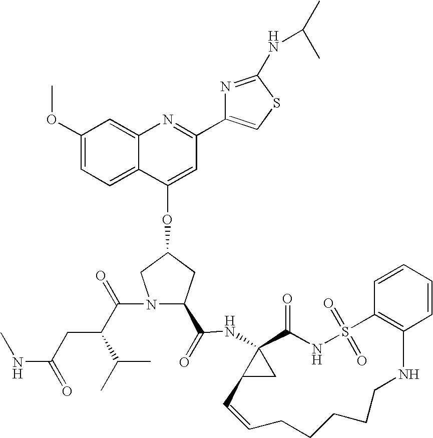 | A-37 |
| 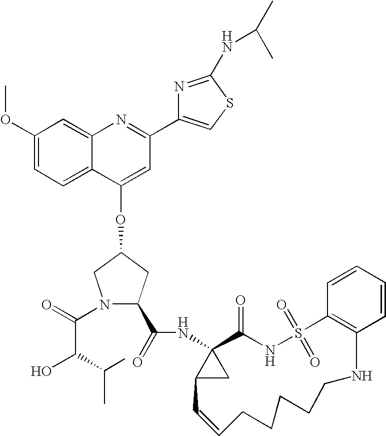 | A-38 |
| 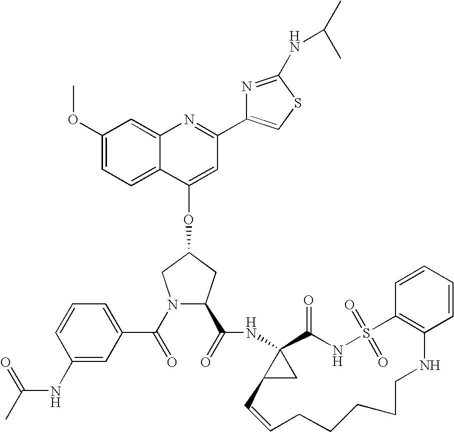 | A-39 |
| 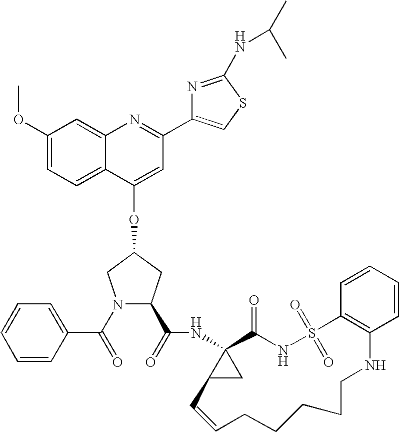 | A-40 |
| 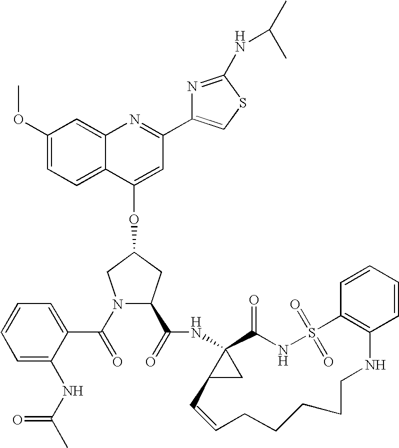 | A-41 |
| 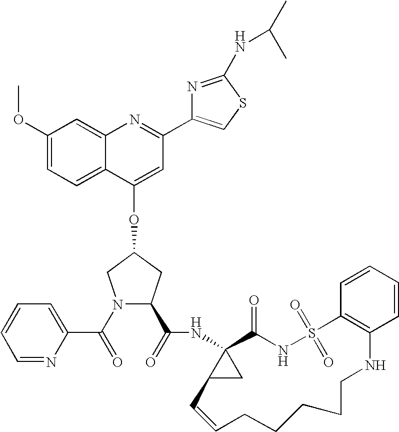 | A-42 |
| 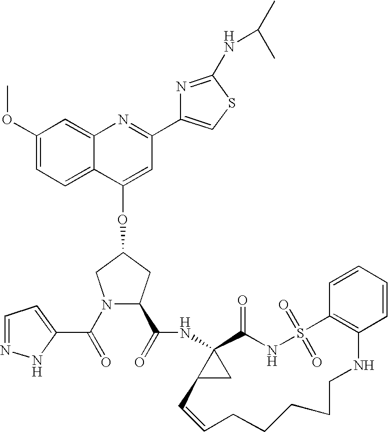 | A-43 |
| 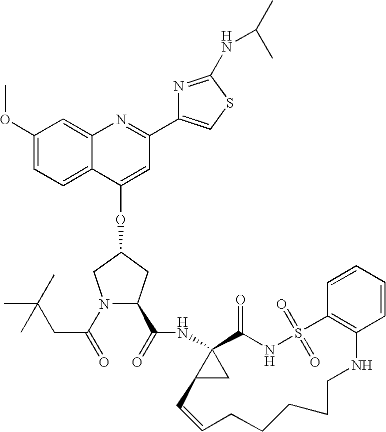 | A-44 |
| 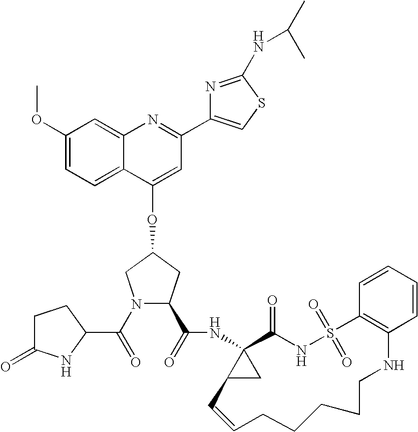 | A-45 |
| 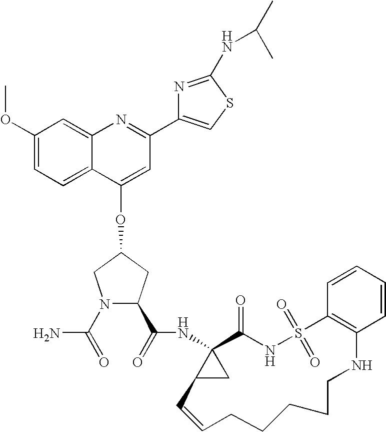 | A-46 |
| 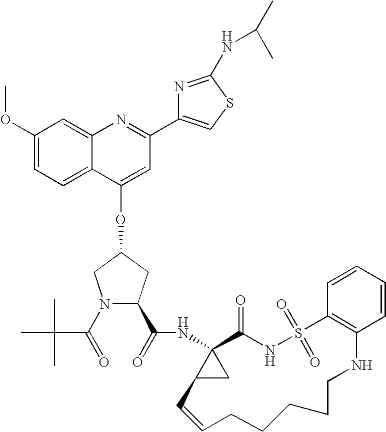 | A-47 |
| 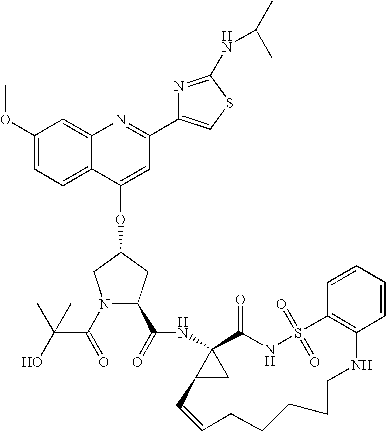 | A-48 |
| 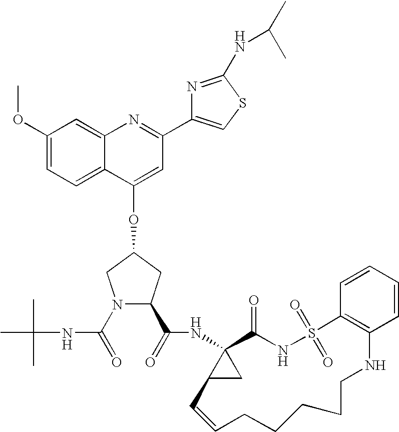 | A-49 |
| 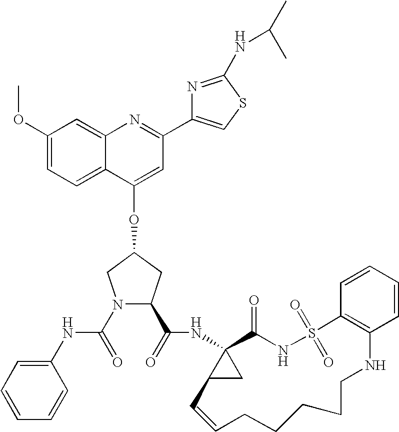 | A-50 |
| 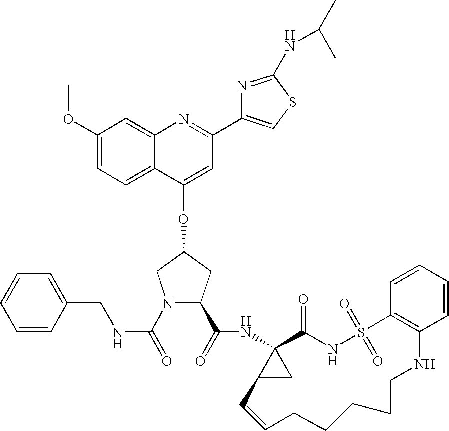 | A-51 |
| 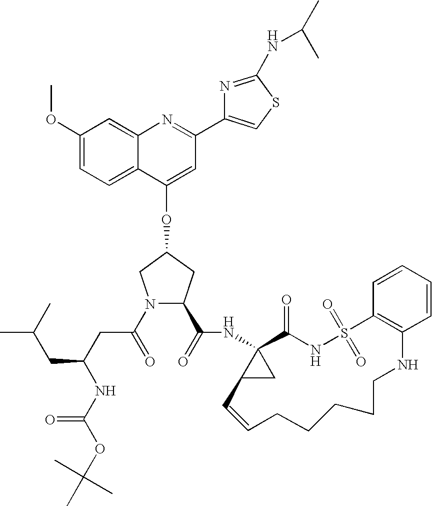 | A-52 |
| 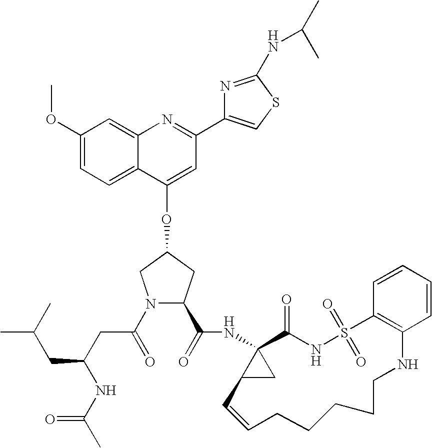 | A-53 |
| 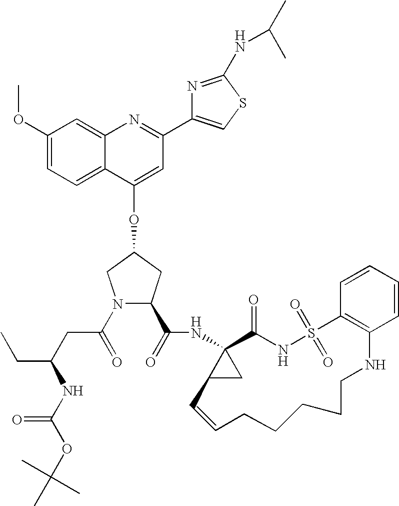 | A-54 |
| 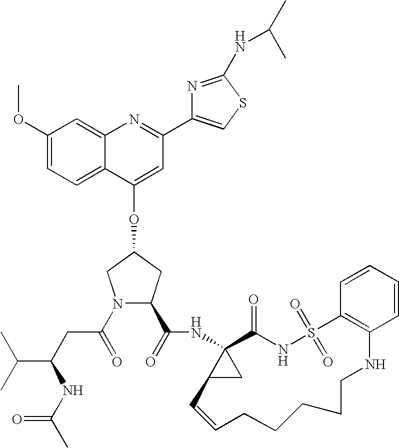 | A-55 |
| 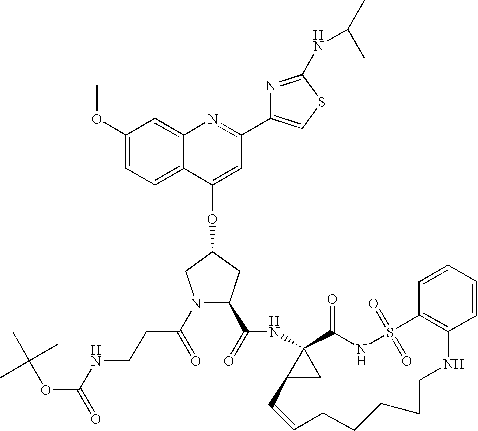 | A-56 |
| 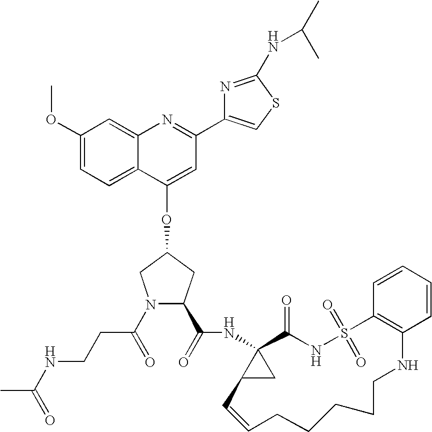 | A-57 |
| 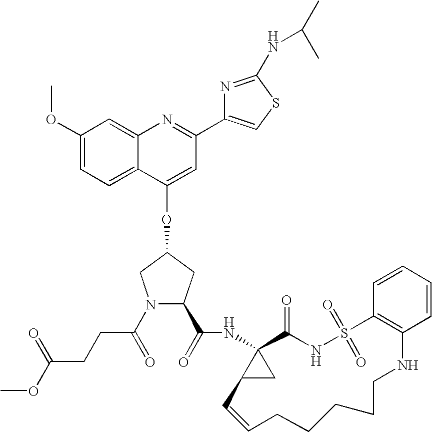 | A-58 |
| 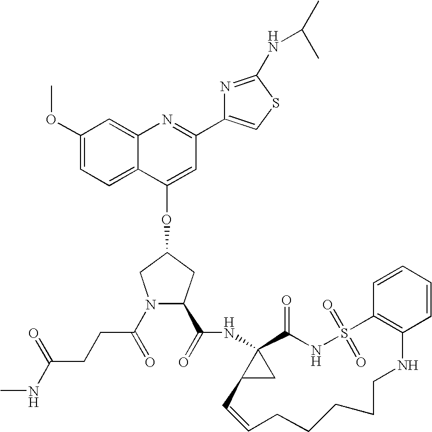 | A-59 |
| 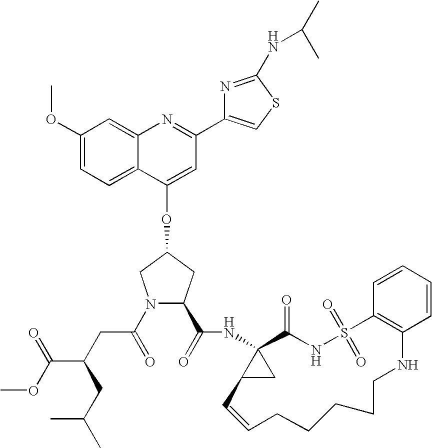 | A-60 |
| 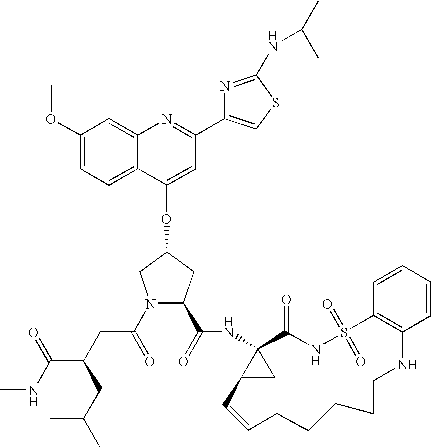 | A-61 |
| 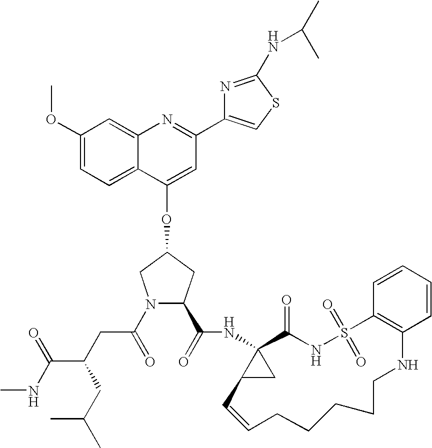 | A-62 |
| 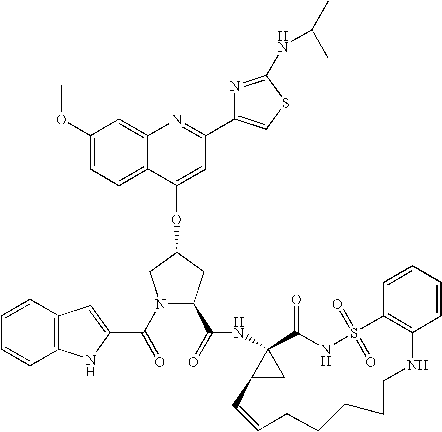 | A-63 |
| 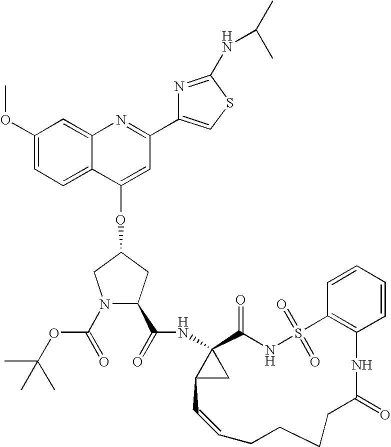 | A-64 |
| 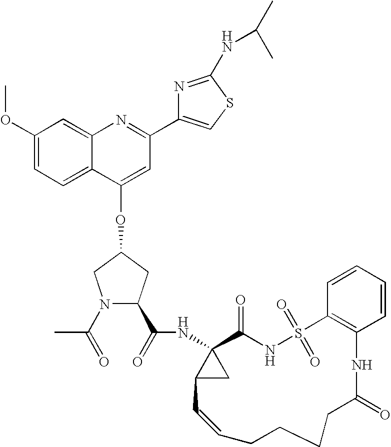 | A-65 |
| 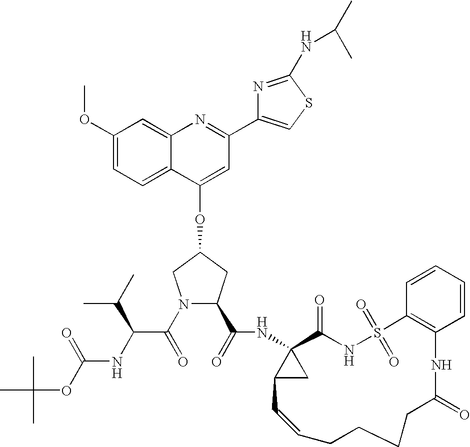 | A-66 |
| 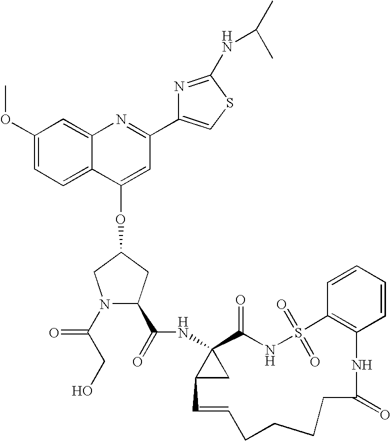 | A-67 |
| 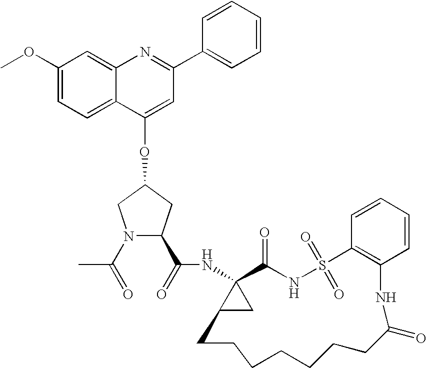 | A-68 |
| 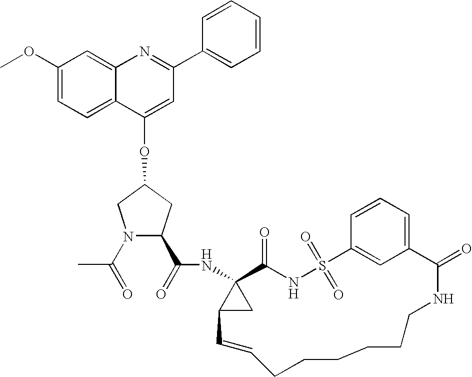 | A-69 |
| 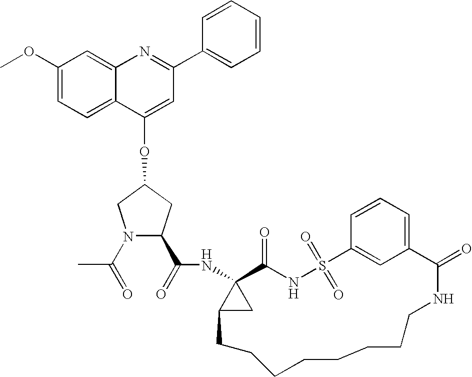 | A-70 |
| 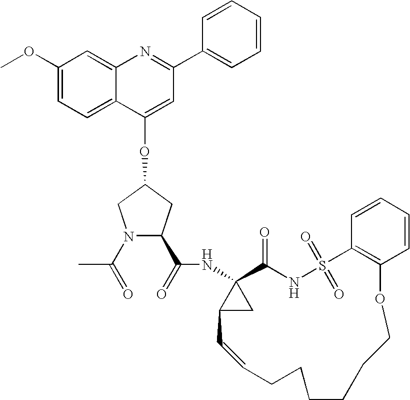 | A-71 |
| 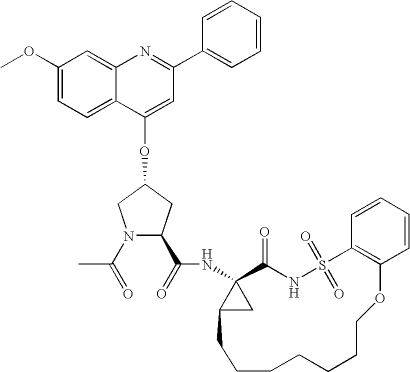 | A-72 |
| 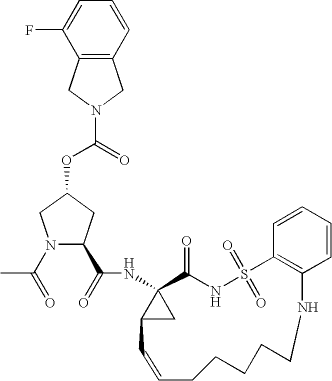 | A-73 |
| 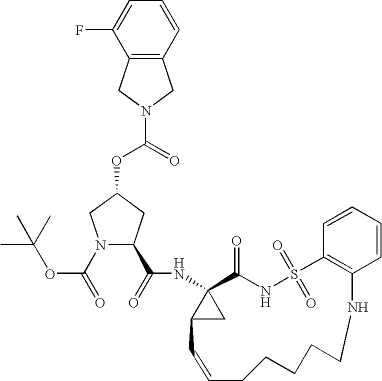 | A-74 |
| 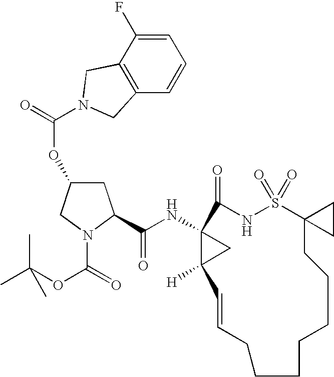 | A-75 |
| 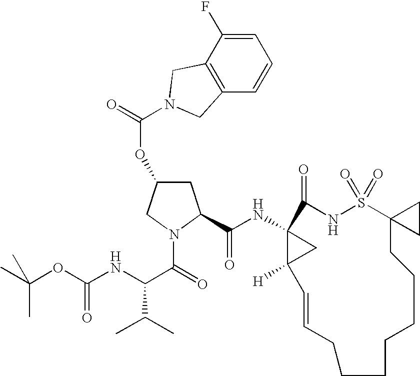 | A-76 |
| 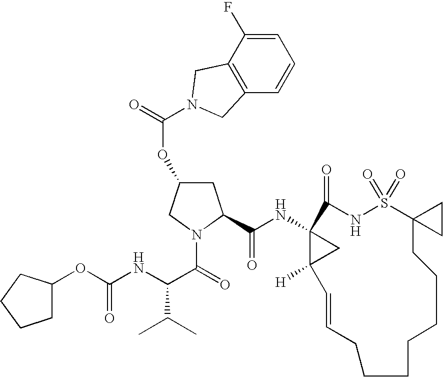 | A-77 |
| 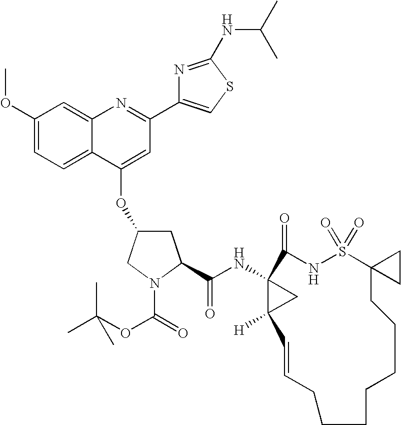 | A-78 |
| 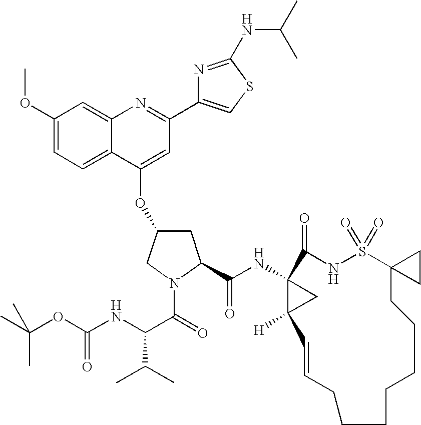 | A-79 |
| 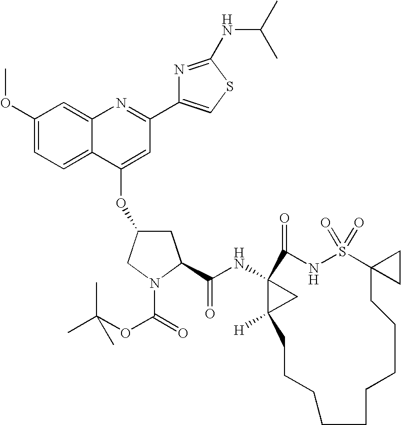 | A-80 |
| 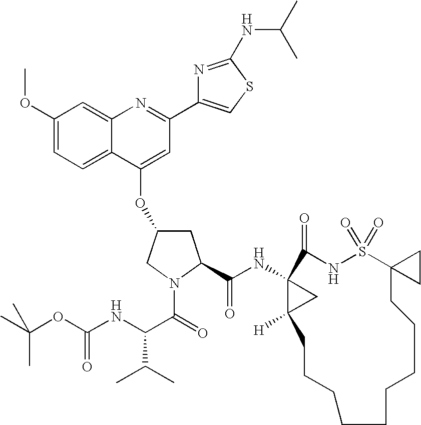 | A-81 |
| 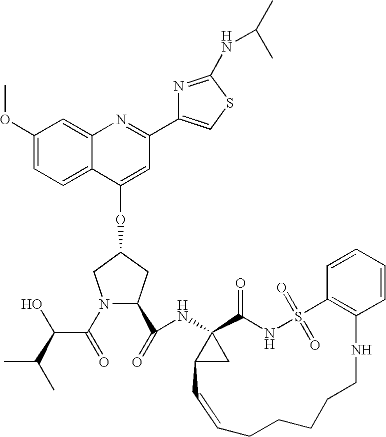 | A-82 |
| 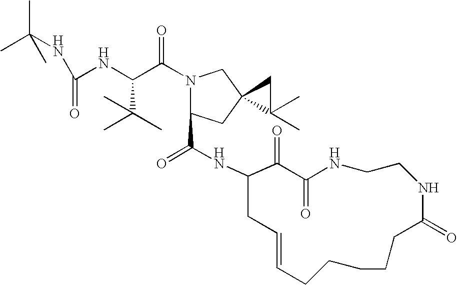 | A-83 |
| 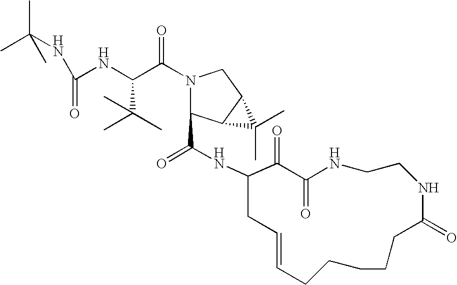 | A-84 |

Certain additional compounds of Formula I, Ia, Ib, and/or Ic (or subformulae thereof) which are contemplated in the present invention include compounds depicted in Table B.

TABLE B

| Structure | Compound No. |
|---|---|
| 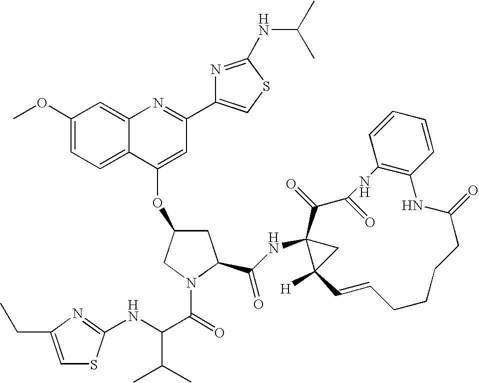 | B-1 |
| 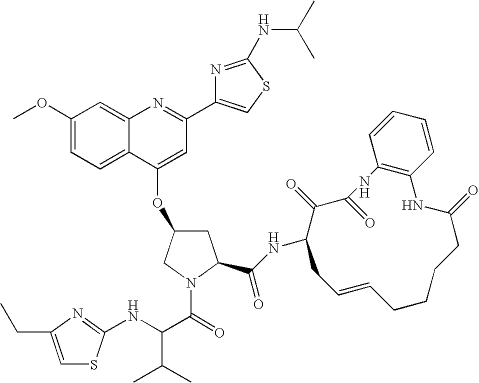 | B-2 |
| 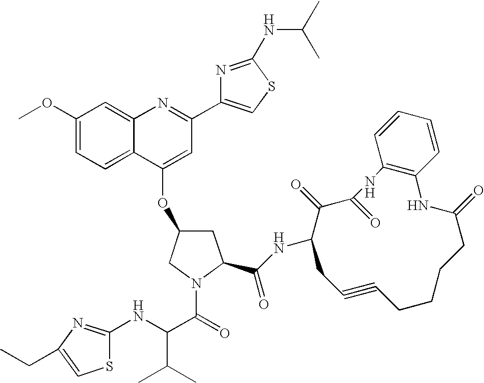 | B-3 |
| 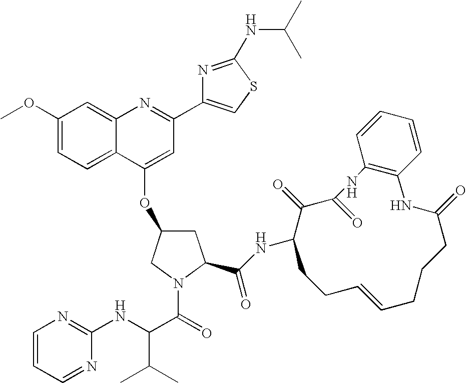 | B-4 |
| 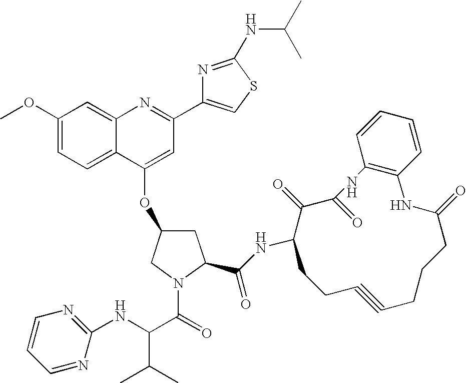 | B-5 |
| 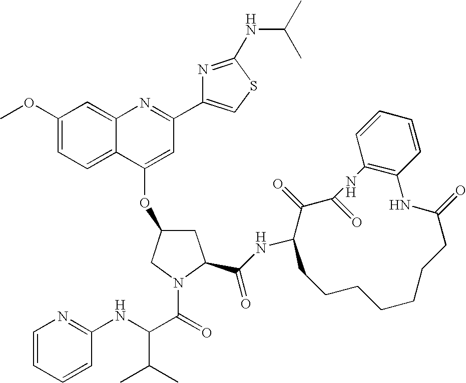 | B-6 |
| 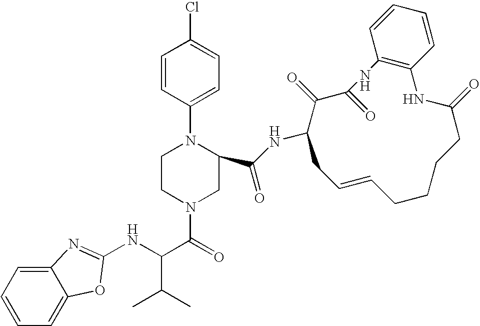 | B-7 |
| 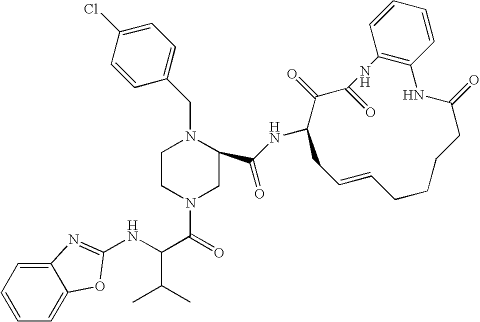 | B-8 |
| 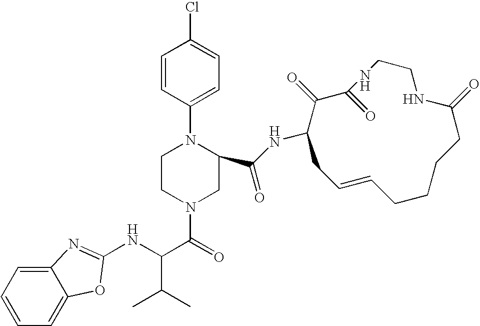 | B-9 |
| 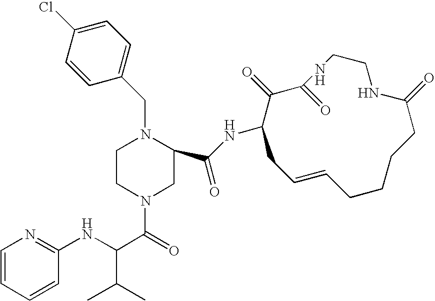 | B-10 |
| 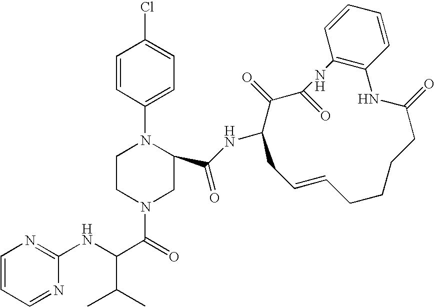 | B-11 |
| 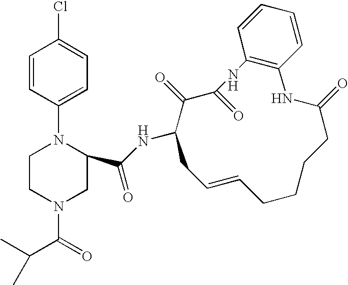 | B-12 |
| 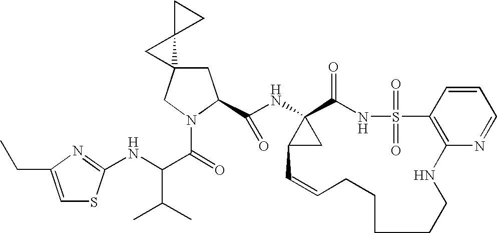 | B-13 |
| 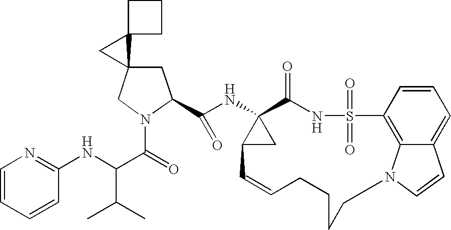 | B-14 |
| 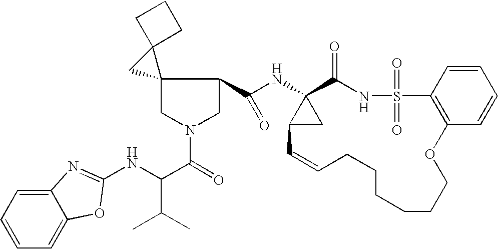 | B-15 |
| 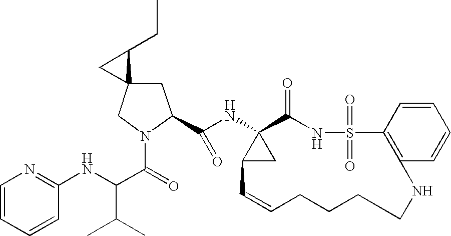 | B-16 |
| 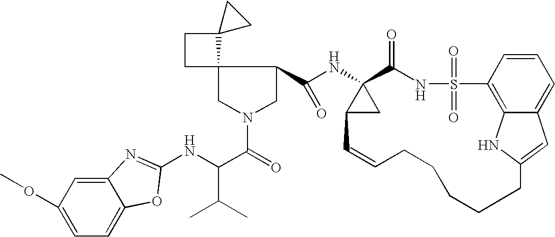 | B-17 |
| 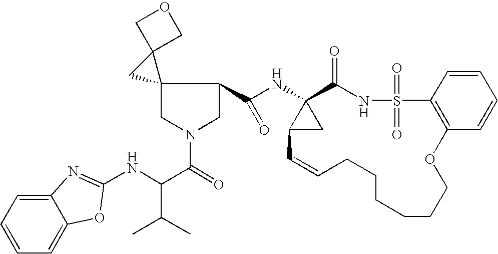 | B-18 |
| 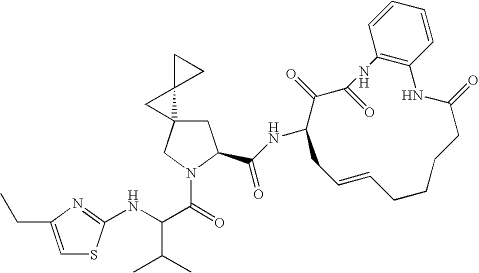 | B-19 |
| 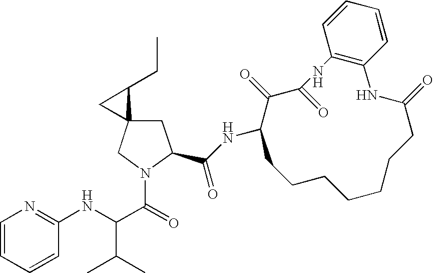 | B-20 |
| 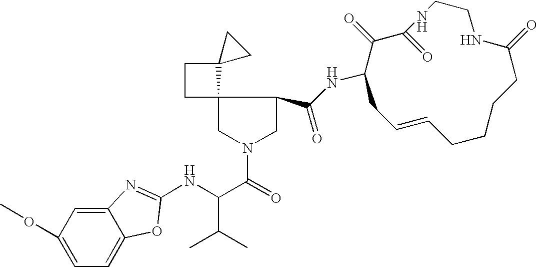 | B-21 |
| 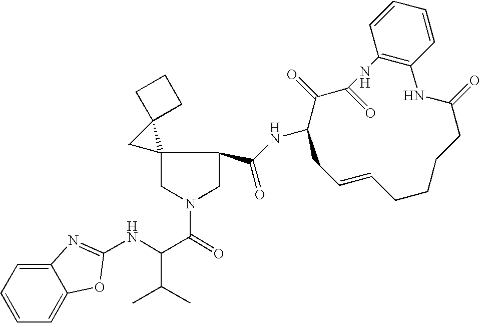 | B-22 |
| 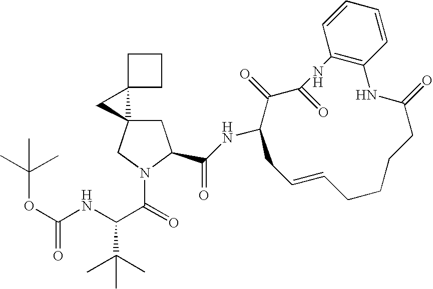 | B-23 |
| 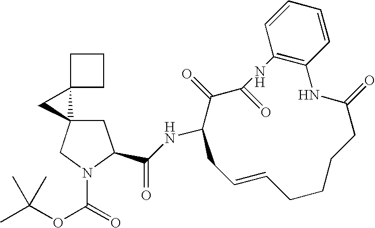 | B-24 |
| 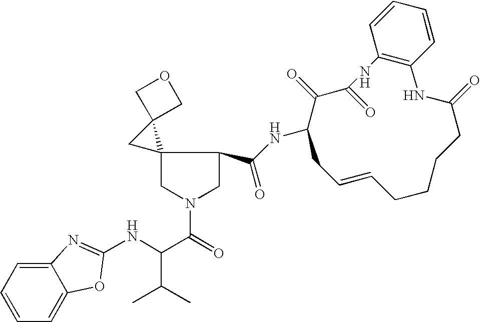 | B-25 |
| 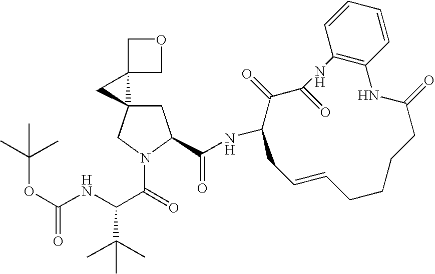 | B-26 |
| 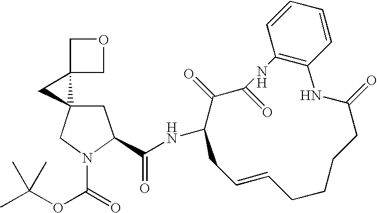 | B-27 |
| 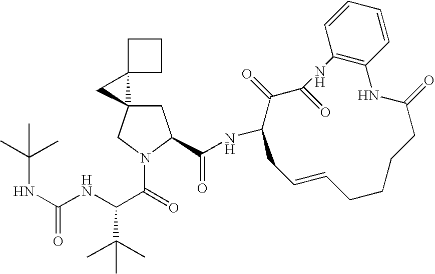 | B-28 |
| 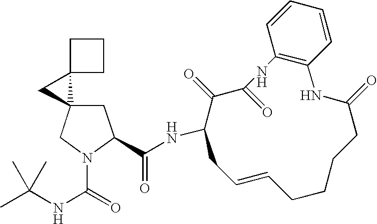 | B-29 |
| 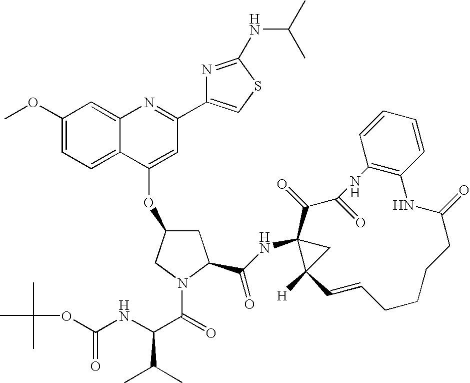 | B-30 |
| 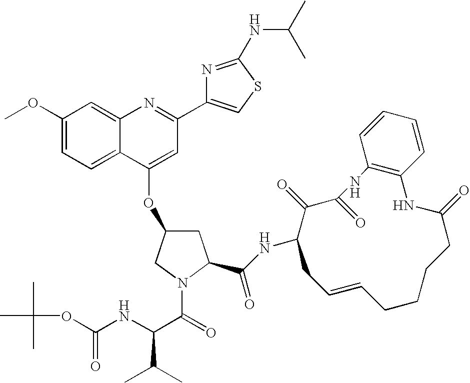 | B-31 |
| 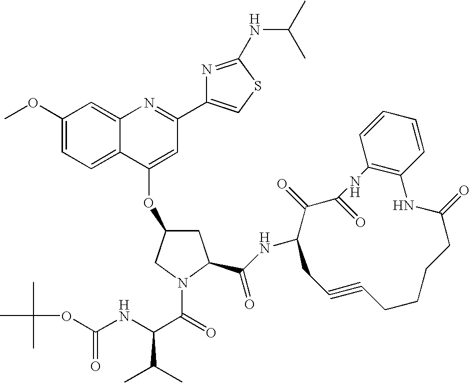 | B-32 |
| 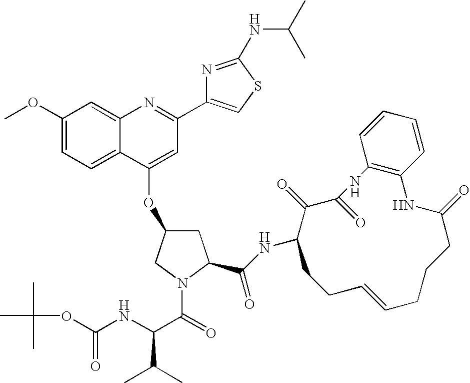 | B-33 |
| 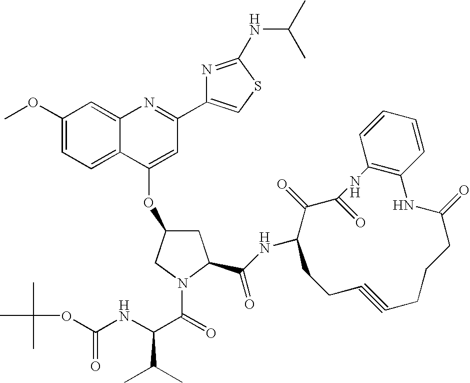 | B-34 |
| 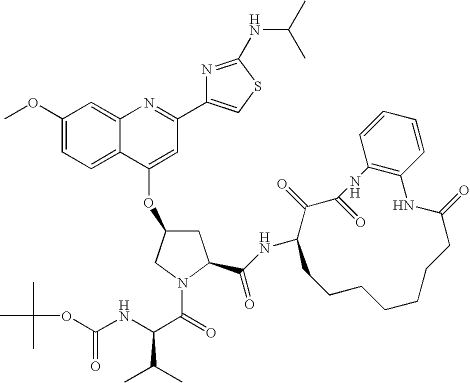 | B-35 |
| 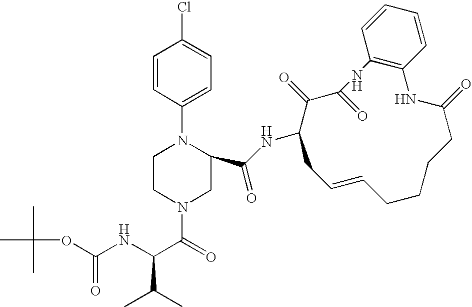 | B-36 |
| 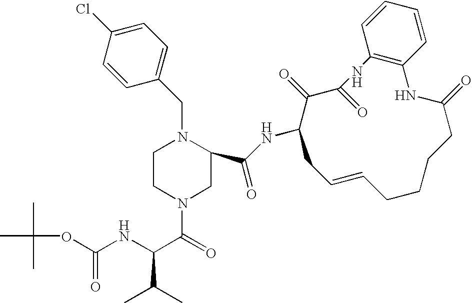 | B-37 |
| 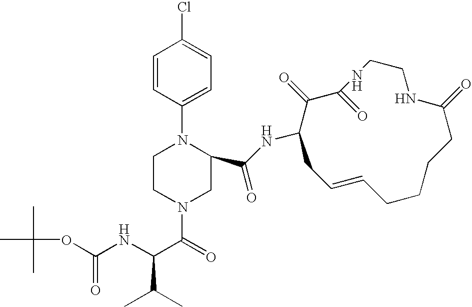 | B-38 |
| 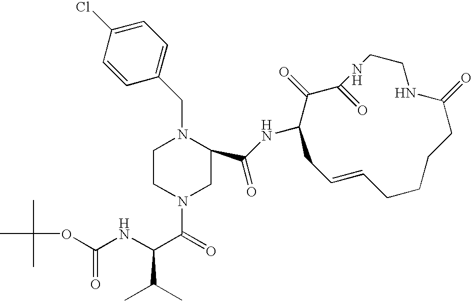 | B-39 |
| 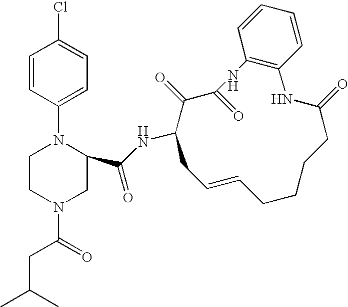 | B-40 |
| 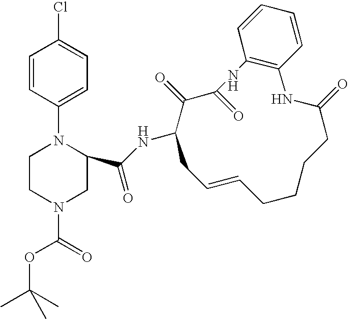 | B-41 |
| 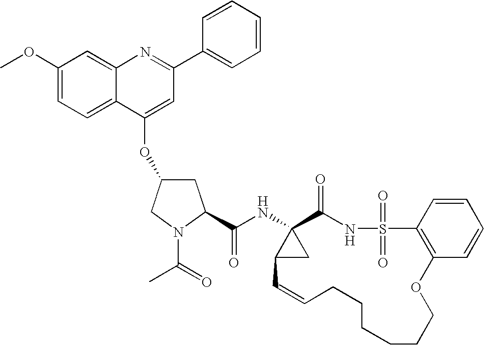 | B-42 |
| 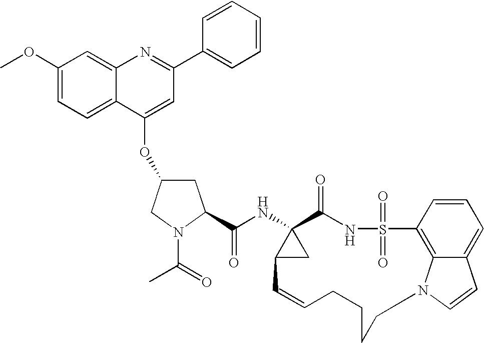 | B-43 |
| 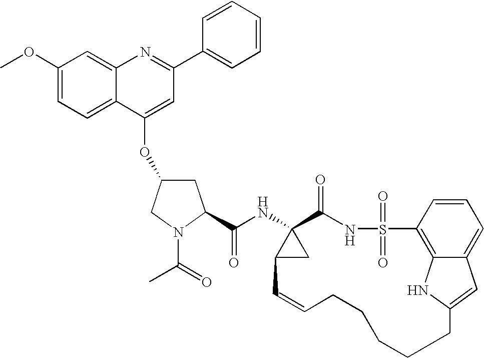 | B-44 |
| 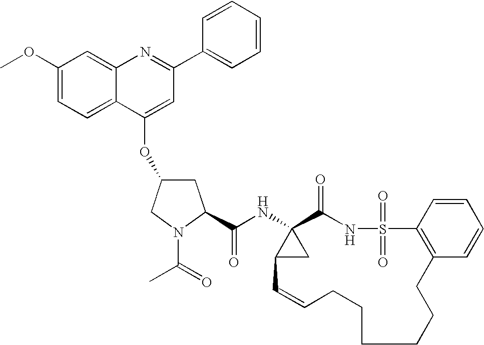 | B-45 |
| 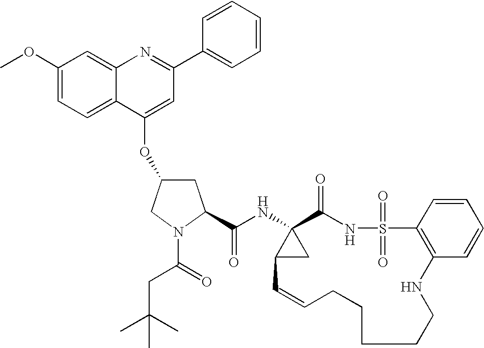 | B-46 |
| 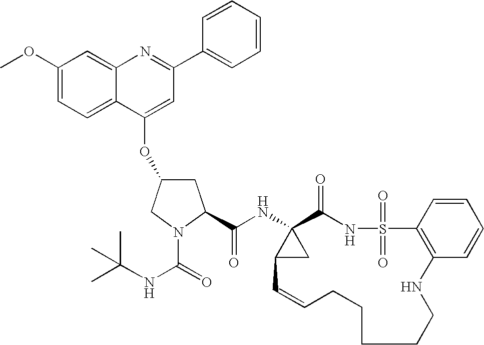 | B-47 |
| 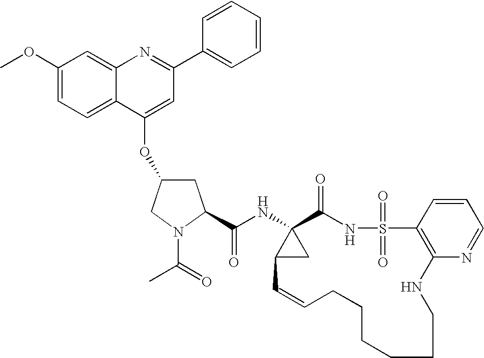 | B-48 |

Certain other compounds of Formula I, Ia, Ib, and/or Ic include those compounds which contain a fragment P1 selected from the residues of Table C and a P2 fragment selected from Table D. Thus, compounds of the invention include all P1-P2 compounds formed by combining all possible permutations of the P1 fragments of Table B with each P2 fragment of Table C wherein the P1 and P2 fragments are coupled by condensation of the amino residue on the P1 fragment with the carboxylic acid residue on the P2 fragment. Although the macrocycles of Table C (P1 Fragments) are drawn in cis geometry, the trans isomer is also contemplated as compounds of the invention. For example, the compound P1(10)-P2(5) is the condensation product of entry 10 in Table B and entry 5 in Table C and has the structure:

TABLE C
(P1 Fragments)

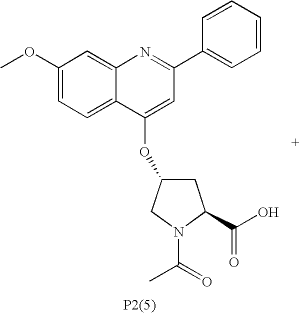

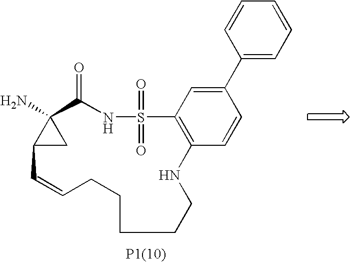

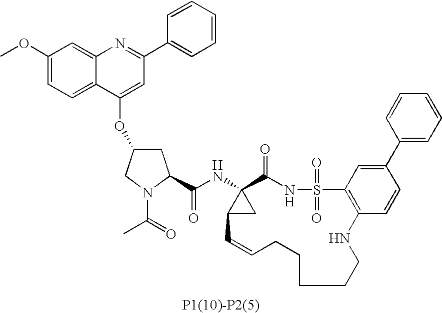

| | |
|---|---|
| | 1 |
| 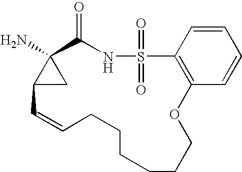 | |
| | 2 |
| 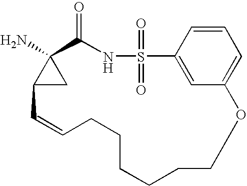 | |
| | 3 |
| 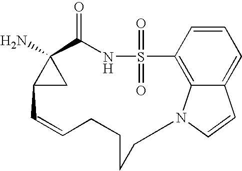 | |
| | 4 |
| 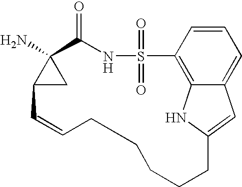 | |
| | 5 |
| 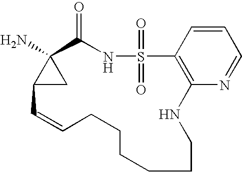 | |
| | 6 |
| 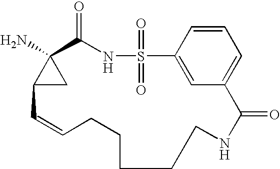 | |
| | 7 |
| 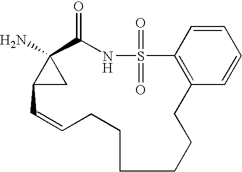 | |
| | 8 |
| 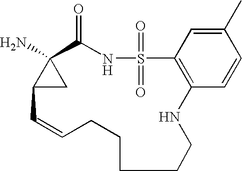 | |
| | 9 |
| 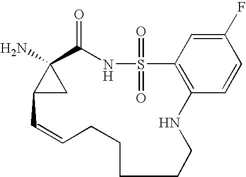 | |
| | 10 |
| 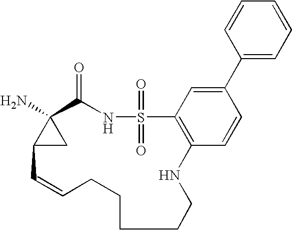 | |
| | 11 |
| 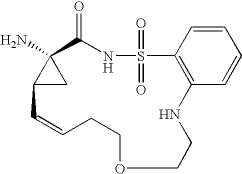 | |
| | 12 |
| 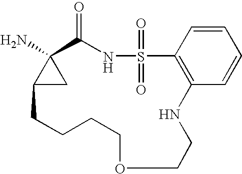 | |
| | 13 |
| 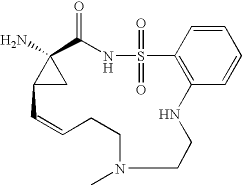 | |
| | 14 |
| 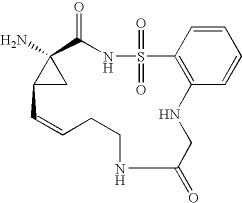 | |
| | 15 |
| 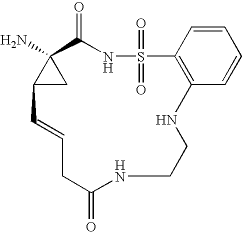 | |
| | 16 |
| 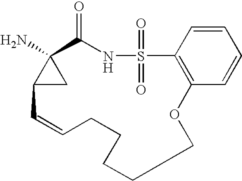 | |
| | 17 |
| 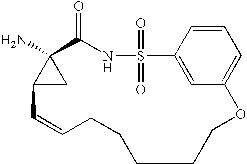 | |
| | 18 |
| 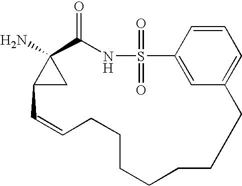 | |
| | 19 |
| 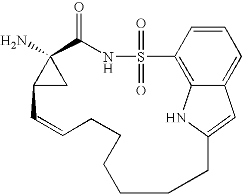 | |
| | 20 |
| 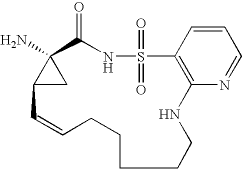 | |
| | 21 |
| 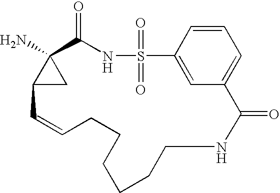 | |
| | 22 |
| 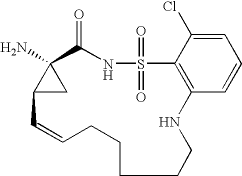 | |
| | 23 |
| 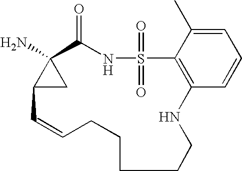 | |
| | 24 |
| 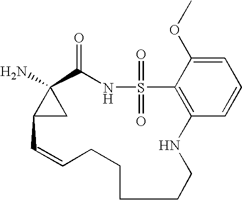 | |
| | 25 |
| 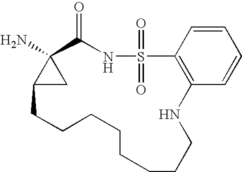 | |
| | 26 |
| 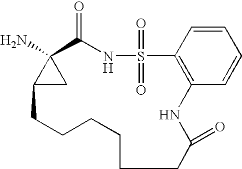 | |
| | 27 |
| 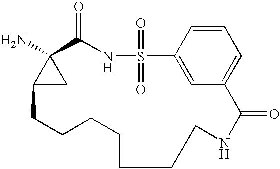 | |
| | 28 |
| 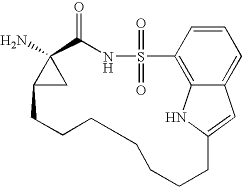 | |
| | 29 |
| 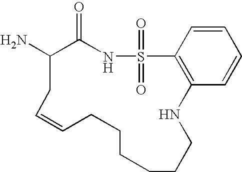 | |
| | 30 |
| 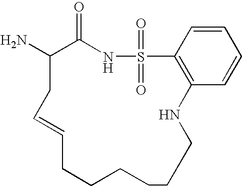 | |
| | 31 |
| 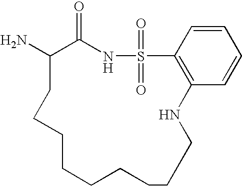 | |
| | 32 |
| 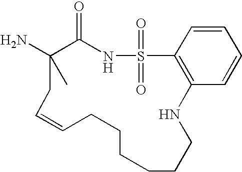 | |
| | 33 |
| 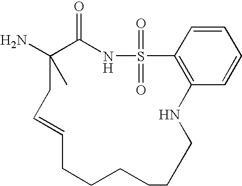 | |
| | 34 |
| 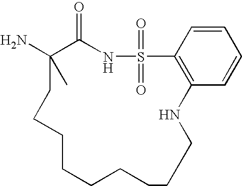 | |
| | 35 |
| 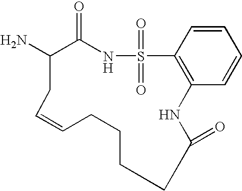 | |
| | 36 |
| 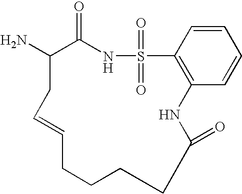 | |
| | 37 |
| 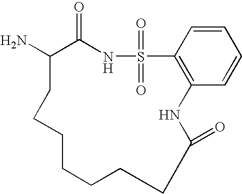 | |
| | 38 |
| 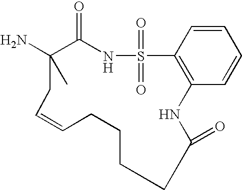 | |
| | 39 |
| 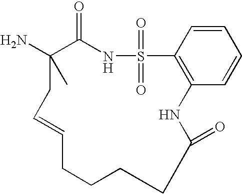 | |
| | 40 |
| 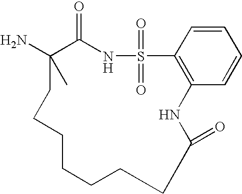 | |
| | 41 |
| 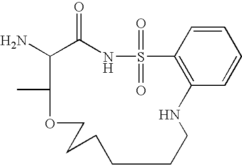 | |
| | 42 |
| 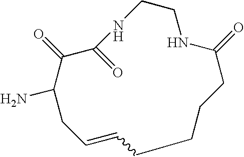 | |
| | 43 |
| 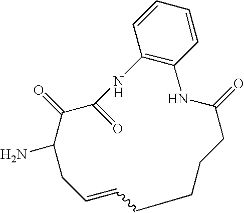 | |
| | 44 |
| 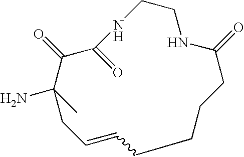 | |
| | 45 |
| 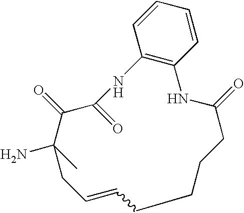 | |
| | 46 |
| 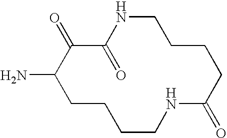 | |
| | 47 |
| 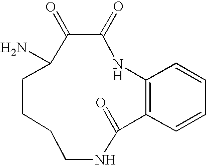 | |
| | 48 |
| 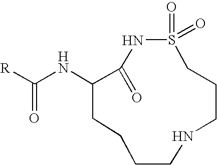 | |
| | 49 |
| 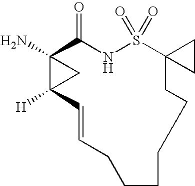 | |
| | 50 |
| 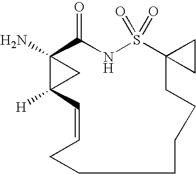 | |
| | 51 |
| 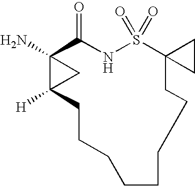 | |
| | 52 |
| 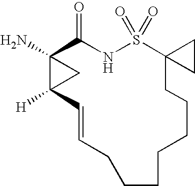 | |
| | 53 |
| 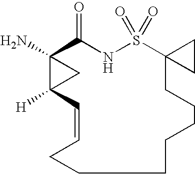 | |
| | 54 |
| 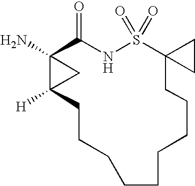 | |
| | 55 |
| 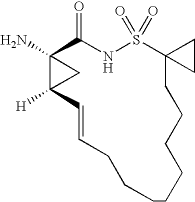 | |
| | 56 |
| 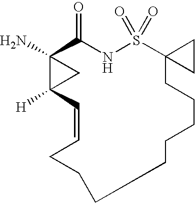 | |
| | 57 |
| 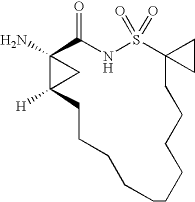 | |
| | 58 |
| 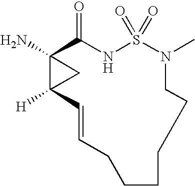 | |
| | 59 |
| 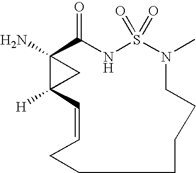 | |
| | 60 |
| 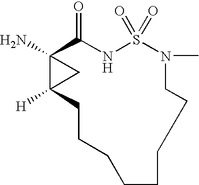 | |
| | 61 |
| 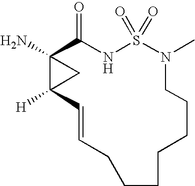 | |
| | 62 |
| 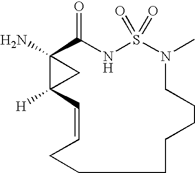 | |
| | 63 |
| 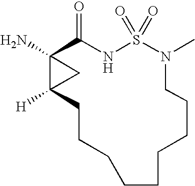 | |
| | 64 |
| 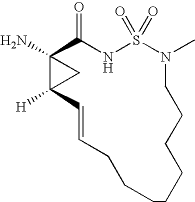 | |
| | 65 |
| 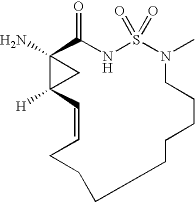 | |
| | 66 |
| 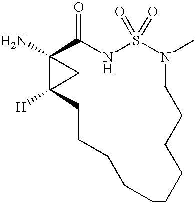 | |

TABLE D
(P2 Fragments)

| | |
|---|---|
| | 1 |
| 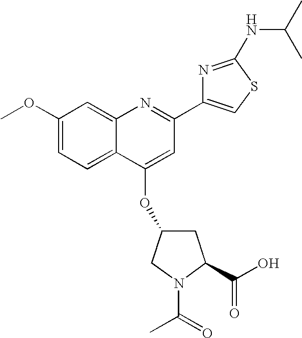 | |
| | 2 |
| 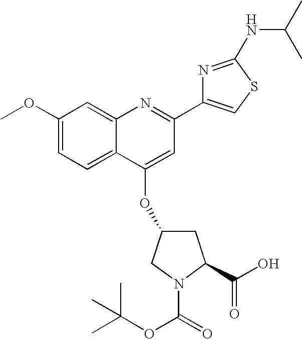 | |
| | 3 |
| 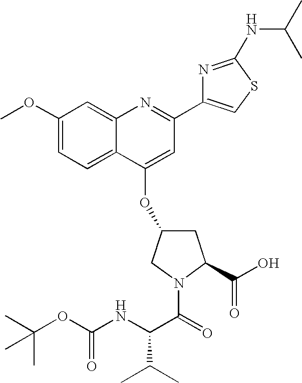 | |
| | 4 |
| 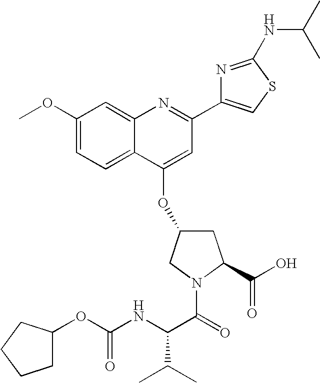 | |
| | 5 |
| 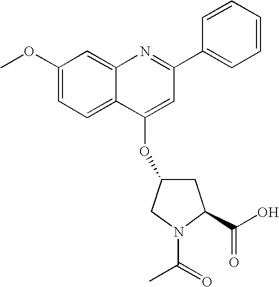 | |
| | 6 |
| 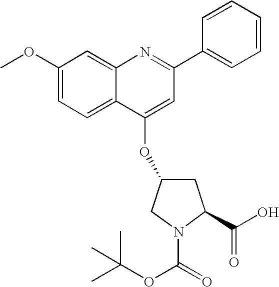 | |
| | 7 |
| 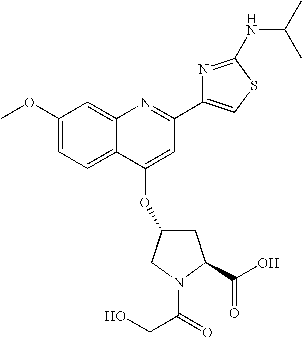 | |
| | 8 |
| 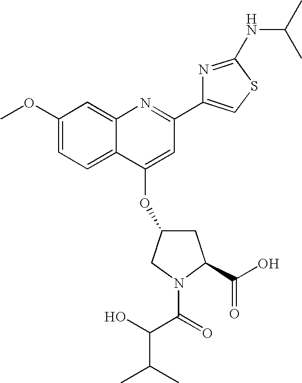 | |
| | 9 |
| 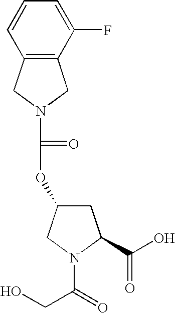 | |
| | 10 |
| 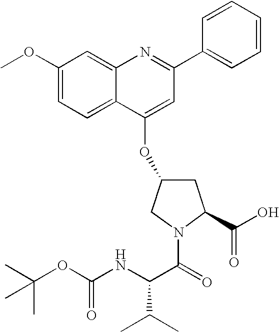 | |
| | 11 |
| 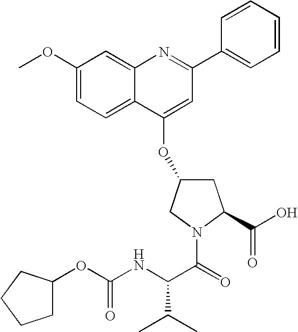 | |
| | 12 |
| 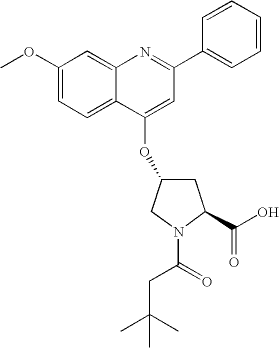 | |
| | 13 |
| 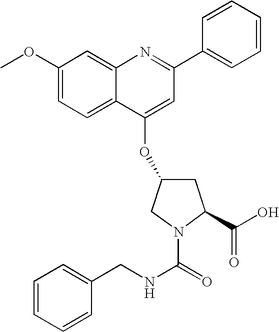 | |
| | 14 |
| 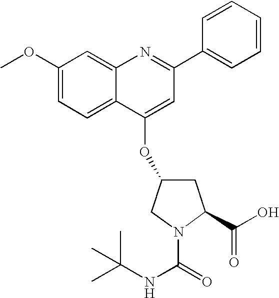 | |
| | 15 |
| 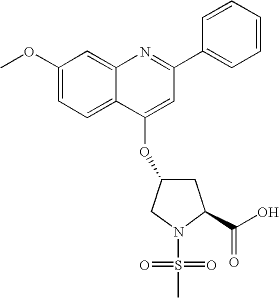 | |
| | 16 |
| 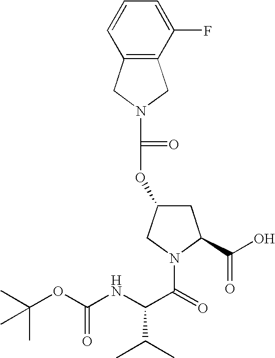 | |
| | 17 |
| 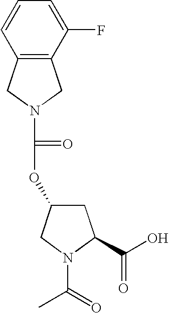 | |
| | 18 |
| 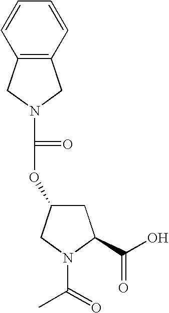 | |
| | 19 |
| 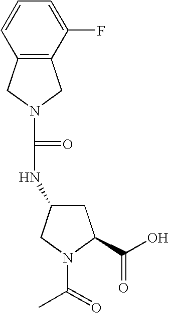 | |
| | 20 |
| 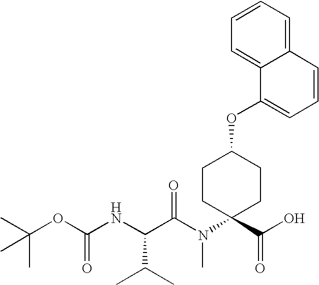 | |
| | 21 |
| 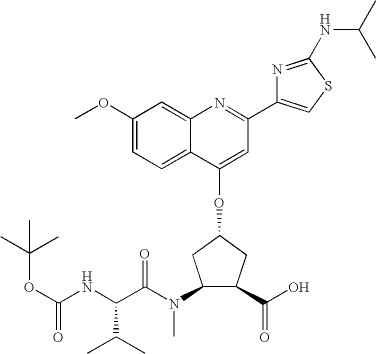 | |
| | 22 |
| 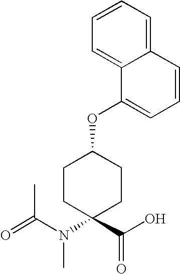 | |
| | 23 |
| 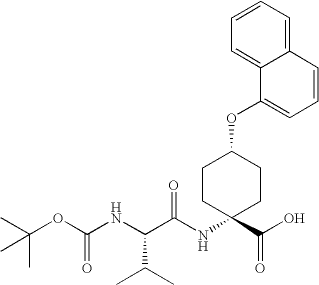 | |
| | 24 |
| 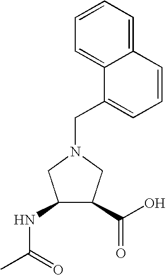 | |
| | 25 |
| 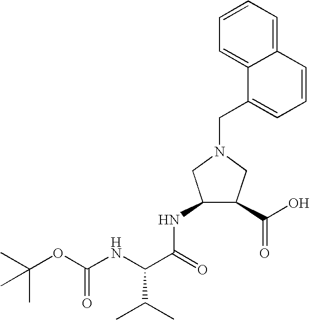 | |
| | 26 |
| 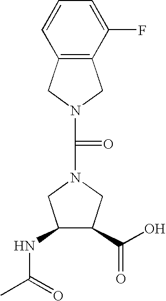 | |
| | 27 |
| 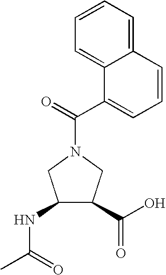 | |
| | 28 |
| 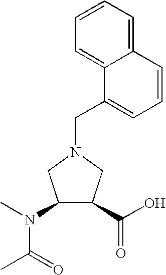 | |
| | 29 |
| 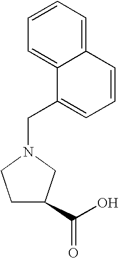 | |
| | 30 |
| 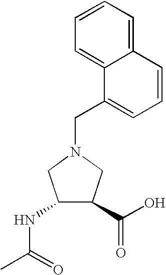 | |
| | 31 |
| 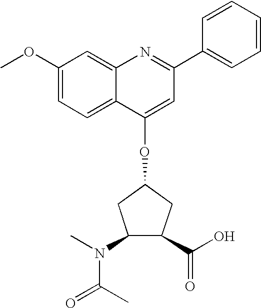 | |
| | 32 |
| 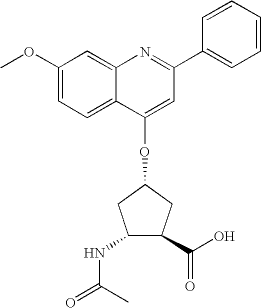 | |
| | 33 |
| 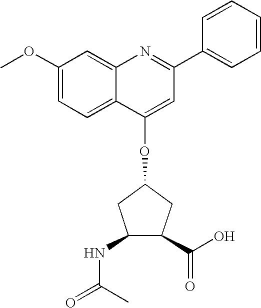 | |
| | 34 |
| 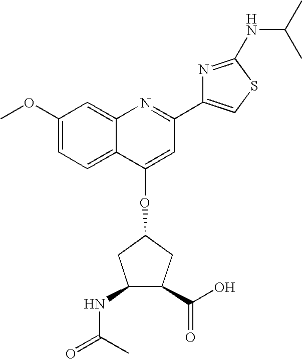 | |
| | 35 |
| 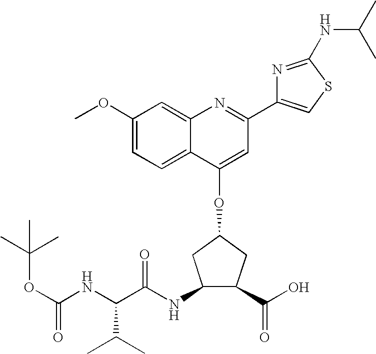 | |
| | 36 |
| 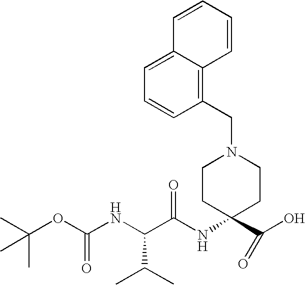 | |
| | 37 |
| 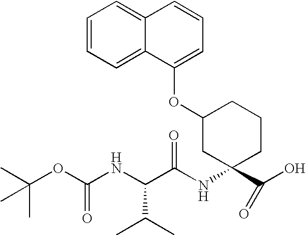 | |
| | 38 |
| 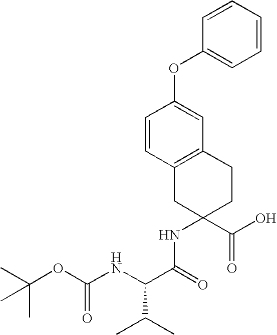 | |
| | 39 |
| 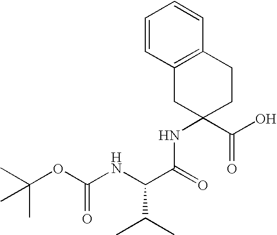 | |
| | 40 |
| 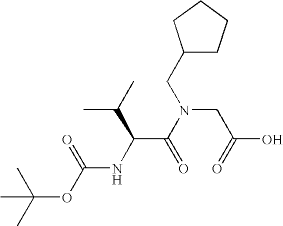 | |
| | 41 |
| 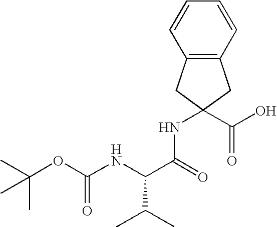 | |
| | 42 |
| 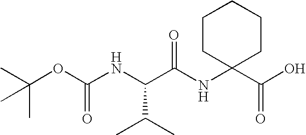 | |
| | 43 |
| 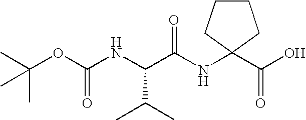 | |
| | 44 |
| 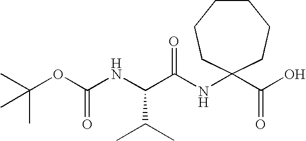 | |
| | 45 |
| 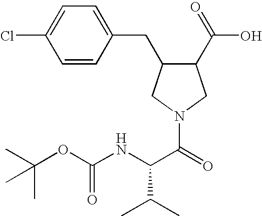 | |
| | 46 |
| 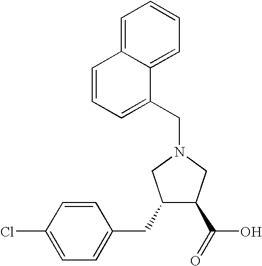 | |
| | 47 |
| 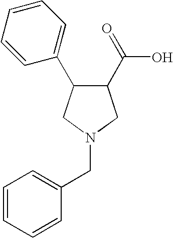 | |
| | 48 |
| 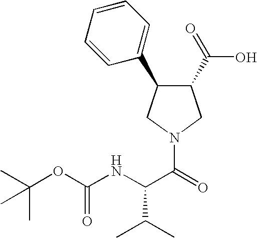 | |
| | 49 |
| 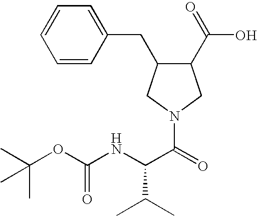 | |
| | 50 |
| 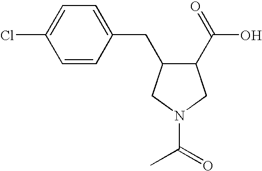 | |
| | 51 |
| 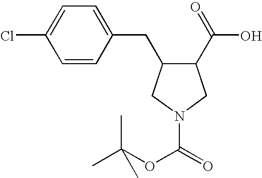 | |
| | 52 |
| 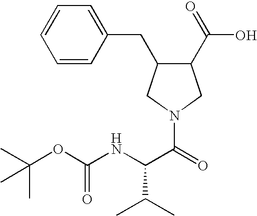 | |
| | 53 |
| 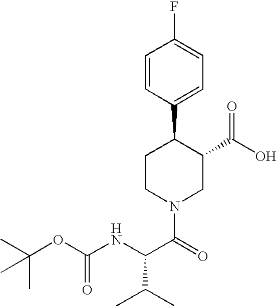 | |
| | 54 |
| 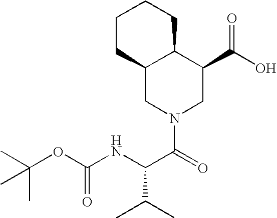 | |
| | 55 |
| 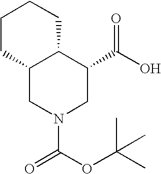 | |
| | 56 |
| 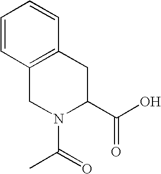 | |
| | 57 |
| 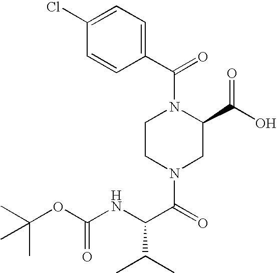 | |
| | 58 |
| 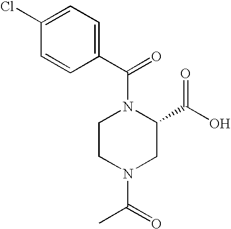 | |
| | 59 |
| 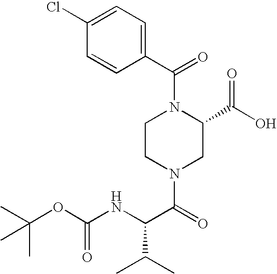 | |
| | 60 |
| 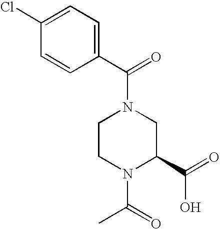 | |
| | 61 |
| 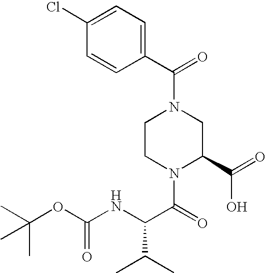 | |
| | 62 |
| 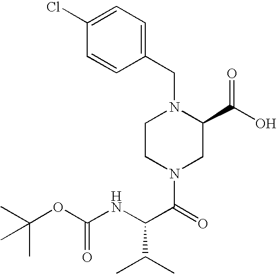 | |
| | 63 |
| 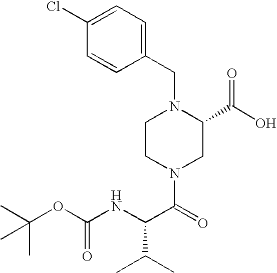 | |
| | 64 |
| 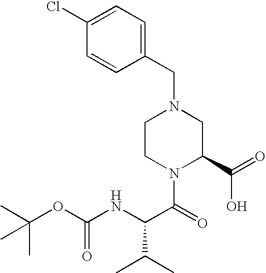 | |
| | 65 |
| 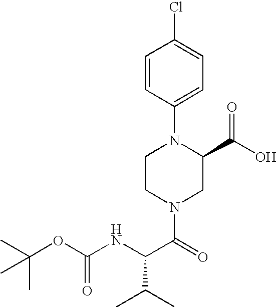 | |
| | 66 |
| 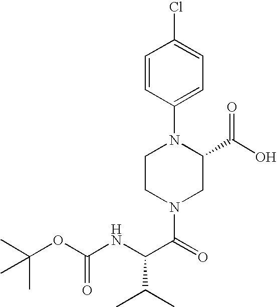 | |
| | 67 |
| 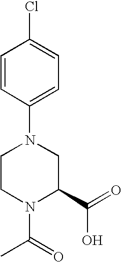 | |
| | 68 |
| 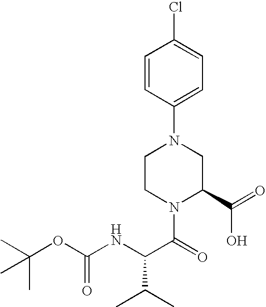 | |
| | 69 |
| 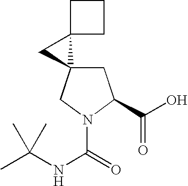 | |
| | 70 |
| 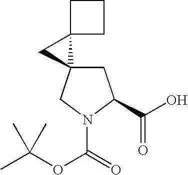 | |
| | 71 |
| 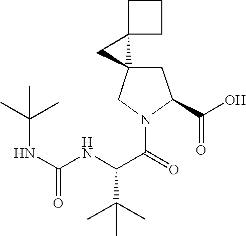 | |
| | 72 |
| 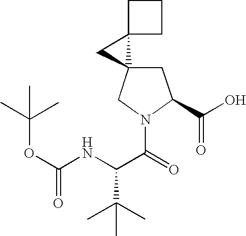 | |
| | 73 |
| 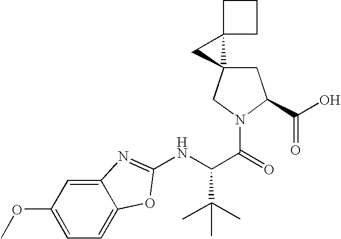 | |
| | 74 |
| 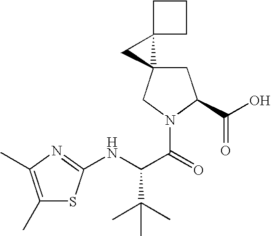 | |
| | 75 |
| 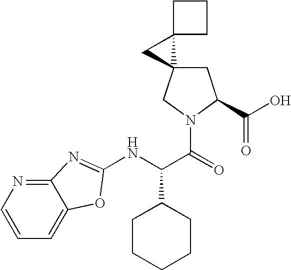 | |
| | 76 |
| 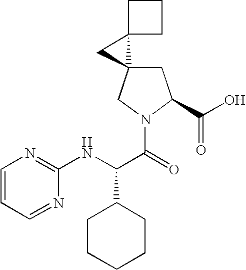 | |
| | 77 |
| 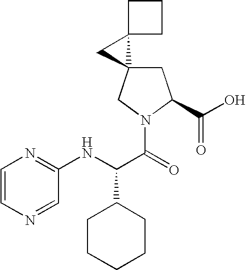 | |
| | 78 |
| 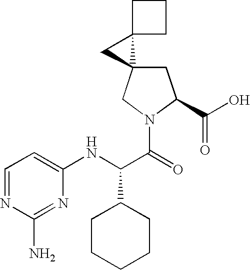 | |
| | 79 |
| 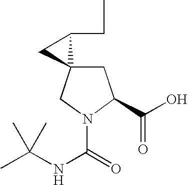 | |
| | 80 |
| 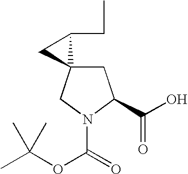 | |
| | 81 |
| 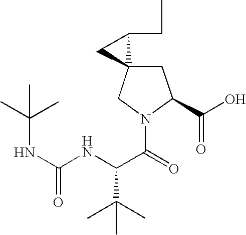 | |
| | 82 |
| 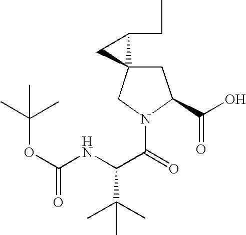 | |
| | 83 |
| 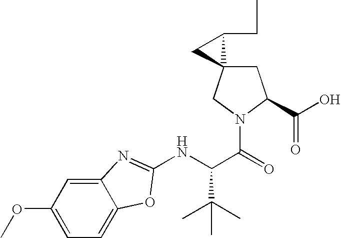 | |
| | 84 |
| 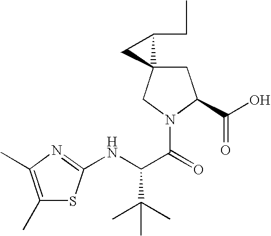 | |
| | 85 |
| 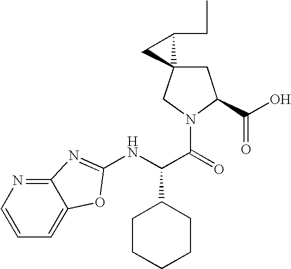 | |
| | 86 |
| 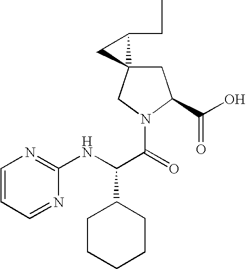 | |
| | 87 |
| 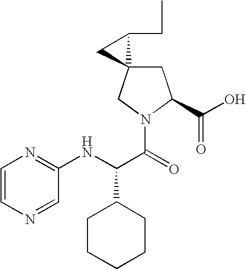 | |
| | 88 |
| 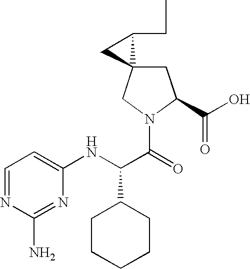 | |
| | 89 |
| 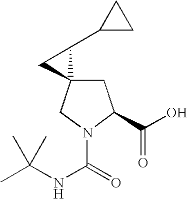 | |
| | 90 |
| 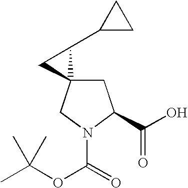 | |
| | 91 |
| 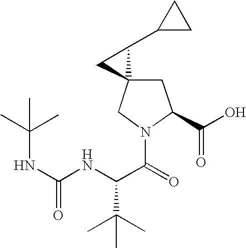 | |
| | 92 |
| 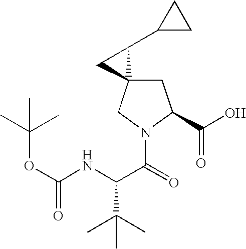 | |
| | 93 |
| 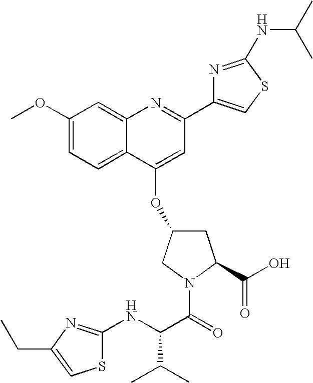 | |
| | 94 |
| 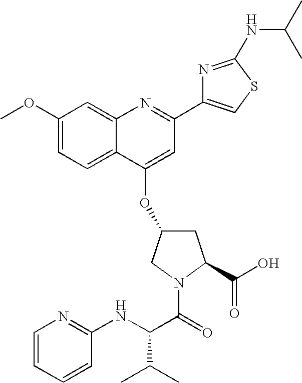 | |
| | 95 |
| 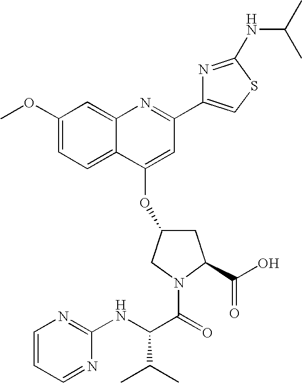 | |
| | 96 |
| 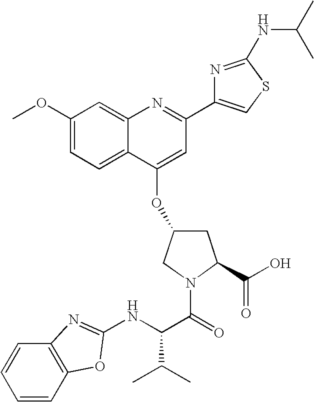 | |
| | 97 |
| 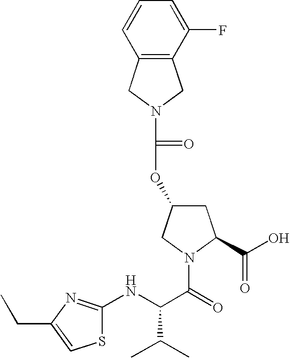 | |
| | 98 |
| 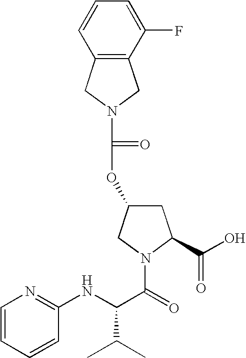 | |
| | 99 |
| 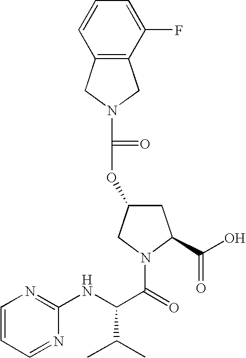 | |

| | |
|---|---|
| | 100 |
| 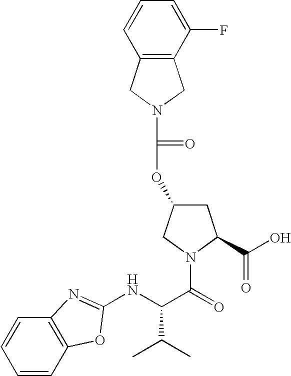 | |
| | 101 |
| 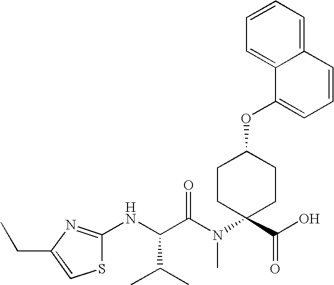 | |
| | 102 |
| 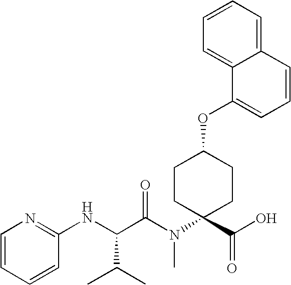 | |
| | 103 |
| 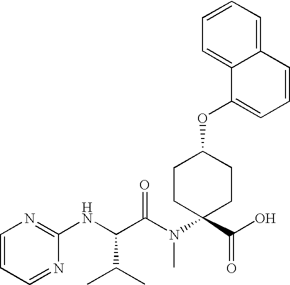 | |
| | 104 |
| 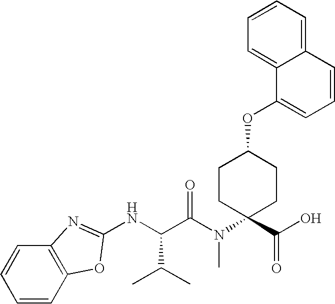 | |
| | 105 |
| 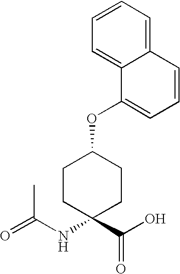 | |
| | 106 |
| 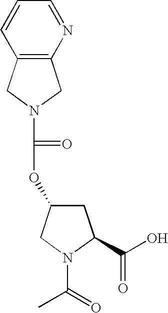 | |
| | 107 |
| 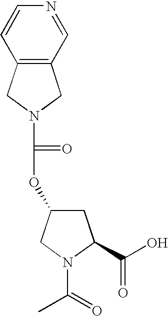 | |
| | 108 |
| 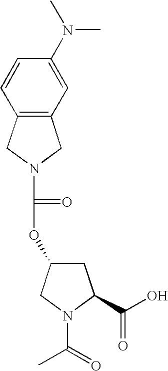 | |
| | 109 |
| 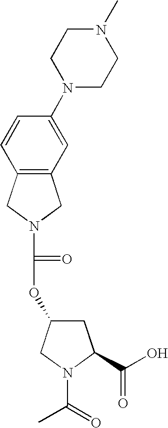 | |
| | 110 |
| 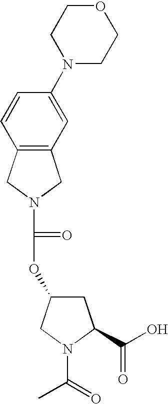 | |
| | 111 |
| 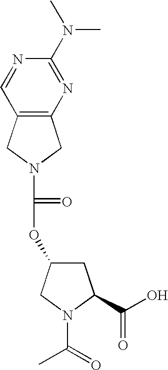 | |
| | 112 |
| 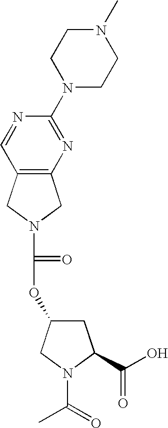 | |
| | 113 |
| 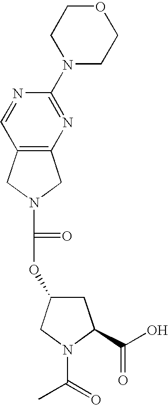 | |
| | 114 |
| 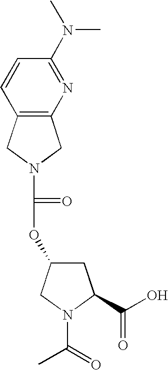 | |
| | 115 |
| 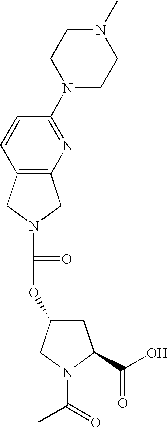 | |
| | 116 |
| 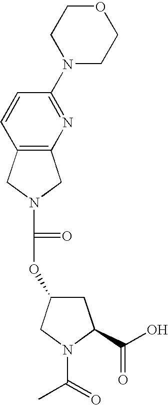 | |
| | 117 |
| 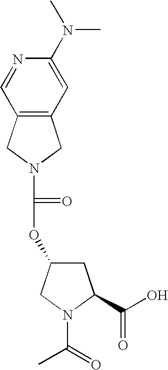 | |
| | 118 |
| 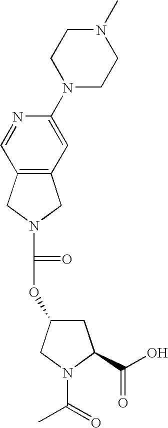 | |
| | 119 |
| 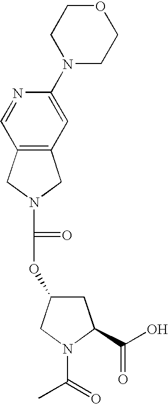 | |
| | 120 |
| 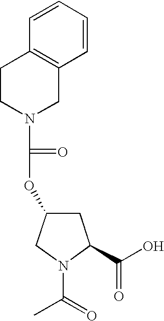 | |
| | 121 |
| 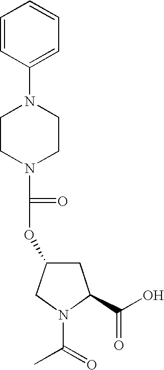 | |
| | 122 |
| 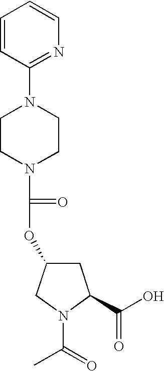 | |
| | 123 |
| 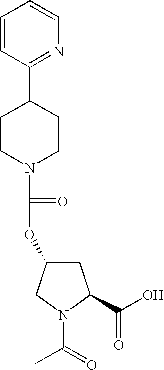 | |
| | 124 |
| 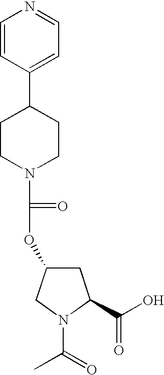 | |
| | 125 |
| 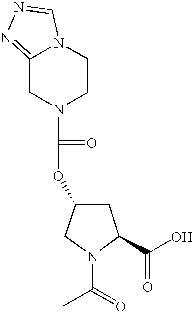 | |
| | 126 |
| 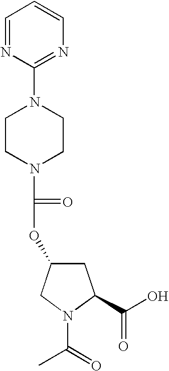 | |
| | 127 |
| 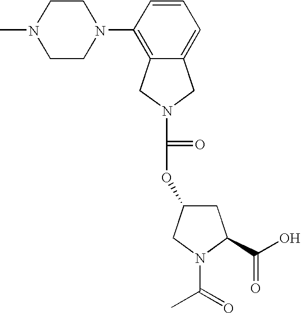 | |
| | 128 |
| 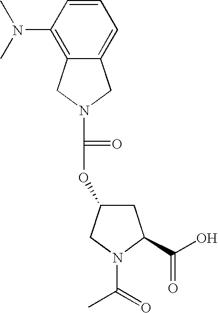 | |
| | 129 |
| 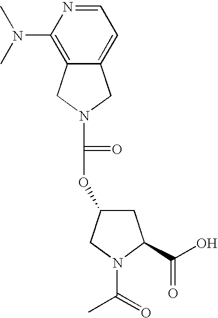 | |
| | 130 |
| 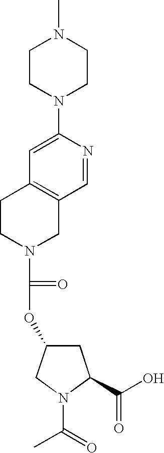 | |
| | 131 |
| 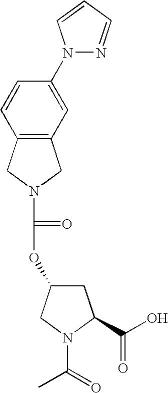 | |
| | 132 |
| 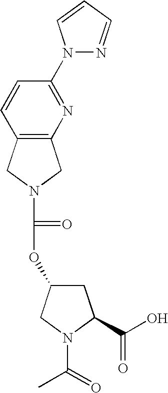 | |
| | 133 |
| 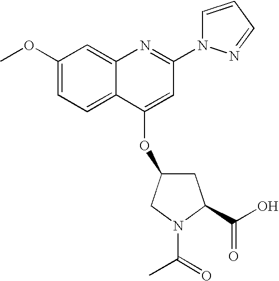 | |
| | 134 |
| 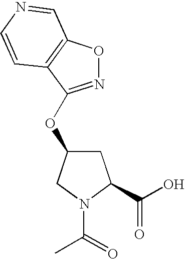 | |
| | 135 |
| 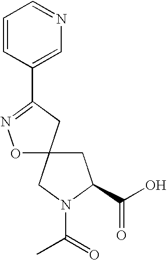 | |
| | 136 |
| 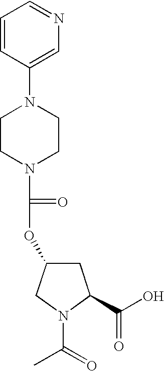 | |
| | 137 |
| 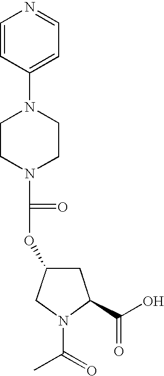 | |
| | 138 |
| 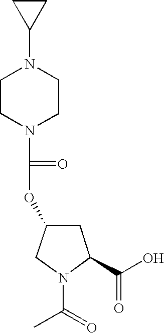 | |
| | 139 |
| 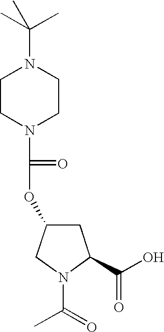 | |
| | 140 |
| 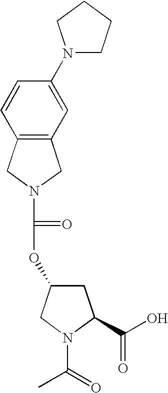 | |
| | 141 |
| 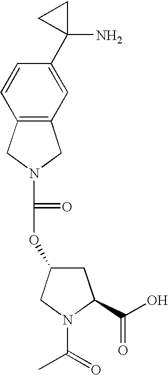 | |
| | 142 |
| 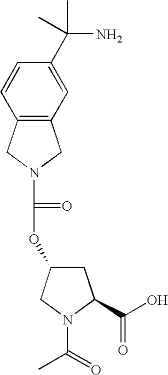 | |
| | 143 |
| 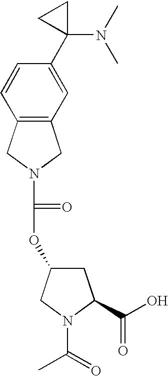 | |
| | 144 |
| 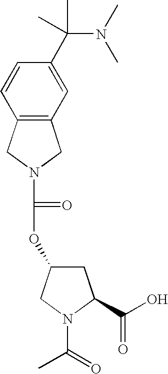 | |
| | 145 |
| 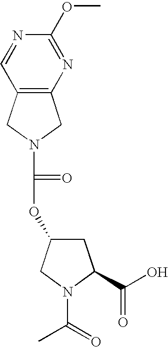 | |
| | 146 |
| 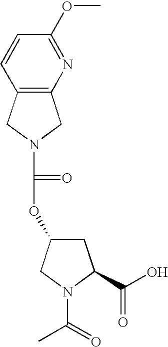 | |
| | 147 |
| 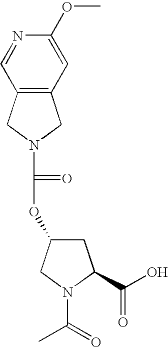 | |
| | 148 |
| 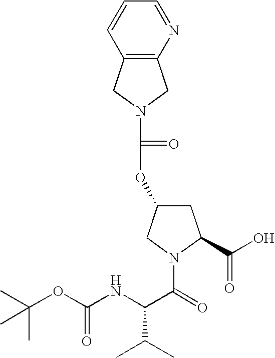 | |
| | 149 |
| 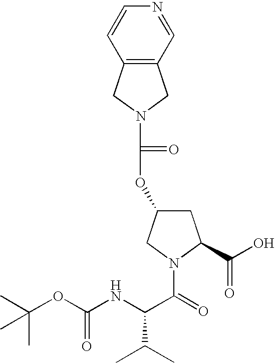 | |
| | 150 |
| 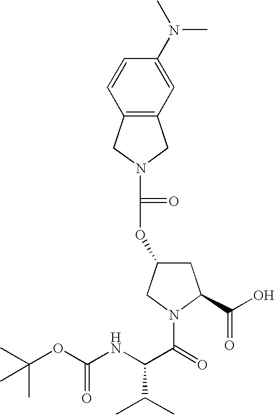 | |
| | 151 |
| 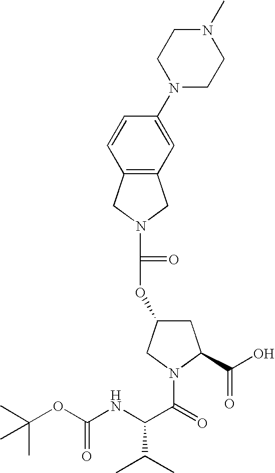 | |
| | 152 |
| 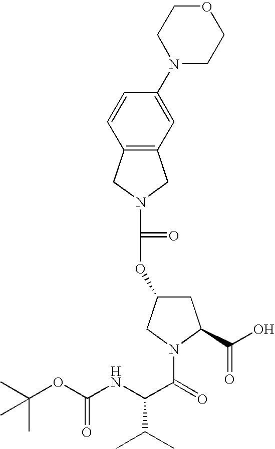 | |
| | 153 |
| 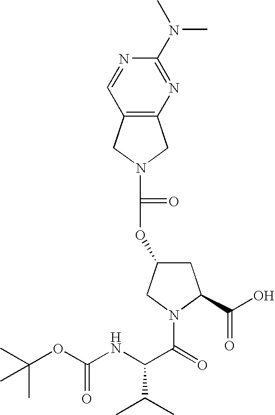 | |
| | 154 |
| 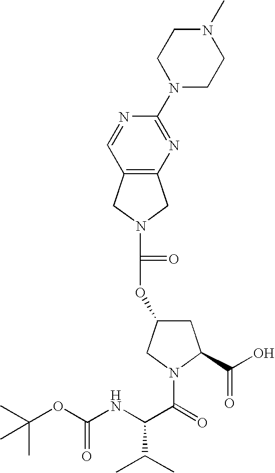 | |
| | 155 |
| 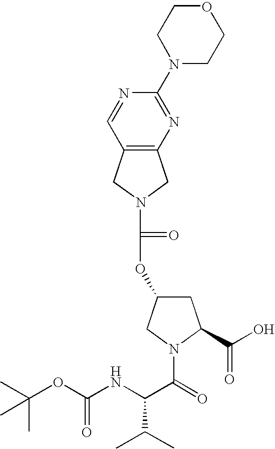 | |
| | 156 |
| 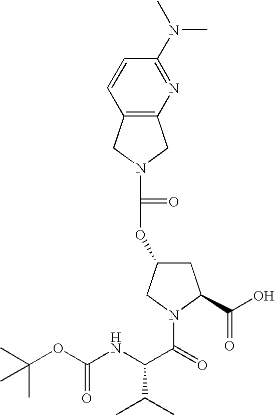 | |
| | 157 |
| 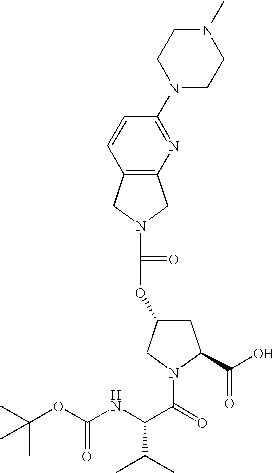 | |
| | 158 |
| 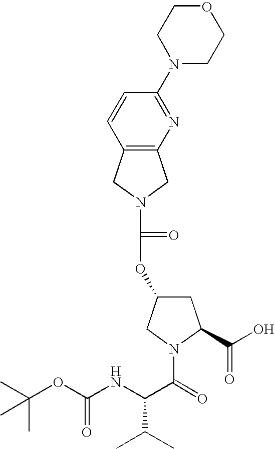 | |
| | 159 |
| 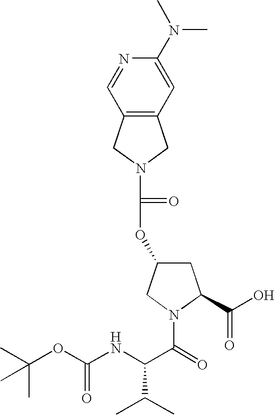 | |
| | 160 |
| 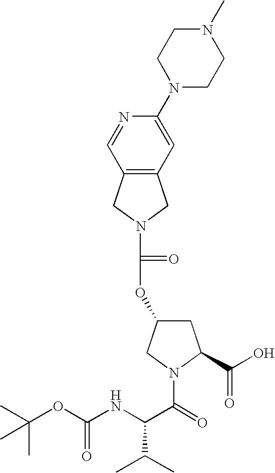 | |
| | 161 |
| 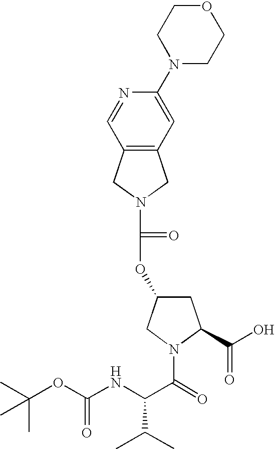 | |
| | 162 |
| 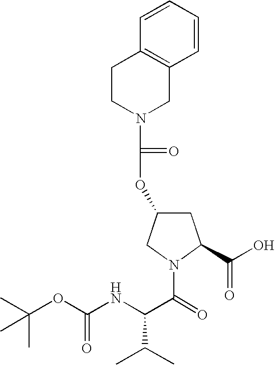 | |
| | 163 |
| 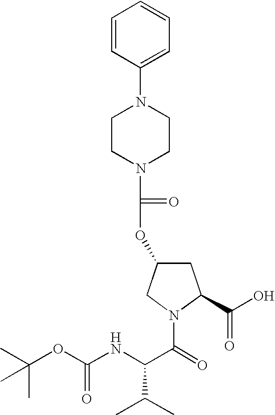 | |
| | 164 |
| 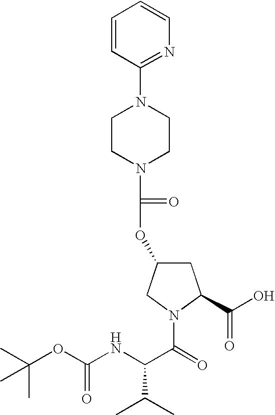 | |
| | 165 |
| 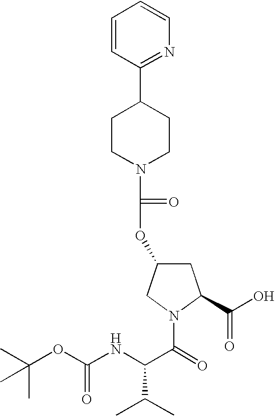 | |
| | 166 |
| 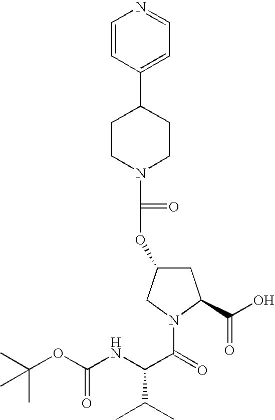 | |
| | 167 |
| 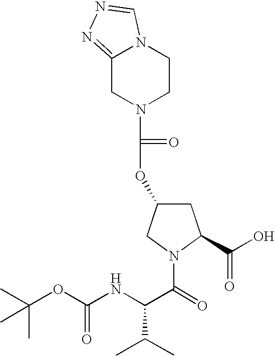 | |
| | 168 |
| 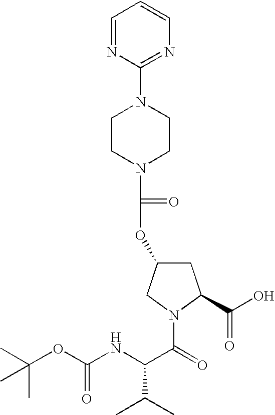 | |
| | 169 |
| 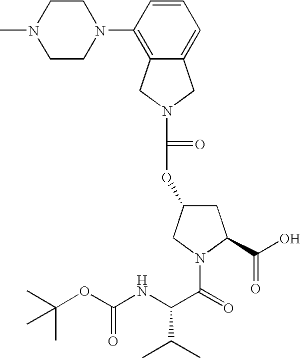 | |
| | 170 |
| 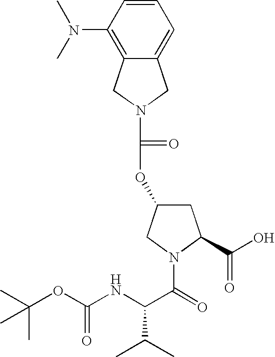 | |
| | 171 |
| 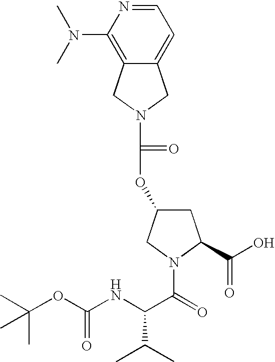 | |
| | 172 |
| 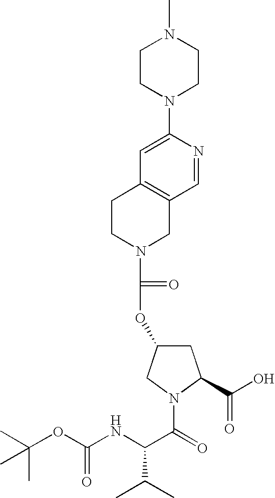 | |
| | 173 |
| 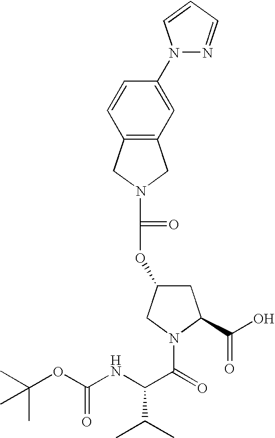 | |
| | 174 |
| 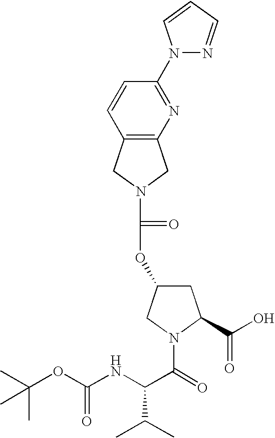 | |
| | 175 |
| 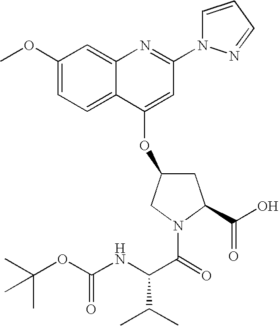 | |
| | 176 |
| 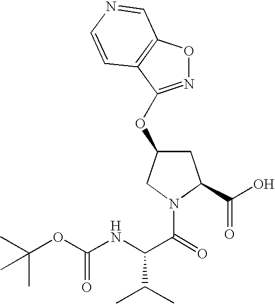 | |
| | 177 |
| 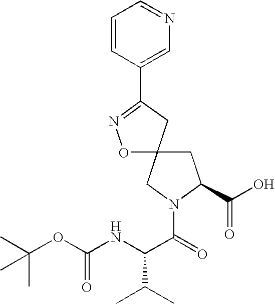 | |
| | 178 |
| 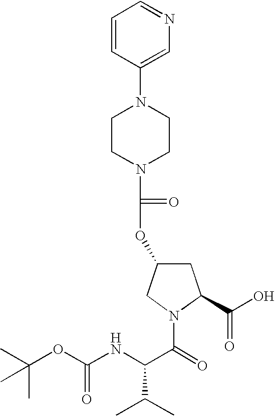 | |
| | 179 |
| 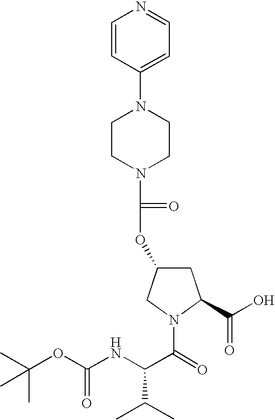 | |
| | 180 |
| 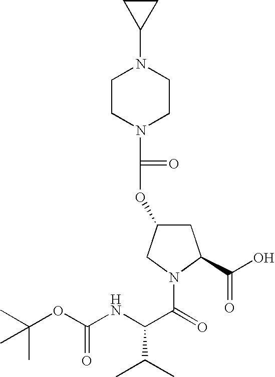 | |
| | 181 |
| 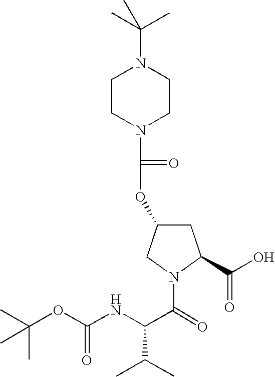 | |
| | 182 |
| 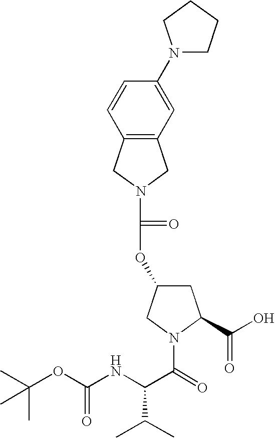 | |
| | 183 |
| 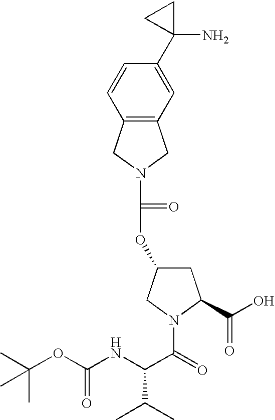 | |
| | 184 |
| 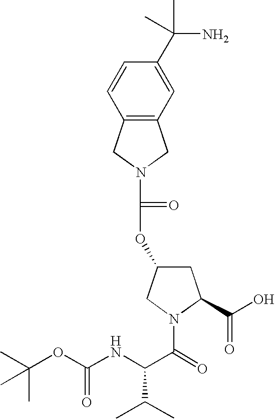 | |
| | 185 |
| 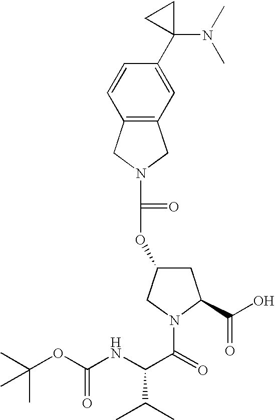 | |
| | 186 |
| 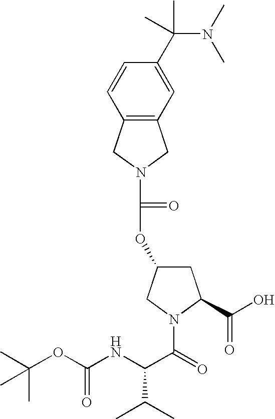 | |
| | 187 |
| 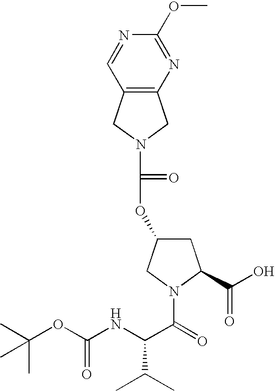 | |
| | 188 |
| 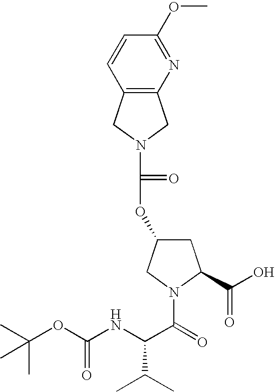 | |
| | 189 |
| 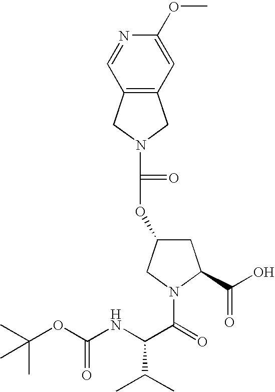 | |

Using the HCV NS3-4A protease and Luciferase-HCV replicon assays described in the exemplification section below, certain compounds of the invention (including compounds of Table A depicted above) are found to show IC₅₀ values for HCV inhibition in the range from 10 to more than 100 μM, or 0.5 to 30 μM, or show IC₅₀ values for HCV inhibition of less than 10 μM.

In certain embodiments, a compound of the present invention is further characterized as a modulator of HCV, including a mammalian HCV, and especially including a human HCV. In a preferred embodiment, the compound of the invention is an HCV inhibitor.

The terms “HCV-associated state” or “HCV-associated disorder” include disorders and states (e.g., a disease state) that are associated with the activity of HCV, e.g., infection of HCV in a subject. HCV-associated states include HCV-infection, liver cirrhosis, chronic liver disease, hepatocellular carcinoma, cryoglobulinaemia, non-Hodgkin's lymphoma, and a suppressed innate intracellular immune response.

HCV-associated states are often associated with the NS3 serine protease of HCV, which is responsible for several steps in the processing of the HCV polyprotein into smaller functional proteins. NS3 protease forms a heterodimeric complex with the NS4A protein, an essential cofactor that enhances enzymatic activity, and is believed to help anchor HCV to the endoplasmic reticulum. NS3 first autocatalyzes hydrolysis of the NS3-NS4A juncture, and then cleaves the HCV polyprotein intermolecularly at the NS4A-NS4B, NS4B-NS5A and NS5A-NS5B intersections. This process is associated with replication of HCV in a subject. Inhibiting or modulating the activity of one or more of the NS3, NS4A, NS4B, NS5A and NS5B proteins will inhibit or modulate replication of HCV in a subject, thereby preventing or treating the HCV-associated state. In a particular embodiment, the HCV-associated state is associated with the activity of the NS3 protease. In another particular embodiment, the HCV-associated state is associated with the activity of NS3-NS4A heterodimeric complex.

In one embodiment, the compounds of the invention are NS3/NS4A protease inhibitors. In another embodiment, the compounds of the invention are NS2/NS3 protease inhibitors.

Without being bound by theory, it is believed that the disruption of the above protein-protein interactions by the compounds of the invention will interfere with viral polyprotein processing by the NS3 protease and thus viral replication.

HCV-associated disorders also include HCV-dependent diseases. HVC-dependent diseases include, e.g., any disease or disorder that depend on or related to activity or misregulation of at least one strain of HCV.

The present invention includes treatment of HCV-associated disorders as described above, but the invention is not intended to be limited to the manner by which the compound performs its intended function of treatment of a disease. The present invention includes treatment of diseases described herein in any manner that allows treatment to occur, e.g., HCV infection.

In a related embodiment, the compounds of the invention can be useful for treating diseases related to HIV, as well as HIV infection and AIDS (Acquired Immune Deficiency Syndrome).

In certain embodiments, the invention provides a pharmaceutical composition of any of the compounds of the present invention. In a related embodiment, the invention provides a pharmaceutical composition of any of the compounds of the present invention and a pharmaceutically acceptable carrier or excipient of any of these compounds. In certain embodiments, the invention includes the compounds as novel chemical entities.

In one embodiment, the invention includes a packaged HCV-associated disorder treatment. The packaged treatment includes a compound of the invention packaged with instructions for using an effective amount of the compound of the invention for an intended use.

The compounds of the present invention are suitable as active agents in pharmaceutical compositions that are efficacious particularly for treating HCV-associated disorders. The pharmaceutical composition in various embodiments has a pharmaceutically effective amount of the present active agent along with other pharmaceutically acceptable excipients, carriers, fillers, diluents and the like. The phrase, “pharmaceutically effective amount” as used herein indicates an amount necessary to administer to a host, or to a cell, issue, or organ of a host, to achieve a therapeutic result, especially an anti-HCV effect, e.g., inhibition of proliferation of the HCV virus, or of any other HCV-associated disease.

In one embodiment, the diseases to be treated by compounds of the invention include, for example, HCV infection, liver cirrhosis, chronic liver disease, hepatocellular carcinoma, cryoglobulinaemia, non-Hodgkin's lymphoma, and a suppressed innate intracellular immune response.

In other embodiments, the present invention provides a method for inhibiting the activity of HCV. The method includes contacting a cell with any of the compounds of the present invention. In a related embodiment, the method further provides that the compound is present in an amount effective to selectively inhibit the activity of one or more of the NS3, NS4A, NS4B, NS5A and NS5B proteins. In another related embodiment, the method provides that the compound is present in an amount effective to diminish the HCV RNA load in a subject.

In other embodiments, the present invention provides a use of any of the compounds of the invention for manufacture of a medicament to treat HCV infection in a subject.

In other embodiments, the invention provides a method of manufacture of a medicament, including formulating any of the compounds of the present invention for treatment of a subject.

### DEFINITIONS

The term “treat,” “treated,” “treating” or “treatment” includes the diminishment or alleviation of at least one symptom associated or caused by the state, disorder or disease being treated. In certain embodiments, the treatment comprises the induction of an HCV-inhibited state, followed by the activation of the HCV-modulating compound, which would in turn diminish or alleviate at least one symptom associated or caused by the HCV-associated state, disorder or disease being treated. For example, treatment can be diminishment of one or several symptoms of a disorder or complete eradication of a disorder.

The term “subject” is intended to include organisms, e.g., prokaryotes and eukaryotes, which are capable of suffering from or afflicted with an HCV-associated disorder. Examples of subjects include mammals, e.g., humans, dogs, cows, horses, pigs, sheep, goats, cats, mice, rabbits, rats, and transgenic non-human animals. In certain embodiments, the subject is a human, e.g., a human suffering from, at risk of suffering from, or potentially capable of suffering from an HCV-associated disorder, and for diseases or conditions described herein, e.g., HCV infection. In another embodiment, the subject is a cell.

The language “HCV-modulating compound,” “modulator of HCV” or “HCV inhibitor” refers to compounds that modulate, e.g., inhibit, or otherwise alter, the activity of HCV. Similarly, an “NS3/NS4A protease inhibitor,” or an “NS2/NS3 protease inhibitor” refers to a compound that modulates, e.g., inhibits, or otherwise alters, the interaction of these proteases with one another. Examples of HCV-modulating compounds include compounds of Formula I, as well as Table A and Table B (including pharmaceutically acceptable salts thereof, as well as enantiomers, stereoisomers, rotamers, tautomers, diastereomers, or racemates thereof).

Additionally, the method includes administering to a subject an effective amount of an HCV-modulating compound of the invention, e.g., HCV-modulating compounds of Formula I, as well as Table A and Table B (including pharmaceutically acceptable salts thereof, as well as enantiomers, stereoisomers, rotamers, tautomers, diastereomers, or racemates thereof).

The term “alkyl” includes saturated aliphatic groups, including straight-chain alkyl groups (e.g., methyl, ethyl, propyl, butyl, pentyl, hexyl, heptyl, octyl, nonyl, decyl, etc.), branched-chain alkyl groups (isopropyl, tert-butyl, isobutyl, etc.), cycloalkyl (alicyclic) groups (cyclopropyl, cyclopentyl, cyclohexyl, cycloheptyl, cyclooctyl), alkyl substituted cycloalkyl groups, and cycloalkyl substituted alkyl groups. The term “alkyl” also includes alkenyl groups and alkynyl groups. Furthermore, the expression “Cₓ-C_{y}alkyl”, wherein x is 1-5 and y is 2-10 indicates a particular alkyl group (straight- or branched-chain) of a particular range of carbons. For example, the expression C₁-C₄-alkyl includes, but is not limited to, methyl, ethyl, propyl, butyl, isopropyl, tert-butyl, isobutyl and sec-butyl. Moreover, the term C₃₋₆-cycloalkyl includes, but is not limited to, cyclopropyl, cyclopentyl, and cyclohexyl. As discussed below, these alkyl groups, as well as cycloalkyl groups, may be further substituted. “C₀-Cₙalkyl” refers to a single covalent bond (C₀) or an alkyl group having from 1 to n carbon atoms; for example “C₀-C₄alkyl” refers to a single covalent bond or a C₁-C₄alkyl group; “C₀-C₈alkyl” refers to a single covalent bond or a C₁-C₈alkyl group. In some instances, a substituent of an alkyl group is specifically indicated. For example, “C₁-C₄hydroxyalkyl” refers to a C₁-C₄alkyl group that has at least one hydroxy substituent.

“Alkylene” refers to a divalent alkyl group, as defined above. C₀-C₄alkylene is a single covalent bond or an alkylene group having from 1 to 4 carbon atoms; and C₀-C₆alkylene is a single covalent bond or an alkylene group having from 1 to 6 carbon atoms. “Alkenylene” and “Alkynylene” refer to divalent alkenyl and alkynyl groups respectively, as defined above.

The term alkyl further includes alkyl groups which can further include oxygen, nitrogen, sulfur or phosphorous atoms replacing one or more carbons of the hydrocarbon backbone. In an embodiment, a straight chain or branched chain alkyl has 10 or fewer carbon atoms in its backbone (e.g., C₁-C₁₀ for straight chain, C₃-C₁₀ for branched chain), and more preferably 6 or fewer carbons.

A “cycloalkyl” is a group that comprises one or more saturated and/or partially saturated rings in which all ring members are carbon, such as cyclopropyl, cyclobutyl, cyclopentyl, cyclohexyl, cycloheptyl, cyclooctyl, adamantyl, decahydro-naphthalenyl, octahydro-indenyl, and partially saturated variants of the foregoing, such as cyclohexenyl. Cycloalkyl groups do not comprise an aromatic ring or a heterocyclic ring. Certain cycloalkyl groups are C₃-C₈cycloalkyl, in which the group contains a single ring with from 3to 8 ring members. A “(C₃-C₈cycloalkyl)C₀-C₄alkyl” is a C₃-C₈cycloalkyl group linked via a single covalent bond or a C₁-C₄alkylene group. In certain aspects, C₃-C₆-cycloalkyl groups are substituted one or more times with substitutents independently selected from a halogen atom, aryl, heteroaryl, trihalomethyl, C₁₋₄alkoxy or C₁₋₄alkyl.

Moreover, alkyl (e.g., methyl, ethyl, propyl, butyl, pentyl, hexyl, etc.) include both “unsubstituted alkyl” and “substituted alkyl”, the latter of which refers to alkyl moieties having substituents replacing a hydrogen on one or more carbons of the hydrocarbon backbone, which allow the molecule to perform its intended function.

The term “substituted” is intended to describe moieties having substituents replacing a hydrogen on one or more atoms, e.g. C, O or N, of a molecule. Such substituents can include, for example, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, morpholino, phenol, benzyl, phenyl, piperazine, cyclopentane, cyclohexane, pyridine, 5H-tetrazole, triazole, piperidine, or an aromatic or heteroaromatic moiety.

Further examples of substituents of the invention, which are not intended to be limiting, include moieties selected from straight or branched alkyl (preferably C₁-C₅), cycloalkyl (preferably C₃-C₈), alkoxy (preferably C₁-C₆), thioalkyl (preferably C₁-C₆), alkenyl (preferably C₂-C₆), alkynyl (preferably C₂-C₆), heterocyclic, carbocyclic, aryl (e.g., phenyl), aryloxy (e.g., phenoxy), aralkyl (e.g., benzyl), aryloxyalkyl. (e.g., phenyloxyalkyl), arylacetamidoyl, alkylaryl, heteroaralkyl, alkylcarbonyl and arylcarbonyl of other such acyl group, heteroarylcarbonyl, or heteroaryl group, (CR′R″)₀₋₃NR′R″ (e.g., —NH₂), (CR′R″)₀₋₃CN (e.g., —CN), —NO₂, halogen (e.g., —F, —Cl, —Br, or —I), (CR′R″)₀₋₃C(halogen)₃ (e.g., —CF₃), (CR′R″)₀₋₃CH(halogen)₂, (CR′R″)₀₋₃CH₂(halogen), (CR′R″)₀₋₃CONR′R″, (CR′R″)₀₋₃(CNH)NR′R″, (CR′R″)₀₋₃S(O)₁₋₂NR′R″, (CR′R″)₀₋₃CHO, (CR′R″)₀₋₃O(CR′R″)₀₋₃H, (CR′R″)₀₋₃S(O)₀₋₃R′ (e.g., —SO₃H, —OSO₃H), (CR′R″)₀₋₃O(CR′R″)₀₋₃H (e.g., —CH₂OCH₃ and —OCH₃), (CR′R″)₀₋₃S(CR′R″)₀₋₃H (e.g., —SH and —SCH₃), (CR′R″)₀₋₃OH (e.g., —OH), (CR′R″)₀₋₃COR′, (CR′R″)₀₋₃ (substituted or unsubstituted phenyl), (CR′R″)₀₋₃(C₃-C₈ cycloalkyl), (CR′R″)₀₋₃CO₂R′ (e.g., —CO₂H), or (CR′R″)₀₋₃OR′ group, or the side chain of any naturally occurring amino acid; wherein R′ and R″ are each independently hydrogen, a C₁-C₅ alkyl, C₂-C₅ alkenyl, C₂-C₅ alkynyl, or aryl group. Such substituents can include, for example, halogen, hydroxyl, alkylcarbonyloxy, arylcarbonyloxy, alkoxycarbonyloxy, aryloxycarbonyloxy, carboxylate, alkylcarbonyl, alkoxycarbonyl, aminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, oxime, sulfhydryl, alkylthio, arylthio, thiocarboxylate, sulfates, sulfonate, sulfamoyl, sulfonamido, nitro, trifluoromethyl, cyano, azido, heterocyclyl, or an aromatic or heteroaromatic moiety. In certain embodiments, a carbonyl moiety (C═O) may be further derivatized with an oxime moiety, e.g., an aldehyde moiety may be derivatized as its oxime (—C═N—OH) analog. It will be understood by those skilled in the art that the moieties substituted on the hydrocarbon chain can themselves be substituted, if appropriate. Cycloalkyls can be further substituted, e.g., with the substituents described above. An “aralkyl” moiety is an alkyl substituted with an aryl (e.g., phenylmethyl (i.e., benzyl)).

The term “alkenyl” includes unsaturated aliphatic groups analogous in length and possible substitution to the alkyls described above, but which contain at least one double bond.

For example, the term “alkenyl” includes straight-chain alkenyl groups (e.g., ethenyl, propenyl, butenyl, pentenyl, hexenyl, heptenyl, octenyl, nonenyl, decenyl, etc.), branched-chain alkenyl groups, cycloalkenyl (alicyclic) groups (cyclopropenyl, cyclopentenyl, cyclohexenyl, cyclohepfenyl, cyclooctenyl), alkyl or alkenyl substituted cycloalkenyl groups, and cycloalkyl or cycloalkenyl substituted alkenyl groups. The term alkenyl further includes alkenyl groups that include oxygen, nitrogen, sulfur or phosphorous atoms replacing one or more carbons of the hydrocarbon backbone. In certain embodiments, a straight chain or branched chain alkenyl group has 6 or fewer carbon atoms in its backbone (e.g., C₂-C₆ for straight chain, C₃-C₆ for branched chain). Likewise, cycloalkenyl groups may have from 3-8carbon atoms in their ring structure, and more preferably have 5 or 6 carbons in the ring structure. The term C₂-C₆ includes alkenyl groups containing 2 to 6 carbon atoms.

Moreover, the term alkenyl includes both “unsubstituted alkenyls” and “substituted alkenyls”, the latter of which refers to alkenyl moieties having substituents replacing a hydrogen on one or more carbons of the hydrocarbon backbone. Such substituents can include, for example, alkyl groups, alkynyl groups, halogens, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbortylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

The term “alkynyl” includes unsaturated aliphatic groups analogous in length and possible substitution to the alkyls described above, but which contain at least one triple bond.

For example, the term “alkynyl” includes straight-chain alkynyl groups (e.g., ethynyl, propynyl, butynyl, pentynyl, hexynyl, heptynyl, octynyl, nonynyl, decynyl, etc.), branched-chain alkynyl groups, and cycloalkyl or cycloalkenyl substituted alkynyl groups. The term alkynyl further includes alkynyl groups that include oxygen, nitrogen, sulfur or phosphorous atoms replacing one or more carbons of the hydrocarbon backbone. In certain embodiments, a straight chain or branched chain alkynyl group has 6 or fewer carbon atoms in its backbone (e.g., C₂-C₆ for straight chain, C₃-C₆ for branched chain). The term C₂-C₆ includes alkynyl groups containing 2 to 6 carbon atoms.

Moreover, the term alkynyl includes both “unsubstituted alkynyls” and “substituted alkynyls”, the latter of which refers to alkynyl moieties having substituents replacing a hydrogen on one or more carbons of the hydrocarbon backbone. Such substituents can include, for example, alkyl groups, alkynyl groups, halogens, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamide, nitro, trifluoromethyl, cyano, azido, heterocyclyl, alkylaryl, or an aromatic or heteroaromatic moiety.

The term “amine” or “amino” should be understood as being broadly applied to both a molecule, or a moiety or functional group, as generally understood in the art, and may be primary, secondary, or tertiary. The term “amine” or “amino” includes compounds where a nitrogen atom is covalently bonded to at least one carbon, hydrogen or heteroatom. The terms include, for example, but are not limited to, “alkylamino,” “arylamino,” “diarylamino,” “alkylarylamino,” “alkylaminoaryl,” “arylaminoalkyl,” “alkaminoalkyl,” “amide,” “amido,” and “aminocarbonyl.” The term “alkyl amino” comprises groups and compounds wherein the nitrogen is bound to at least one additional alkyl group. The term “dialkyl amino” includes groups wherein the nitrogen atom is bound to at least two additional alkyl groups. The term “arylamino” and “diarylamino” include groups wherein the nitrogen is bound to at least one or two aryl groups, respectively. The term “alkylarylamino,” “alkylaminoaryl” or “arylaminoalkyl” refers to an amino group which is bound to at least one alkyl group and at least one aryl group. The term “alkaminoalkyl” refers to an alkyl, alkenyl, or alkynyl group bound to a nitrogen atom which is also bound to an alkyl group.

The term “amide,” “amido” or “aminocarbonyl” includes compounds or moieties which contain a nitrogen atom which is bound to the carbon of a carbonyl or a thiocarbonyl group. The term includes “alkaminocarbonyl” or “alkylaminocarbonyl” groups which include alkyl, alkenyl, aryl or alkynyl groups bound to an amino group bound to a carbonyl group. It includes arylaminocarbonyl and arylcarbonylamino groups which include aryl or heteroaryl moieties bound to an amino group which is bound to the carbon of a carbonyl or thiocarbonyl group. The terms “alkylaminocarbonyl,” “alkenylaminocarbonyl,” “alkynylaminocarbonyl,” “arylaminocarbonyl,” “alkylcarbonylamino,” “alkenylcarbonylamino,” “alkynylcarbonylamino,” and “arylcarbonylamino” are included in term “amide.” Amides also include urea groups (aminocarbonylamino) and carbamates (oxycarbonylamino).

The term “aryl” includes groups, including 5- and 6-membered single-ring aromatic groups that may include from zero to four heteroatoms, for example, phenyl, pyrrole, furan, thiophene, thiazole, isothiazole, imidazole, triazole, tetrazole, pyrazole, oxazole, isoxazole, pyridine, pyrazine, pyridazine, and pyrimidine, and the like. Furthermore, the term “aryl” includes multicyclic aryl groups, e.g., tricyclic, bicyclic, e.g., naphthalene, benzoxazole, benzodioxazole, benzothiazole, benzoimidazole, benzothiophene, methylenedioxyphenyl, quinoline, isoquinoline, anthryl, phenanthryl, napthridine, indole, benzofuran, purine, benzofuran, deazapurine, or indolizine. Those aryl groups having heteroatoms in the ring structure may also be referred to as “aryl heterocycles”, “heterocycles,” “heteroaryls” or “heteroaromatics.” The aromatic ring can be substituted at one or more ring positions with such substituents as described above, as for example, alkyl, halogen, hydroxyl, alkoxy, alkylcarbonyloxy, arylcarbonyloxy, alkoxycarbonyloxy, aryloxycarbonyloxy, carboxylate, alkylcarbonyl, alkylaminoacarbonyl, aralkylaminocarbonyl, alkenylaminocarbonyl, alkylcarbonyl, arylcarbonyl, aralkylcarbonyl, alkenylcarbonyl, alkoxycarbonyl, aminocarbonyl, alkylthiocarbonyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety. Aryl groups can also be fused or bridged with alicyclic or heterocyclic rings which are not aromatic so as to form a polycycle (e.g., tetralin).

Certain aryl groups recited herein are C₆-C₁₀arylC₀-C₈alkyl groups (i.e., groups in which a 6- to 10-membered carbocyclic group comprising at least one aromatic ring is linked via a single covalent bond or a C₁-C₈alkylene group). Such groups include, for example, phenyl and indanyl, as well as groups in which either of the foregoing is linked via C₁-C₈alkylene, preferably via C₁-G₄alkylene. Phenyl groups linked via a single covalent bond or C₁-C₆alkylene group are designated phenylC₀-C₆alkyl (e.g., benzyl, 1-phenyl-ethyl, 1-phenyl-propyl and 2-phenyl-ethyl).

“Arylene” refers to a divalent aryl group, as defined above. Arylene is intended to encompass divalent residues of phenyl, naphthyl and biphenyl. “Heteroarylene” refers to divalent heteroaryl groups as defined infra.

The term “heteroaryl”, as used herein, represents a stable monocyclic or bicyclic ring of up to 7 atoms in each ring, wherein at least one ring is aromatic and contains from 1 to 4heteroatoms selected from the group consisting of O, N and S. Heteroaryl groups within the scope of this definition include but are not limited to: acridinyl, carbazolyl, cinnolinyl, quinoxalinyl, pyrrazolyl, indolyl, isoindoline, benzotriazolyl, furanyl, thienyl, benzothienyl, benzofuranyl, quinolinyl, isoquinolinyl, oxazolyl, isoxazolyl, indolyl, pyrazinyl, pyridazinyl, pyridinyl, pyrimidinyl, pyrrolyl, tetrahydroquinoline. As with the definition of heterocycle below, “heteroaryl” is also understood to include the N-oxide derivative of any nitrogen-containing heteroaryl. In cases where the heteroaryl substituent is bicyclic and one ring is non-aromatic or contains no heteroatoms, it is understood that attachment is via the aromatic ring or via the heteroatom containing ring, respectively.

The term “heterocycle” or “heterocyclyl” as used herein is intended to mean a 5- to 10-membered aromatic or nonaromatic heterocycle containing from 1 to 4 heteroatoms selected from the group consisting of O, N and S, and includes bicyclic groups. “Heterocyclyl” therefore includes the above mentioned heteroaryls, as well as dihydro and tetrahydro analogs thereof. Further examples of “heterocyclyl” include, but are not limited to the following: benzoimidazolyl, benzofuranyl, benzofurazanyl, benzopyrazolyl, benzotriazolyl, benzpthiophenyl, benzoxazolyl, carbazolyl, carbolinyl, cinnolinyl, furanyl, imidazolyl, indolinyl, indolyl, indolazinyl, indazolyl, isobenzofuranyl, isoindolyl, isoquinolyl, isothiazolyl, isoxazolyl, naphthpyridinyl, oxadiazolyl, oxazolyl, oxazoline, isoxazoline, oxetanyl, pyranyl, pyrazinyl pyrazolyl, pyridazinyl, pyridopyridinyl, pyridazinyl, pyridyl, pyrimidyl, pyrrolyl, quinazolinyl, quinolyl, quinoxalinyl, tetrahydropyranyl, tetrazolyl, tetrazolopyridyl, thiadiazolyl, thiazolyl, thienyl, triazolyl, azetidinyl, 1,4-dioxanyl, hexahydroazepinyl, piperazinyl, piperidinyl, pyridin-2-onyl, pyrrolidinyl, morpholinyl, thiomorpholinyl, dihydrobenzoimidazolyl, dihydrobenzofuranyl, dihydrobenzothiophenyl, dihydrobenzoxazolyl, dihydrofuranyl, dihydroimidazolyl, dihydroindolyl, dihydroisooxazolyl, dihydroisothiazolyl, dihydrooxadiazolyl, dihydrooxazolyl, dihydropyrazinyl, dihydropyrazolyl, dihydropyridinyl, dihydropyrimidinyl, dihydropyrrolyl, dihydroquinolinyl, dihydrotetrazolyl, dihydrothiadiazolyl, dihydrothiazolyl, dihydrothienyl, dihydrotriazolyl, dihydroazetidinyl, methylenedioxybenzoyl, tetrahydrofuranyl, and tetrahydrothienyl, and N-oxides thereof. Attachment of a heterocyclyl substituent can occur via a carbon atom or via a heteroatom.

A “heterocycleC₀-C₈alkyl” is a heterocyclic group linked via a single covalent bond or C₁-C₈alkylene group. A (4- to 7-membered heterocycle)C₀-C₈alkyl is a heterocyclic group (e.g., monocyclic or bicyclic) having from 4 to 7 ring members linked via a single covalent bond or an alkylene group having from 1 to 8 carbon atoms. A “(6-membered heteroaryl)C₀-C₆alkyl” refers to a heteroaryl group linked via a direct bond or C₁-C₆alkyl group.

The term “acyl” includes compounds and moieties which contain the acyl radical (CH₃CO—) or a carbonyl group. The term “substituted acyl” includes acyl groups where one or more of the hydrogen atoms are replaced by for example, alkyl groups, alkynyl groups, halogens, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamide, nitro, trifluoromethyl, cyano, azido, heterocyclyl, alkylaryl, or an aromatic or heteroaromatic moiety.

The term “acylamino” includes moieties wherein an acyl moiety is bonded to an amino group. For example, the term includes alkylcarbonylamino, arylcarbonylamino, carbamoyl and ureido groups.

The term “alkoxy” includes substituted and unsubstituted alkyl, alkenyl, and alkynyl groups covalently linked to an oxygen atom. Examples of alkoxy groups include methoxy, ethoxy, isopropyloxy, propoxy, butoxy, and pentoxy groups and may include cyclic groups such as cyclopentoxy. Examples of substituted alkoxy groups include halogenated alkoxy groups. The alkoxy groups can be substituted with groups such as alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moieties. Examples of halogen substituted alkoxy groups include, but are not limited to, fluoromethoxy, difluoromethoxy, trifluoromethoxy, chloromethoxy, dichloromethoxy, trichloromethoxy, etc.

The term “carbonyl” or “carboxy” includes compounds and moieties which contain a carbon connected with a double bond to an oxygen atom, and tautomeric forms thereof. Examples of moieties that contain a carbonyl include aldehydes, ketones, carboxylic acids, amides, esters, anhydrides, etc. The term “carboxy moiety” or “carbonyl moiety” refers to groups such as “alkylcarbonyl” groups wherein an alkyl group is covalently bound to a carbonyl group, “alkenylcarbonyl” groups wherein an alkenyl group is covalently bound to a carbonyl group, “alkynylcarbonyl” groups wherein an alkynyl group is covalently bound to a carbonyl group, “arylcarbonyl” groups wherein an aryl group is covalently attached to the carbonyl group. Furthermore, the term also refers to groups wherein one or more heteroatoms are covalently bonded to the carbonyl moiety. For example, the term includes moieties such as, for example, aminocarbonyl moieties, (wherein a nitrogen atom is bound to the carbon of the carbonyl group, e.g., an amide), aminocarbonyloxy moieties, wherein an oxygen and a nitrogen atom are both bond to the carbon of the carbonyl group (e.g., also referred to as a “carbamate”). Furthermore, aminocarbonylamino groups (e.g., ureas) are also include as well as other combinations of carbonyl groups bound to heteroatoms (e.g., nitrogen, oxygen, sulfur, etc. as well as carbon atoms). Furthermore, the heteroatom can be further substituted with one or more alkyl, alkenyl, alkynyl, aryl, aralkyl, acyl, etc. moieties.

The term “thiocarbonyl” or “thiocarboxy” includes compounds and moieties which contain a carbon connected with a double bond to a sulfur atom. The term “thiocarbonyl moiety” includes moieties that are analogous to carbonyl moieties. For example, “thiocarbonyl” moieties include aminothiocarbonyl, wherein an amino group is bound to the carbon atom of the thiocarbonyl group, furthermore other thiocarbonyl moieties include, oxythiocarbonyls (oxygen bound to the carbon atom), aminothiocarbonylamino groups, etc.

The term “ether” includes compounds or moieties that contain an oxygen bonded to two different carbon atoms or heteroatoms. For example, the term includes “alkoxyalkyl” which refers to an alkyl, alkenyl, or alkynyl group covalently bonded to an oxygen atom that is covalently bonded to another alkyl group.

The term “ester” includes compounds and moieties that contain a carbon or a heteroatom bound to an oxygen atom that is bonded to the carbon of a carbonyl group. The term “ester” includes alkoxycarboxy groups such as methoxycarbonyl, ethoxycarbonyl, propoxycarbonyl, butoxycarbonyl, pentoxycarbonyl, etc. The alkyl, alkenyl, or alkynyl groups are as defined above.

The term “thioether” includes compounds and moieties which contain a sulfur atom bonded to two different carbon or hetero atoms. Examples of thioethers include, but are not limited to alkthioalkyls, alkthioalkenyls, and alkthioalkynyls. The term “alkthioalkyls” include compounds with an alkyl, alkenyl, or alkynyl group bonded to a sulfur atom that is bonded to an alkyl group. Similarly, the term “alkthioalkenyls” and alkthioalkynyls” refer to compounds or moieties wherein an alkyl, alkenyl, or alkynyl group is bonded to a sulfur atom which is covalently bonded to an alkynyl group.

The term “hydroxy” or “hydroxyl” includes groups with an —OH or —O.

The term “halogen” includes fluorine, bromine, chlorine, iodine, etc. The term “perhalogenated” generally refers to a moiety wherein all hydrogens are replaced by halogen atoms.

The terms “polycyclyl” or “polycyclic radical” include moieties with two or more rings (e.g., cycloalkyls, cycloalkenyls, cycloalkynyls, aryls and/or heterocyclyls) in which two or more carbons are common to two adjoining rings, e.g., the rings are “fused rings”. Rings that are joined through non-adjacent atoms are termed “bridged” rings. Each of the rings of the polycycle can be substituted with such substituents as described above, as for example, halogen, hydroxyl, alkylcarbonyloxy, arylcarbonyloxy, alkoxycarbonyloxy, aryloxycarbonyloxy, carboxylate, alkylcarbonyl, alkoxycarbonyl, alkylaminoacarbonyl, aralkylaminocarbonyl, alkenylaminocarbonyl, alkylcarbonyl, arylcarbonyl, aralkylcarbonyl, alkenylcarbonyl, aminocarbonyl, alkylthiocarbonyl, alkoxyl, phosphate, phosphonato, phosphinato, cyano, amino (including alkyl, amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkyl, alkylaryl, or an aromatic or heteroaromatic moiety.

The term “heteroatom” includes atoms of any element other than carbon or hydrogen. Preferred heteroatoms are nitrogen, oxygen, sulfur and phosphorus.

Additionally, the phrase “any combination thereof implies that any number of the listed functional groups and molecules may be combined to create a larger molecular architecture. For example, the terms “phenyl,” “carbonyl” (or “═O”), “—O—,” “—OH,” and C₁₋₆ (i.e., —CH₃ and —CH₂CH₂CH₂—) can be combined to form a 3-methoxy-4-propoxybenzoic acid substituent. It is to be understood that when combining functional groups and molecules to create a larger molecular architecture, hydrogens can be removed or added, as required to satisfy the valence of each atom.

It is to be understood that all of the compounds of the invention described above will further include bonds between adjacent atoms and/or hydrogens as required to satisfy the valence of each atom. That is, bonds and/or hydrogen atoms are added to provide the following number of total bonds to each of the following types of atoms: carbon: four bonds; nitrogen: three bonds; oxygen: two bonds; and sulfur: two bonds.

Groups that are “optionally substituted” are unsubstituted or are substituted by other than hydrogen at one or more available positions, typically 1, 2, 3,4 or 5 positions, by one or more suitable groups (which may be the same or different). Optional substitution is also indicated by the phrase “substituted with from 0 to X substituents,” where X is the maximum number of possible substituents. Certain optionally substituted groups are substituted with from 0 to 2, 3 or 4 independently selected substituents (i.e., are unsubstituted or substituted with up to the recited maximum number of substitutents).

It will be noted that the structures of some of the compounds of this invention include asymmetric carbon atoms. It is to be understood accordingly that the isomers arising from such asymmetry (e.g., all enantiomers, stereoisomers, rotamers, tautomers, diastereomers, or racemates) are included within the scope of this invention. Such isomers can be obtained in substantially pure form by classical separation techniques and by stereochemically controlled synthesis. Furthermore, the structures and other compounds and moieties discussed in this application also include all tautomers thereof. Compounds described herein may be obtained through art recognized synthesis strategies.

It will also be noted that the substituents of some of the compounds of this invention include isomeric cyclic structures. It is to be understood accordingly that constitutional isomers of particular substituents are included within the scope of this invention, unless indicated otherwise. For example, the term “tetrazole” includes tetrazole, 2H-tetrazole, 3H-tetrazole, 4H-tetrazole and 5H-tetrazole.

### Use in HCV-Associated Disorders

The compounds of the present invention have valuable pharmacological properties and are useful in the treatment of diseases. In certain embodiments, compounds of the invention are useful in the treatment of HCV-associated disorders, e.g., as drugs to treat HCV infection.

The term “use” includes any one or more of the following embodiments of the invention, respectively: the use in the treatment of HCV-associated disorders; the use for the manufacture of pharmaceutical compositions for use in the treatment of these diseases, e.g., in the manufacture of a medicament; methods of use of compounds of the invention in the treatment of these diseases; pharmaceutical preparations having compounds of the invention for the treatment of these diseases; and compounds of the invention for use in the treatment of these diseases; as appropriate and expedient, if not stated otherwise. In particular, diseases to be treated and are thus preferred for use of a compound of the present invention are selected from HCV-associated disorders, including those corresponding to HCV-infection, as well as those diseases that depend on the activity of one or more of the NS3, NS4A, NS4B, NS5A and NS5B proteins, or a NS3-NS4A, NS4A-NS4B, NS4B-NS5A or NS5A-NS5B complex. The term “use” further includes embodiments of compositions herein which bind to an HCV protein sufficiently to serve as tracers or labels, so that when coupled to a fluoro tag, or made radioactive, can be used as a research reagent or as a diagnostic or an imaging agent.

In certain embodiments, a compound of the present invention is used for treating HCV-associated diseases, and use of the compound of the present invention as an inhibitor of any one or more HCV's. It is envisioned that a use can be a treatment of inhibiting one or more strains of HCV.

### Assays

The inhibition of HCV activity may be measured as using a number of assays available in the art. An example of such an assay can be found in Anal Biochem. 1996 240(1): 60-7; which is incorporated by reference in its entirety. Assays for measurement of HCV activity are also described in the experimental section below.

### Pharmaceutical Compositions

The language “effective amount” of the compound is that amount necessary or sufficient to treat or prevent an HCV-associated disorder, e.g. prevent the various morphological and somatic symptoms of an HCV-associated disorder, and/or a disease or condition described herein. In an example, an effective amount of the HCV-modulating compound is the amount sufficient to treat HCV infection in a subject. In another example, an effective amount of the HCV-modulating compound is the amount sufficient to treat HCV infection, liver cirrhosis, chronic liver disease, hepatocellular carcinoma, cryoglobulinaemia, non-Hodgkin's lymphoma, and a suppressed innate intracellular immune response in a subject. The effective amount can vary depending on such factors as the size and weight of the subject, the type of illness, or the particular compound of the invention. For example, the choice of the compound of the invention can affect what constitutes an “effective amount.” One of ordinary skill in the art would be able to study the factors contained herein and make the determination regarding the effective amount of the compounds of the invention without undue experimentation.

The regimen of administration can affect what constitutes an effective amount. The compound of the invention can be administered to the subject either prior to or after the onset of an HCV-associated state. Further, several divided dosages, as well as staggered dosages, can be administered daily or sequentially, or the dose can be continuously infused, or can be a bolus injection. Further, the dosages of the compound(s) of the invention can be proportionally increased or decreased as indicated by the exigencies of the therapeutic or prophylactic situation.

Compounds of the invention may be used in the treatment of states, disorders or diseases as described herein, or for the manufacture of pharmaceutical compositions for use in the treatment of these diseases. Methods of use of compounds of the present invention in the treatment of these diseases, or pharmaceutical preparations having compounds of the present invention for the treatment of these diseases.

The language “pharmaceutical composition” includes preparations suitable for administration to mammals, e.g., humans. When the compounds of the present invention are administered as pharmaceuticals to mammals, e.g., humans, they can be given per se or as a pharmaceutical composition containing, for example, 0.1 to 99.5% (more preferably, 0.5 to 90%) of active ingredient in combination with a pharrnaceutically acceptable carrier.

The phrase “pharmaceutically acceptable carrier” is art recognized and includes a pharmaceutically acceptable material, composition or vehicle, suitable for administering compounds of the present invention to mammals. The carriers include liquid or solid filler, diluent, excipient, solvent or encapsulating material, involved in carrying or transporting the subject agent from one organ, or portion of the body, to another organ, or portion of the body. Each carrier must be “acceptable” in the sense of being compatible with the other ingredients of the formulation and not injurious to the patient. Some examples of materials which can serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oiland soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: water soluble antioxidants, such as ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propyl gallate, α-tocopherol, and the like; and metal chelating agents, such as citric acid, ethylenediamine tetraacetic acid (EDTA), sorbitol, tartaric acid, phosphoric acid, and the like.

Formulations of the present invention include those suitable for oral, nasal, topical, transdermal, buccal, sublingual, rectal, vaginal and/or parenteral administration. The formulations may conveniently be presented in unit dosage form and may be prepared by any methods well known in the art of pharmacy. The amount of active ingredient that can be combined with a carrier material to produce a single dosage form will generally be that amount of the compound that produces a therapeutic effect. Generally, out of one hundred per cent, this amount will range from about 1 per cent to about ninety-nine percent of active ingredient, preferably from about 5 per cent to about 70 per cent, most preferably from about. 10 per cent to about 30 per cent.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A compound of the present invention may also be administered as a bolus, electuary or paste.

In solid dosage forms of the invention for oral administration (capsules, tablets, pills, dragees, powders, granules and the like), the active ingredient is mixed with one or more pharmaceutically acceptable carriers, such as sodium citrate or dicalcium phosphate, and/or any of the following: fillers or extenders, such as starches, lactose, sucrose, glucose, mannitol, and/or silicic acid; binders, such as, for example, carboxymethylcellulose, alginates, gelatin, polyvinyl pyrrolidone, sucrose and/or acacia; humectants, such as glycerol; disintegrating agents, such as agar-agar, calcium carbonate, potato or tapioca starch, alginic acid, certain silicates, and sodium carbonate; solution retarding agents, such as paraffin; absorption accelerators, such as quaternary ammonium compounds; wetting agents, such as, for example, cetyl alcohol and glycerol monostearate; absorbents, such as kaolin and bentonite clay; lubricants, such a talc, calcium stearate, magnesium stearate, solid polyethylene glycols, sodium lauryl sulfate, and mixtures thereof; and coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant* inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release Of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms for oral administration of the compounds of the invention include pharmaceutically acceptable emulsions, microemulsions, solutions, suspensions, syrups and elixirs. In addition to the active ingredient, the liquid dosage forms may contain inert diluent commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions of the invention for rectal or vaginal administration may be presented as a suppository, which maybe prepared by mixing one or more compounds of the invention with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Formulations of the present invention which are suitable for vaginal administration also include pessaries, tampons, creams, gels, pastes, foams or spray formulations containing such carriers as are known in the art to be appropriate.

Dosage forms for the topical or transdermal administration of a compound of this invention include powders, sprays, ointments, pastes, creams, lotions, gels, solutions, patches and inhalants. The active compound may be mixed under sterile conditions with a pharmaceutically acceptable carrier, and with any preservatives, buffers, or propellants that may be required.

The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the compound in the proper medium. Absorption enhancers can also be used to increase the flux of the compound across the skin. The rate of such flux can be controlled by either providing a rate controlling membrane or dispersing the active compound in a polymer matrix or gel.

Ophthalmic formulations, eye ointments, powders, solutions and the like, are also

contemplated as being within the scope of this invention.

Pharmaceutical compositions of this invention suitable for parenteral administration comprise one or more compounds of the invention in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

The preparations of the present invention may be given orally, parenterally, topically, or rectally. They are of course given by forms suitable for each administration route. For example, they are administered in tablets or capsule form, by injection, inhalation, eye lotion, ointment, suppository, etc., administration by injection, infusion or inhalation; topical by lotion or ointment; and rectal by suppositories. Oral administration is preferred.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

These compounds may be administered to humans and other animals for therapy by any suitable route of administration, including orally, nasally, as by, for example, a spray, rectally, intravaginally, parenterally, intracistemally and topically, as by powders, ointments or drops, including buccally and sublingually.

Regardless of the route of administration selected, the compounds of the present invention, which may be used in a suitable hydrated form, and/or the pharmaceutical compositions of the present invention, are formulated into pharmaceutically acceptable dosage forms by conventional methods known to those of skill in the art.

Actual dosage levels of the active ingredients in the pharmaceutical compositions of this invention may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular compound of the present invention employed, or the ester, salt or amide thereof, the route of administration, the time of administration, the rate of excretion of the particular compound being employed, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In general, a suitable daily dose of a compound of the invention will be that amount of the compound that is the lowest dose effective to produce a therapeutic effect. Such an effective dose will generally depend upon the factors described above. Generally, intravenous and subcutaneous doses of the compounds of this invention for a patient, when used for the indicated analgesic effects, will range from about 0.0001 to about 100 mg per kilogram of body weight per day, more preferably from about 0.01 to about 50 mg per kg per day, and still more preferably from about 1.0 to about 100 mg per kg per day. An effective amount is that amount treats an HCV-associated disorder.

If desired, the effective daily dose of the active compound may be administered as two, three, four, five, six or more sub-doses administered separately at appropriate intervals throughout the day, optionally, in unit dosage forms.

While it is possible for a compound of the present invention to be administered alone, it is preferable to administer the compound as a pharmaceutical composition.

### Synthetic Procedure

Compounds of the present invention are prepared from commonly available compounds using procedures known to those skilled in the art, including any one or more of the following conditions without limitation:

Within the scope of this text, only a readily removable group that is not a constituent of the particular desired end product of the compounds of the present invention is designated a “protecting group,” unless the context indicates otherwise. The protection of functional groups by such protecting groups, the protecting groups themselves, and their cleavage reactions are described for example in standard reference works, such as e.g., Science of Synthesis: Houben-Weyl Methods of Molecular Transformation. Georg Thieme. Verlag, Stuttgart, Germany. 2005. 41627 pp. (URL: http://www.science-of-synthesis.com (Electronic Version, 48 Volumes)); J. F. W. McOmie, “Protective Groups in Organic Chemistry”, Plenum Press, London and New York 1973, in T. W. Greene and P. G. M. Wuts, “Protective Groups in Organic Synthesis”, Third edition, Wiley, New York 1999, in “The Peptides”; Volume 3 (editors: E. Gross and J. Meienhofer), Academic Press, London and New York. 1981, in “Methoden der organischen Chemie” (Methods of Organic Chemistry), Houben Weyl, 4th edition, Volume 15/I, Georg Thieme Verlag, Stuttgart 1974, in H.-D. Jakubke and H. Jeschkeit, “Aminosauren, Peptide, Proteine” (Amino acids, Peptides, Proteins), Verlag Chemie, Weinheim, Deerfield Beach, and Basel 1982, and in Jochen Lehmann, “Chemie der. Kohlenhydrate: Monosaccharide und Derivate” (Chemistry of Carbohydrates: Monosaccharides and Derivatives), Georg Thieme Verlag, Stuttgart 1974. A characteristic of protecting groups is that they can be removed readily (i.e., without the occurrence of undesired secondary reactions) for example by solvolysis, reduction, photolysis or alternatively under physiological conditions (e.g., by enzymatic cleavage).

Salts of compounds of the present invention having at least one salt-forming group may be prepared in a manner known per se. For example, salts of compounds of the present invention having acid groups may be formed, for example, by treating the compounds with metal compounds, such as alkali metal salts of suitable organic carboxylic acids, e.g., the sodium salt of 2-ethylhexanoic acid, with organic alkali metal or alkaline earth metal compounds, such as the corresponding hydroxides, carbonates or hydrogen carbonates, such as sodium or potassium hydroxide, carbonate or hydrogen carbonate, with corresponding calcium compounds or with ammonia or a suitable organic amine, stoichiometric amounts or only a small excess of the salt-forming agent preferably being used. Acid addition salts of compounds of the present invention are obtained in customary manner, e.g., by treating the compounds with an acid or a suitable anion exchange reagent. Internal salts of compounds of the present invention containing acid and basic salt-forming groups, e.g., a free carboxy group and a free amino group, may be formed, e.g., by the neutralization of salts, such as acid addition salts, to the isoelectric point, e.g., with weak bases, or by treatment with ion exchangers.

Salts can be converted in customary manner into the free compounds; metal and ammonium salts can be converted, for example, by treatment with suitable acids, and acid addition salts, for example, by treatment with a suitable basic agent.

Mixtures of isomers obtainable according to the invention can be separated in a manner known per se into the individual isomers; diastereoisomers can be separated, for example, by partitioning between polyphasic solvent mixtures, recrystallization and/or chromatographic separation, for example over silica gel or by, e.g., medium pressure liquid chromatography over a reversed phase column, and racemates can be separated, for example, by the, formation of salts with optically pure salt-forming reagents and separation of the mixture of diastereoisomers so obtainable, for example by means of fractional crystallization, or by chromatography over optically active column materials.

Intermediates and final products can be worked up and/or purified according to standard methods, e.g., using chromatographic methods, distribution methods, (re-) crystallization, and the like.

### General Process Conditions

The following applies in general to all processes mentioned throughout this disclosure.

The process steps to synthesize the compounds of the invention can be carried out under reaction conditions that are known per se, including those mentioned specifically, in the absence or, customarily, in the presence of solvents or diluents, including, for example, solvents or diluents that are inert towards the reagents used and dissolve them, in the absence or presence of catalysts, condensation or neutralizing agents, for example ion exchangers, such as cation exchangers, e.g., in the H₊ form, depending on the nature of the reaction and/or of the reactants at reduced, normal or elevated temperature, for example in a temperature range of from about −100° C. to about 190° C., including, for example, from approximately −80° C. to approximately 150° C., for example at from −80 to −60° C., at room temperature, at from −20 to 40° C. or at reflux temperature, under atmospheric pressure or in a closed vessel, where appropriate under pressure, and/or in an inert atmosphere, for example under an argon or nitrogen atmosphere.

At all stages of the reactions, mixtures of isomers that are formed can be separated into the individual isomers, for example diastereoisomers or enantiomers, or into any desired mixtures of isomers, for example racemates or mixtures of diastereoisomers, for example analogously to the methods described in Science of Synthesis: Houben-Weyl Methods of Molecular Transformation. Georg Thieme Verlag, Stuttgart, Germany. 2005.

The solvents from which those solvents that are suitable for any particular, reaction may be selected include those mentioned specifically or, for example, water, esters, such as lower alkyl-lower alkanoates, for example ethyl acetate, ethers, such as aliphatic ethers, for example diethyl ether, or cyclic ethers, for example tetrahydrofuran or dioxane, liquid aromatic hydrocarbons, such as benzene or toluene, alcohols, such as methanol, ethanol or 1- or 2-propanol, nitriles, such as acetonitrile, halogenated hydrocarbons, such as methylene chloride or chloroform, acid amides, such as dimethylformamide or dimethyl acetamide, bases, such as heterocyclic nitrogen bases, for example pyridine or N-methylpyrrolidin-2-one, carboxylic acid anhydrides, such as lower alkanoic acid anhydrides, for example acetic anhydride, cyclic, linear or branched hydrocarbons, such as cyclohexane, hexane or isopentane, or mixtures of those solvents, for example aqueous solutions, unless otherwise indicated in the description of the processes. Such solvent mixtures may also be used in working up, for example by chromatography or partitioning.

The compounds, including their salts, may also be obtained in the form of hydrates, or their crystals may, for example, include the solvent used for crystallization. Different crystalline forms may be present.

The invention relates also to those forms of the process in which a compound obtainable as an intermediate at any stage of the process is used as starting material and the remaining process steps are carried out, or in which a starting material is formed under the reaction conditions or is used in the form of a derivative, for example in a protected form or in the form of a salt, or a compound obtainable by the process according to the invention is produced under the process conditions and processed further in situ.

### Pro-Drugs

The present invention also relates to pro-drugs of a compound of the present invention that are converted in vivo to the compounds of the present invention as described herein. Any reference to a compound of the present invention is therefore to be understood as referring also to the corresponding pro-drugs of the compound of the present invention, as appropriate and expedient.

### Combinations

A compound of the present invention may also be used in combination with other agents, e.g., an additional HCV-modulating compound that is or is not of the formula I, for treatment of and HCV-associated disorder in a subject.

By the term “combination”, is meant either a fixed combination in one dosage unit form, or a kit of parts for the combined administration where a compound of the present invention and a combination partner may be administered independently at the same time or separately within time intervals that especially allow that the combination partners show a cooperative, e.g., synergistic, effect, or any combination thereof.

For example, WO 2005/042020, incorporated herein by reference in its entirety, describes the combination of various HCV inhibitors with a cytochrome P450 (“CYP”) inhibitor. Any CYP inhibitor that improves the pharmacokinetics of the relevant NS3/4A protease may be used in combination with the compounds of this invention. These CYP inhibitors include, but are not limited to, ritonavir (WO 94/14436, incorporated herein by reference in its entirety), ketoconazole, troleandomycin, 4-methyl pyrazole, cyclosporin, clomethiazole, cimetidine, itraconazole, fluconazole, miconazole, fluvoxamine, fluoxetine, nefazodone, sertraline, indinavir, nelfinavir, amprenavir, fosamprenavir, saquinavir, lopinavir, delavirdine, erythromycin, VX-944, and VX-497. Preferred CYP inhibitors include ritonavir, ketoconazole, troleandomycin, 4-methyl pyrazole, cyclosporin, and clomethiazole.

Methods for measuring the ability of a compound to inhibit CYP activity are known (see, e.g., U.S. Pat. No. 6,037,157 and Yun, et al. Drug Metabolism & Disposition, vol. 21, pp. 403-407 (1993); incorporated herein by reference). For example, a compound to be evaluated may be incubated with 0.1, 0.5, and 1.0 mg protein/ml, or other appropriate concentration of human hepatic microsomes (e. g., commercially available, pooled characterized hepatic microsomes) for 0, 5, 10, 20, and 30 minutes, or other appropriate times, in the presence of an NADPH-generating system. Control incubations may be performed in the absence of hepatic microsomes for 0 and 30 minutes (triplicate). The samples may be analyzed for the presence of the compound. Incubation conditions that produce a linear rate of compound metabolism will be used a guide for further studies. Experiments known in the art can be used to determine the kinetics of the compound metabolism (Kₘ and Vₘₐₓ). The rate of disappearance of compound may be determined and the data analyzed according to Michaelis-Menten kinetics by using Lineweaver-Burk, Eadie-Hofstee, or nonlinear regression analysis.

Inhibition of metabolism experiments may then be performed. For example, a compound (one concentration, <Kₘ) may be incubated with pooled human hepatic microsomes in the absence or presence of a CYP inhibitor (such as ritonavir) under the conditions determined above. As would be recognized, control incubations should contain the same concentration of organic solvent as the incubations with the CYP inhibitor. The concentrations of the compound in the samples may be quantitated, and the rate of disappearance of parent compound may be determined, with rates being expressed as a percentage of control activity.

Methods for evaluating the influence of co-administration of a compound of the invention and a CYP inhibitor in a subject are also known (see, e.g., US2004/0028755; incorporated herein by reference). Any such methods could be used in connection with this invention to determine the pharmacokinetic impact of a combination. Subjects that would benefit from treatment according to this invention could then be selected.

Accordingly, one embodiment of this invention provides a method for administering an inhibitor of CYP3A4 and a compound of the invention. Another embodiment of this invention provides a method for administering an inhibitor of isozyme 3A4 (“CYP3A4”), isozyme 2C19 (“CYP2C19”), isozyme 2D6 (“CYP2D6”), isozyme 1A2 (“CYP1A2”), isozyme 2C9 (“CYP2C9”), or isozyme 2E1 (“CYP2E1”). In embodiments where the protease inhibitor is VX-950 (or a sterereoisomer thereof), the CYP inhibitor preferably inhibits CYP3A4.

As would be appreciated, CYP3A4 activity is broadly observed in humans. Accordingly, embodiments of this invention involving inhibition of isozyme 3A4 would be expected to be applicable to a broad range of patients.

Accordingly, this invention provides methods wherein the CYP inhibitor is administered together with the compound of the invention in the same dosage form or in separate dosage forms.

The compounds of the invention (e.g., compound of Formula I or subformulae thereof) may be administered as the sole ingredient or in combination or alteration with other antiviral agents, especially agents active against HCV. In combination therapy, effective dosages of two or more agents are administered together, whereas in alternation or sequential-step therapy, an effective dosage of each agent is administered serially or sequentially. In general, combination therapy is typically preferred over alternation therapy because it induces multiple simultaneous stresses on the virus. The dosages given will depend on absorption, inactivation and excretion rate of the drug as well as other factors. It is to be noted that dosage values will also vary with the severity of the condition to be alleviated. It is to be further understood that for any particular subject, specific dosage regimens and schedules should be adjusted over time according to the individual need and the professional judgment of the person administering or supervising the administration of the compositions. The efficacy of a drug against the viral infection can be prolonged, augmented, or restored by administering the compound in combination or alternation with a second, and perhaps third antiviral compound that induces a different gene mutation than that caused by the principle drug in a drug resistant virus. Alternatively, the pharmacokinetic, biodistribution or other parameters of the drug can be altered by such combination or alternation therapy.

Daily dosages required in practicing the method of the present invention will vary depending upon, for example, the compound of the invention employed, the host, the mode of administration, the severity of the condition to be treated. A preferred daily dosage range is about from 1 to 50 mg/kg per day as a single dose or in divided doses. Suitable daily dosages for patients are on the order of from e.g. 1 to 20 mg/kg p.o or i.v. Suitable unit dosage forms for oral administration comprise from ca. 0.25 to 10 mg/kg active ingredient, e.g. compound of Formula I or any subformulae thereof, together with one or more pharmaceutically acceptable diluents or carriers therefor. The amount of co-agent in the dosage form can vary greatly, e.g., 0.00001 to 1000 mg/kg active ingredient.

Daily dosages with respect to the co-agent used will vary depending upon, for example, the compound employed, the host, the mode of administration and the severity of the condition to be treated. For example, lamivudine may be administered at a daily dosage of 100 mg. The pegylated interferon may be administered parenterally one to three times per week, preferably once a week, at a total weekly dose ranging from 2 to 10 million IU, more preferable 5 to 10 million IU, most preferable 8 to 10 million IU. Because of the diverse types of co-agent that may be used, the amounts can vary greatly, e.g., 0001 to 5,000 mg/kg per day.

The current standard of care for treating hepatitis C is the combination of pegylated interferon alpha with ribavirin, of which the recommended, doses are 1.5 μg/kg/wk peginterferon alfa-2b or 180 μg/wk peginterferon alfa-2a, plus 1,000 to 1,200 mg daily of ribavirin for 48 weeks for genotype I patients, or 800 mg daily of ribavirin for 24 weeks for genotype ⅔ patients.

The compound of the invention (e.g., compound of Formula I or subformulae thereof) and co-agents of the invention may be administered by any conventional route, in particular enterally, e.g. orally, for example in the form of solutions for drinking, tablets or capsules or parenterally, for example in the form of injectable solutions or suspensions. Certain preferred pharmaceutical compositions may be e.g. those based on microemulsions as described in UK 2,222,770 A.

The compound of the invention (e.g., compound of Formula I or subformulae thereof) are administered together with other drugs (co-agents) e.g. a drug which has anti-viral activity, especially anti-Flaviviridae activity, most especially anti-HCV activity, e.g. an interferon, e.g. interferon-α-2a or interferon-α-2b, e.g. Intron^{R} A, Roferon^{R}, Avonex^{R}, Rebif^{R} or Betaferon^{R}, or an interferon conjugated to a water soluble polymer or to human albumin, e.g. albuferon, an anti-viral agent, e.g. ribavirin, lamivudine, the compounds disclosed in U.S. Pat. No. 6,812,219 and WO 2004/002422 A2 (the disclosures of which are incorporated herein by reference in their entireties), an inhibitor of the HCV or other Flaviviridae virus encoded factors like the NS3/4A protease, helicase or RNA polymerase or a prodrug of such an inhibitor, an anti-fibrotic agent, e.g. a N-phenyl-2-pyrimidine-amine derivative, e.g. imatinib, an immune modulating agent, e.g. mycophenolic acid, a salt or a prodrug thereof, e.g. sodium mycophenolate or mycophenolate mofetil, or a SIP receptor agonist, e.g. FTY720 or an analogue thereof optionally phosphorylated, e.g. as disclosed in EP627406A1, EP778263A1, EP1002792A1, WO02/18395, WO02/76995, WO 02/06268, JP2002316985, WO03/29184, WO03/29205, WO03/62252 and WO03/62248, the disclosures of which are incorporated herein by reference in their entireties.

Conjugates of interferon to a water-soluble polymer are meant to include especially conjugates to polyalkylene oxide homopolymers such as polyethylene glycol (PEG) or polypropylene glycols, polyoxyethylenated polyols, copolymers thereof and block copolymers thereof. As an alternative to polyaikylene oxide-based polymers, effectively non-antigenic materials such as dextran, polyvinyl pyrrolidones, polyacrylamides, polyvinyl alcohols, carbohydrate-based polymers and the like can be used. Such interferon-polymer conjugates are described in U.S. Pat. Nos. 4,766,106, 4,917,888, European Patent Application No. 0 236 987, European Patent Application No. 0 510 356 and International Application Publication No. WO 95/13090, the disclosures of which are incorporated herein by reference in their entireties. Since the polymeric modification sufficiently reduces antigenic responses, the foreign interferon need not be completely autologous. Interferon used to prepare polymer conjugates may be prepared from a mammalian extract, such as human, ruminant or bovine interferon, or recombinantly produced. Preferred are conjugates of interferon to polyethylene glycol, also known as pegylated interferons.

Especially preferred conjugates of interferon are pegylated alfa-interferons, for example pegylated interferon-α-2a, pegylated interferon-α-2b; pegylated consensus interferon or pegylated purified interferon-α product. Pegylated interferon-α-2a is described e.g. in European Patent 593,868 (incorporated herein by reference in its entirety) and commercially available e. g. under the tradename PEGASYS® (Hoffmann-La Roche). Pegylated interferon-α-2b is described, e.g. in European Patent 975,369 (incorporated herein by reference in its entirety) and commercially available e.g. under the tradename PEG-INTRON A® (Schering Plough). Pegylated consensus interferon is described in WO 96/11953 (incorporated herein by reference in its entirety). The preferred pegylated α-interferons are pegylated interferon-α-2a and pegylated interferon-α-2b. Also preferred is pegylated consensus interferon.

Other preferred co-agents are fusion proteins of an interferon, for example fusion proteins of interferon-α-2a, interferon-α-2b; consensus interferon or purified interferon-α product, each of which is fused with another protein. Certain preferred fusion proteins comprise an interferon (e.g., interferon-α-2b) and an albumin as described in U.S. Pat. No. 6,973,322 and international publications WO02/60071, WO05/003296 and WO05/077042(Human Genome Sciences). A preferred interferon conjugated to a human albumin is Albuferon (Human Genome Sciences).

Cyclosporins which bind strongly to cyclophilin but are not immunosuppressive include those cyclosporins recited in U.S. Pat. Nos. 5,767,069 and 5,981,479 and are incorporated herein by reference. MeIle4-Cyclosporin is a preferred non-immunosuppressive cyclosporin. Certain other cyclosporin derivatives are described in WO2006039668 (Scynexis) and WO2006038088 (Debiopharm SA) and are incorporated herein by reference. A cyclosporin is considered to be non-immunosuppressive when it has an activity in the Mixed Lymphocyte Reaction (MLR) of no more than 5%, preferably no more than 2%, that of cyclosporin A. The Mixed Lymphocyte Reaction is described by T. Meo in “Immunological Methods”, L. Lefkovits and B. Peris, Eds., Academic Press, N.Y. pp. 227-239 (1979). Spleen cells (0.5×10⁶) from Balb/c mice (female, 8-10 weeks) are co-incubated for 5 days with 0.5×10⁶ irradiated (2000 rads) or mitomycin C treated spleen cells from CBA mice (female, 8-10 weeks). The irradiated allogeneic cells induce a proliferative response in the Balb c spleen cells which can be measured by labeled precursor incorporation into the DNA. Since the stimulator cells are irradiated (or mitomycin C treated) they do not respond to the Balb/c cells with proliferation but do retain their antigenicity. The IC₅₀ found for the test compound in the MLR is compared with that found for cyclosporin A in a parallel experiment. In addition, non-immunosuppressive cyclosporins lack the capacity of inhibiting CN and the downstream NF-AT pathway. [MeIle]4-ciclosporin is a preferred non-immunosuppressive cyclophilin-binding cyclosporin for use according to the invention.

Ribavirin (1-β-D-ribofuranosyl-1-1,2,4-triazole-3-caroxamide) is a synthetic, non-interferon-inducing, broad spectrum antiviral nucleoside analog sold under the trade name, Virazole (The Merck Index, 11^{th} edition, Editor: Budavar, S, Merck & Co., Inc., Rahway, N.J.,

p1304, 1989). U.S. Pat. No. 3,798,209 and RE29,835 (incorporated herein by reference in their entireties) disclose and claim ribavirin. Ribavirin is structurally similar to guanosine, and has in vitro activity against several DNA and RNA viruses including Flaviviridae (Gary L. Davis, Gastroenterology 118:S104-S114, 2000).

Ribavirin reduces serum amino transferase levels to normal in 40% of patients, but it does not lower serum levels of HCV-RNA (Gary L. Davis, Gastroenterology 118:S104-S114, 2000). Thus, ribavirin alone is not effective in reducing viral RNA levels. Additionally, ribavirin has significant toxicity and is known to induce anemia. Ribavirin is not approved for monotherapy against HCV; it is approved in combination with interferon alpha-2a or interferon alpha-2b for the treatment of HCV.

A further preferred combination is a combination of a compound of the invention (e.g., a compound of Formula I or any subformulae thereof) with a non-immundsuppressive cyclophilin-binding cyclosporine, with mycophenolic acid, a salt or a prodrug thereof, and/or with a SIP receptor agonist, e.g. FTY720.

Additional examples of compounds that can be used in combination or alternation treatments include:

(1) Interferons, including interferon alpha 2a or 2b and pegylated (PEG) interferon alpha 2a or 2b, for example:
(a) Intron-A®, interferon alfa-2b (Schering Corporation, Kenilworth, N.J.);
(b) PEG-Intron®, peginteferon alfa-2b (Schering Corporation, Kenilworth, N.J.);
(c) Roferon®, recombinant interferon alfa-2a (Hoffmann-La Roche, Nutley, N.J.);
(d) Pegasys®, peginterferon alfa-2a (Hoffmann-La Roche, Nutley, N.J.);
(e) Berefor®, interferon alfa 2 available (Boehringer Ingelheim Pharmaceutical, Inc., Ridgefield, Conn.);
(f) Sumiferon®, a purified blend of natural alpha interferons (Sumitomo, Japan)
(g) Wellferon®, lymphoblastoid interferon alpha n1 (GlaxoSmithKline);
(h) Infergen®, consensus alpha interferon (InterMune Pharmaceuticals, Inc., Brisbane, Calif.);
(i) Alferon®, a mixture of natural alpha interferons (Interferon Sciences, and Purdue Frederick Co., CT);
(j) Viraferon®;
(k) Consensus alpha interferon from Amgen, Inc., Newbury Park, Calif.,

Other forms of interferon include: interferon beta, gamma, tau and omega, such as. Rebif (Interferon beta 1a) by Serono, Omniferon (natural interferon) by Viragen, REBIF (interferon beta-1a) by Ares-Serono, Omega Interferon by BioMedicines; oral Interferon Alpha by Amarillo Biosciences; an interferon conjugated to a water soluble polymer or to a human albumin, e.g., Albuferon (Human Genome Sciences), an antiviral agent, a consensus interferon, ovine or bovine interferon-tau

Conjugates of interferon to a water-soluble polymer are meant to include especially conjugates to polyalkylene oxide homopolymers such as polyethylene glocol (PEG) or polypropylene glycols, polyoxyethylenated polyols, copolymers thereof and block copolymers thereof. As an alternative to polyalkylene oxid-based polymers, effectively non-antigehic materials such as dextran, polyvinyl pyrrolidones, polyacrylamides, polyvinyl alcohols, carbohydrate-based polymers and the like can be used. Since the polymeric modification sufficiently reduces antigenic response, the foreign interferon need not be completely autologous. Interferon used to prepare polymer conjugates may be prepared from a mammalian extract, such as human, ruminant or bovine interferon, or recombinantly produced. Preferred are conjugates of interferon to polyethylene glycol, also known as pegylated interferons.

(2) Ribavirin, such as ribavirin (1-beta-D-ribofurariosyl-1H-1,2,4-triazple-3-carboxamide) from Valeant Pharmaceuticals, Inc., Costa Mesa, Calif.); Rebetol® from Schering Corporation, Kenilworth, N.J., and Copegus® from Hoffmann-La Roche, Nutley, N.J.; and new ribavirin analogues in development such as Levovirin and Viramidine by Valeant,

(3) Thiazolidine derivatives which show relevant inhibition in a reverse-phase HPLC assay with an NS3/4A fusion protein and NS5A/5B substrate (Sudo K. et al., Antiviral Research, 1996, 32, 9-18), especially compound RD-1-6250, possessing a fused cinnamoyl moiety substituted with a long alkyl chain, RD4 6205 and RD4 6193;

(4) Thiazolidines and benzanilides identified in Kakiuchi N. et al. J. FEBS Letters 421,217-220; Takeshita N. et al. Analytical Biochemistry, 1997,247, 242-246;

(5) A phenan-threnequinone possessing activity against protease in a SDS-PAGE and autoradiography assay isolated from the fermentation culture broth of Streptomyces sp., Sch 68631 (Chu M. et al., Tetrahedron Letters, 1996, 37, 7229-7232), and Sch 351633, isolated from the fungus Penicillium griseofulvum, which demonstrates activity in a scintillation proximity assay (Chu M. et al, Bioorganic and Medicinal Chemistry Letters 9, 1949-1952);

(6) Protease inhibitors.

Examples include substrate-based NS3 protease inhibitors (Attwood et al., Antiviral peptide derivatives, PCT WO 98/22496, 1998; Attwood et al., Antiviral Chemistry and Chemotherapy 1999, 10, 259-273; Attwood et al, Preparation and use of amino acid derivatives as anti-viral agents, German Patent Pub. DE 19914474; Tung et al. Inhibitors of serine proteases, particularly hepatitis C virus NS3 protease; PCT WO 98/17679), including alphaketoamides and hydrazinoureas, and inhibitors that terminate in an electrophile such as a boronic acid or phosphonate (Llinas-Brunet et al. Hepatitis C inhibitor peptide analogues, PCT WO 99/07734) are being investigated.

Non-substrate-based NS3 protease inhibitors such as 2,4,6-trihydroxy-3-nitro-benzamide derivatives (Sudo K. et al., Biochemical and Biophysical Research Communications, 1997, 238 643-647; Sudo K. et al. Antiviral Chemistry and Chemotherapy, 1998, 9, 186), including RD3-4082 and RD3-4078, the former substituted on the amide with a 14 carbon chain and the latter processing a para-phenoxyphenyl group are also being investigated.

Sch 68631, a phenanthrenequinone, is an HCV protease inhibitor (Chu M et al., Tetrahedron Letters 37: 7229-7232, 1996). In another example by the same authors, Sch 351633, isolated from the fungus Penicillium grieofulvum, was identified as a protease inhibitor (Chu M. et al., Bioorganic and Medicinal Chemistry Letters 9: 1949-1952). Nanomolar potency against the HCV NS3 protease enzyme has been achieved by the design of selective inhibitors based on the macromolecule eglin c. Eglin c, isolated from leech, is a potent inhibitor of several serine proteases such as S. griseus proteases A and B, ∀-chymotrypsin, chymase and subtilisin. Qasim M. A. et al., Biochemistry 36: 1598-1607, 1997.

U.S. patents disclosing protease inhibitors for the treatment of HCV include, for example, U.S. Pat. No. 6,004,933 to Spruce et al (incorporated herein by reference in its entirety) which discloses a class of cysteine protease inhibitors for inhibiting HCV endopeptidase 2; U.S. Pat. No. 5,990,276 to Zhang et al.(incorporated herein by reference in its entirety) which discloses synthetic inhibitors of hepatitis C virus NS3 protease; U.S. Pat. No. 5,538,865 to Reyes et al. (incorporated herein by reference in its entirety). Peptides as NS3 serine protease inhibitors of HCV are disclosed in WO 02/008251 to Corvas International, Inc., and WO 02/08187 and WO 02/008256 to Schering Corporation (incorporated herein by reference in their entireties). HCV inhibitor tripeptides are disclosed in U.S. Pat. Nos. 6,534,523, 6,410,531 and 6,420,380 to Boehringer Ingelheim and WO 02/060926 to Bristol Myers Squibb (incorporated herein by reference in their entireties). Diaryl peptides as NS3 serine protease inhibitors of HCV are disclosed in WO 02/48172 to Schering Corporation (incorporated herein by reference). Imidazoleidinones as NS3 serine protease inhibitors of HCV are disclosed in WO 02/18198 to Schering Corporation and WO 02/48157 to Bristol Myers Squibb (incorporated herein by reference in their entireties). WO 98/17679 to Vertex Pharmaceuticals and WO 02/48116 to Bristol Myers Squibb also disclose HCV protease inhibitors (incorporated herein by reference in their entireties).

HCV NS3-4A serine protease inhibitors including BILN 2061 by Boehringer Ingelheim, VX-950 by Vertex, 1TMN-191 by Intermune, SCH 6/7 by Schering-Plough, and other compounds currently in preclinical development;

Substrate-based NS3 protease inhibitors, including alphaketoamides and hydrazinoureas, and inhibitors that terminate in an elecrophile such as a boronic acid or phosphonate; Non-substrate-based NS3 protease inhibitors such as 2,4,6-trihydroxy-3-nitro-benzamide derivatives including RD3-4082 and RD3-4078, the former substituted on the amide with a 14 carbon chain and the latter processing a para-phenoxyphenyl group; and Sch68631, a phenanthrenequinone, an HCV protease inhibitor.

Sch 351633, isolated from the fungus Penicillium griseofulvum was identified as a protease inhibitor. Eglin c, isolated from leech is a potent inhibitor of several serine proteases such as S. griseus proteases A and B, a-chymotrypsin, chymase and subtilisin.

U.S. Pat. No. 6,004,933 (incorporated herein by reference in its entirety) discloses a class of cysteine protease inhibitors from inhibiting HCV endopeptidase 2; synthetic inhibitors of HCV NS3 protease (pat), HCV inhibitor tripeptides (pat), diaryl peptides such as NS3 serine protease inhibitors of HCV (pat), Imidazolidindiones as NS3 serine protease inhibitors of HCV (pat).

Thiazolidines and benzanilides (ref). Thiazolidine derivatives which show relevant inhibition in a reverse-phase HPLC assay with an NS3/4A fusion protein and NS5A/5B-substrate especially compound RD-16250 possessing a fused cinnamoyl moiety substituted with a long alkyl chain, RD4 6205 and RD4 6193

Phenan-threnequinone possessing activity against proteasein a SDS-PAGE and autoradiography assay isolated from the fermentation culture broth of Streptomyces sp, Sch68631 and Sch351633, isolated from the fungus Penicillium griseofulvum, which demonstrates activity in a scintillation proximity assay.

(7) Nucleoside or non-nucleoside inhibitors of HCV NS5B RNA-dependent RNA polymerase, such as 2′-C-methyl-3′-O-L-valine ester ribofuranosyl cytidine (Idenix) as disclosed in WO 2004/002422 A2 (incorporated herein by reference in its entirety), R803(Rigel), JTK-003 (Japan Tabacco), HCV-086 (ViroPharma/Wyeth) and other compounds currently in preclinical development;

gliotoxin (ref) and the natural product cerulenin;

2′-fluoronucleosides;

other nucleoside analogues as disclosed in WO 02/057287 A2, WO 02/057425 A2, WO 01/90121, WO 01/92282, and U.S. Pat. No. 6,812,219, the disclosures of which are incorporated herein by reference in their entirety.

Idenix Pharmaceuticals discloses the use of branched nucleosides in the treatment of flaviviruses (including HGV) and pestiviruses in International Publication Nos. WO 01/90121 and WO 01/92282 (incorporated herein by reference in their entireties). Specifically, a method for the treatment of hepatitis C infection (and flaviviruses and pestiviruses) in humans and other host animals is disclosed in the Idenix publications that includes administering an effective amount of a biologically active 1′, 2′, 3′ or 4′-branced B-D or B-L nucleosides or a pharmaceutically acceptable salt or prodrug thereof, administered either alone or in combination with another antiviral agent, optionally in a pharmaceutically acceptable carrier. Certain preferred biologically active 1′, 2′, 3′, or 4′ branched B-D or B-L nucleosides, including Telbivudine, are described in U.S. Pat. Nos. 6,395,716 and 6,875,751, each of which are incorporated herein by reference.

Other patent applications disclosing the use of certain nucleoside analogs to treat hepatitis C virus include: PCTCA00/01316 (WO 01/32153; filed Nov. 3, 2000) and PCT/CA01/00197 (WO 01/60315; filed Feb. 19, 2001) filed by BioChem Pharma, Inc., (now Shire Biochem, Inc.); PCT/US02/01531 (WO 02/057425; filed Jan. 18, 2002) and PCT/US02/03086 (WO 02/057287; filed Jan. 18, 2002) filed by Merck & Co., Inc., PCT/EP01/09633 (WO 02/18404; published Aug. 21, 2001) filed by Roche, and PCT Publication Nos. WO 01/79246 (filed Apr. 13, 2001), WO 02/32920 (filed Oct. 18, 2001) and WO 02/48165 by Pharmasset, Ltd. (the disclosures of which are incorporated herein by reference in their entireties)

PCT Publication No. WO 99/43691 to Emory University (incorporated herein by reference in its entirety), entitled “2′-Fluoronucleosides” discloses the use of certain 2′-fluoronucleosides to treat HCV.

Eldrup et al. (Oral Session V, Hepatitis C Virus, Flaviviridae; 16^{th} International Conference on Antiviral Research (Apr. 27, 2003, Savannah, Ga.)) described the structure activity relationship of 2′-modified nucleosides for inhibition of HCV.

D Bhat et al. (Oral Session V, Hepatitis C Virus, Flaviviridae, 2003 (Oral Session V, Hepatitis C Virus, Flaviviridae; 16^{th} International conference on Antiviral Research (Apr. 27, 2003, Savannah, Ga.); p A75) describes the synthesis and pharmacokinetic properties of nucleoside analogues as possible inhibitors of HCV RNA replication. The authors report that 2′-modified nucleosides demonstrate potent inhibitory activity in cell-based replicon assays.

Olsen et al. (Oral Session V, Hepatitis C Virus, Flaviviridae; 16^{th} International Conference on Antiviral Research (Apr. 27, 2003, Savannah, Ga.)p A76) also described the effects of the 2′-modified nucleosides on HCV RNA replication.

(8) Nucleotide polymerase inhibitors and gliotoxin (Ferrari R. et al. Journal of Virology, 1999, 73, 1649-1654), and the natural product cerulenin (Lohmann V. et al. Virology, 1998, 249, 108-118);

(9) HCV NS3 helicase inhibitors, such as VP_{—}50406 by ViroPhama and compounds from Vertex. Other helicase inhibitors (Diana G. D. et al., Compounds, compositions and methods for treatment of hepatitis C, U.S. Pat. No. 5,633,358 (incorporated herein by reference in its entirety); Diana G. D. et al., Piperidine derivatives, pharmaceutical compositions thereof and their use in the treatment of hepatitis C, PCT WO 97/36554);

(10) Antisense phosphorothioate oligodeoxynucleotides (S-ODN) complementary to sequence stretches in the 5′ non-coding region (NCR) of the virus (Alt M. et al., Hepatology, 1995, 22, 707-717), or nucleotides 326-348 comprising the 3′ end of the NCR and nucleotides 371-388 located in the core coding region of the HCV RNA (Alt M. et al., Archives of Virology, 1997, 142, 589-599; Galderisi U. et al., Journal of Cellular Physiology, 199, 181, 251-257); such as ISIS 14803 by Isis Pharm/Elan, antisense by Hybridon, antisense by AVI bioPharma,

(11) Inhibitors of IRES-dependent translation (Ikeda N et al., Agent for the prevention and treatment of hepatitis C, Japanese Patent Pub. JP-08268890; Kai Y et al. Prevention and treatment of viral diseases, Japanese Patent Pub. JP-10101591); such as ISIS 14803 by Isis Pharm/Elan, IRES inhibitor by Anadys, IRES inhibitors by Immusol, targeted RNA chemistry by PTC Therapeutics

(12) Ribozymes, such as nuclease-resistant ribozymes (Maccjak, D. J. et al., Hepatology 1999, 30, abstract 995) and those directed in U.S. Pat. No. 6,043,077 to Barber et al., and U.S. Pat. Nos. 5,869,253 and 5,610,054 to Draper et al (incorporated herein by reference in their entireties) for example, HEPTAZYME by RPI

(13) siRNA directed against HCV genome

(14) HCV replication inhibitor of any other mechanisms such as by VP50406ViroPharama/Wyeth, inhibitors from Achillion, Arrow

(15) An inhibitor of other targets in the HCV life cycle including viral entry, assembly and maturation

(16) An immune modulating agent such as an 1MPDH inhibitor, mycophenolic acid, a salt or a prodrug thereof sodium mycophenolate or mycophenolate mofetil, or Merimebodib (VX-497); thymosin alpha-1 (Zadaxih, by SciClone); or a SIP receptor agonist, e.g. FTY720 or analogue thereof optionally phosphorylated.

(17) An anti-fibrotic agent, such as a N-phenyl-2-pyrimidine-amine derivative, imatinib (Gleevac), IP-501 by Indevus, and Interferon gamma 1b from InterMune

(18) Therapeutic vaccine by Intercell, Epimmune/Genecor, Merix, Tripep (Chron-VacC), immunotherapy (Therapore) by Avant, T cell therapy by CellExSys, monoclonal antibody XTL-002 by STL, ANA 246 and ANA 246 BY Anadys,

(19) Other miscellaneous compounds including 1-amino-alkylcyclohexahes (U.S. Pat. No. 6,034,134 to Gold et al.), alkyl lipids (U.S. Pat. No. 5,922,757 to Chojkier et al.), vitamin E and other anti-oxidants (U.S. Pat. No. 5,922,757 to Chojkier et al.), amantadine, bile acids (U.S. Pat. No. 5,846,999,64 to Ozeki et al.), N-(phosphonoacetl)-L-aspartic acid,) U.S. Pat. No. 5,830,905 to Diana et al.), benzenedicarboxamides (U.S. Pat. No. 5,633,388 to Diane et al.), polyadenylic acid derivatives (U.S. Pat. No. 5,496,546 to Wang et al.), 2′3′-dideoxyinosine (U.S. Pat. No. 5,026,687 to Yarchoan et al.), benzimidazoles (U.S. Pat. No. 5,891,874 to Colacino et al.), plant extracts (U.S. Pat. No. 5,837,257 to Tsai et al., U.S. Pat. No. 5,725,859 to Omer et al., and U.S. Pat. No. 6,056,961) and piperidines (U.S. Pat. No. 5,830,905 to Diana et al.); the disclosures of which are incorporated herein by reference in their entireties. Also, squalene, telbivudine, N-(phosphonoacetyl)-L-aspartic acid, benzenedicarboxamides, polyadenylic acid derivatives, glycosylation inhibitors, and nonspecific cytoprotective agents that block cell injury caused by the virus infection.

(20) Any other compound currently in preclinical or clinical development for the treatment of HCV, including Interleukin-10 (Schering-Plough), AMANTADINE (Symmetrel) by Endo Labs Solvay, caspase inhibitor IDN-6556 by Idun Pharma, HCV/MF59 by Chiron, CIVACIR (Hepatitis C Immune Globulin) by NABI, CEPLENE (histamine dichloride) by Maxim, IDN-6556 by Idun PHARM, T67, a beta-tubulin inhibitor, by Tularik, a therapeutic vaccine directed to E2 by Innogenetics, FK788 by Fujisawa Helathcare, IdB1016 (Siliphos, oral silybin-phosphatidyl choline phytosome), fusion inhibitor by Trimeris, Dication by Immtech, hemopurifier by Aethlon Medical, UT 231B by United Therapeutics.

(21) Purine nucleoside analog antagonists of T1R7 (toll-like receptors) developed by Anadys, e.g., Isotorabine (ANA245) and its prodrug (ANA975), which are described in European applications EP348446 and EP636372, International Publications WO03/045968, WO05/121162 and WO05/25583, and U.S. Pat. No. 6,973,322, each of which is incorporated by reference.

(21) Non-nucleoside inhibitors developed by Genelabs and described in International Publications WO2004/108687, WO2005/12288, and WO2006/076529, each of which is incorporated by reference.

(22) Other co-agents (e.g., non-immunomodulatory or immunomodulatory compounds) that may be used in combination with a compound of this invention include, but are not limited to, those specified in WO 02/18369, which is incorporated herein by reference.

Methods of this invention may also involve administration of another component comprising an additional agent selected from an immunomodulatory agent; an antiviral agent; an inhibitor of HCV protease; an inhibitor of another target in the HCV life cycle; a CYP inhibitor; or combinations thereof.

Accordingly, in another embodiment, this invention provides a method comprising administering a compound of the invention and another anti-viral agent, preferably an anti-HCV agent. Such anti-viral agents include, but are not limited to, immunomodulatory agents, such as α, β, and δ interferons, pegylated derivatized interferon-a compounds, and thymosin; other anti-viral agents, such as ribavirin, amantadine, and telbivudine; other inhibitors of hepatitis C proteases (NS2-NS3 inhibitors and NS3-NS4A inhibitors); inhibitors of other targets in the HCV life cycle, including helicase, polymerase, and metalloprotease inhibitors; inhibitors of internal ribosome entry; broad-spectrum viral inhibitors, such as IMPDH inhibitors (e.g., compounds of U.S. Pat. Nos. 5,807,876, 6,498,178, 6,344,465, 6,054,472, WO 97/40028, WO 98/40381, WO 00/56331, and mycopheholic acid and derivatives thereof, and including, but not limited to VX-497, VX-148, and/or VX-944); or combinations of any of the above.

In accordance with the foregoing the present invention provides in a yet further aspect:
A pharmaceutical combination comprising a) a first agent which is a compound of the invention, e.g. a compound of formula I or any subformulae thereof, and b) a co-agent, e.g. a second drug agent as defined above.
A method as defined above comprising co-administration, e.g. concomitantly or in

I sequence, of a therapeutically effective amount of a compound of the invention, e.g. a compound of formula I or any subformulae thereof, and a co-agent, e.g. a second drug agent as defined above.

The terms “co-administration” or “combined administration” or the like as utilized herein are meant to encompass administration of the selected therapeutic agents to a single patient, and are intended to include treatment regimens in which the agents are not necessarily administered by the same route of administration or at the same time. Fixed combinations are also within the scope of the present invention. The administration of a pharmaceutical combination of the invention results in a beneficial effect, e.g. a synergistic therapeutic effect, compared to a monotherapy applying only one of its pharmaceutically active ingredients.

Each component of a combination according to this invention may be administered separately, together, or in any combination thereof. As recognized by skilled practitioners, dosages of interferon are typically measured in IU (e.g., about 4 million IU to about 12million IU).

If an additional agent is selected from another CYP inhibitor, the method would, therefore, employ two or more CYP inhibitors. Each component may be administered in one or more dosage forms. Each dosage form may be administered to the patient in any order.

The compound of the invention and any additional agent may be formulated in separate dosage forms. Alternatively, to decrease the number of dosage forms administered to a patient, the compound of the invention and any additional agent may be formulated together in any combination. For example, the compound of the invention inhibitor may be formulated in one dosage form and the additional agent may be formulated together in another dosage form. Any separate dosage forms may be administered at the same time or different times.

Alternatively, a composition of this invention comprises an additional agent as described herein. Each component may be present in individual compositions, combination compositions, or in a single composition.

### Exemplification of the Invention

The invention is further illustrated by the following examples, which should not be construed as further limiting. The assays used throughout the Examples are accepted. Demonstration of efficacy in these assays is predictive of efficacy in subjects.

The following abbreviations are used throughout the examples and the specification;
API-MS Atmospheric Pressure Ionization Mass Spectrometry
CDl Carbonyldiimidazole
DABCO 1,4-Diazabicyclo[2.2.2]octane
DBU 1,8-Diazabicyclo[5.4.0]-undec-7-ene
DIPEA N-Ethyldiisopropylamine
DMF N,N′-pimethylformamide
DMSO Dimethylsulfoxide
Grubbs II catalyst Benzylidene[1,3-bis(2,4,6-trimethylphenyl)-2-imazolidinylidene]dichloro(tricyclohexylphosphine)ruthenium
HBTU O-(Benzotriazol-1-yl)-N,N,N′N′-tetramethyluronium hexafluorophosphate
HPLC High Performance Liquid Chromatography
LC-MS Liquid Chromatography Mass Spectrometry
Rf Retention factor
RT Room temperature
Rt Retention time
Teoc 2-Trimethylsilylethoxycarbonyl
THF Tetrahydrofuran
TLC Thin layer chromatography

### Analytical Methods:

LC-MS (method A):
Instrument: Agilent system
Column: Waters symmetry, 3.5 micron, 50×2.1 mm, 5 min, 20% to 95% CH_{CN}
solvent: CH₃CN (0.1% HCO₂H); H₂O (0.1% HCO₂H)
gradient: 0-3.5 min: 20-95% CH₃CN, 3.5-5 min: 95% CH₃CN, 5.5-5.55 min 95% to 20% CH₃CN

### HPLC (Method B):

Instrument: Kontron, Kroma-System
Column: Macherey-Nagel, Lichrosphere 100-5 RP 18
Solvent: CH₃CN (0.1% CF₃CO₂H); H₂O (0.1% CF₃CO₂H)
Gradient: 0-5 min: 10-100% CH₃CN; 5-7.5 min: 100% CH₃CN (Flow 1.5 mL/min)

### HPLC (Method C):

Instrument: Agilent system
column: waters symmetry C18, 3.5 micron, 2.1×50 mm, flow 0.6 ml/min
solvent: CH₃CN (0.1% CF₃CO₂H); H₂O (0.1% CF₃CO₂H)
gradient: 0-3.5 min: 20-95% CH₃CN, 3.5-5 min: 95% CH₃CN, 5.5-5.55 min 95% to 20% CH₃CN

### MS (Method D):

Instrument: Agilent 1100 Series
Detection: API-ES, positive/negative

### Preparative HPLC (Method E):

Instrument: Gilson system
column: waters C18 ODB, 5 micron, 50×19 mm
solvent: CH₃CN (0.1% HCO₂H); H₂O (0.1% HCO₂H)

### Preparative HPLC (Method F):

Instrument: Gilson
Column: Sun-Fire prep C18 OBD 5 micron, Column 19×50 mm (flow 20 mL/min) or
Column 30×100 mm (flow 40 mL/min)

Solvent: CH₃CN (0.1% CF₃CO₂H) and H₂O (0.1% CF₃CO₂H)
Gradient: 0-20 min: 5-100% CH₃CN

### General Synthetic Methods

### Acyl-Sulfonamide Macrocycles

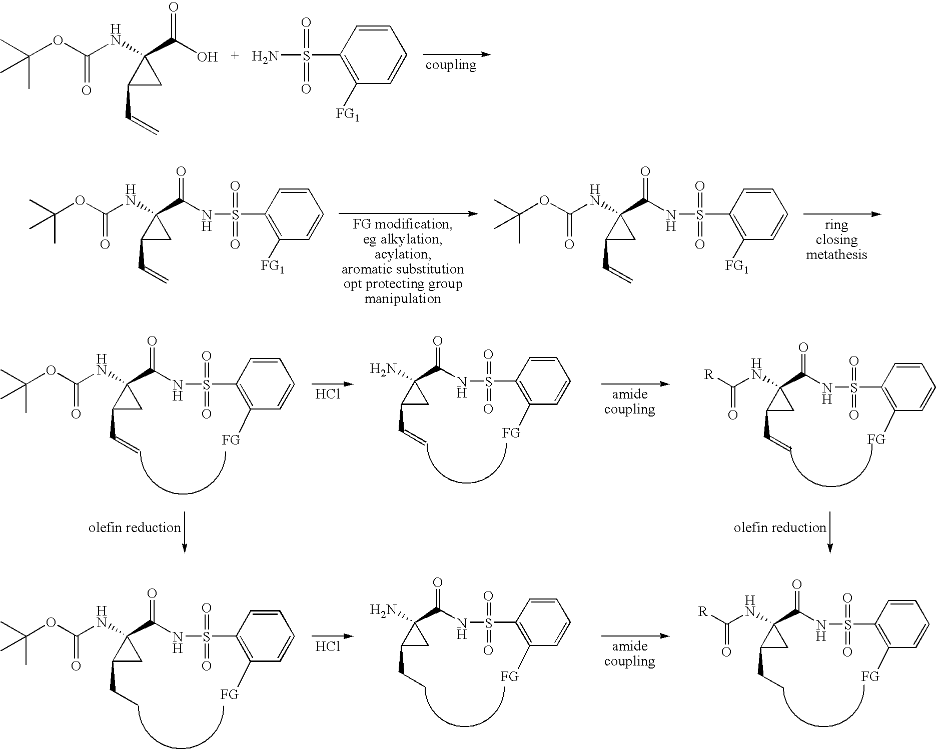

Acyl-Amide Macrocycles (Synthesis of Compounds in which L₁-FG-L₂-L₃ is an Alkylene-Amide-Alkylene Residue)

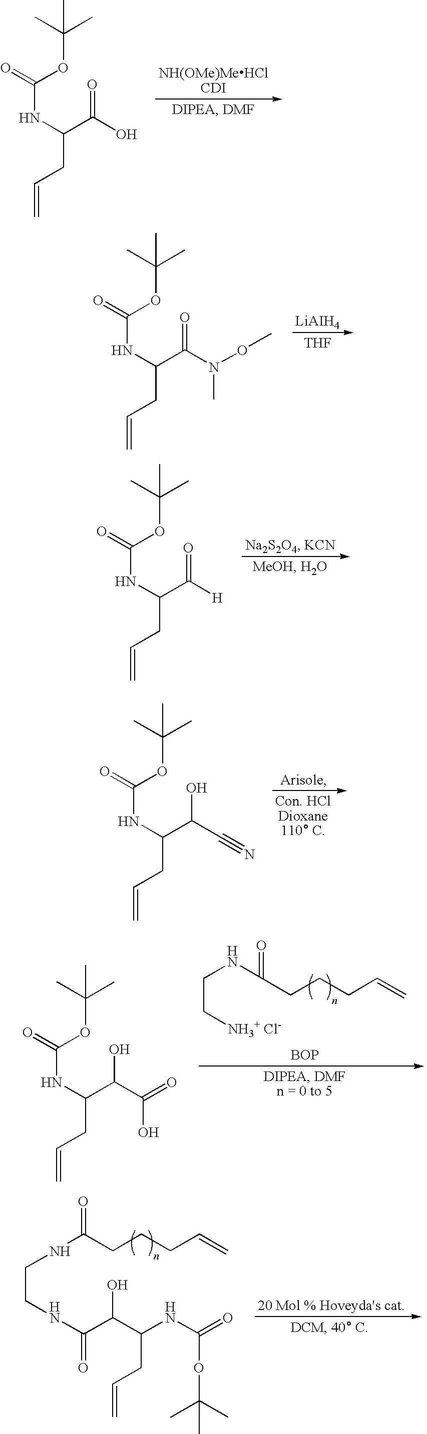

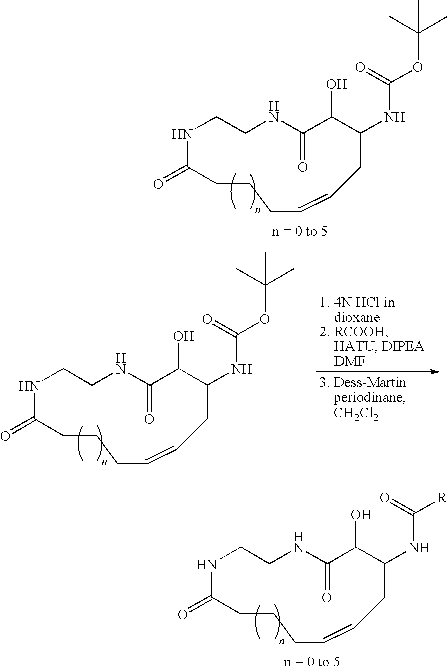

Acyl-Amide Macrocycles (Synthesis of Compounds in which L₁-FG-L₂-L₃ is an Arylene-Amide-Alkylene Residue)

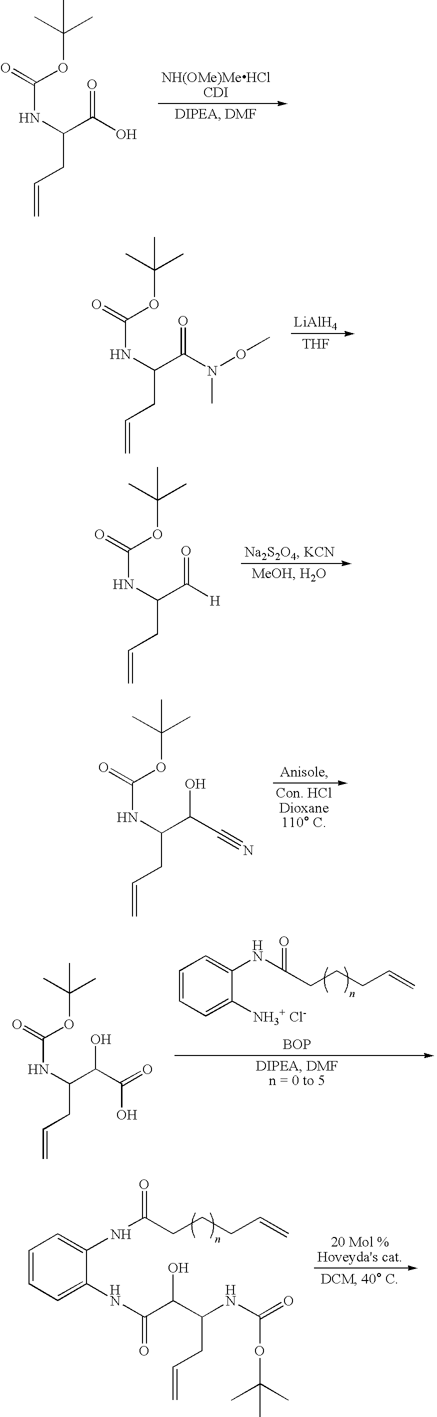

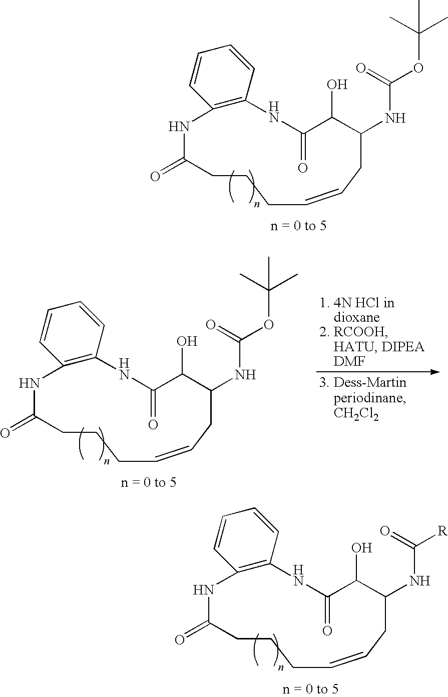

### Example 1

### (2S,4R)-1-Acetyl-4-(7-methoxy-2-phenyl-quinolin-4-yloxy)-pyrrolidine-2-carboxylic acid ((Z)-(5R,7S)-2,2,4,14-tetraoxo-2Λ⁶-thia-3,15-diaza-tricyclo[14.4.0.0^{5,7}]icosa-1(16),8,17,19-tetraen-5-yl)-amide

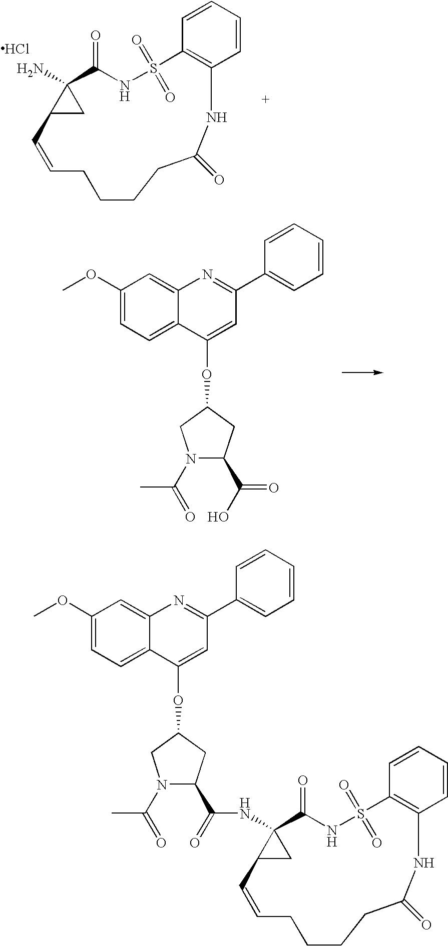

To a solution of 28 mg (0.069 mmol) of (Z)-(5R,7S)-5-Amino-2,2-dioxo-2Λ⁶-thia-3,15-diaza-tricyclo[14.4.0.0^{5,7}]icosa-1(16),8,17,19-tetraene-4,14-dione hydrochloride, 36 mg (0.082 mmol) of (2S,4R)-1-Acetyl-4-(7-methoxy-2-phenyl-quinolin-4-yloxy)-pyrrolidine-2-carboxylic acid and 0.048 mL (0.28 mmol) of DIPEA in 0.3 mL DMF is added 40 mg (0.103 mmol) HBTU at 0° C. The reaction mixture is allowed to warm to RT and is stirred for 12 hours. 10 mL EtOAc are added and the organic phase is washed once with 1N HCl and twice with saturated aqueous NaHCO₃. The organic layer is dried with MgSO₄ arid concentrated in vacuo. The residue is purified by preparative reverse phase HPLC (Method E) to afford (2S,4R)-1-Acetyl-4-(7-methoxy-2-phenyl-quinolin-4-yloxy)-pyrrolidine-2-carboxylic acid ((Z)-(5R,7S)-2,2,4,14-tetraoxo-2Λ⁶-thia-3,15-diaza-tricyclo[14.4.0.0^{5,7}]icosa-1(16),8,17,19-tetraen-5-yl)-amide as a white solid. LC-MS (Method A): Rt=3.00 min; M+H=752.2

### Preparation of (2S,4R)-1-Acetyl-4-(7-methoxy-2-phenyl-quinolin-4-yloxy)-pyrrolidine-2-carboxylic acid

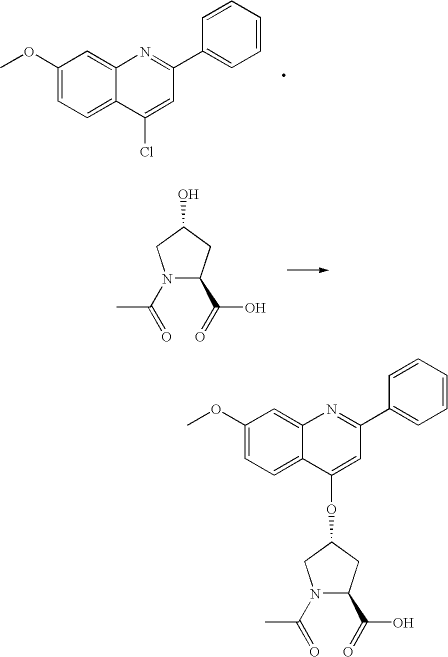

A solution of 6.42 g (37 mmol) of N-acetyl-L-hydroxyproline in 100 mL of DMSO is treated with 11.44 g (102 mmol) of sodium-tert-butylate while maintaining the temperature at RT with an ice-bath. After 90 min at RT 100 mL of DMSO is added, followed by 10 g (37 mmol) of 4-chloro-7-methoxy-2-phenyl-quinoline in 3 portions over a 45-min period. Upon completion of the reaction the resulting dark solution is taken up in water, neutralized with HCl, and saturated with NaCl. Extraction with CH₂Cl₂ affords the crude product as an oil that is chromatographed on SiO₂ (eluant CH₂Ch/MeOH 7:3). The resulting material is triturated and washed with EtOAc and dried to afford (2S,4R)-1-Acetyl-4-(7-methoxy-2-phenyl-quinolin-4-yloxy)-pyrrolidine-2-carboxylic acid as a light brown powder. Rt (HPLC; Method B)=4.49 min; MS (Method D): M−1=405.

### Preparation of (Z)-(5R,7S)-5-Amino-2,2-dioxo-2Λ⁶-thia-3,15-diaza-tricyclo[14.4.0.0^{5,7}]icosa-1(16),8,17,19-tetraene-4,14-dione hydrochloride

### Step 1

### (2-Sulfamoyl-phenyl)-carbamic acid 2-trimethylsilanyl-ethyl ester

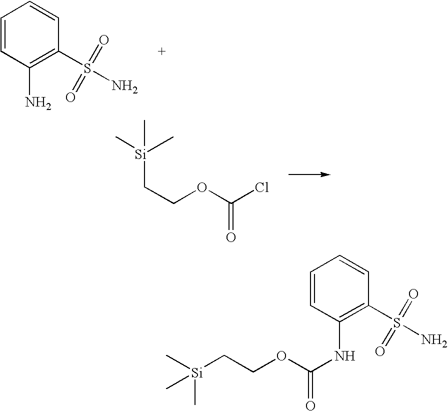

To a solution of 27.0 g (0.157 m°l) of 2-Aminobenzenesulfonamide and 17.0 g (0.160 mol) Na₂CO₃ in a mixture of 150 mL dioxane and 150 mL H₂O is added a solution of 28.9 g (0.160 mol) Teoc-Cl in 50 mL dioxane at 0° C. and the resulting mixture is stirred for 18 hours at RT 200 mL of 1N HCl and 300 mL ether are added. The organic phase is separated and the aqueous phase is extracted twice with 300 mL Et₂O each. The combined organic phases are dried with MgSO₄ and concentrated in vacuo. The residue is chromatographed on SiO₂ (eluant hexanes/EtOAc 6:1 to hexanes/EtOAc 2:1) to give (2-Sulfamoyl-phenyl)-carbamic acid 2-trimethylsilanyl-ethyl ester as a white solid. LC-MS (Method A): Rt=4.13 min; M+Na=339.0, M−1=315.1

### Step 2

### [2-[((1R,2S)-1-tert-Butoxycarbonylamino-2-vinyl-cyclopropanecarbonyl)-sulfamoyl]-phenyl]-carbamic acid 2-trimethylsilanyl-ethyl ester

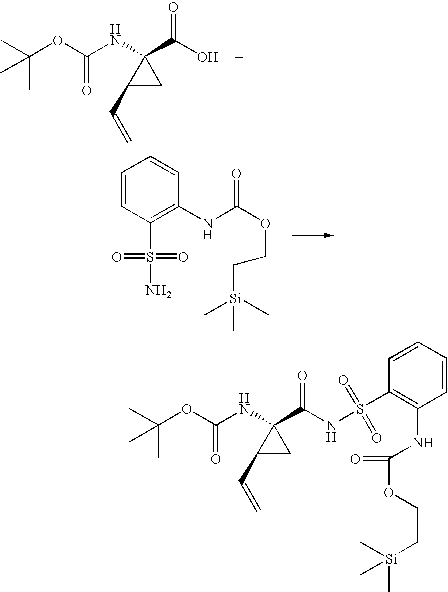

To a solution of 8.6 g (37.8 mmol) (1R,2S)-1-tert-Butoxycarbonylamino-2-vinyl-cyclopropanecarboxylic acid in 120 mL THF is added 9.69 g (56.8 mmol) CDI and the mixture is stirred at 70° C. for 2 hours. The mixture is allowed to cool to RT and 12.8 g (40.5 mmol) (2-Sulfamoyl-phenyl)-carbamic acid 2-trimethylsilanyl-ethyl ester and 8.6 mL (56.8 mmol) DBU are added. The reaction mixture is stirred at RT for 12 hours. 400 mL EtOAc are added and the mixture is washed twice with 150 mL 0.5 N HCl each: The organic layer is dried with MgSO₄ and concentrated in vacuo. The residue is chromatographed on SiO₂ (hexanes/EtOAc 6:1 to EtOAc) to give [2-[((1R,2S)-1-tert-Butoxycarbonylamino-2-vinyl-cyclopropanecarbonyl)-sulfamoyl]-phenyl]-carbamic acid 2-trimethylsilanyl-ethyl ester as a colorless oil. LC-MS (Method A): Rt=4.97 min; M+Na=548.2, M−1=524.2

### Step 3

### [(1R,2S)-1-(2-Amino-benzenesulfonylaminocarbonyl)-2-vinyl-cyclopropyl]-carbamic acid tert-butyl ester

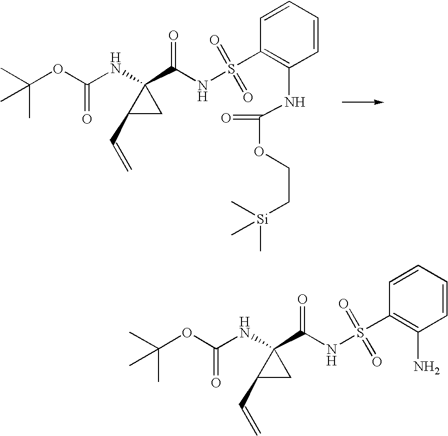

A mixture of 10 g (19.0 mmol) [2-[((1R,2S)-1-tert-Butoxycarbonylamino-2-vinyl-cyclopropanecarbonyl)-sulfamoyl]-phenyl]-carbamic acid 2-trimethylsilanyl-ethyl ester and 8.5 g (57.1 mmol) tetraethyl ammonium fluoride in 150 mL acetonitrile is stirred at 90° C. for 1.5 hours! The reaction mixture is concentrated in vacuo and the residue is chromatographed on SiO₂ (CH₂Cl₂/MeOH 98:2 to 9:1) to give [(1R,2S)-1-(2-Amino-benzenesulfonylaminocarbonyl)-2-vinyl-cyclopropyl]-carbamic acid tert-butyl ester as a white solid. LC-MS (Method A): Rt=3.75 min; M+Na=404.0, M−1=380.0

### Step 4

### [(1R,2S)-1-(2-Hept-6-enoylamino-benzenesulfonylaminocarbonyl)-2-vinylcyclopropyl]-carbamic acid tert-butyl ester

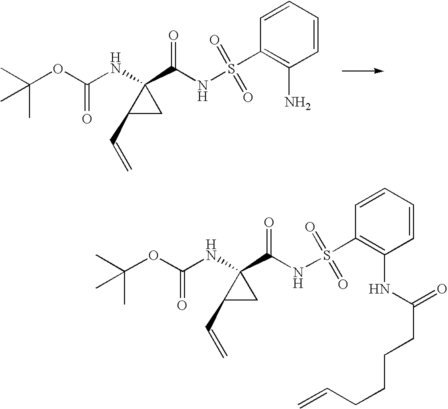

To a mixture of 0.45 g (1.18 mmol) [(1R,2S)-1-(2-Amino-benzenesulfonylaminocarbonyl)-2-vinyl-cyclopropyl]-carbamic acid tert-butyl ester and 0.25 g Na₂CO₃ (2.36 mmol) in 1.5 mL dioxane and 1.5 mL H₂O is slowly added 0.18 g (1.20 mmol) 6-heptenoic acid chloride at 0° C. The solution is allowed to warm to RT and is stirred for 12 hours. To drive the reaction to completion 0.18 g (1.20 mmol) 6-heptenoic acid chloride are added and the solution is stirred for another 4 hours. 20 mL EtOAc are added and the mixture is washed with 10 mL 1N HCl. The organic layer is dried with MgSO₄ and concentrated in vacuo. The residue is chromatographed on SiO₂ (eluant hexanes/EtOAc 100:0 to hexanes/EtOAc 0:100) to give [(1R,2S)-1-(2-Hept-6-enoylamino-benzenesulfonylaminocarbonyl)-2-vinylcyclopropyl]-carbamic acid tert-butyl ester. LC-MS (Method A): Rt=4.49 min; M−H=490.1

### Step 5

### ((Z)-(5R,7S)-2,2,4,14-Tetraoxo-2Λ⁶-thia-3,15-diaza-tricyclo[14.4.0.0^{5,7}]icosa-1(16),8,17,19-tetraen-5-yl)-carbamic acid tert-butyl ester/((E)-(5R,7S)-2,2,4,14-Tetraoxo-2Λ⁶-thia-3,15-diaza-tricyclo[14.4.0.0^{5,7}]icosa-1(16),8,17,19-tetraen-5-yl)-carbamic acid tert-butyl ester

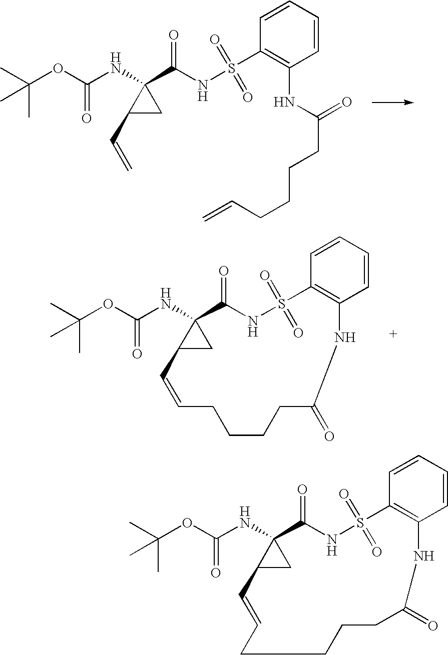

A solution of 450 mg (0.92 mmol) [(1R,2S)-1-(2-Hept-6-enoylamino-benzenesulfonylaminocarbonyl)-2-vinylcyclopropyl]-carbamic acid tert-butyl ester and 0.12 g (0.18 mmol) Grubbs II catalyst in 270 mL CH₂Cl₂ is heated to reflux for 12 hours. The reaction mixture is concentrated in vacuo and the residue is purified by preparative reverse phase HPLC (Method E) to afford ((Z)-(5R,7S)-2,2,4,14-Tetraoxo-2Λ⁶-thia-3,15-diaza-tricyclo[14.4.0.0^{5,7}]icosa-1(16),8,17,19-tetraen-5-yl)-carbamic acid tert-butyl ester and ((E)-(5R,7S)-2,2,4,14-Tetraoxo-2Λ⁶-thia-3,15-diaza-tricyclo[14.4.0.0^{5,7}]icosa-1(16)8,17,19-tetraen-5-yl)-carbamic acid tert-butyl ester, both as white powders. (Z) isomer: LC-MS (Method A): Rt=4.17 min; M−H=462.1; (E) isomer: LC-MS (Method A): Rt=3.99 min; M−H=462.1

### Step 6

### (Z)-(5R,7S)-5-Amino-2,2-dioxo-2Λ⁶-thia-3,15-diaza-tricyclo[14.4.0.0^{5,7}]icosa-1(16),8,17,19-tetraene-4,14-dione hydrochloride

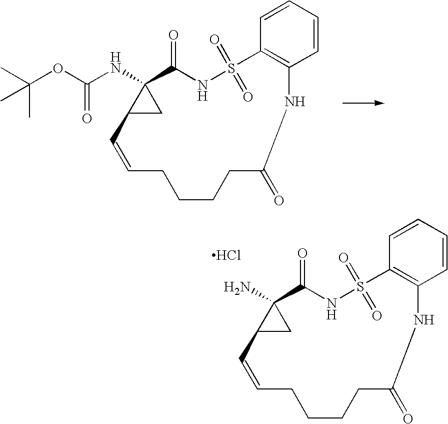

To a solution of 39 mg (0.084 mmol) ((Z)-(5R,7S)-2,2,4,14-Tetraoxo-2Λ⁶-thia-3,15-diaza-tricyclo[14.4.0.0^{5,7}]icosa-1(16),8,17,19-tetraen-5-yl)-carbamic acid tert-butyl ester in 0.21 mL dioxane is added 0.21 mL HCl in dioxane (4N) and the mixture is stirred at RT for 1 hour. The reaction mixture is concentrated in vacuo to afford (Z)-(5R,7S)-5-Amino-2,2-dioxo-2Λ⁶-thia-3,15-diaza-tricyclo[14.4.0.0^{5,7}]icosa-1(16),8,17,19-tetraene-4,14-hydrochloride. HPLC (Method C): Rt=0.63 min; LC-MS (Method A): M+H=364.1

### Example 2

### (2S,4R)-1-Acetyl-4-(7-methoxy-2-phenyl-quinolin-4-yloxy)-pyrrolidine-2-carboxylic acid ((E)-(5R,7S)-2,2,4,14-tetraoxo-2Λ⁶-thia-3,15-diaza-tricyclo[14.4.0.0^{5,7}]icosa-1(16)8,17,19-tetraen-5-yl)-amide

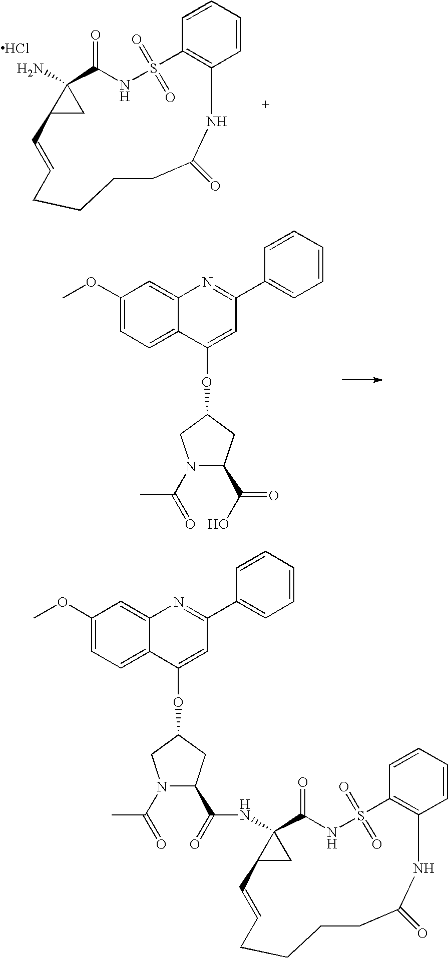

To a solution of 180 mg (0.44 mmol) (E)-(5R,7S)-5-Amino-2,2-dioxo-2Λ⁶-thia-3,15-diaza-tricyclo[14.4.0.0^{5,7}]icosa-1(16),8,17,19-tetraene-4,14-dione hydrochloride, 213 mg (0.53 mmol) (2S,4R)-1Acetyl-4-(7-methoxy-2-phenyl-quinolin-4-yloxy)-pyrrolidine-2-carboxylic acid and 0.309 mL (1.77 mmol) DIPEA in 1.4 mL DMF is added 257 mg (0.53 mmol) HBTU at 0° C. The reaction mixture is allowed to warm to RT and is stirred for 12 hours. 20 mL EtOAc are added and the organic phase is washed once with 1N HCl arid twice with saturated aqueous NaHCO₃. The organic layer is dried with MgSO₄ and concentrated in vacuo. The residue is purified by preparative reverse phase HPLC (Method E) to afford (2S,4R)-1-Acetyl-4-(7-methoxy-2-phenyl-quinolin-4-yloxy)-pyrrolidine-2-carboxylic acid ((E)-(5R,7S)-2,2,4,14-tetraoxo-2Λ⁶-thia-3,15-diaza-tricyclo[14.4.0.0^{5,7}]icosa-1(16)8,17,19-tetraen-5-yl)-amide as a white solid. LC-MS (Method A): Rt=3.03 min; M+H=752.2

### Preparation of (E)-(5R,7S)-5-Amino-2,2-dioxo-2Λ⁶-thia-3,15-diaza-tricyclo[14.4.0.0^{5,7}]icosa-1(16),8,17,19-tetraene-4,14-dione hydrochloride

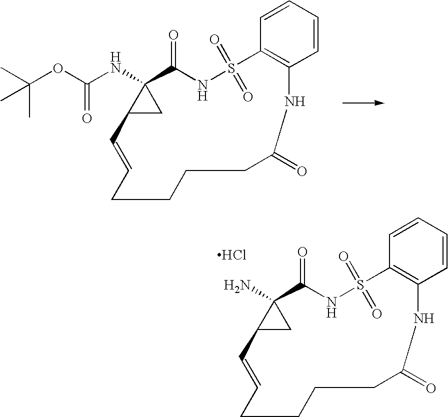

To a solution of 205 mg (0.44 mmol) ((E)-(5R,7S)-2,2,4,14-Tetraoxo-2Λ⁶-thia-3,15-diaza-tricyclo[14.4.0.0^{5,7}]icosa-1(16),8,17,19-tetraen-5-yl)-carbamic acid tert-butyl ester (Example 1, step 5) in 1.1 mL dioxane is added 1.1 mL HCl in dioxane (4N) and the mixture is stirred at RT for 1 hour. The reaction mixture is concentrated in vacuo to afford (E)-(5R,7S)-5-Amino-2,2-dioxo-2Λ⁶-thia-3,15-diaza-tricyclo[14.4.0.0^{5,7}]icosa-1(16),8,17,19-tetraene-4,14-dione hydrochloride. LC-MS (Method A): M+H=364.1

### Example 3

### (2S,4R)-1-Acetyl-4-(7-methoxy-2-phenyl-quinolin-4-yloxy)-pyrrolidine-2-carboxylic acid ((5R,7R)-2,2,4,14-tetraoxo-2Λ⁶-thia-3,15-diaza-tricyclo[4.4.0.0^{5,7}]icosa-1(16),17,19-trien-5-yl)-amide

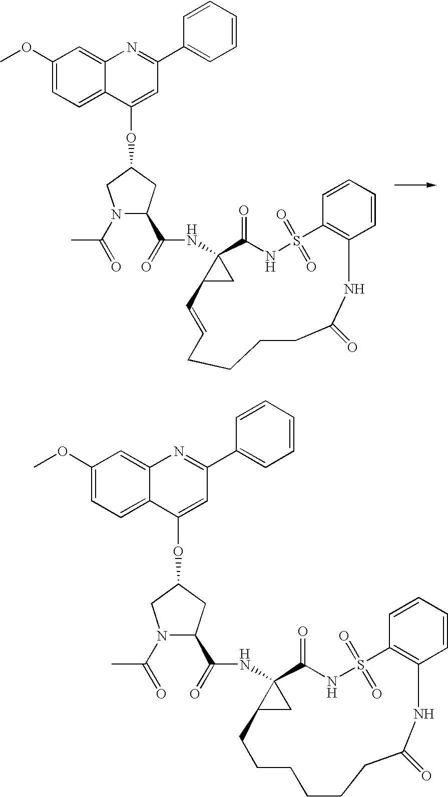

To a mixture of 50 mg (0.067 mmol) (2S,4R)-1-Acetyl-4-(7-methoxy-2-phenyl-quinolin-4-yloxy)-pyrrolidine-2-carboxylic acid ((E)-(5R,7S)-2,2,4,14-tetraoxo-2Λ⁶-thia-3,15-diaza-tricyclo[14.4.0.0^{5,7}]icosa-1(16)8,17,19-tetraen-5-yl)-amide and 310 mg (1.60 mmol) potassium diazodicarboxylate in 6 mL CH₂Cl₂ is added 1.7 mL AcOH (0.5 M in CH₂Cl₂) and the reaction is stirred at 45° C. for 96 hours. CH₂Cl₂ is added and the reaction mixture is washed with 1N HCl, dried with Na₂SO₄ and concentrated in vacuo. The residue is purified by preparative reverse phase HPLC (Method E) to afford (2S,4R)-1-Acetyl-4-(7-methoxy-2-phenyl-quinolin-4-yloxy)-pyrrolidine-2-carboxylic acid ((5R,7R)-2,2,4,14-tetraoxo-2Λ⁶-thia-3,15-diaza-tricyclo[14.4.0.0^{5,7}]icosa-1(16),17,19-trien-5-yl)-amide as a white solid. LC-MS (Method A): Rt=3.04 min; M+H=754.3

### Example 4

### (2S,4R)-1-Acetyl-4-(7-methoxy-2-phenyl-quinolin-4-yloxy)-pyrrolidine-2-carboxylic acid ((Z)-(5R,7S)-2,2,4-trioxo-2Λ⁶-thia-3,15-diaza-tricyclo[4.4.0.0^{5,7}]icosa-1(16),8,17,19-tetraen-5-yl)-amide

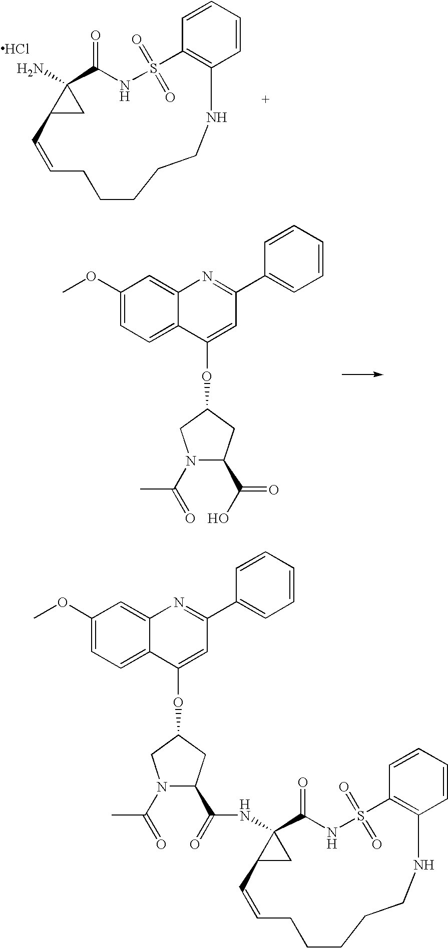

To a solution of 35 mg (0.091 mmol) (Z)-(5R,7S)-5-Amino-2,2-dioxo-2Λ⁶-thia-3,15-diaza-tricyclo[14.4.0.0^{5,7}]icosa-1(16),8,17,19-tetraen-4-one hydrochloride, 40 mg (0.098 mmol) (2S,4R)-1-Acetyl-4-(7-methoxy-2-phenyl-quinolin-4-yloxy)-pyrrolidine-2-carboxylic acid and 0.052 mL (0.30 mmol) DIPEA in 0.5 mL DMF is added 45 mg (0.118 mmol) HBTU at 0° C. The reaction mixture is allowed to warm to RT and is stirred for 12 hours. It is then concentrated in vacuo and taken up in 10 mL EtOAc and 10 mL 1N HCl. The phases are separated and the aqueous phase is extracted with 10 mL EtOAc. The combined organic phases are washed with 5% aqueous NaHCO₃ and brine, dried with Na₂SO₄ and concentrated in vacuo. The residue is purified by preparative reverse phase HPLC (Method E) to afford (2S,4R)-1-Acetyl-4-(7-methoxy-2-phenyl-quinolin-4-yloxy)-pyrrolidine-2-carboxylic acid ((Z)-(5R,7S)-2,2,4-trioxo-2Λ⁶-thia-3,15-diaza-tricyclo[14.4.0.0^{5,7}]icosa-1(16),8,17,19-tetraen-5-yl)-amide as a white solid. LCMS (Method A): Rt=3.22 min; M+H=738.2

### Preparation of (Z)-(5R,7S)-5-Amino-2,2-dioxo-2Λ⁶-thia-3,15-diaza-tricyclo[14.4.0.0^{5,7}]icosa-1(16),8,17,19-tetraen-4-one hydrochloride

### Step 1

### [(1R,2S)-1-(2-Hept-6-enylamino-benzenesulfonylaminocarbonyl)-2-vinyl-cyclopropyl]-carbamic acid tert-butyl ester

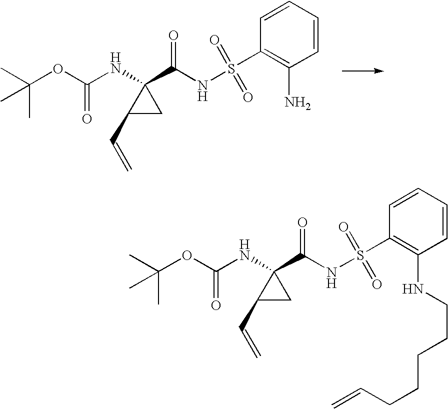

To a suspension of 300 mg (0.79 mmol) [(1R,2S)-1-(2-Amino-benzenesulfonylaminocarbonyl)-2-vinylcyclopropyl]-carbamic acid tert-butyl ester and 109 mg (0.79 mmol) K₂CO₃ in 0.8 mL DMF is added 0.24 mL (1.57 mmol) 7-bromohept-1-ene and the reaction mixture is stirred at 50° C. for 18 hours. The reaction mixture is concentrated in vacuo and the residue is chromatographed on SiO₂ (eluant hexanes/EtOAc 100:0 to hexanes/EtOAc 0:100) to afford [(1R,2S)-1-(2-Hept-6-enylamino-benzenesulfonylaminocarbonyl)-2-vinyl-cyclopropyl]-carbamic acid tert-butyl ester as an off-white solid. LC-MS (Method A): Rt=4.79 min; M+H=478.1

### Step 2

### ((Z)-(5R,7S)-2,2,4-Trioxo-2Λ⁶-thia-3,15-diaza-tricyclo[14.4.0.^{5,7}]icosa-1(16),8,17,19-tetraen-5-yl)-carbamic acid tert-butyl ester/((E)-(5R,7S)-2,2,4-Trioxo-2Λ⁶-thia-3,15-diaza-tricyclo[14.4.0.0^{5,7}]icosa-1(16),8,17,19-tetraen-5-yl)-carbamic acid tert-butyl ester

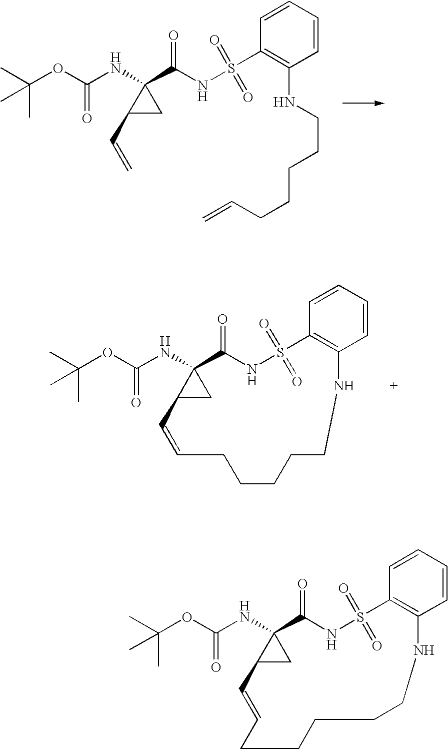

A solution of 250 mg (0.52 mmol) [(1R,2S)-1-(2-Hept-6-enylamino-benzenesulfonylaminocarbonyl)-2-vinyl-cyclopropyl]-carbamic acid tert-butyl ester and 89 mg (20 mol-%) Grubbs II catalyst in 150 mL CH₂Cl₂ is heated to reflux for 12 hours. The reaction is concentrated in vacuo and the residue is purified by preparative reverse phase HPLC (Method E) to afford ((Z)-(5R,7S)-2,2,4-Trioxo-2Λ⁶-thia-3,15-diaza-tricyclo[14.4.0.0^{5,7}]icosa-1(16),8,17,19-tetraen-5-yl)-carbamic acid tert-butyl ester and ((E)-(5R,7S)-2,2,4-Trioxo-2Λ⁶-thia-3,15-diaza-tricyclo[14.4.0.0^{5,7}]icosa-1(16),8,17,19-yl)-carbamic acid tert-butyl ester both as white powders. (Z) isomer: LC-MS (Method A): Rt=4.20 min; M+H=450.0, M+Na=472.1, M−H=448.1; (E) isomer: LC-MS (Method A): Rt=4.40 min; M+H=450.0, M−H=448.1

### Step 3

### (Z)-(5R,7S)-5-Amino-2,2-dioxo-2Λ⁶-thia-3,15-diaza-tricyclo[14.4.0.0^{5,7}]icosa-1(16),8,17,19-tetraen-4-one hydrochloride

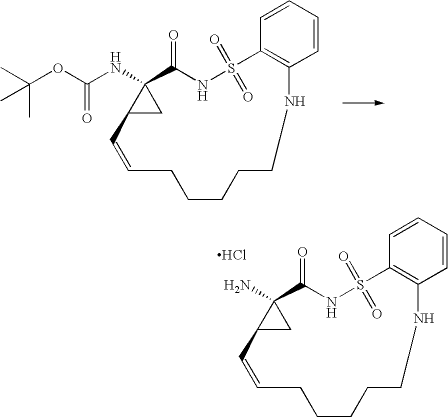

To a solution of 30 mg (0.067 mmol) ((Z)-(5R,7S)-2,2,4-Trioxo-2Λ⁶-thia-3,15-diaza-tricyclo[14.4.0.0^{5,7}]icosa-1(16),8,17,19-tetraen-5-yl)-carbamic acid tert-butyl ester in 0.5 mL dioxane is added 0.24 mL HCl in dioxane (4N) and the mixture is stirred at RT for 6 hours. The reaction mixture is concentrated in vacuo to afford (Z)-(5R,7S)-5-Amino-2,2-dioxo-2Λ⁶-thia-3,15-diaza-tricyclo[ 14.4.0.0^{5,7}]icosa-1(16),8,17,19-tetraen-4-one hydrochloride. LC-MS (Method A): Rt=2.56 min; M+H=350.0

### Example 5

### (2S,4R)-1-Acetyl-4-(7-methoxy-2-phenyl-quinolin-4-yloxy)-pyrrolidine-2-carboxylic acid ((E)-(5R,7S)-2,2,4-trioxo-2Λ⁶-thia-3,15-diaza-tricyclo[14.4.0.0^{5,7}]icosa-1(16),8,17,19-tetraen-5-yl)-amide

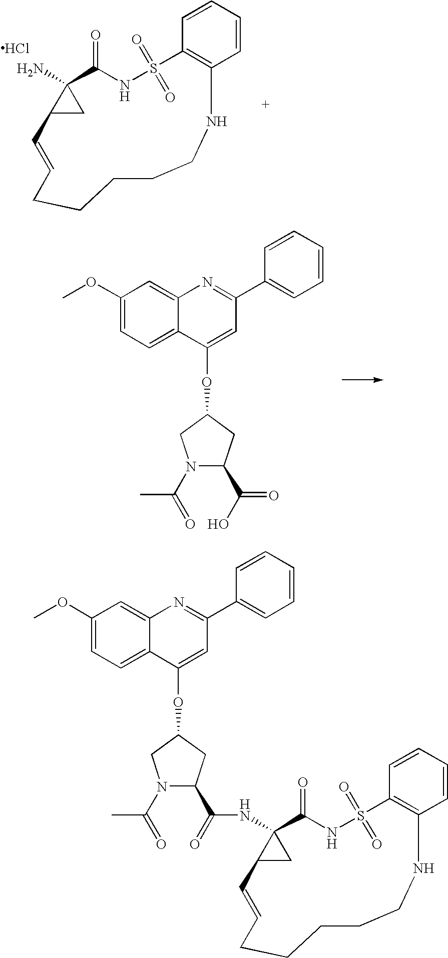

To a solution of 115 mg (0.30 mmol) (E)-(5R,7S)-5-Amino-2,2-dioxo-2Λ⁶-thia-3,15-tricyclo[14.4.0.0^{5,7}]icosa-1(16),8,17,19-tetraen-4-one hydrochloride (Example4, step2), 100 mg (0.25 mmol) (2S,4R)-1-Acetyl-4-(7-methoxy-2-phenyl-quinolin-4-yloxy)-pyrrolidine-2-carboxylic acid and 0.129 mL (0.74 mmol) DIPEA in 3 mL DMF is added 112 mg (0.30 mmol) HBTU at 0° C. The reaction mixture is allowed to warm to RT and is stirred for 12 hours. It is then concentrated in vacuo and taken up in 10 mL EtOAc and 10 mL 1N HCl. The phases are separated and the aqueous phase is extracted with 10 mL EtOAc. The combined organic phases are washed with 5% aqueous NaHCOj and brine, dried with Na₂SO₄ and concentrated in vacuo. The residue is purified by preparative reverse phase HPLC (Method E) to afford (2S,4R)-1-Acetyl-4-(7-methoxy-2-phenyl-quinolin-4-yloxy)-pyrrolidine-2-carboxylic acid ((E)-(5R,7S)-2,2,4-trioxo-2Λ⁶-thia-3,15-diaza-tricyclo[ 14.4.0.0^{5,7}]icosa-1(16),8,17,19-tetraen-5-yl)-amide as a white solid. LC-MS (Method A) Rt=3.31 min; M+H=738.2

### Preparation of (E)-(5R,7S)-5-Amino-2,2-dioxo-2Λ⁶-thia-3,15-diaza-tricyclo[14.4.0.0^{5,7}]icosa-1(16),8,17,19-tetraen-4-one hydrochloride

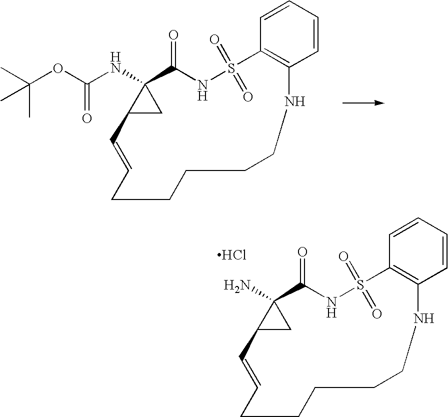

To a solution of 135 mg (0.29 mmol) ((E)-(5R,7S)-2,2,4-Trioxo-2Λ⁶-thia-3,15-diaza-tricyclo[14.4.0.0^{5,7}]icosa-1(16),8,17,19-tetraen-5-yl)-carbamic acid tert-butyl ester in 2.2 mL dioxane is added 1.1 mL HCl in dioxane (4N) and the mixture is stirred at RT for 6 hours. The reaction is concentrated in vacuo to afford (E)-(5R,7S)-5-Amino-2,2-dioxo-2Λ⁶-thia-3,15-diaza-tricyclo[14.4.0.0^{5,7}]icosa-1 (16),8,17,19-tetraen-4-one hydrochloride. LC-MS (Method A) Rt=2.81 min; M+H=350.1

### Example 6

### (2S,4R)-1-Acetyl-4-(7-methoxy-2-phenyl-quinolin-4-yloxy)-pyrrolidine-2-carboxylic acid ((5R,7R)-2,2,4-trioxo-2Λ⁶-thia-3,15-diaza-tricyclo[14.4.0.0^{5,7}]icosa-1(16),17,19-trien-5-yl)-amide

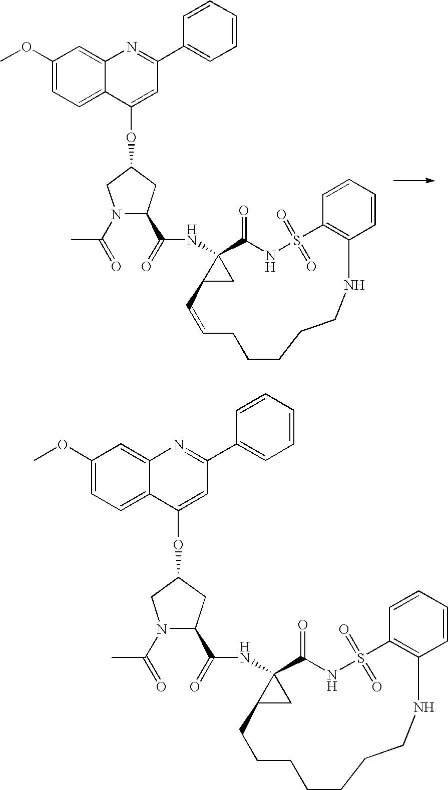

A mixture of 20 mg (0.027 mmol) (2S,4R)-1-Acetyl-4-(7-methoxy-2-phenyl-quinolin-4-yloxy)-pyrrolidine-2-carboxylic acid ((Z)-(5R,7S)-2,2,4-trioxo-2Λ⁶-thia-3,15-diaza-tricyclo[14.4.0.0^{5,7}]icosa-1(16),8,17,19-tetraen-5-yl)amide and 0.5 mg Pd/C (10%) in 4 mL EtOH is stirred under an H₂-atmosphere for 12 hours. The mixture is filtered through Celite and concentrated in vacuo. The residue is purified by preparative reverse phase HPLC (Method E) to afford (2S,4R)-1-Acetyl-4-(7-methoxy-2-phenyl-quinolin-4-yloxy)-pyrrolidine-2-carboxylic acid ((5R,7R)-2,2,4-trioxo-2Λ⁶-thia-3,15-diaza-tricyclo[14.4.0.0^{5,7}]icosa-](16),17,19-trien-5-yl)-amide as a white solid. LC-MS (Method A) Rt=3.17 min; M+H=740.2

### Example 7

The following compounds are prepared according to the same procedures described in Examples 1-6

### (2S,4R)-1-Acetyl-4-(7-methoxy-2-phenyl-quinolin-4-yloxy)-pyrrolidine-2-carboxylic acid ((E)-(5R,7S)-2,2,4,15-tetraoxo-2Λ⁶-thia-3,16-diaza-tricyclo[15.4.0.0^{5,7}]henicosa-1(17),8,18,20-tetraen-5-yl)-amide

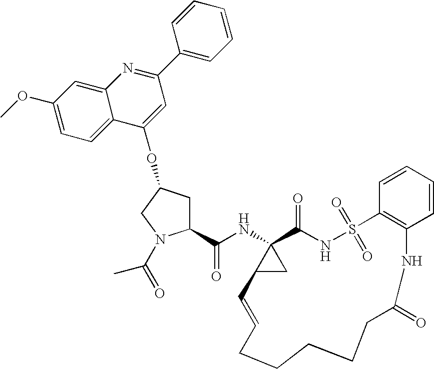

HPLC (method C): Rt=3.22 min; LC-MS (method A): M+H=766.3 ; M−H=764.0

### (2S,4R)-1-Acetyl-4-(7-methoxy-2-phenyl-quinolin-4-yloxy)-pyrrolidine-2-carboxylic acid ((5R,7R)-2,2,4,15-tetraoxo-2Λ⁶-thia-3,16-diaza-tricyclo[15.4.0.0^{5,7}]henicosa-1(17),18,20-trien-5-yl)-amide

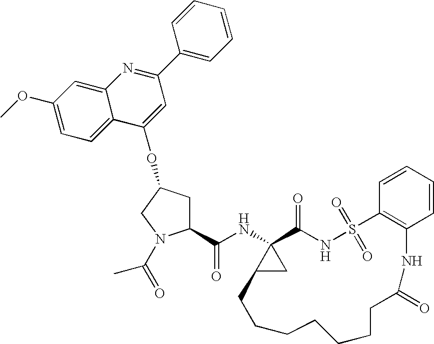

HPLC (method C): Rt=3.22 min; LC-MS (method A): M+H=767.9 M−H=765.8

### (2S,4R)-1-Acetyl-4-(7-methoxy-2-phenyl-quinolin-4-yloxy)-pyrrolidine-2-carboxylic acid ((Z)-(5R,7S)-2,2,4-trioxo-2Λ⁶-thia-3,16-diaza-tricyclo[15.4.0.0^{5,7})henicosa-1(17),8,18,20-tetraen-5-yl)-amide

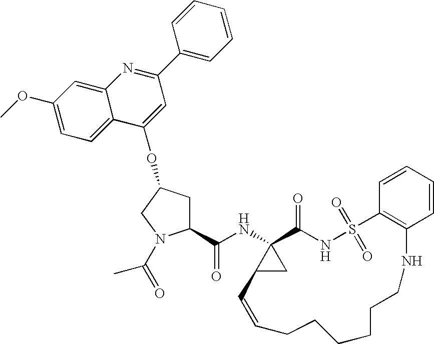

HPLC (method C): Rt=3.46 min; LC-MS (method A): M+H=752.2; M−H=750.0

### (2S,4R)-1-Acetyl-4-(7-methoxy-2-phenyl-quinolin-4-yloxy)-pyrrolidine-2-carboxylic acid ((E)-(5R,7S)-2,2,4-trioxo-2Λ⁶-thia-3,16-diaza-tricyclo[15.4.0.0^{5,7}]henicosa-1(17),8,18,20-tetraen-5-yl)-amide

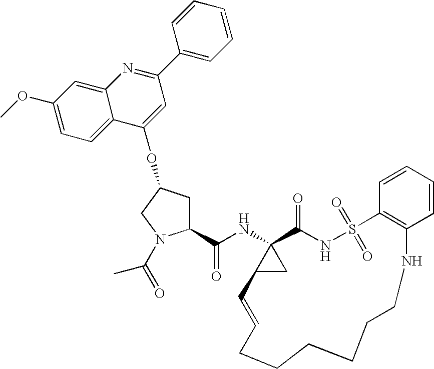

HPLC (method C): Rt=3.52 min; LC-MS (method A): M+H=752.2; M−H=750.0

### (2S,4R)-1-Acetyl-4-(7-methoxy-2-phenyl-quinolin-4-yloxy)-pyrrolidine-2-carboxylic acid ((5R,7R)-2,2,4-trioxo-2Λ⁶-thia-3,16-diaza-tricyclo[15.4.0.0^{5,7}]henicosa-1(17),18,20-trien-5-yl)-amide

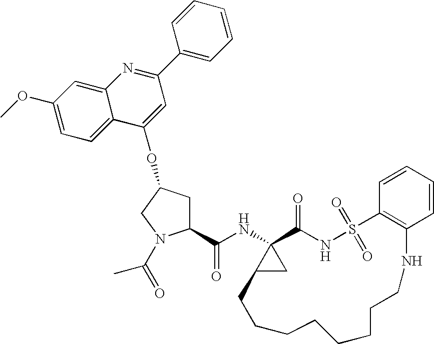

HPLC (method C): Rt=3.55 min; LC-MS (method A): M+H=754.2; M−H=752.0

### (2S,4R)-1-Acetyl-4-(7-methoxy-2-phenyl-quinolin-4-yloxy)-pyrrolidine-2-carboxylic acid ((Z)-(5R,7S)-2,2,4,13-tetraoxo-2Λ⁶-thia-3,14-diaza-tricyclo[13.4.0.0^{5,7}]nonadeca-1(15),8,16,18-tetraen-5-yl)-amide

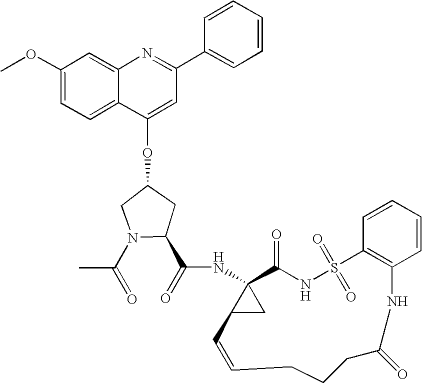

HPLC (method C): Rt=2.91 min; LC-MS (method A): M+H=738.3; M−H=736.0

### (2S,4R)-1-Acetyl-4-(7-methoxy-2-phenyl-quinolin-4-yloxy)-pyrrolidine-2-carboxylic acid ((E)-(5R,7S)-2,2,4,13-tetraoxo-2Λ⁶-thia-3,14-diaza-tricyclo[13.4.0.0^{5,7}]nonadeca-1(15),8,16,18-tetraen-5-yl)-amide

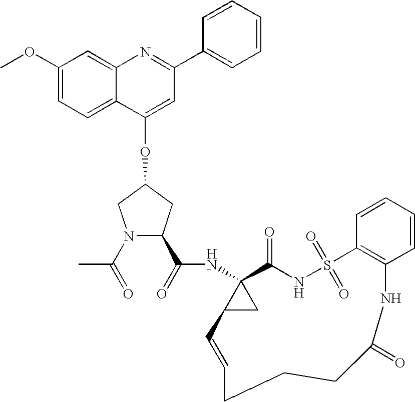

HPLC (method C): Rt=3.00 min; LC-MS (method A): M+H=738.3; M−H=736.0

### (2S,4R)-1-Acetyl-4-(7-methoxy-2-phenyl-quinolin-4-yloxy)-pyrrolidine-2-carboxylic acid ((5R,7R)-2,2,4,13-tetraoxo-2Λ⁶-thia-3,14-diaza-tricyclo[13.4.0.0^{5,7}]nonadeca-1(15),16,18-trien-5-yl)-amide

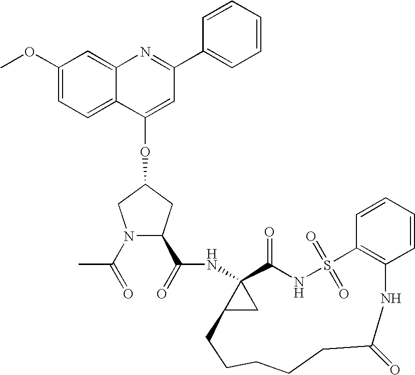

HPLC (method C): Rt=2.89 min; LC-MS (method A): M+H=739.8; M−H=737.8

### (2S,4R)-1-Acetyl-4-(7-methoxy-2-phenyl-quinolin-4-yloxy)-pyrrolidine-2-carboxylic acid ((Z)-(5R,7S)-2,2,4-trioxo-2Λ⁶-thia-3,14-diaza-tricyclo[13.4.0.0^{5,7}]nonadeca-1(15),8,16,18-tetraen-5-yl)-amide

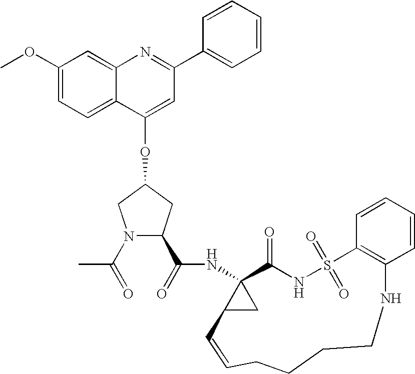

HPLC (method C): Rt=3.15 min; LC-MS (method A): M+H=724.2 ; M−H=722.0

### 2S,4R)-1-Acetyl-4-(7-methoxy-2-phenyl-quinolin-4-yloxy)-pyrrolidine-2-carboxylic acid ((E)-(5R,7S)-2,2,4-trioxo-2Λ⁶-thia-3,14-diaza-tricyclo[13.4.0.0^{5,7}]nonadeca-1(15),8,16,18-tetraen-5-yl)-amide

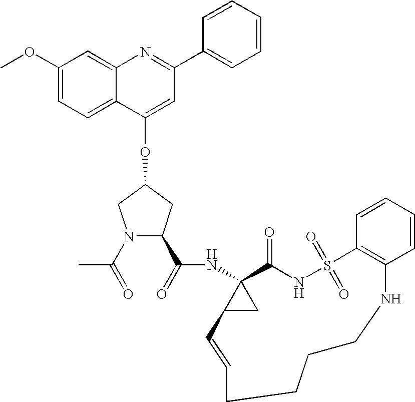

HPLC (method C): Rt=3.22 min; LC-MS (method A): M+H=724.2; M−H=722.0

### (2S,4R)-1-Acetyl-4-(7-methoxy-2-phenyl-quinolin-4-yloxy)-pyrrolidine-2-carboxylic acid ((5R,7R)-2,2,4-trioxo-2Λ⁶-thia-3,14-diaza-tricyclo[13.4.0.0^{5,7}]nonadeca-1(15),16,18-trien-5-yl)-amide

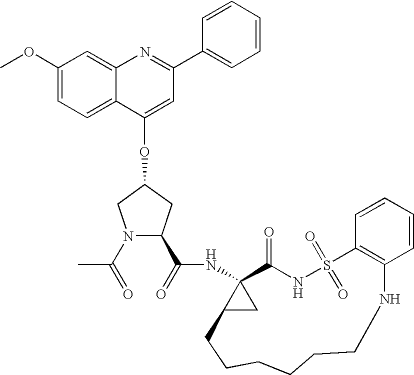

HPLC (method C): Rt=3.22 min; LC-MS (method A): M+H=726; M−H=724

### Example 8

### {(S)-1-[(2S,4R)-4-[2-(2-isopropylamino-thiazol-4-yl)-7-methoxy-quinolin-4-yloxy]-2-((E)-(5R,7S)-2,2,4,14-tetraoxo-2Λ⁶-thia-3,15-diaza-tricyclo[14.4.0.0^{5,7}]icosa-1(16),8,17,19-tetraen-5-ylcarbamoyl)-pyrrolidine-1-carbonyl]-2-methyl-propyl}-carbamic acid tert-butyl ester

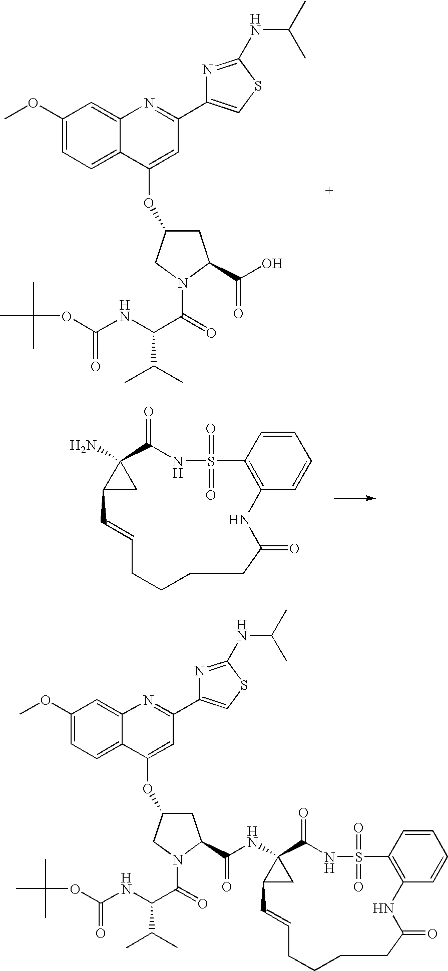

A solution of 40 mg of (2S,4R)-1-((S)-2-tert-Butoxycarbonylamino-3-methyl-butyryl)-4-[2-(2-isopropylamino-thiazol-4-yl)-7-methoxy-quinolin-4-yloxy]-pyrrolidine-2-carboxylic acid (0.064 mmol) in 0.5 mL of DMF is treated with 28 mg of (E)-(5R,7S)-5-Amino-2,2-dioxo-2Λ⁶-thia-3,15-diaza-tricyclo[14.4.0.0^{5,7}]icosa-1(16),8,17,19-tetraene-4,14-dione hydrochloride (0.070 mmol) and 0.044 mL of DIPEA (0.26 mmol), cooled to 0° C., and treated with 30 mg of HBTU (0.08 mmol). After 1 hour at 0° C., the reaction mixture is stirred at RT for 16 hours, washed twice with saturated aqueous Na2CC<3, water, dried over Na₂SO₄, and concentrated in vacuo. The residue is purified by preparative reverse phase HPLC (Method F), followed by a basic quench with NaHCO₃, extraction with ethyl acetate, and lyophilization to afford {(S)-1-[(2S,4R)-4-[2-(2-isopropylamino-thiazol-4-yl)-7-methoxy-quinolin-4-yloxy]-2-((E)-(5R,7S)-2,2,4,14-tetraoxo-2Λ⁶-thia-3,15-diaza-tricyclo[14.4.0.0^{5,7}]icosa-1(16),8,17,19-tetraen-5-ylcarbamoyl)-pyrrolidine-1-carbonyl]-2-methyl-propyl}-carbamic acid tert-butyl ester as a solid. HPLC (Method B): Rt=5.52 min; MS (Method D): M+H=974

### Preparation of (2S,4R)-1-((S)-2-tert-Butoxycarbonylamino-3-methyl-butyryl)-4-[2-(2-isopropylamino-thiazol-4-yl)-7-methoxy-quinolin-4-yloxy]-pyrrolidine-2-carboxylic acid

### Step 1

### (2S,4S)-1-((S)-2-tert-Butoxycarbonylamino-3-methyl-butyryl)-4-hydroxy-pyrrolidine-2-carboxylic acid methyl ester

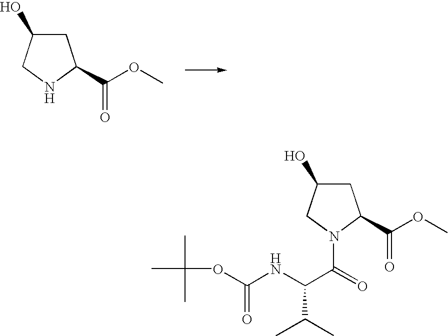

A solution of 2 g of cis-hydroxyproline methyl ester hydrochloride (11.01 mmol) in 20 mL of DMF is treated with 2.632 g of Boc-L-valine (12.11 mmol) and 7.54 mL of DIPEA (44.05 mmol). The reaction mixture is treated with 5.221 g of HBTU (13.66 mmol) at 0° C., stirred for 1 hour, and stirred at RT for 16 hours. The reaction mixture is taken up in EtOAc, washed with NaHCO₃, 0.1N HCl and concentrated. The residue is chromatographed on SiO₂ (eluant hexanes/EtOAc 1:1 to EtOAc) to give (2S,4S)-1-((S)-2-tert-butoxycarbonylamino-3-methyl-butyryl)-4-hydroxy-pyrrolidine-2-carboxylic acid methyl ester as a foam. TLC (EtOAc): Rf 0.40; MS (Method D): M+1=345

### Step 2

### (2S,4S)-4-(4-Bromo-benzenesulfonyloxy)-1-((S)-2-tert-butoxycarbonylamino-3-methyl-butyryl)-pyrrolidine-2-carboxylic acid methyl ester

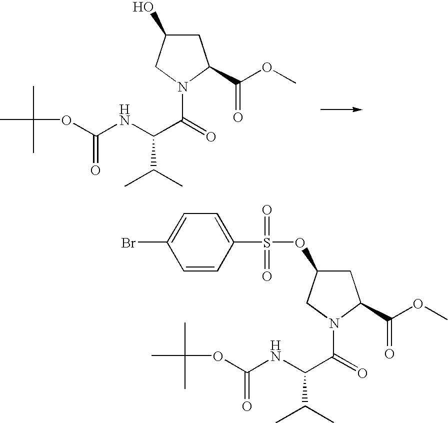

A solution of 1 g of (2S,4S)-1-((S)-2-tert-butoxycarbonylamino-3-methyl-butyryl)-4-hydroxy-pyrrolidine-2-carboxylic acid methyl ester (2.904 mmol) in 8 mL of toluene is treated with 0.521 g of DABCO (4.646 mmol), followed by 1.039 g of 4-bromobenzenesulfonyl chloride (4.066 mmol) in 6 mL of toluene, while cooling with an ice-bath. The resulting suspension is stirred at RT for 2 hours. The reaction mixture is taken up in EtOAc, washed with half-saturated aqueous Na₂CO₃ and 0.5N HCl, dried over Na₂SO₄, and concentrated to afford (2S,4S)-4-(4-Bromo-benzenesulfonyloxy)-1-((S)-2-tert-butoxycarbonylamino-3-methyl-butyryl)-pyrrolidine-2-carboxylic acid methyl ester as a foam. HPLC (method B): Rt=3.54 min; MS (method D): M+1=563

### Step 3

### (2S,4R)-1-((S)-2-tert-Butoxycarbonylamino-3-methyl-butyryl)-4-[2-(2-isopropylamino-thiazol-4-yl)-7-methoxy-quinolin-4-yloxy]-pyrrolidine-2-carboxylic acid methyl ester

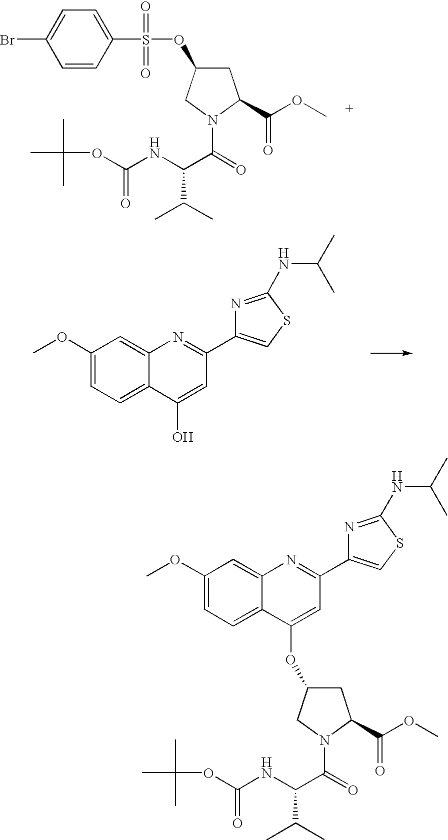

A mixture of 1.008 g of (2S,4S)-4-(4-Bromo-benzenesulfonyloxy)-1-((S)-2-tert-butoxycarbonylamino-3-methyl-butyryl)-pyrrolidine-2-carboxylic acid methyl ester (1.789 mmol), 0.564 g of 2-(2-Isopropylamino-thiazol-4-yl)-7-methoxy-quinolin-4-ol (1.789 mmol) and 0.583 g of Cs₂CO₃ (1.789 mmol) in 20 mL of N-methyl-pyrrolidone is stirred at 90° C. for 9 hours. The reaction mixture is taken up in EtOAc, washed with 1N NaCO₃, water, and dried over Na₂SO₄. After concentration the residue is chromatographed on SiO₂ (eluant CH₂Cl₂/EtOH 95:5 with 0.1% NH₄OH) to give (2S,4R)-1-((S)-2-tert-Butoxycarbonylamino-3-methyl-butyryl)-4-[2-(2-isopropylamino-thiazol-4-yl)-7-methoxy-quinolin-4-yloxy]-pyrrolidine-2-carboxylic acid methyl ester as a foam. TLC (CH₂Cl₂/EtOH 95:5 with 0.1% NH₄OH) Rf 0.20; MS (method D): M+1=642

### Step 4

### (2S,4R)-1-((S)-2-tert-Butoxycarbonylamino-3-methyl-butyryl)-4-[2-(2-isopropylamino-thiazol-4-yl)-7-methoxy-quinolin-4-yloxy]-pyrrolidine-2-carboxylic acid

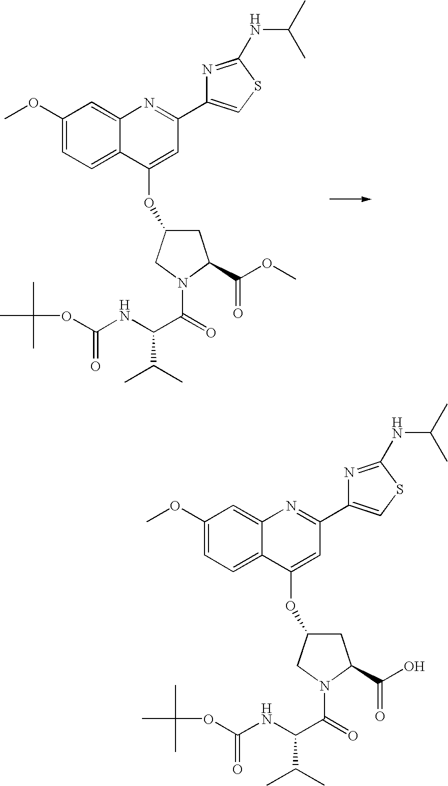

A solution of 0.46 g of (2S,4R)-1-((S)-2-tert-Butoxycarbonylamino-3-methyl-butyryl)-4-[2-(2-isopropylamino-thiazol-4-yl)-7-methoxy-quinolin-4-yloxy]-pyrrolidine-2-carboxylic acid methyl ester (0.717 mmol) in 5 mL of THF is treated with 5 mL of MeOH/water (1:1 vol) and 0.12 g of LiOH.H₂O (2.868 mmol). The reaction mixture is stirred at RT for 16 hours, quenched with citric acid to pH 6-7, taken up in CH₂Cl₂, and dried over Na₂SO₄. Concentration in vacuo affords (2S,4R)-1-((S)-2-tert-Butoxycarbonylamino-3-methyl-butyryl)-4-[2-(2-isopropylamino-thiazol-4-yl)-7-methoxy-quinolin-4-yloxy]-pyrrolidine-2-carboxylic acid as a foam. HPLC (method B): Rt=4.39 min; MS (method D): M+1=628

### Example 9

The following compound is prepared according to the same procedures described in Example 8.

### ((S)-1-[(2S,4R)-4-[2-(2-isopropylamino-thiazol-4-yl)-7-methoxy-quinolin-4-yloxy]-2-((Z)-(5R,7S)-2,2,4-trioxo-2Λ⁶-thia-3,15-diaza-tricyclo[14.4.0.0^{5,7}]icosa-1(16),8,17,19-tetraen-5-ylcarbamoyl)-pyrrolidine-1-carbonyl]-2-methyl-propyl)-carbamic acid tert-butyl ester

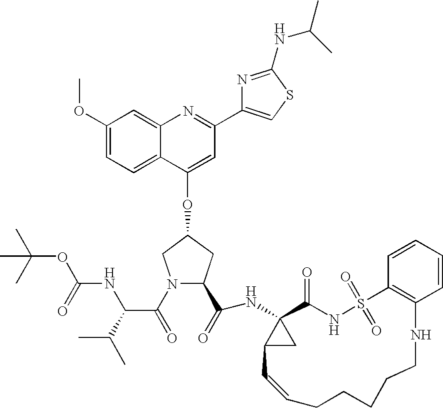

HPLC (method B): Rt=3.73 min ; MS (method D): M−H=958.8

### Example 10

### (2S,4R)-4-[2-(2-Isopropylamino-thiazol-4-yl)-7-methoxy-quinolin-4-yloxy]-2-((E)-(5R,7S)-2,2,4,14-tetraoxo-2Λ⁶-thia-3,15-diaza-tricyclo[14.4.0.0^{5,7}]icosa-1(16),8,17,19-tetraen-5-ylcarbamoyl)-pyrrolidine-1-carboxylic acid tert-butyl ester

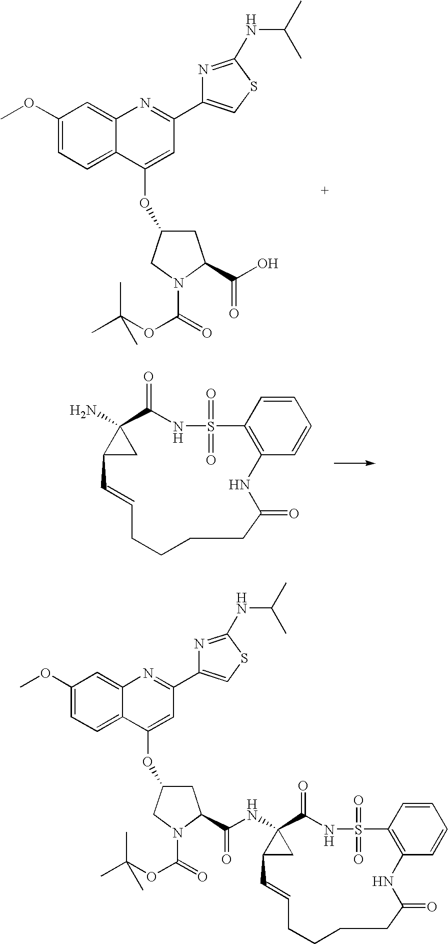

A solution of 40 mg of (2S,4R)-4-[2-(2-Isopropylamino-thiazol-4-yl)-7-methoxy-quinolin-4-yloxy]-pyrrolidine-1,2-dicarboxylic acid 1-tert-butyl ester (0.076 mmol) in 0.5 mL of DMF is treated with 34 mg of (E)-(5R,7S)-5-Amino-2,2-dioxo-2Λ⁶-thia-3,15-diaza-tricyclo[14.4.0.0^{5,7}]icosa-1(16),8,17,19-tetraene-4,14-dione hydrochloride (0.095 mmol) and 0.052 mL of DIPEA (0.304 mmol), cooled to 0° C., and treated with 36 mg of HBTU (0.08 mmol). After 1 hour at 0° C., the reaction mixture is stirred at RT for 1.6 hours, washed twice with saturated aqueous Na₂CO₃, water, dried over Na₂SO₄, and concentrated in vacuo. The residue is purified by preparative reverse phase HPLC (Method F), followed by a basic quench with NaHCO₃, extraction with EtOAc, and lyophilization to afford (2S,4R)-4-[2-(2-Isopropylamino-thiazol-4-yl)-7-methoxy-quinolin-4-yloxy]-2-((E)-(5R,7S)-2,2,4,14-tetraoxo-2Λ⁶-thia-3,15-diaza-tricyclo[14.4.0.0^{5,7}]icosa-1(16),8,17,19-tetraen-5-ylcarbamoyl)-pyrrolidine-1-carboxylic acid tert-butyl ester as a solid. HPLC (Method B): Rt=5.44 min; M+H=875

### (2S,4R)-4-[2-(2-Isopropylamino-thiazol-4-yl)-7-methoxy-quinolin-4-yloxy]-pyrrolidine-1,2-dicarboxylic acid 1-tert-butyl ester is prepared according to the procedures described in Example 8

### Step 1

### (2S,4S)-4-(4-Bromo-benzenesulfonyloxy)-pyrrolidine-1,2-dicarboxylic acid 1-tert-butyl ester 2-methyl ester

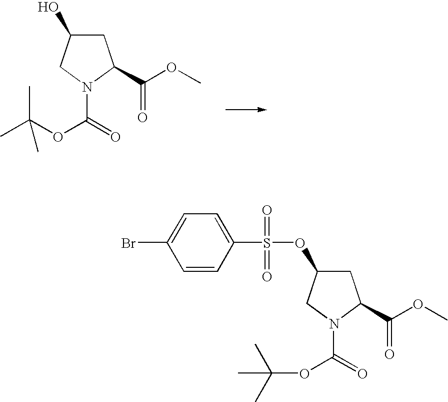

HPLC(Method B): Rt=4.76 min ; MS (Method D): M+1=464

### Step 2

### (2S,4R)-4-[2-(2-Isopropylamino-thiazol-4-yl)-7-methoxy-quinolin-4-yloxy]-pyrrolidine-1,2-dicarboxylic acid 1-tert-butyl ester 2-methyl ester

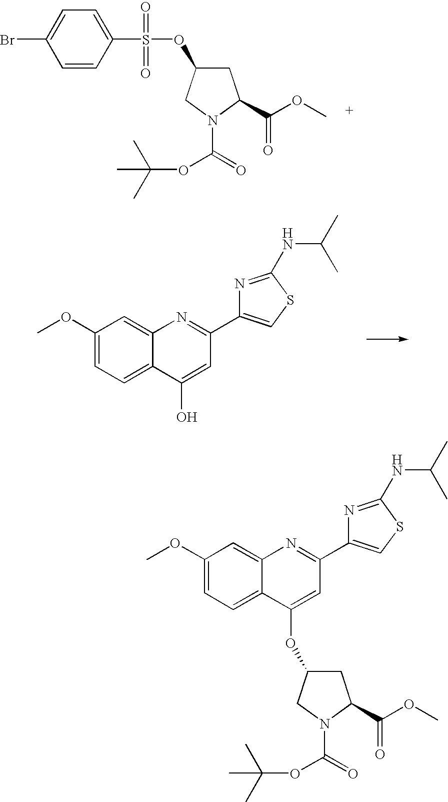

HPLC (Method B): Rt=6.05 min ; MS (Method D); M+1=543

### Step 3

### (2S,4R)-4-[2-(2-Isopropylamino-thiazol-4-yl)-7-methoxy-quinolin-4-yloxy]-pyrrolidine-1,2-dicarboxylic acid 1-tert-butyl ester

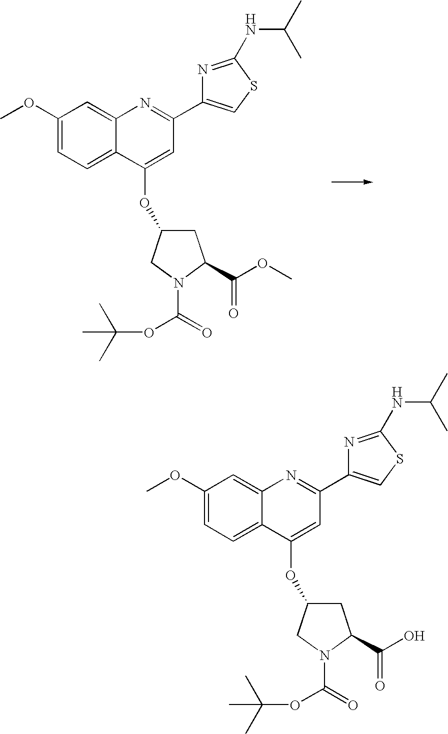

HPLC (Method B): Rt=4.28 min ; MS (Method D): M+1=529

The following compound is prepared likewise:

### (2S,4R)-4-(2-(2-Isopropylamino-thiazol-4-yl)-7-methoxy-quinolin-4-yloxy]-2-((Z)-(5R,7S)-2,2,4-trioxo-2Λ⁶-thia-3,15-diaza-tricyclo[14.4.0.0^{5,7}]icosa-1(16),8,17,19-tetraen-5-ylcarbamoyl)-pyrrolidine-1-carboxylic acid tert-butyl ester

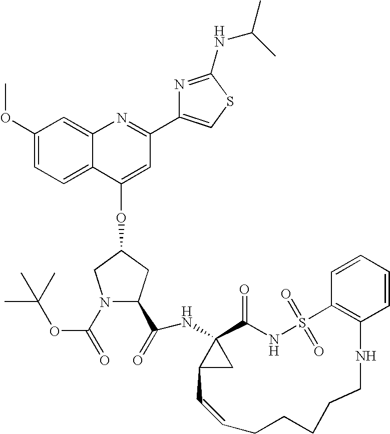

HPLC (Method C): Rt=3.79 min ; LC-MS (Method A): M−H=858.5

### Example 11

The following compound is prepared according to the same procedures described in Example 10 from (2S,4R)-4-(7-Methoxy-2-phenyl-quinolin-4-yloxy)-pyrrolidine-1,2-dicarboxylic acid 1-tert-butyl ester

### (2S,4R)-4-(7-Methoxy-2-phenyl-quinolin-4-yloxy)-2-((Z)-(5R,7S)-2,2,4-trioxo-2Λ⁶-thia-3,15-diaza-tricyclo[14.4.0.0^{5,7}]icosa-1(16),8,17,19-tetraen-5-ylcarbamoyl)-pyrrolidine-1-carboxylic acid tert-butyl ester

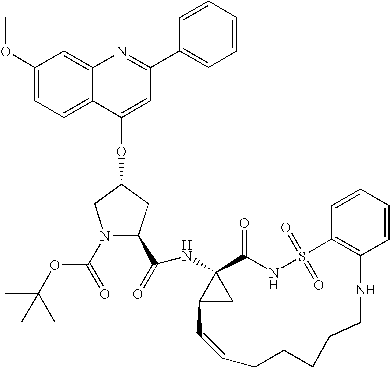

LC-MS (Method A): Rt=3.60 min; M+H=796.0; M−H=793.9

### (2S,4R)-4-(7-Methoxy-2-phenyl-quinolin-4-yloxy)-pyrrolidine-1,2-dicarboxylic acid 1-tert-butyl ester is prepared according to the procedure described in Example 1

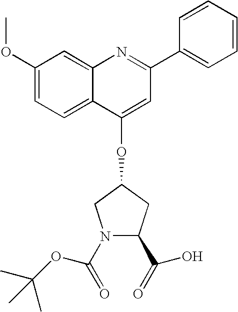

HPLC (Method C): Rt=2.48 min; LC-MS (Method A):: M−H=463.3

### Example 12

The following compound is prepared according to the same procedures as described in Example 8 from (2S,4R)-1-((S)-2-tert-Butoxycarbonylamino-3-methyl-butyryl)-4-(7-methoxy-2-phenyl-quinolin-4-yloxy)-pyrrolidine-2-carboxylic acid (WO2002060926 A2)

### ((S)-1-[(2S,4R)-4-(7-Methoxy-2-phenyl-quinolin-4-yloxy)-2-((Z)-(5R,7S)-2,2,4-trioxo-2Λ⁶-thia-3,15-diaza-tricyclo[14.4.0.0^{5,7}]icosa-1(16),8,17,19-tetraen-5-ylcarbamoyl)-pyrrolidine-1-carbonyl]-2-methyl-propyl)-carbamic acid tert-butyl ester

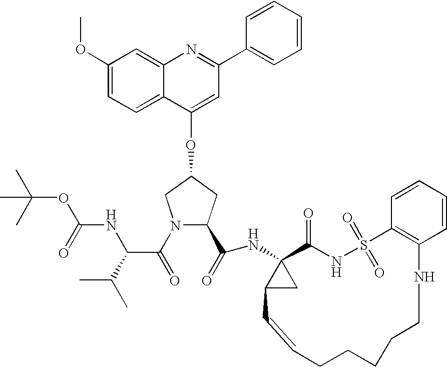

HPLC (method C): Rt=3.60 min ; LC-MS (Method A): M−H=894.8

### Example 13

### (2S,4R)-4-(7-Methoxy-2-phenyl-quinolin-4-yloxy)-1-phenylacetyl-pyrrolidine-2-carboxylic acid ((Z)-(5R,7S)-2,2,4-trioxo-2Λ⁶-thia-3,15-diaza-tricyclo[14.4.0.0^{5,7}]icosa-1(16),8,17,19-tetraen-5-yl)-amide

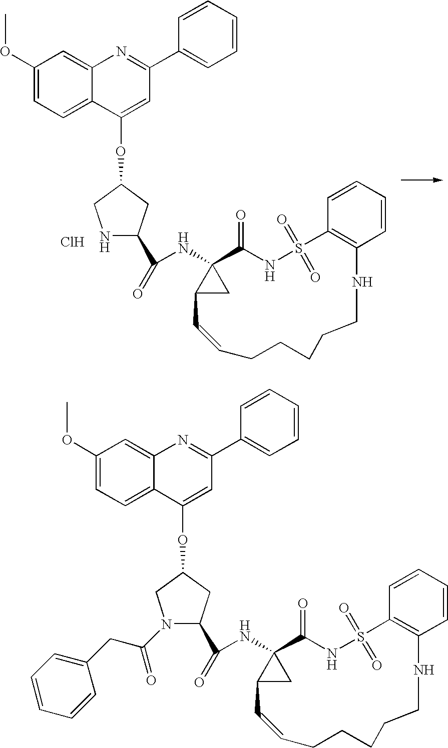

To a solution of 17 mg (0.023 mmol) (2S,4R)-4-(7-Methoxy-2-phenyl-quinolin-4-yloxy)-pyrrolidine-2-carboxylic acid ((Z)-(5R,7S)-2,2,4-trioxo-2Λ⁶-thia-3,15-diaza-tricyclo[14.4.0.0^{5,7}]icosa-1(16),8,17,19-tetraen-5-yl)-amide hydrochloride, 3.8 mg (0.028 mmol) phenyl acetic acid and 0.0122 mL (0.070 mmol) DIPEA in 0.5 mL DMF is added 10.6 mg (0.028 mmol) HBTU at 0° C. The reaction mixture is allowed to warm to RT and is stirred for 12 hours. It is then concentrated in vacuo and taken up in 5 mL EtOAc and 5 mL 1N HCl. The phases are separated and the aqueous phase is extracted with 5 mL EtOAc. The combined organic phases are washed with 5% aqueous NaHCO₃ and brine, dried with Na₂SO₄ and concentrated in vacuo. The residue is purified by preparative reverse phase HPLC (Method E) to afford (2S,4R)-4-(7-Methoxy-2-phenyl-quinolin-4-yloxy)-1-phenylacetyl-pyrrolidine-2-carboxylic acid ((Z)-(5R,7S)-2,2,4-trioxo-2Λ⁶-thia-3,15-diaza-tricyclo[14.4.0.0^{5,7}]icosa-1(16),8,17,19-tetraen-5-yl)-amide as a white solid. HPLC (Method C): Rt=3.48 min; LC-MS (Method A): 813.0

### Preparation of (2S,4R)-4-(7-Methoxy-2-phenyl-quinolin-4-yloxy)-pyrrolidine-2-carboxylic acid ((Z)-(5R,7S)-2,2,4-trioxo-2Λ⁶-thia-3,15-diaza-tricyclo[14.4.0.0^{5,7}]icosa-1(16),8,17,19-tetraen-5-yl)-amide hydrochloride

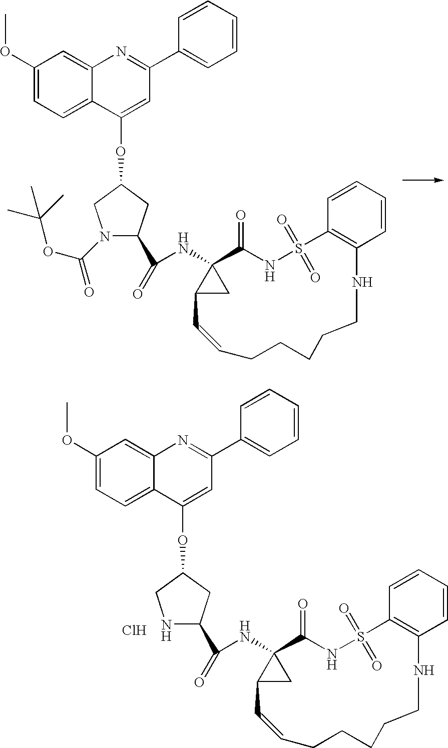

To a solution of 50 mg of (2S,4R)-4-(7-Methoxy-2-phenyl-quinolin-4-yloxy)-2-((Z)-(5R,7S)-2,2,4-trioxo-2Λ⁶-thia-3,15-diaza-tricyclo[14.4.0.0^{5,7}]icosa-1(16),8,17,19-tetraen-5 mL dioxane ylcarbamoyl)-pyrrolidine-1-carboxylic acid tert-butyl ester (0.063 mmol) in 0.5 mL dioxane is added 0.25 mL HQ in dioxane (4N) and the mixture is stirred at RT for 18 hours. The reaction is concentrated in vacuo to afford (2S,4R)-4-(7-Methoxy-2-phenyl-quinolin-4-yloxy)-pyrrolidine-2-carboxylicacid ((Z)-(5R,7S)-2,2,4-trioxo-2Λ⁶-thia-3,15-diaza-tricyclo[14.4.0.0^{5,7}]icosa-1(16),8,17,19-tetraen-5-yl)-amide hydrochloride. LC-MS (method A): Rt 3.50 min; M+H=695.9, M−H=693.8

### Example 14

### 2S,4R)-1-Methanesulfonyl-4-(7-methoxy-2-phenyl-quinolin-4-yloxy)-pyrrolidine-2-carboxylic acid ((Z)-(5R,7S)-2,2,4-trioxo-2Λ⁶-thia-3,15-diaza-tricyclo[14.4.0.0^{5,7}]icosa-1(16),8,17,19-tetraen-5-yl)-amide

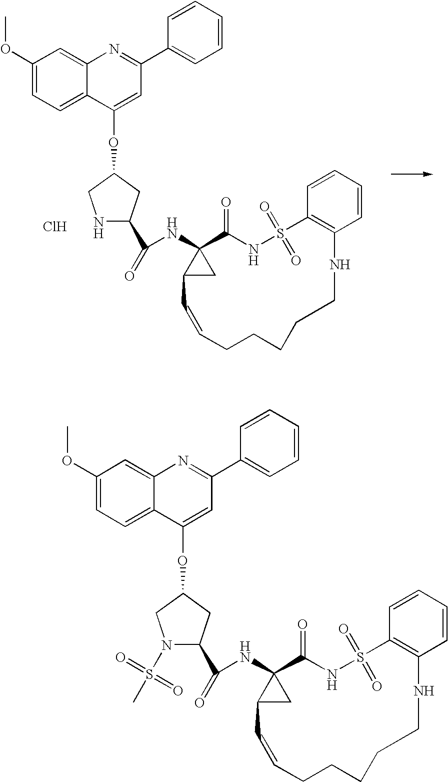

To a solution of 17 mg (0.023 mmol) (2S,4R)-4-(7-Methoxy-2-phenyl-quinolin-4-yloxy)-pyrrolidine-2-carboxylic acid ((Z)-(5R,7S)-2,2,4-trioxo-2Λ⁶-thia-3,15-diaza-tricyclo[14.4.0.0^{5,7}]icosa-1(16),8,17,19-tetraen-5-yl)-amide hydrochloride, 0.0065 mL (0.046 mmol) triethylamine in 0.25 mL CH₂Cl₂ is added 0.002.0 mL (0.028 mmol) methanesulfonylchloride at 0° C. The reaction mixture is allowed to warm to RT and is stirred for 12 hours. 5 mL EtOAc and 5 mL 1N HCl are added and the phases are separated. The aqueous phase is extracted with 5 mL EtOAc and the combined organic phases are washed with 5% aqueous NaHCO₃ and brine, dried.with Na₂SO₄ and concentrated in vacuo. The residue is purified by preparative reverse phase HPLC (Method E) to afford (2S,4R)-1-Methanesulfonyl-4-(7-methoxy-2-phenyl-quinolin-4-yloxy)-pyrrolidine-2-carboxylic acid ((Z)-(5R,7S)-2,2,4-trioxo-2Λ⁶-thia-3,15-diaza-tricyclo[14.4.0.0^{5,7}]icosa-1(16),8,17,19-tetraen-5-yl)-amide as a white solid. HPLC (Method C): Rt=3.48 min ; LC-MS (Method A): M+H=774.8; M−H=772.9

### Example 15

### (2S,4R)1-Acetyl-4-(7-methoxy-2-phenyl-quinolin-4-yloxy)-pyrrolidine-2-carboxylic acid ((E)-(5R,7S)-2,2,4-trioxo-16-oxa-2Λ⁶-thia-3-aza-tricyclo[15.3.1.0^{5,7}]henicosa-1(20),8,17(21),18-tetraen-5-yl)-amide

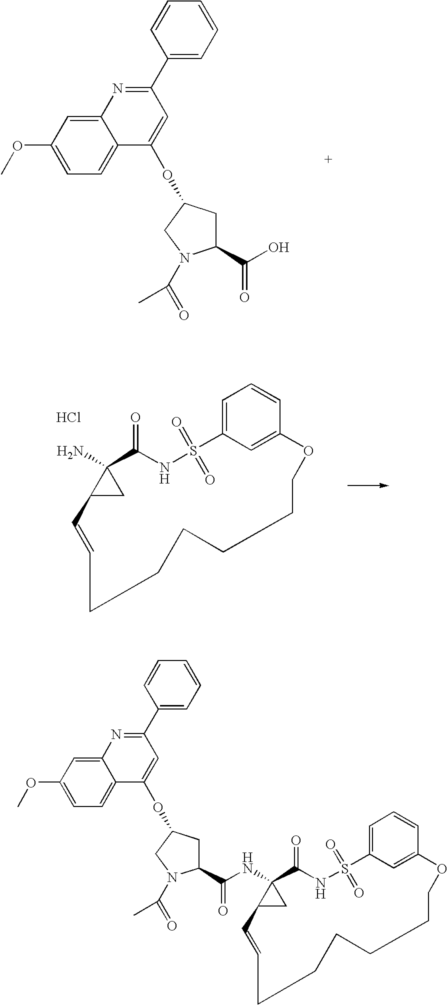

To a solution of 65 mg (0.16 mmol) (E)-(5R,7S)-5-Amino-2,2-dioxo-16-oxa-2Λ⁶-thia-3-aza-tricyclo[15.3.1.0^{5,7}]henicosa-1(20),8,17(21),18-tetraen-4-one hydrochloride, 60 mg (0.15 mmol) (2S,4R)-1-Acetyl-4-(7-methoxy-2-phenyl-quinolin-4-yloxy)-pyrrolidine-2-carboxylic acid and 0.08 mL (0.44 mmol) DIPEA in 3 mL DMF is added 67 mg (0.18 mmol) HBTU at 0° C. The reaction mixture is allowed to warm to room temperature and is stirred for 12 hours. It is then concentrated in vacuo and taken up in 10 mL EtOAc and 10 mL 1N HCl. The phases are separated and the aqueous phase is extracted with 10 mL EtOAc. The combined organic phases are washed with 5% aqueous NaHCO₃ and brine, dried with Na₂SO₄ and concentrated in vacuo. The residue is purified by preparative reverse phase HPLC (Method E) to afford (2S,4R)-1-Acetyl-4-(7-methoxy-2-phenyl-quinolin-4-yloxy)-pyrrolidine-2-carboxylic acid ((E)-(5R,7S)-2,2,4-trioxo-16-oxa-2Λ⁶-thia-3-aza-tricyclo[15.3.1.0^{5,7}]henicosa-1(20),8,17(21),18-tetraen-5-yl)-amide as a white solid. LC-MS (Method A):=3.38 min; M+H=754.0, M−H=751.9

### Preparation of (E)-(5R,7S)-5-Amino-2-dioxo-16-oxa-2Λ⁶-thia-3-aza-tricyclo[15.3:1.0^{5,7}]henicosa-1(20),8,17(21),18-tetraen-4-one hydrochloride

### Step 1

### 3-Oct-7-enyloxy-benzenesulfonamide

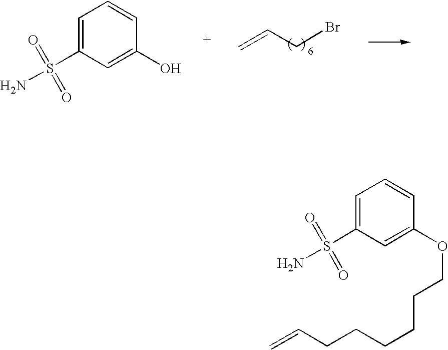

A mixture of 50 mg (2.89 mmol) 3-Hydroxybenzenesulfonamide, 0.58 mL (3.46 mmol) 8-bromo-1-octene and 524 mg (3.75 mmol) K₂CO₃ in 4 mL DMF is heated to 55° C. for 12 hours. The reaction mixture is concentrated in vacuo and the residue is taken up with 20 mL EtOAc and 10 mL 1N HCl. The phases are separated and the aqueous phase is extracted with EtOAc. The combined organic phases are dried with MgSO₄, concentrated in vacuo and the residue is chromatographed on SiO₂ (hex/EtOAc 100:0 to 0:100) to give 3-Oct-7-enyloxy-benzenesulfonamide as a white solid. LC-MS (method A): Rt=4.05 min; M+Na=305.9

### Step 2

### ((1R,2S)-1-(3-Oct-7-enyloxy-benzenesulfonylaminocarbonyl)-2-vinyl-cyclopropyl)-carbamic acid tert-butyl ester

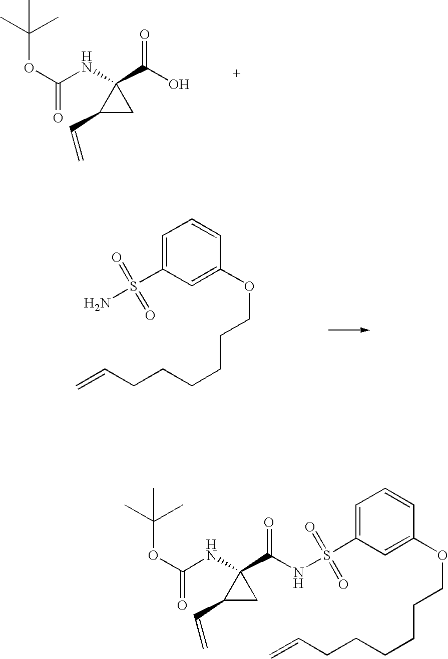

To a solution of 300 mg (1.32 mmol) (1R,2S)-1-tert-Butoxycarbonylamino-2-vinyl-cyclopropanecarboxylic acid in 3 mL THF is added 338 mg (1.98 mmol) CDI and the mixture is stirred at 70° C. for 2 hours. The mixture is allowed to cool to room temperature and 449 mg (1.58 mmol) 3-Oct-7-enyloxy-benzenesulfonamide and 0.30 mL (1.98 mmol) DBU are added. The reaction is stirred at room temperature for 12 hours. 10 mL EtOAc are added and the mixture is washed with 5 mL 1 N HCl. The organic layer is dried with MgSO₄ and concentrated in vacuo. The residue is purified by preparative reverse phase HPLC (Method E) to afford ((1R,2S)-1-(3-Oct-7-enyloxy-benzenesulfonylaminocarbonyl)-2-vinyl-cyclopropyl)-carbamic acid tert-butyl ester as an off-white solid. HPLC (Method C): Rt=4.42 min ; LC-MS (Method A): M−H=491.5

### Step 3

### ((E)-(5R,7S)-2,2,4-Trioxo-16-oxa-2Λ⁶-thia-3-aza-tricyclo[15.3.1.0^{5,7}]henicosa-1(20),8,17(21),18-tetraen-5-yl)-carbamic acid tert-butyl ester

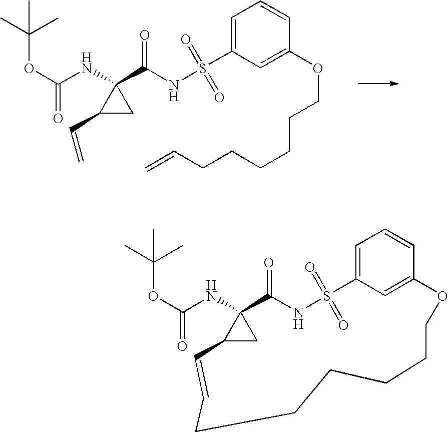

A solution of 290 mg (0.59 mmol) ((1R,2S)-1-(3-Oct-7-enyloxy-benzenesulfonylaminocarbonyl)-2-vinyl-cyclopropyl)-carbamic acid tert-butyl ester and 74 mg (20 mol-%) Grubbs II catalyst in 160 mL CH₂Cl₂ is refluxed for 12 hours. The reaction is 1 concentrated in vacuo and the residue is purified by preparative reverse phase HPLC (Method E) to afford ((E)-(5R,7S)-2,2,4-Trioxo-16-oxa-2Λ⁶-thia-3-aza-tricyclo[15.3.1.0^{5,7}]henicosa-1(20),8,17(21),18-tetraen-5-yl)-carbamic acid tert-butyl ester as an off-white solid. LC-MS (method A): Rt=3.83 min; M−H=463.1

### Step 4

### (E)-(5R,7S)-5-Amino-2,2-dioxo-16-oxa-2Λ⁶-thia-3-aza-tricyclo[15.3.1.0^{5,7}]henicosa-1(20),8,17(21),18-tetraen-4-one hydrochloride

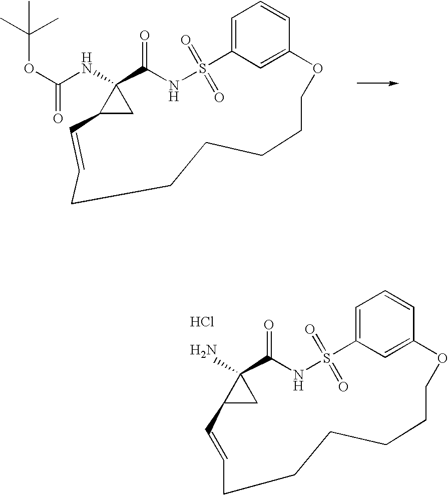

To a solution of 200 mg (0.43 mmol) of ((E)-(5R,7S)-2,2,4-Trioxo-16-oxa-2Λ⁶-thia-3-aza-tricyclo[15.3.1.0^{5,7}]henicosa-1(20),8,17(21),18-tetraen-5-yl)-carbamic acid tert-butyl ester in 2 mL dioxane is added 1.6 mL HCl in dioxane (4M) and the mixture is stirred at room temperature for 2 hours. The reaction is concentrated in vacuo to afford (E)-(5R,7S)-5-Amino-2,2-dioxo-16-oxa-2Λ⁶-thia-3-aza-tricyclo[15.3.1.0^{5,7}]henicosa-1 (20),8,17(21),18-tetraen-4-one hydrochloride. LC-MS (method A): Rt=2.77 min; M+H=365.0, M−H=363.0 The following two compounds are prepared according to the same procedure as described above. (2S,4R)-1-Acetyl-4-(7-methoxy-2-phenyl-quinolin-4-yloxy)-pyrrolidine-2-carboxylic acid ((Z)-(5R,7S)-2,2,4-trioxo-15-oxa-2Λ⁶-thia-3-aza-tricyclo[14.3.1.0^{5,7}]icosa-1(20),8,16,18-tetraen-5-yl)-amide

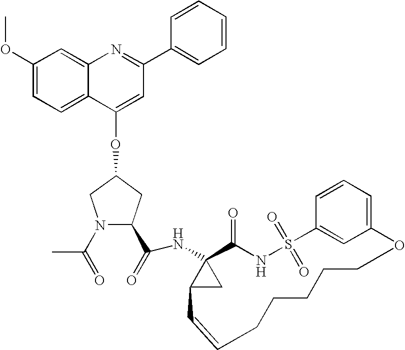

HPLC (Method C): Rt=3.29 min ; LC-MS (Method A): M−H=738.0

### (2S,4R)-1-Acetyl-4-(7-methoxy-2-phenyl-quinolin-4-yloxy)-pyrrolidine-2-carboxylic acid ((E)-(5R,7S)-2,2,4-trioxo-15-oxa-2Λ⁶-thia-3-aza-tricyclo[14.3.1.0^{5,7})icosa-1(20),8,16,18-tetraen-5-yl)-amide

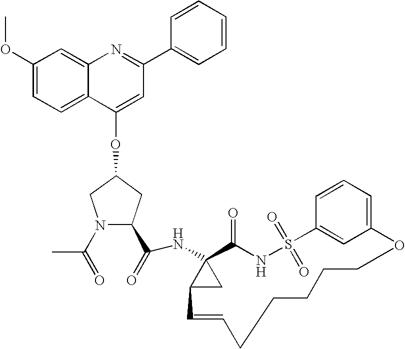

HPLC (Method C): Rt=3.31 min ; LC-MS (Method A): M−H=738.0

### Example 16

The following compound is prepared according to the same procedure described in Example 3 starting from (2S,4R)-1-Acetyl-4-(7-methoxy-2-phenyl-quinolin-4-yloxy)-pyrrolidine-2-carboxylic acid ((E)-(5R,7S)-2,2,4-trioxo-16-oxa-2Λ⁶-thia-3-aza-tricyclo[15.3.1.0^{5,7}]henicosa-1 (20),8,17(21),18-tetraen-5-yl)-amide.

### 2S,4R)-1-Acetyl-4-(7-methoxy-2-phenyl-quinolin-4-yloxy)-pyrrolidine-2-carboxylic acid ((5R,7R)-2,2,4-trioxo-16-oxa-2Λ⁶-thia-3-aza-tricyclo[15.3.1.0^{5,7}]henicosa-1(20),18-trien-5-yl)-amide

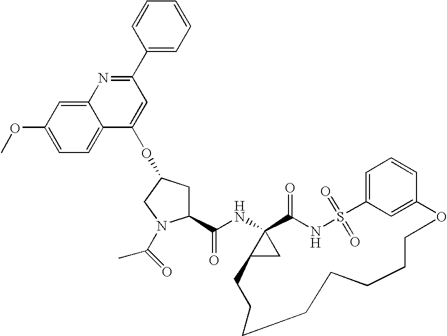

HPLC (Method C): Rt=3.46 min ; LC-MS (Method A): M−H=753.8

### Example 17

### (2S,4R)-1-Acetyl-4-(7-methoxy-2-phenyl-quinolin-4-yloxy)-pyrrolidine-2-carboxylic acid ((Z)-(5R,7S)-2,2,4-trioxo-15-oxa-2Λ⁶-thia-3-aza-tricyclo[14.4.0.0^{5,7}]icosa-1(16),8,17,19-tetraen-5-yl)-amide

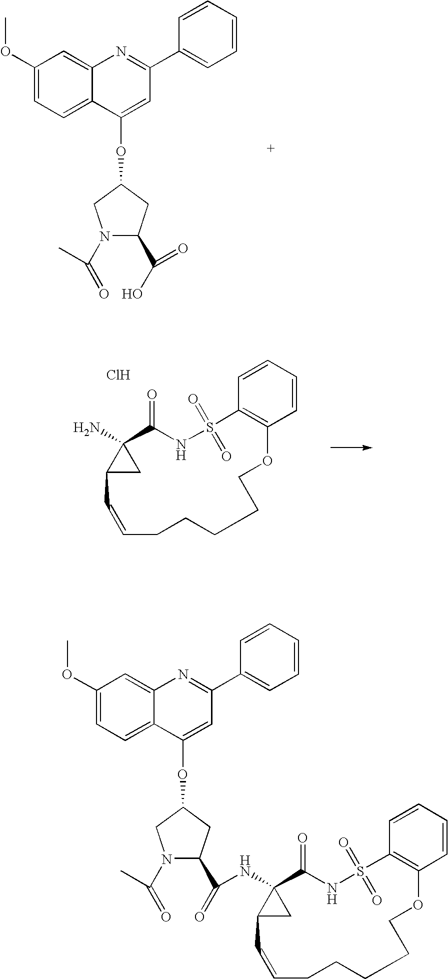

To a solution of 86 mg (0.22 mmol) (Z)-(5R,7S)-5-Amino-2,2-dioxo-15-oxa-2Λ⁶-thia-3-aza-tricyclo[14.4.0.0^{5,7}]icosa-1(16),8,17,19-tetraen-4-one hydrochloride, 90 mg (0.22 mmol) (2S,4R)-1Acetyl-4-(7-methoxy-2-phenyl-quinolin-4-yloxy)-pyrrolidine-2-carboxylic acid and 0.1 mL (0.66 mmol) DIPEA in 6 mL DMF is added 101 mg (0.27 mmol) HBTU at 0° C. The reaction mixture is allowed to warm to room temperature, stirred for 48 hours and concentrated in vacuo. The residue is taken up in 1 N HCl and extracted twice with EtOAc. The combined organic layers are washed with saturated aqueous NaHCO₃ and brine and are dried with Na₂SO₄ and concentrated in vacuo. The residue is purified by preparative reverse phase HPLC (Method E) to afford 2S,4R)-1-Acetyl-4-(7-methoxy-2-phenyl-quinolin-4-yloxy)-pyrrolidine-2-carboxylic acid ((Z)-(5R,7S)-2,2,4-trioxo-15-oxa-2Λ⁶-thia-3-aza-tricyclo[14.4.0.0^{5,7}]icosa-1(16),8,17,19-tetraen-5-yl)-amide as an off-white solid. HPLC (Method C): Rt=3.07 min; LC-MS (Method A): M+H=739.5

### Preparation of (Z)-(5R,7S)-5-Amino-2,2-dioxo-15-oxa-2Λ⁶-thia-3-aza-tricyclo[4.4.0.0^{5,7}]icosa-1 (16),8,17,19-tetraen-4-one hydrochloride

### Step 1

### [(1R,2S)-1-(2-Hept-6-enyloxy-benzenesulfonylaminocarbonyl)-2-vinyl-cyclopropyl]-carbamic acid tert-butyl ester

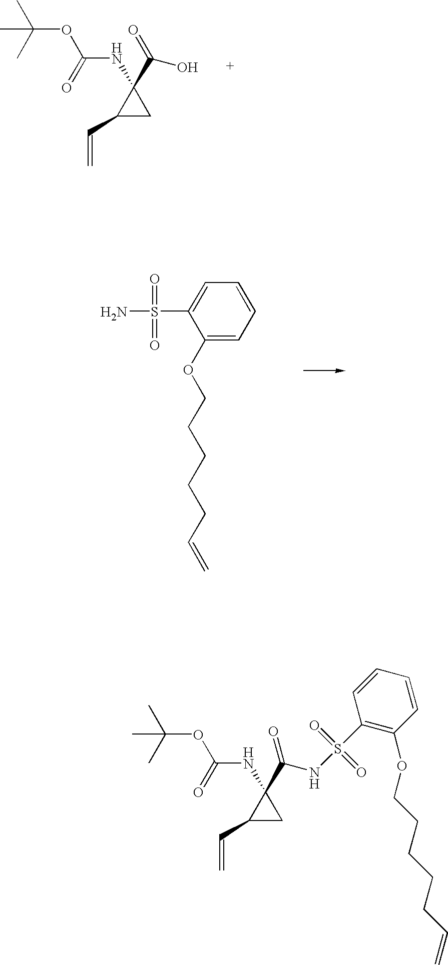

To a solution of 1.1 g (4.8 mmol) (1R,2S)-1-tert-Butoxycarbonylamino-2-vinyl-cyclopropanecarboxylic acid in 10 mL THF is added 1.24 g (7.3 mmol) CDI and the mixture is stirred at 65° C. for 1 hour. The mixture is allowed to cool to room temperature and 1.43 g (5.3 mmol) 2-Hept-6-enyloxy-benzenesulfonamide (prepared according to the same procedure as described for Example 15, step 1) and 1.1 mL (7.3 mmol) DBU are added. The reaction is stirred at room temperature for 5 days. 1 N HCl is added and the mixture is extracted twice with EtOAc. The organic layer is washed with water, dried with Na₂SO₄ and concentrated in vacuo. The residue is purified by preparative reverse phase HPLC (Method E) to give [(1R,2S)-1-(2-Hept-6-enyloxy-benzenesulfonylaminocarbonyl)-2-vinyl-cyclopropyl]-carbamic acid tert-butyl ester as a orange oil. HPLG (Method C): Rt=4.21 min; LC-MS (Method A): M−H=477.0

### Step 2

### ((Z)-(5R,7S)-2,2,4-Trioxo-15-oxa-2Λ⁶-thia-3-aza-tricyclo[14.4.0.0^{5,7}]icosa-1(16),8,17,19-tetraen-5-yl)-carbamic acid tert-butyl ester

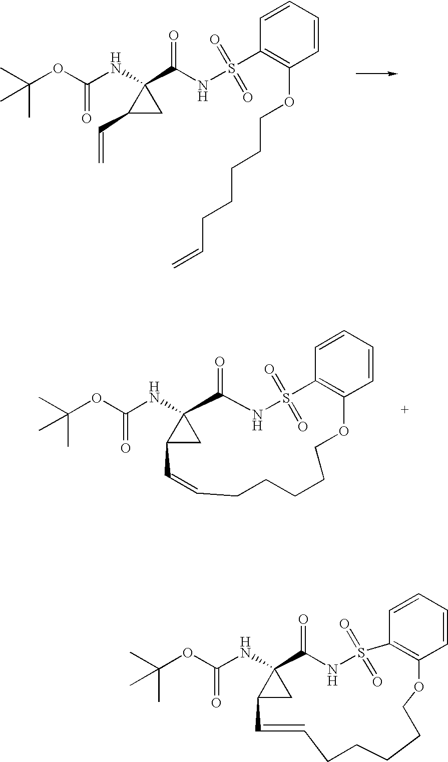

A solution of 1.46 g (3.05 mmol) [(1R,2S)-1-(2-Hept-6-enyloxy-benzenesulfonyl-aminocarbonyl)-2-vinyl-cyclopropyl]-carbamic acid tert-butyl ester and 382 mg (20 mol-%) Grubbs 11 catalyst in 800 mL CH₂Cl₂ is refluxed for 12 hours. The reaction is concentrated in vacuo and the residue is purified by preparative reverse phase HPLC (Method E) to afford ((Z)-(5R,7S)-2,2,4-Trioxo-15-oxa-2Λ⁶-thia-3-aza-tricyclo[14.4.0.0^{5,7}]icosa-1(16),8,17,19-tetraen-5-yl)-carbamic acid tert-butyl ester and ((E)-(5R,7S)-2,2,4-Trioxo-15-oxa-2Λ⁶-thia-3-aza-tricyclo[14.4.0.0^{5,7}]icosa-1(16),8,17,19-tetraen-5-yl)-carbamic acid tert-butyl ester. (Z) isomer: LC-MS (Method A): Rt=3.59 min; M−H=448.9; (E) isomer: LC-MS (Method A): Rt=3.73 min; M−H=448.9

### Step 3

### (Z)-(5R,7S)-5-Amino-2,2-dioxo-15-oxa-2Λ⁶-thia-3-aza-tricyclo[14.4.0.0^{5,7}]icosa-1(16),8,17,19-tetraen-4-one hydrochloride

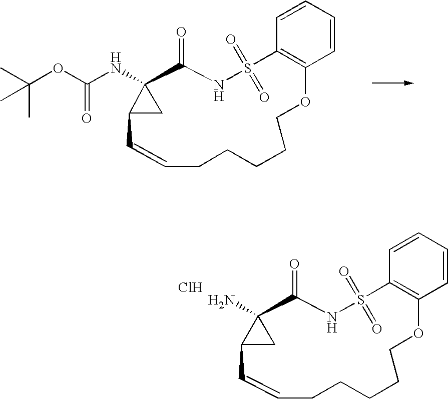

To a solution of 100 mg (0.22 mmol) ((Z)-(5R,7S)-2,2,4-Trioxo-15-oxa-2Λ⁶-thia-3-aza-tricyclo[14.4.0.0^{5,7}]icosa-1(16),8,17,19-tetraen-5-yl)-carbamic acid tert-butyl ester in 1.7 mL dioxane is added 0.8 mL HCl in dioxane (4M) at 40° C. and the mixture is stirred this temperature overnight. The reaction is concentrated in vacuo to afford (Z)-(5R,7S)-5-Amino-2,2-dioxo-15-oxa-2Λ⁶-thia-3-aza-tricyclo[14.4.0.0^{5,7}]icosa-1 (16),8,17,19-tetraen-4-one hydrochloride. LC-MS (Method A): Rt=3.94 min; M+H=350.9

### Example 18

The following compound is prepared according to the same procedure described in Example 17 starting from ((E)-(5R,7S)-2,2,4-Trioxo-15-oxa-2Λ⁶-thia-3-aza-tricyclo[14.4.0.0^{5,7}]icosa-1(16),8,17,19-tetraen-5-yl)-carbamic acid tert-butyl ester.

### (2S,4R)-1-Acetyl-4-(7-methoxy-2-phenyl-quinolin-4-yloxy)-pyrrolidine-2-carboxylic acid ((E)-(5R,7S)-2,2,4-trioxo-15-oxa-2Λ⁶-thia-3-aza-tricyclo[14.4.0.0^{5,7}]icosa-1(16),8,17,19-tetraen-5-yl)-amide

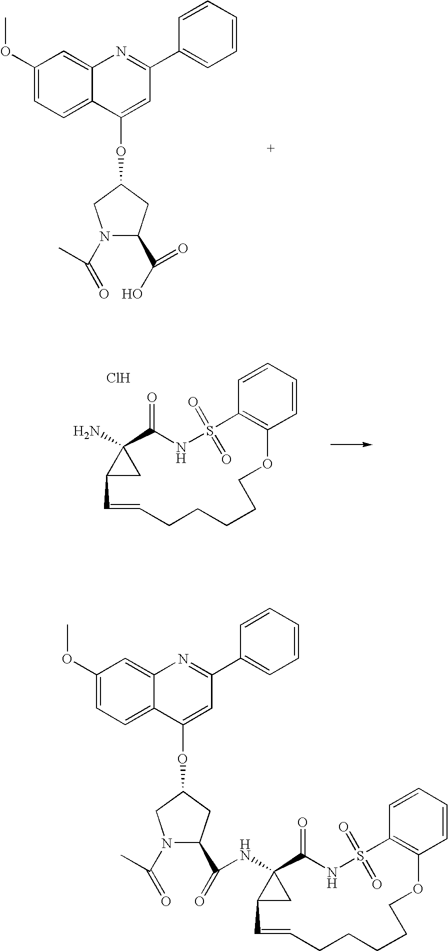

HPLC (Method C): Rt=3.09 min ; LC-MS (Method A): M−H=738.0, M+H=740.0

### Example 19

The following compound is prepared according to the same procedure described in Example 1.

### (2S,4R)-1-Acetyl-4-(7-methoxy-2-phenyl-quinolin-4-yloxy)-pyrrolidine-2-carboxylic acid ((Z)-(5R,7S)-2,2,4,16-tetraoxo-2Λ⁶-thia-3,15-diaza-tricyclo[15.3.1.0^{5,7}]henicosa-1(20),8,17(21),18-tetraen-5-yl)-amide

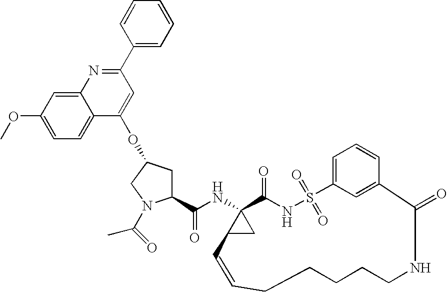

HPLC (Method C): Rt=2.97 min ; LC-MS (Method A): M−H=763.9, M+H=766.0

### Preparation of (Z)-(5R,7S)-5-Amino-2,2-dioxo-2Λ⁶-thia-3,15-diaza-tricyclo[15.3.1.0^{5,7}]henicosa-1(20),8,17(21),18-tetraene-4,16-dione hydrochloride

### Step 1

N-Hept-6-enyl-3-sulfamoyl-benzamide

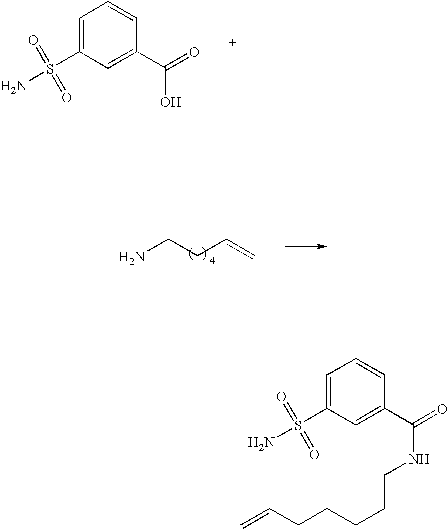

To a solution of 0.40 g (1.99 mmol) 3-carboxybenzenesulfonamide, 0.50 g 1-Amino-6-heptene trifluoroacetate (Hu et al., J. Med Chem. 2003, 47, 4941) and 1.42 mL (8.2 mmol) DIPEA in 10 mL DMF is added 0.98 g (2.6 mmol) HBTU at 0° C. The reaction mixture is allowed to warm to room temperature and is stirred for 12 hours. It is then concentrated in vacuo and taken up in 10 mL EtOAc and 10 mL 1N HCl. The phases are separated and the aqueous phase is extracted with 10 mL EtOAc. The combined organic phases are washed with 5% a. NaHCO₃ and brine, dried with Na₂SO₄ and concentrated in vacuo. The residue is purified by preparative reverse phase HPLC (Method E) to afford N-Hept-6-enyl-3-sulfamoyl-benzamide as a white solid. LC-MS (Method A): Rt=3.41 min; M+H=311.0, M−H=309.0

### Step 2

### ((1R,2S)-1-(3-Hept-6-enylcarbamoyl-benzenesulfonylaminocarbonyl)-2-vinyl-cyclopropyl)-carbamic acid tert-butyl ester

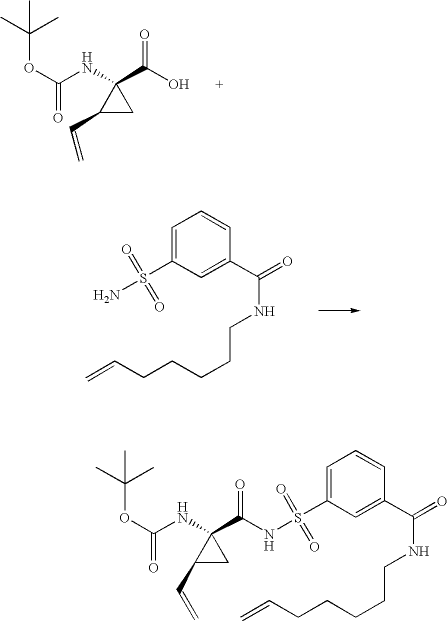

To a solution of 240 mg (1.06 mmol) (1R,2S)-1-tert-Butoxycarbonylamino-2-vinyl-cyclopropanecarboxylic acid in 3 mL THF is added 270 mg (1.58 mmol) CDI and the mixture is stirred at 70° C. for 2 hours. The mixture is allowed to cool to room temperature and 313 mg (1.06 mmol) N-Hept-6-enyl-3-sulfamoyl-benzamide and 0.24 mL (1.58 mmol) DBU are added. The reaction is stirred at room temperature for 12 hours. 10 mL EtOAc are added and the mixture is washed with 5 mL 1 N HCl. The organic layer is dried with MgSO₄and concentrated in vacuo. The residue is purified by preparative reverse phase HPLC (Method E) to afford ((1R,2S)-1-(3-Hept-6-enylcarbamoyl-benzenesulfonylaminocarbonyl)-2-vinyl-cyclopropyl)-carbamic acid tert-butyl ester as an off-white solid. HPLC (Method C): Rt=3.73 min ; LC-MS (Method A): M−H=504.0

### Step 3

### ((Z)-(5R,7S)-2,2,4,16-Tetraoxo-2Λ⁶-thia-3,15-diaza-tricyclo[15.3.1.0^{5,7}]henicosa-1(20),8,17(21),18-tetraen-5-yl)-carbamic acid tert-butyl ester/((E)-(5R,7S)-2,2,4,16-Tetraoxo-2Λ⁶-thia-3,15-diaza-tricyclo[15.3.1.0^{5,7}]henicosa-1(20),8,17(21),18-tetraen-5-tetraen-5-yl)-carbamic acid tert-butyl ester

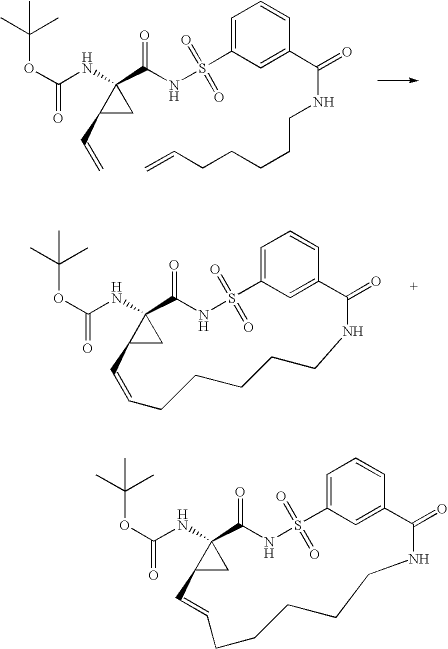

A solution of 260 mg (0.51 mmol) ((1R,2S)-1-(3-Hept-6-enylcarbamoyl-benzenesulfonylaminocarbonyl)-2-vinyl-cyclopropyl)-carbamic acid tert-butyl ester and 64 mg (20 mol-%) Grubbs II catalyst in 140 mL CH₂Cl₂ is refluxed for 12 hours. The reaction is concentrated in vacuo and the residue is purified by preparative reverse phase HPLC (Method E) to afford ((Z)-(5R,7S)-2,2,4,16-Tetraoxo-2Λ⁶-thia-3,15-diaza-tricyclo[15.3.1.0^{5,7}]henicosa-1(20),8,17(21),18-tetraen-5-yl)-carbamic acid tert-butyl ester and ((E)-(5R,7S)-2,2,4,16-Tetraoxo-2Λ⁶-thia-3,15-diaza-tricyclo[15.3.1.0^{5,7}]henicosa-1(20),8,17(21),18-tetraen-5-yl)-carbamic acid tert-butyl ester both as off-white solids. (Z) isomer: LC-MS (Method A): Rt=3.31 min; M−H=475.9; (E) isomer: LC-MS (Method A): Rt=3,16 min; M−H=475.9

### Step 4

### (Z)-(5R,2S)-5-Amino-2,2-dioxo-2Λ⁶-thia-3,15-diaza-tricyclo[15.3.1.0^{5,7}]henicosa-1(20),8,17(21),18-tetraene-4,16-dione hydrochloride

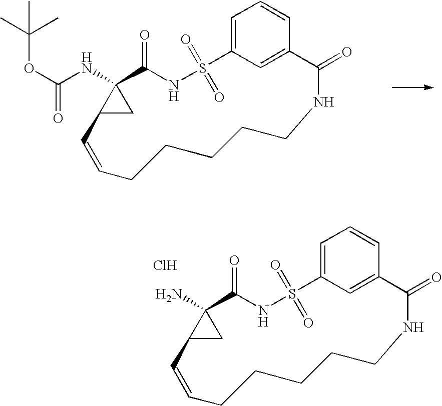

To a solution of 31 mg (0.07 mmol) ((Z)-(5R,7S)-2,2,4,16-Tetraoxo-2Λ⁶-thia-3,15-diaza-tricyclo[15.3.1.0^{5,7}]henicosa-1(20),8,17(21),18-tetraen-5yl)-carbamic acid tert-butyl ester in 0.5 mL dioxane is added 0.2 mL HCl in dioxane (4M) at 40° C. and the mixture is stirred this temperature for 1 h. The reaction is concentrated in vacuo to afford (Z)-(5R,7S)-5-Amino-2,2-dioxo-2Λ⁶-thia-3,15-diaza-tricyclo[15.3.1.05,7]henicosa-1(20),8,17(21),18-tetraene-4,16-dione hydrochloride. LC-MS (Method A): Rt=3.66 min; M−H=376.0

### Example 20

The following compound is prepared according to the same procedure described in Example 19.

### (2S,4R)-1-Acetyl-4-(7-methoxy-2-phenyl-quinolin-4-yloxy)-pyrrolidine-2-carboxylic acid ((E)-(5R,7S)-2,2,4,16-tetraoxo-2Λ⁶-thia-3,15-diaza-tricyclo[15.3.1.0^{5,7}]henicosa-1(20),8,17(21),18-tetraen-5-yl)-amide

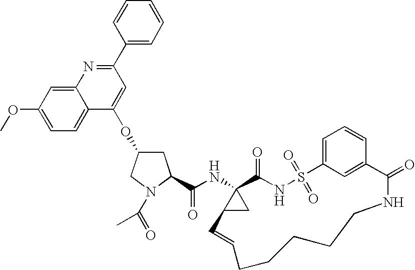

HPLC (Method C): Rt=2.89 min ; LC-MS (Method A): M+H=766.0

### Example 21

### (2S,4R)-1-(N′-Acetyl-N-isopropyl-hydrazinocarbonyl)-4-[2-(2-isopropylamino-thiazol-4-yl)-7-methoxy-quinolin-4-yloxy]-pyrrolidine-2-carboxylic acid ((Z)-(5R,7S)-2,2,4-trioxo-2Λ⁶-thia-3,15-diaza-tricyclo[14.4.0.0 5,7]icosa-1(16),8,17,19-tetraen-5-yl)-amide

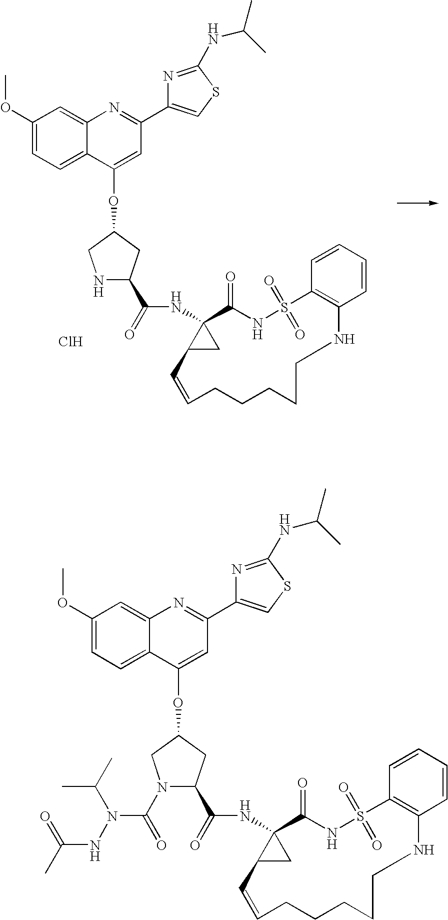

To a solution of 40 mg (0.050 mmol) (2S,4R)-4-[2-(2-Isopropylamino-thiazol-4-yl)-7-methoxy-quinolin-4-yloxy]-pyrrolidine-2-carboxylic acid ((Z)-(5R,7S)-2,2,4-trioxo-2Λ⁶-thia-3,15-diaza-tricyclo[14.4.0.0^{5,7}]icosa-1(16),8,17,19-tetraen-5-yl)-amide hydrochloride and 14.1 μL (0.1 mmol) triethylamine in 0.3 mL of dichloromethane are added 10.8 mg (0.0603 mmol) acetic acid N′-isopropyl-N′-chlorocarbonyl hydrazide at 0° C. The reaction mixture is allowed to warm to room temperature and is stirred for 12 hours. It is then concentrated in vacuo and taken up in ethyl acetate and 1N HCl. The organic phase is dried and concentrated in vacuo. The residue is purified by preparative reverse phase HPLC (Method E) to afford (2S,4R)-1-(N′-Acetyl-N-isopropyl-hydrazinocarbonyl)-4-[2-(2-isopropylamino-thiazol-4-yl)-7-methoxy-quinolin-4-yloxy]-pyrrolidine-2-carboxylic acid ((Z)-(5R,7S)-2,2,4-trioxo-2Λ⁶-thia-3,15-diaza-tricyclo[14.4.0.0^{5,7}]icosa-1(16),8,17,19-tetraen-5-yl)-amide as a yellow solid.

HPLC (Method C): Rt=3.66 min ; LC-MS (Method A): M+H=902.3

### Preparation of (2S,4R)-4-[2-(2-Isopropylamino-thiazol-4-yl)-7-methoxy-quinolin-4-yloxy]-pyrrolidine-2-carboxylic acid ((Z)-(5R,7S)-2,2,4-trioxo-2Λ⁶-thia-3,15-diaza-tricyclo[14.4.0.0^{5,7}]icosa-1(16),8,17,19-tetraen-5-yl)-amide hydrochloride

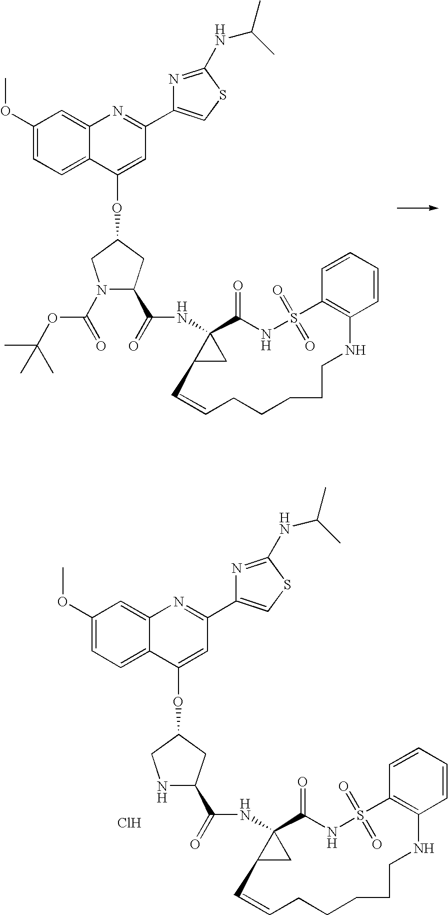

To a solution of 460 mg (0.535 mmol) (2S,4R)-4-[2-(2-Isopropylamino-thiazol-4-yl)-7-methoxy-quinolin-4-yloxy]-2-((Z)-(5R,7S)-2,2,4-trioxo-2Λ⁶-thia-3,15-diaza-tricyclo[14.4.0.0^{5,7}]icosa-1(16),8,17,19-tetraen-5-ylcarbamoyl)-pyrrolidine-1-carboxylic acid tert-butyl ester in 1.5 mL of dioxane are added 1.3 mL HCl in dioxane (4N) and the mixture is stirred at RT for 3 hours. The reaction is concentrated in vacuo to afford (2S,4R)-4-[2-(2-Isopropylamino-thiazol-4-yl)-7-methoxy-quinolin-4-yloxy]-pyrrolidine-2-carboxylic acid ((Z)-(5R,7S)-2,2,4-trioxo-2Λ⁶-thia-3,15-diaza-tricyclo[14.4.0.0^{5,7}]icosa-1(16),8,17,19-tetraen-5-yl)-amide hydrochloride as a yellow solid. LC-MS (method A): Rt 2.620 min; M+H=760.2

### Preparation of Acetic acid N′-isopropyl-N′-chlorocarbonyl hydrazide

### Step 1

### Acetic acid N′-isopropyl-hydrazide

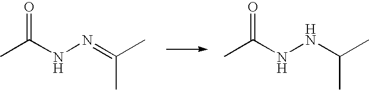

To a solution of 1.5 g (13.1 mmol) acetic acid isopropylidene-hydrazide in 50 mL of ethanol are added 0.5 g of palladium on charcoal and the mixture is stirred at 50° C. for 56hours in a H₂-atmosphere. The reaction mixture is filtered through Celite and concentrated in vacuo. The residue is purified by flash chromatography on silica gel (dichloromethane/methanol: 100/0 to 9/1) to afford acetic acid N′-isopropyl-hydrazide as a white solid. M+H=117.1

### Step 2

### Acetic acid N′-isopropyl-N′-chlorocarbonyl hydrazide

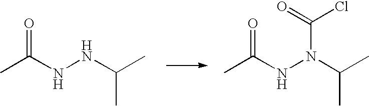

To a solution of 640 μL (1.29 mmol) of phosgene in toluene (20-wt-%) is added a solution of 50 mg (0.43 mmol) acetic acid N′-isopropyl-hydrazide in 0.3 mL of toluene at 0° C. The reaction mixture is allowed to warm to room temperature and is stirred for 12 hours. It is then concentrated in vacuo to afford acetic acid N′-isopropyl-N′-chlorocarbonyl hydrazide as a white solid.

### Example 22

### (2S,4R)-4-[2-(2-isopropylamino-thiazol-4-yl)-7-methoxy-quinolin-4-yloxy]-1-((S)-3-methyl-2-methylcarbamoylmethyl-butyryl)-pyrrolidine-2-carboxylic acid ((Z)-(5R,7S)-2,2,4-trioxo-2Λ⁶-thia-3,15-diaza-tricyclo[14.4.0.0^{5,7}]icosa-1(16),8,17,19-tetraen-5-yl)-amide

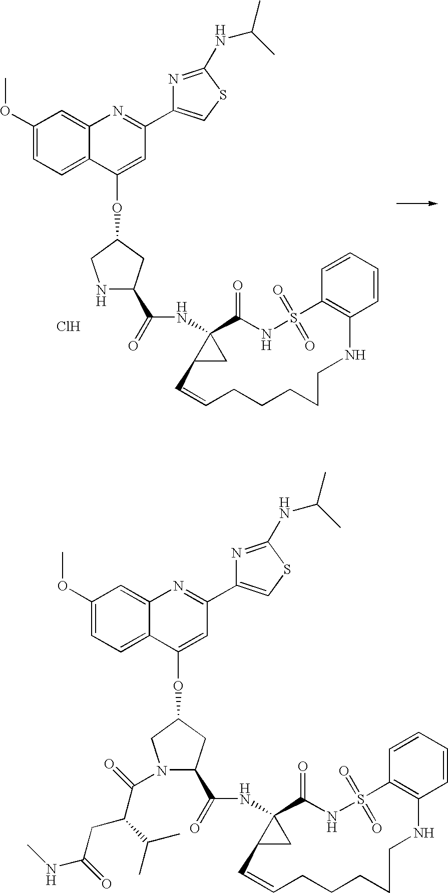

To a solution of 40 mg (0.0273 mmol, purity 54%) (2S,4R)-4-[2-(2-Isopropylamino-thiazol-4-yl)-7-methoxy-quinolin-4-yloxy]-pyrrolidine-2-carboxylic acid ((Z)-(5R,7S)-2,2,4-trioxo-2Λ⁶-thia-3,15-diaza-tricyclo[14.4.0.0^{5,7}]icosa-1(16),8,17,19-tetraen-5-yl-amide hydrochloride, 7.16 mg (0.0409 mmol) (S)-3-Methyl-2-methylcarbamoylmethyl-butyric acid and 19.1 μL (0.109 mmol) DIPEA in 0.5 mL of DMF are added 15.8 mg (0.0409 mmol). HBTU at 0° C. The reaction mixture is allowed to warm to room temperature and is stirred for 12 hours. It is then diluted with ethyl acetate and 1N HCl. The organic phase is dried and concentrated in vacuo. The residue is purified by preparative reverse phase HPLC (Method E) to afford (2S,4R)-4-[2-(2-isopropylamino-thiazol-4-yl)-7-methoxy-quinolin-4-yloxy]-1-((S)-3-methyl-2-methylcarbamoylmethyl-butyryl)-pyrrolidine-2-carboxylic acid ((Z)-(5R,7S)-2,2,4-trioxo-2Λ⁶-thia-3,15-diaza-tricyclo[14.4.0.0^{5,7}]icosa-1(16),8,17,19-tetraen-5-yl)-amide as a yellow solid. HPLC (Method C): Rt 3.51 min; LC-MS (Method A): M+H=915.3

### Preparation of (S)-3-Methyl-2-methylcarbamoylmethyl-butyric acid

### Step 1

### (S)-3-Methyl-2-methylcarbamoylmethyl-butyric acid methyl ester

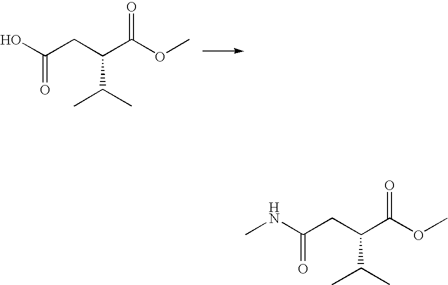

To a solution of 100 mg (0.574 mmol) (S)-2-isopropylsuccinic acid-1-methylester, 46.5 mg (0.689 mmol) methylamine hydrochloride and 401 μL (2.3 mmol) DIPEA in 2 mL of DMF are added 333 mg (0.0.861 mmol) HBTU at 0° C. The reaction mixture is allowed to warm to room temperature and is stirred for 12 hours. It is then diluted with ethyl acetate and 1N HCl. The organic phase is dried and concentrated in vacuo. The residue is by flash chromatography on silica gel (dichloromethane/methanol: 100/0 to 9/1) to afford (S)-3-methyl-2-methylcarbamoylmethyl-butyric acid methyl ester as a yellow oil. LC-MS (Method A′ (5%)): Rt 2.912 min; M+H=188.1

### Step 2

### (S)-3-Methyl-2-methylcarbamoylmethyl-butyric acid

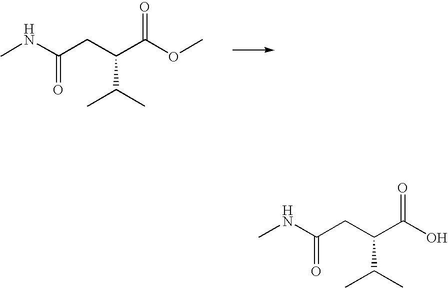

To a solution of 198 mg (0.574 mmol) (S)-3-Methyl-2-methylcarbamoylmethyl-butyric acid methyl ester in 2 mL of THF, 1 mL of MeOH and 1 mL of water are added 73 mg of lithium hydroxide hydrate (1.72 mmol). The reaction mixture is stirred at RT for 12 hours. It is then concentrated in vacuo and taken up in ethyl acetate and 1N HCl. The organic phase is dried and concentrated in vacuo to afford (S)-3-methyl-2-methylcarbamoylmethyl-butyric acid as a yellow solid. LC-MS (Method A′ (5%)): Rt 1.783 min; M+H=174.1

### Example 23

The following compound is prepared according to the same procedure described in Example 22 using L-α-hydroxyisovaleric acid instead of (S)-3-methyl-2-methylcarbamoylmethyl-butyric acid.

### (2S,4R)-1-(2-Hydroxy-3-methyl-butyryl)-4-[2-(2-isopropylamino-thiazol-4-yl)-7-methoxy-quinolin-4-yloxy]-pyrrolidine-2-carboxylic acid ((Z)-(5R,7S)-2,2,4-trioxo-2Λ⁶-thia-3,15-diaza-tricyclo[14.4.0.0^{5,7}]icosa-1(16),8,17,19-tetraen-5-yl)-amide

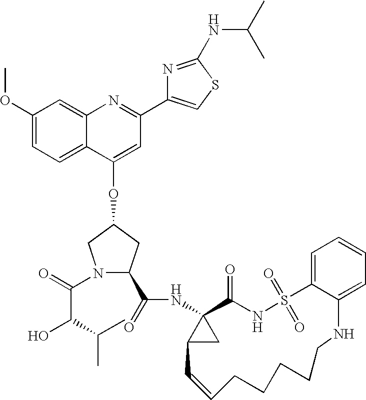

HPLC (Method C): Rt 3.53 min; LC-MS (Method A): M+H=861, M−1=859.3

### Example 24

The following compound is prepared according to the same procedure described in Example 22 using 3-acetylamino-benzoic acid instead of (S)-3-methyl-2-methylcarbamoylmethyl-butyric acid.

### (2S,4R)-1-(3-Acetylamino-benzoyl)-4-[2-(2-isopropylamino-thiazol-4-yl)-7-methoxy-quinolin-4-yloxy]-pyrrolidine-2-carboxylic acid ((Z)-(5R,7S)-2,2,4-trioxo-2Λ⁶-thia-3,15-diaza-tricyclo[14.4.0.0^{5,7}]icosa-1(16),8,17,19-tetraen-5-yl)-amide

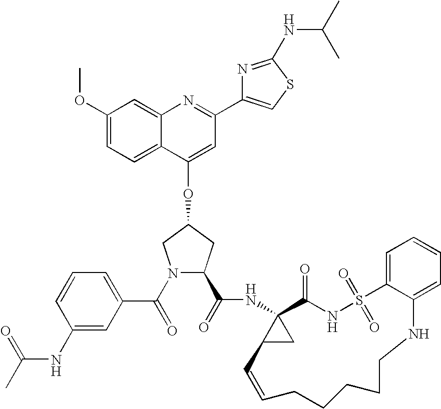

HPLC (Method C): Rt 3.44 min; LC-MS (Method A): M+H=922.3

### Example 25

The following compound is prepared according to the same procedure described in Example 22 using benzoic acid instead of (S)-3-methyl-2-methylcarbamoylmethyl-butyric acid.

### (2S,4R)-1-benzoyl-4-[2-(2-isopropylamino-thiazol-4-yl)-7-methoxy-quinolin-4-yloxy]-pyrrolidine-2-carboxylic acid ((Z)-(5R,7S)-2,2,4-trioxo-2Λ⁶-thia-3,15-diaza-tricyclo[14.4.0.0^{5,7}]icosa-1(16),8,17,19-tetraen-5-yl)-amide

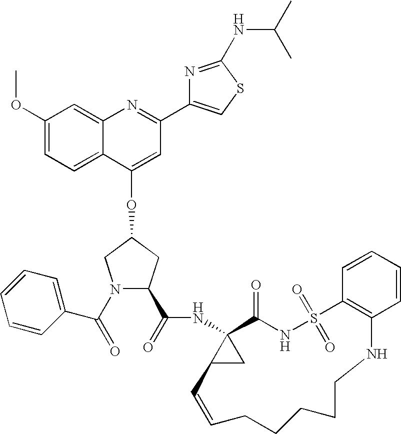

HPLC (Method C): Rt 3.64 min; LC-MS (Method A): M−H=863.2

### Example 26

The following compound is prepared according to the same procedure described in Example 22 using 2-Acetylamino-benzoic acid instead of (S)-3-methyl-2-methylcarbamoylmethyl-butyric acid.

### (2S,4R)-1-(2-Acetylamino-benzoyl-4-[2-(2-isopropylamino-thiazol-4-yl)-7-methoxy-quinolin-4-yloxy]-pyrrolidine-2-carboxylic acid ((Z)-(5R,7S)-2,2,4-trioxo-2Λ⁶-thia-3,15-diaza-tricyclo[14.4.0.0^{5,7}]icosa-1(16),8,17,19-tetraen-5-yl)-amide

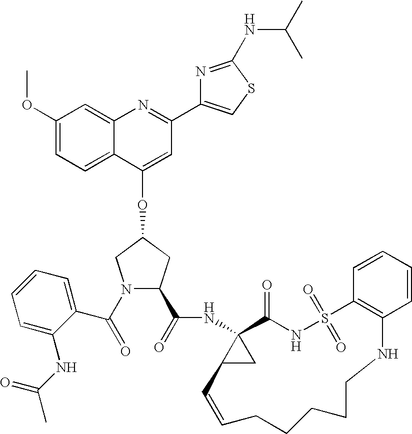

HPLC (Method C): Rt 3.57 min; LC-MS (Method A): M+H=921.3

### Example 27

The following compound is prepared according to the same procedure described in Example 22 using 2-picolinic acid instead of (S)-3-methyl-2-methylcarbamoylmethyl-butyric acid.

### (2S,4R)-4-[2-(2-Isopropylamino-thiazol-4-yl)-7-methoxy-quinolin-4-yloxy]-1-(pyridine-2-carbonyl)-pyrrolidine-2-carboxylic acid ((Z)-(5R,7S)-2,2,4-trioxo-2Λ⁶-thia-3,15-diaza-tricyclo[14.4.0.0^{5,7}]icosa-1(16),8,17,19-tetraen-5-yl)-amide

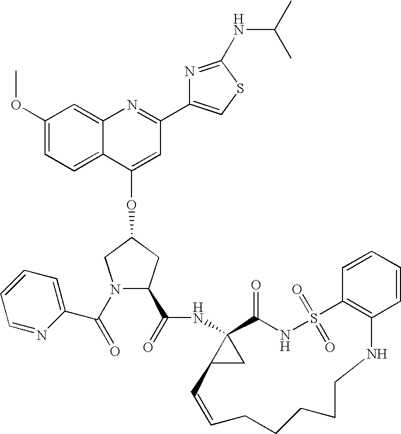

HPLC (Method C): Rt 3.52 min; LC-MS (Method A): M−H=864.3

### Example 28

The following compound is prepared according to the same procedure described in Example 22 using 2H-pyrazole-3-carboxylic acid instead of (S)-3-methyl-2-methylcarbamoylmethyl-butyric acid.

### (2S,4R)-4-[2-(2-Isopropylamino-thiazol-4-yl)-7-methoxy-quinolin-4-yloxy]-1-(2H-pyrazole-3-carbonyl)-pyrrolidine-2-carboxylic acid ((Z)-(5R,7S)-2,2,4-trioxo-2Λ⁶-thia-3,15-diaza-tricyclo[14.4.0.0^{5,7}]icosa-1(16),8,17,19-tetraen-5-yl)-amide

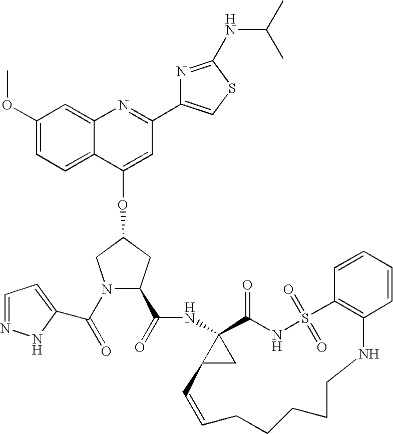

HPLC (Method C): Rt 3.45 min; LC-MS (Method A): M+H=855.3

### Example 29

The following compound is prepared according to the same procedure described in Example 22 using tert-butylacetic acid instead of (S)-3-methyl-2-methylcarbamoylmethyl-butyric acid.

### (2S,4R)-1-(3,3-Dimethyl-butyryl)-4-[(2-(2-isopropylamino-thiazol-4-yl)-7-methoxy-quinolin-4-yloxy]-pyrrolidine-2-carboxylic acid ((Z)-(5R,7S)-2,2,4-trioxo-2A⁶-thia-3,15-diaza-tricyclo[14.4.0.0^{5,7}]icosa-1(16),8,17,19-tetraen-5-yl)-amide

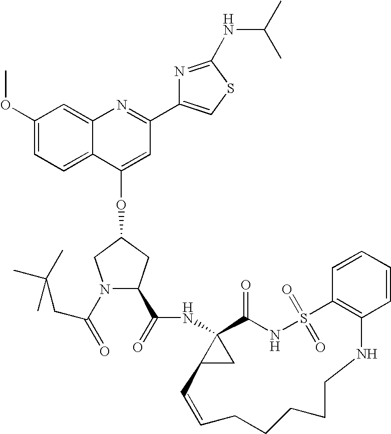

HPLC (Method C): Rt 3.82 min; LC-MS (Method A): M+H=859.3

### Example 30

The following compound is prepared according to the same procedure described in Example 22 using L-pyroglutamic acid instead of (S)-3-methyl-2-methylcarbamoylmethyl-butyric acid.

### (2S,4R)-4-[2-(2-Isopropylamino-thiazol-4-yl)-7-methoxy-quinolin-4-yloxy]-1-((S)-5-oxo-pyrrolidine-2-carbonyl)-pyrrolidine-2-carboxylic acid ((Z)-(5R,7S)-2,2,4-trioxo-2A⁶-thia-3,15-diaza-tricyclo[14.4.0.0^{5,7}]icosa-1(16),8,17,19-tetraen-5-yl)-amide

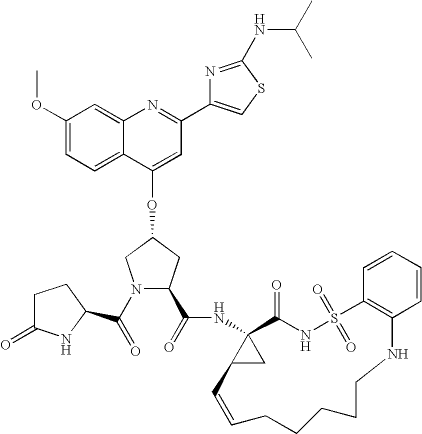

HPLC (Method C): Rt 3.27 min; LC-MS (Method A): M+H=872.3

### Example 31

### (2S,4R)-4-[2-(2-Isopropylamino-thiazol-4-yl)-7-methoxy-quinolin-4-yloxy]-pyrrolidine-1,2-dicarboxylic acid 1-amide 2-[((Z)-(5R,7S)-2,2,4-trioxo-2A⁶-thia-3,15-diaza-tricyclo[14.4.0.0^{5,7}]icosa-1(16),8,17,19-tetraen-5-yl)-amide]

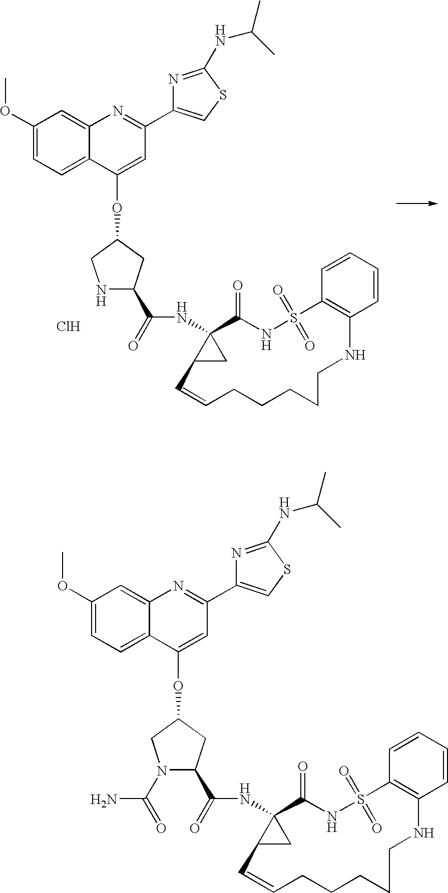

To a solution of 50 mg (0.063 mmol) (2S,4R)-4-[2-(2-isopropylamino-thiazol-4-yl)-7-methoxy-quinolin-4-yloxy]-pyrrolidine-2-carboxylic acid ((Z)-(5R,7S)-2,2,4-trioxo-2A⁶-thia-3,15-diaza-tricyclo[14.4.0.0^{5,7}]icosa-1(16),8,17,19-tetraen-5-yl)-amide hydrochloride in 0.130 mL of acetic acid are added 4.08 mg (0.063 mmol) sodium cyanate. The reaction mixture is stirred at RT for 3 hours. It is then diluted with ethyl acetate and 1N NaOH. The organic phase is dried and concentrated in vacuo. The residue is purified by preparative reverse phase HPLC (Method E) to afford (2S,4R)-4-[2-(2-Isopropylamino-thiazol-4-yl)-7-methoxy-quinolin-4-yloxy]-pyrrolidine-1,2-dicarboxylic acid 1-amide 2-[((Z)-(5R,7S)-2,2,4-trioxo-2A⁶-thia-3,15-diaza-tricyclo[14.4.0.0^{5,7}]icosa-1(16),8,17,19-tetraen-5-yl)-amide] as a yellow solid. HPLC (Method C): Rt 3.37 min; LC-MS (Method A): M+H=803.2

### Example 32

The following compound is prepared according to the same procedure described in

### Example 22 using pivalic acid instead of (S)-3-methyl-2-methylcarbamoylmethyl-butyric acid.

### (2S,4R)-1-(2,2-Dimethyl-propionyl)-4-[2-(2-isopropylamino-thiazol-4-yl)-7-methoxy-quinolin-4-yloxy]-pyrrolidine-2-carboxylic acid ((Z)-(5R,7S)-2,2,4-trioxo-2A⁶-thia-3,15-diaza-tricyclo[14.4.0.0^{5,7}]icosa-1(16),8,17,19-tetraen-5-yl)-amide

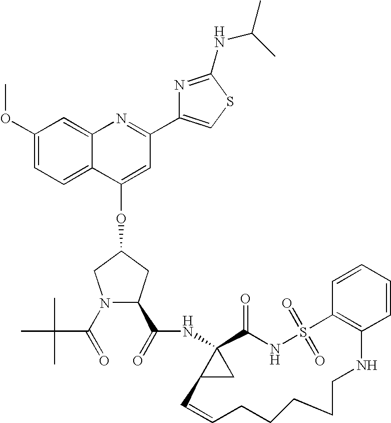

HPLC (Method C): Rt 3.74 min; LC-MS (Method A): M+H=845.3

### Example 33

The following compound is prepared according to the same procedure described in Example 22 using α-hydroxyisobutyric acid instead of (S)-3-methyl-2-methylcarbamoylmethyl-butyric acid.

### (2S,4R)-1-(2-Hydroxy-2-methyl-propionyl)-4-[2-(2-isopropylamino-thiazol-4-yl)-7-methoxy-quinolin-4-yloxy]-pyrrolidine-2-carboxylic acid ((Z)-(5R,7S)-2,2,4-trioxo-2A⁶-thia-3,15-diaza-tricyclo[14.4.0.0^{5,7}]icosa-1(16),8,17,19-tetraen-5-yl)-amide

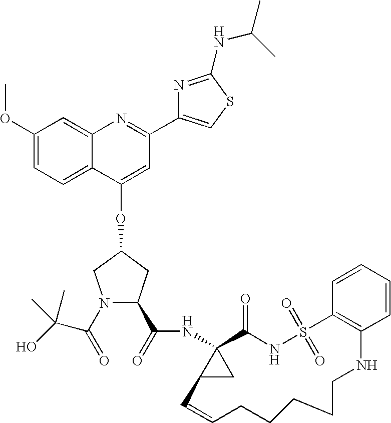

HPLC (Method C): Rt 3.52 min; LC-MS (Method A): M+H=847.3

### Example 34

### (2S,4R)-4-[2-(2-Isopropylamino-thiazol-4-yl)-7-methoxy-quinolin-4-yloxy]-pyrrolidine-1,2-dicarboxylic acid 1-tert-butylamide 2-[((Z)-(5R,7S)-2,2,4-trioxo-2A⁶-thia-3,15-diaza-tricyclo[14.4.0.0^{5,7}]icosa-1(16),8,17,19-tetraen-5-yl)-amide]

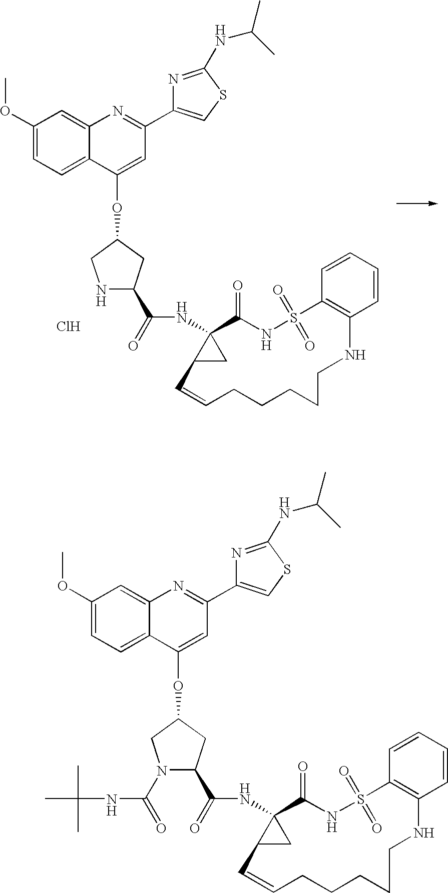

To a solution of 50 mg (0.063 mmol) (2S,4R)-4-[2-(2-isopropylamino-thiazol-4-yl)-7-methoxy-quinolin-4-yloxy]-pyrrolidine-2-carboxylic acid ((Z)-(5R,7S)-2,2,4-trioxo-2A⁶-thia-3,15-diaza-tricyclo[14.4.0.0^{5,7}]icosa-1(16),8,17,19-tetraen-5-yl)-amide hydrochloride and 26.3 μL (0.188 mmol) triethylamine in 0.3 mL of dichloromethane are added 7.79 μL (0.069mmol) tert-butyl isocyanate at 0° C. The reaction mixture is allowed to warm to room temperature and is stirred for 2 hours. It is then concentrated in vacuo. The residue is purified by preparative reverse phase HPLC (Method E) to afford (2S,4R)-4-[2-(2-Isopropylamino-thiazol-4-yl)-7-methoxy-quinolin-4-yloxy]-pyrrolidine-1,2-dicarboxylic acid 1-tert-butylamide 2-[((Z)-(5R,7S)-2,2,4-trioxo-2A⁶-thia-3,15-diaza-tricyclo[14.4.0.0^{5,7}]icosa-1(16),8,17,19-tetraen-5-yl)-amide] as a yellow solid. HPLC (Method C): Rt 3.77 min; LC-MS (Method A): M+H=860.3

### Example 35

The following compound is prepared according to the same procedure described in example 34 using phenyl isocyanate instead of tert-butyl isocyanate.

### (2S,4R)-4-[2-(2-Isopropylamino-thiazol-4-yl)-7-methoxy-quinolin-4-yloxy]-pyrrolidine-1,2-dicarboxylic acid 1-phenylamide 2-[((Z)-(5R,7S)-2,4-trioxo-2A⁶-thia-3,15-diaza-tricyclo[14.4.0.0^{5,7}]icosa-1(16),8,17,19-tetraen-5-yl)-amide]

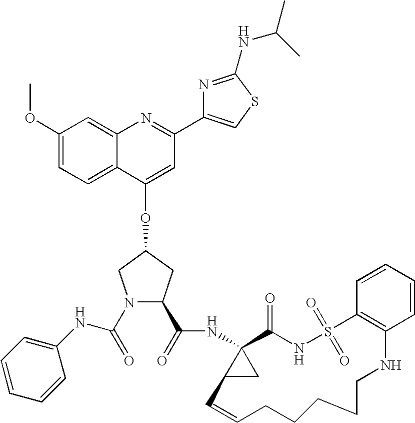

HPLC (Method C): Rt 3.73 min; LC-MS (Method A): M+H=879.3

### Example 36

The following compound is prepared according to the same procedure described in example 34 using benzyl isocyanate instead of tert-butyl isocyanate.

### (2S,4R)-4-[2-(2-Isopropylamino-thiazol-4-yl)-7-methoxy-quinolin-4-yloxy]-pyrrolidine-1,2-dicarboxylic acid 1-benzylamide 2-[((Z)-(5R,7S)-2,2,4-trioxo-2A⁶-thia-3,15-diaza-tricyclo[14.4.0.0^{5,7}]icosa-1(16),8,17,19-tetraen-5-yl)-amide]

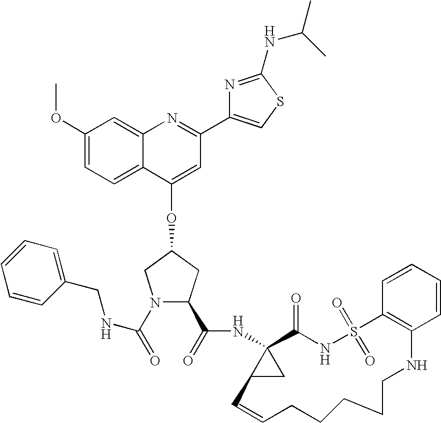

HPLC (Method C): Rt 3.73 min; LC-MS (Method A): M+H=894.3

### Example 37

The following compound is prepared according to the same procedure described in Example 22 using acetic acid instead of (S)-3-methyl-2-methylcarbamoylmethyl-butyric acid.

### (2S,4R)-1-Acetyl-4-[2-(2-isopropylamino-thiazol-4-yl)-7-methoxy-quinolin-4-yloxy]-pyrrolidine-2-carboxylic acid ((Z)-(5R,7S)-2,2,4-trioxo-2A⁶-thia-3,15-diaza-tricyclo[14.4.0.0^{5,7}]icosa-1(16),8,17,19-tetraen-5-yl)-amide

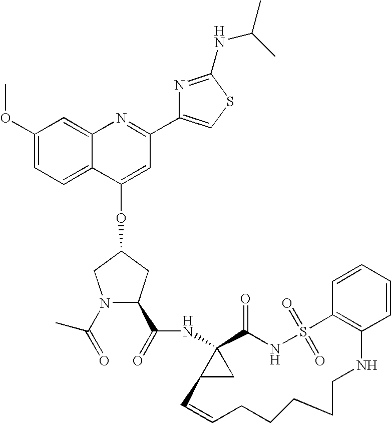

HPLC (Method C): Rt 3.47 min; LC-MS (Method A): M+H=802.3

### Example 38

The following compound is prepared according to the same procedure described in Example 36 using (2S,4R)-4-(7-methoxy-2-phenyl-quinolin-4-yloxy)-pyrrolidine-2-carboxylic acid ((Z)-(5R,7S)-2,2,4-trioxo-2A⁶-thia-3,15-diaza-tricyclo[14.4.0.0^{5,7}]icosa-1(16),8,17,19-tetraen-5-yl)-amide hydrochloride instead of (2S,4R)-4-[2-(2-Isopropylamino-thiazol-4-yl)-7-methoxy-quinolin-4-yloxy]-pyrrolidine-2-carboxylic acid ((Z)-(5R,7S)-2,2,4-trioxo-2A⁶-thia-3,15-diaza-tricyclo[14.4.0.0^{5,7}]icosa-l(16),8,17,19-tetraen-5-yl)-amide hydrochloride.

### (2S,4R)-4-(7-Methoxy-2-phenyl-quinolin-4-yloxy)-pyrrolidine-1,2-dicarboxylic acid 1-benzylamide 2-[((Z)-(5R,7S)-2,4-trioxo-2]ambda*6*-thia-3,15-diaza-tricyclo[14.4.0.0*5,7*]icosa-1(16),8,17,19-tetraen-5-yl)-amide]

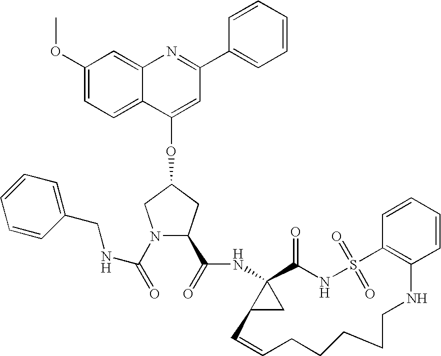

HPLC (Method C): Rt 3.62 min; LC-MS (Method A): M−H=828.0

### Example 39

The following compound is prepared according to the same procedure described in Example 22 using Boc-L-homoleucine instead of (S)-3-methyl-2-methylcarbamoylmethyl-butyric acid.

### ((S)-1-{2-[(2S,4R)-4-[2-(2-isopropylamino-thiazol-4-yl)-7-methoxy-quinolin-4-yloxy]-2-((Z)-(5R,7S)-2,2,4-trioxo-2A⁶-thia-3,15-diaza-tricyclo[14.4.0.0^{5,7}]icosa-1(16),8,17,19-tetraen-5-ylcarbamoyl)-pyrrolidin-1-yl]-2-oxo-ethyl}-3-methyl-butyl)-carbamic acid tert-butyl ester

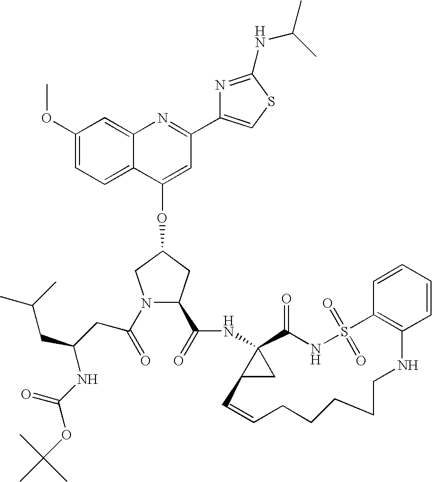

HPLC (Method C): Rt 3.950 min; LC-MS (Method A): M+H=987.3

### Example 40

### (2S,4R)-1-((S)-3-Acetylamino-5-methyl-hexanoyl)-4-[2-(2-isopropylamino-thiazol-4-yl)-7-methoxy-quinolin-4-yloxy]-pyrrolidine-2-carboxylic acid ((Z)-(5R,7S)-2,2,4-trioxo-2A⁶-thia-3,15-diaza-tricyclo[14.4.0.0^{5,7}]icosa-1(16),8,17,19-tetraen-5-yl)-amide

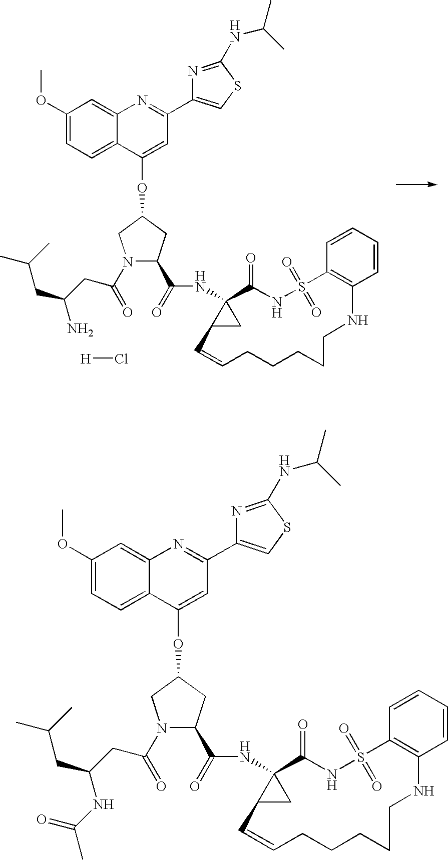

To a solution of 17 mg (0.015 mmol) (2S,4R)-1-((S)-3-Amino-5-methyl-hexanoyl)-4-[2-(2-isopropylamino-thiazol-4-yl)-7-methoxy-quinolin-4-yloxy]-pyrrolidine-2-carboxylic acid ((Z)-(5R,7S)-2,2,4-trioxo-2A⁶-thia-3,15-diaza-tricyclo[14.4.0.0^{5,7}]icosa-1(16),8,17,19-tetraen-5-yl)-amide hydrochloride, 1.28 μL (0.022 mmol) acetic acid and 10.4 μL (0.059 mmol) DIPEA in 0.5 mL of DMF are added 8.65 mg (0.022 mmol) HBTU at 0° C. The reaction mixture is allowed to warm to room temperature and is stirred for 12 hours. It is then diluted with ethyl acetate and 1N HCl. The organic phase is dried and concentrated in vacuo. The residue is purified by preparative reverse phase HPLC (Method E) to afford (2S,4R)-1-((S)-3-Acetylamino-5-methyl-hexanoyl)-4-[2-(2-isopropylamino-thiazol-4-yl)-7-methoxy-quinolin-4-yloxy)-pyrrolidine-2-carboxylic acid ((Z)-(5R,7S)-2,2,4-trioxo-2A⁶-thia-3,15-diaza-tricyclo[14.4.0.0^{5,7}]icosa-1(16),8,17,19-tetraen-5-yl)-amide as a yellow solid. HPLC (Method C): Rt 3.58 min; LC-MS (Method A): M+H=930.3

### Preparation of (2S,4R)-1-((S)-3-Amino-5-methyl-hexanoyl)-4-[2-(2-isopropylamino-thiazol-4-yl)-7-methoxy-quinolin-4-yloxy]-pyrrolidine-2-carboxylic acid ((Z)-(5R,7S)-2,2,4-trioxo-2A⁶-thia-3,15-diaza-tricyclo[14.4.0.0^{5,7}]icosa-1(16),8,17,19-tetraen-5-yl)-amide hydrochloride

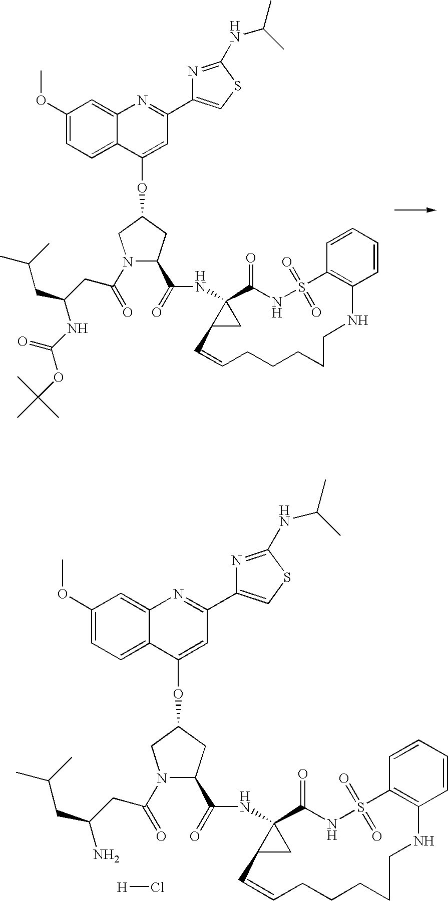

To a solution of 15 mg (0.015 mmol) ((S)-1-{2-[(2S,4R)-4-[2-(2-isopropylamino-thiazol-4-yl)-7-methoxy-quinolin-4-yloxy]-2-((Z)-(5R,7S)-2,2,4-trioxo-2A⁶-thia-3,15-diaza-tricyclo[14.4.0.0^{5,7}]icosa-1(16),8,17,19-tetraen-5-ylcarbamoyl)-pyrrolidin-1-yl]-2-oxo-ethyl}-3-methyl-butyl)-carbamic acid tert-butyl ester in 0.5 mL. of dioxane are added 0.5 mL HCl in dioxane (4N) and the mixture is stirred at RT for 1 hour. The reaction is concentrated in vacuo to afford (2S,4R)-1-((S)-3-Amino-5-methyl-hexanoyl)-4-[2-(2-isopropylamino-thiazol-4-yl)-7-methoxy-quinolin-4-yloxy]-pyrrolidine-2-carboxylic acid ((Z)-(5R,7S)-2,2,4-trioxo-2A⁶-thia-3,15-diaza-tricyclo[14.4.0.0^{5,7}]icosa-1(16),8,17,19-tetraen-5-yl)-amide hydrochloride as a yellow solid. LC-MS (method A): Rt 2.710 min; M+H=887.3

### Example 41

The following compound is prepared according to the same procedure described in Example 22 using Boc-L-β-leucine instead of (S)-3-methyl-2-methylcarbamoylmethyl-butyric acid.

### ((R)-1-{2-[(2S,4R)-4-[2-(2-isopropylamino-thiazol-4-yl)-7-methoxy-quinolin-4-yloxy]-2-((Z)-(5R,7S)-2,2,4-trioxo-2A⁶-thia-3,15-diaza-tricyclo[14.4.0.0^{5,7}]icosa-1(16),8,17,19-tetraen-5-ylcarbamoyl)-pyrrolidin-1-yl]-2-oxo-ethyl}-2-methyl-propyl)-carbamic acid tert-butyl ester

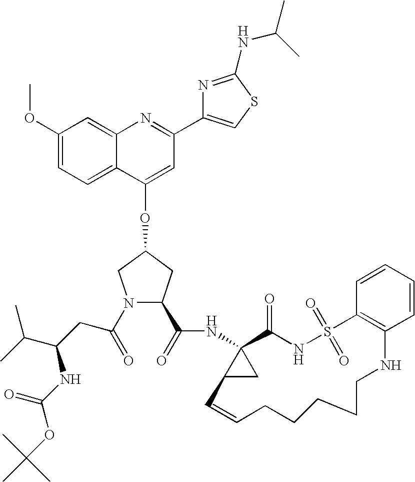

HPLC (Method C): Rt 3.91 min; LC-MS (Method A): M−H=972.4

### Example 42

The following compound is prepared according to the same procedures described in Example 40 using ((R)-1-{2-[(2S,4R)-4-[2-(2-isopropylamino-thiazol-4-yl)-7-methoxy-quinolin-4-yloxy]-2-((Z)-(5R,7S)-2,2,4-trioxo-2A⁶-thia-3,15-diaza-tricyclo[14.4.0.0^{5,7}icosa-1(16),8,17,19-tetraen-5-ylcarbamoyl)-pyrrolidin-1-yl]-2-oxo-ethyl}-2-methyl-propyl)-carbamic acid tert-butyl ester instead of ((S)-1-{2-[(2S,4R)-4-[2-(2-isopropylamino-thiazol-4-yl)-7-methoxy-quinolin-4-yloxy]-2-((Z)-(5R,7S)-2,2,4-trioxo-2A⁶-thia-3,15-diaza-tricyclo[14.4.0.0^{5,7}]icosa-1(16),8,17,19-tetraen-5-ylcarbamoyl)-pyrrolidin-1-yl]-2-oxo-ethyl}-3-methyl-butyl)-carbamic acid tert-butyl ester.

### (2S,4R)-1-((R)-3-Acetylamino-4-methyl-pentanoyl)-4-[2-(2-isopropylamino-thiazol-4-yl)-7-methoxy-quinolin-4-yloxy]-pyrrolidine-2-carboxylic acid ((Z)-(5R,7S)-2,2,4-trioxo-2A⁶-thia-3,15-diaza-tricyclo[14.4.0.0^{5,7}]icosa-1(16),8,17,19-tetraen-5-yl)-amide

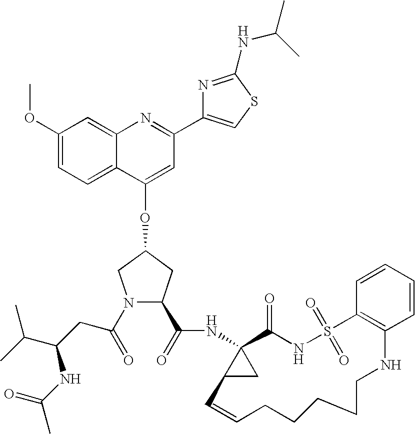

HPLC (Method C): Rt 3.51 min; LC-MS (Method A): M−H=914.3

### Example 43

The following compound is prepared according to the same procedure described in

### Example 22 using Boc-β-glycine instead of (S)-3-methyl-2-methylcarbamoylmethyl-butyric acid.

### (3-[(2S,4R)-4-[2-(2-isopropylamino-thiazol-4-yl)-7-methoxy-quinolin-4-yloxy]-2-((Z)-(5R,7S)-2,4-trioxo-2A⁶-thia-3,15-diaza-tricyclo[14.4.0.0^{5,7}]icosa-1(16),8,17,19-tetraen-5-ylcarbamoyl)-pyrrolidin-1-yl]-3-oxo-propyl}-carbamic acid tert-butyl ester

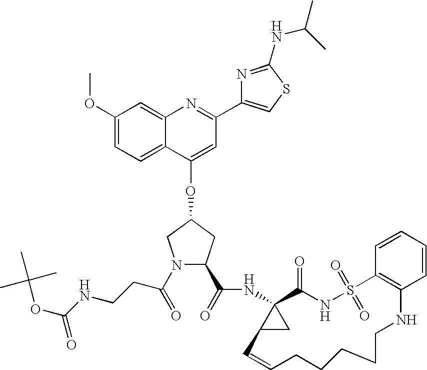

HPLC (Method C): Rt 3.60 min; LC-MS (Method A): M−H=930.3

### Example 44

The following compound is prepared according to the same procedures described in Example 40 using {3-[(2S,4R)-4-[2-(2-isopropylamino-thiazol-4-yl)-7-methoxy-quinolin-4-yloxy]-2-((Z)-(5R,7S)-2,2,4-trioxo-2A⁶-thia-3,15-diaza-tricyclo[14.4.0.0^{5,7}]icosa-1(16),8,17,19-tetraen-5-ylcarbamoyl)-pyrrolidin-1-yl]-3-oxo-propyl}-carbamic acid tert-butyl ester instead of ((S)-1-{2-[(2S,4R)-4-[2-(2-isopropylamino-thiazol-4-yl)-7-methoxy-quinolin-4-yloxy]-2-((Z)-(5R,7S)-2,2,4-trioxo-2A⁶-thia-3,15-diaza-tricyclo[14.4.0.0^{5,7}]icosa-1(16),8,17,19-tetraen-5-ylcarbamoyl)-pyrrolidin-1-yl]-2-oxo-ethyl}-3-methyl-butyl)-carbamic acid tert-butyl ester.

### (2S,4R)-1-(3-Acetylamino-propionyl)-4-[2-(2-isopropylamino-thiazol-4-yl)-7-methoxy-quinolin-4-yloxy]-pyrrolidine-2-carboxylic acid ((Z)-(5R,7S)-2,2,4-trioxo-2A⁶-thia-3,15-diaza-tricyclo[14.4.0.0^{5,7}]icosa-1(16),8,17,19-tetraen-5-yl)-amide

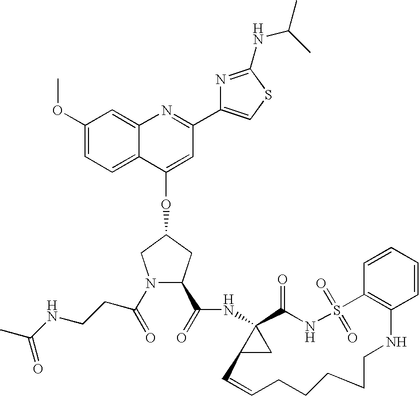

HPLC (Method C): Rt 3.53 min; LC-MS (Method A): M−H=871.3

### Example 45

The following compound is prepared according to the same procedure described in Example 22 using monomethyl succinate instead of (S)-3-methyl-2-methylcarbamoylmethyl-butyric acid.

### 4-[(2S,4R)-4-[2-(2-Isopropylamino-thiazol-4-yl)-7-methoxy-quinolin-4-yloxy]-2-((Z)-(5R,7S)-2,2,4-trioxo-2A⁶-thia-3,15-diaza-tricyclo[14.4.0.0^{5,7}]icosa-1(16),8,17,19-tetraen-5-ylcarbamoyl)-pyrrolidin-1-yl]-4-oxo-butyric acid methyl ester

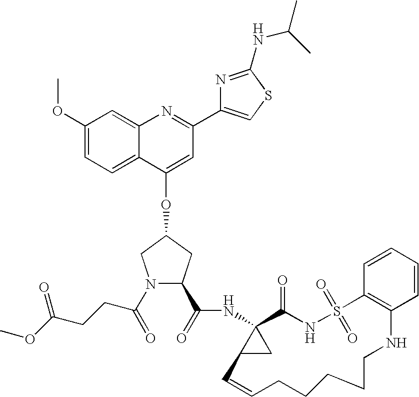

HPLC (Method C): Rt 3.50 min; LC-MS (Method A): M+H=874.3

### Example 46

### (2S,4R)-4-[2-(2-Isopropylamino-thiazol-4-yl)-7-methoxy-quinolin-4-yloxy]-1-(3-methylcarbamoyl-propionyl)-pyrrolidine-2-carboxylic acid ((Z)-(5R,7S)-2,2,4-trioxo-2A⁶-thia-3,15-diaza-tricyclo[14.4.0.0^{5,7}]icosa-1(16),8,17,19-tetraen-5-yl)-amide

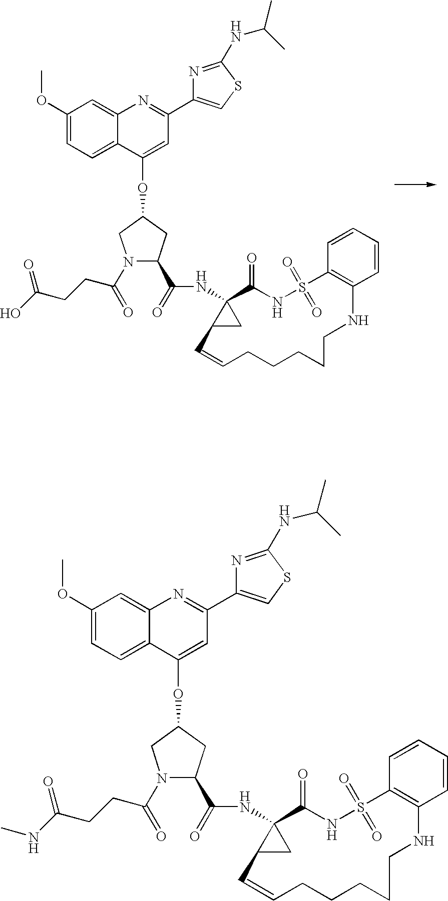

To a solution of 45 mg (0.034 mmol) 4-[(2S,4R)-4-[2-(2-Isopropylamino-thiazol-4-yl)-7-methoxy-quinolin-4-yloxy]-2-((Z)-(5R,7S)-2,2,4-trioxo-2A⁶-thia-3,15-diaza-tricyclo[14.4.0.0^{5,7}]icosa-1(16),8,17,19-tetraen-5-ylcarbamoyl)-pyrrolidin-1-yl]-4-oxo-butyric acid, 4.6 mg (0.068 mmol) methylamine hydrochloride and 23.8 μL (0.136 mmol) DIPEA in 0.5 mL of DMF are added 26.3 mg (0.0682 mmol) HBTU at 0° C. The reaction mixture is allowed to warm to room temperature and is stirred for 12 hours. It is then diluted with ethyl acetate and 1N HCl. The organic phase is dried and concentrated in vacuo. The residue is purified by preparative reverse phase HPLC (Method E) to afford (2S,4R)-4-[2-(2-Isopropylamino-thiazol-4-yl)-7-methoxy-quinolin-4-yloxy]-1-(3-methylcarbamoyl-propionyl)-pyrrolidine-2-carboxylic acid ((Z)-(5R,7S)-2,2,4-trioxo-2A⁶-thia-3,15-diaza-tricyclo[14.4.0.0^{5,7}]icosa-1(16),8,17,19-tetraen-5-yl)-amide as a yellow solid. HPLC (Method C): Rt 3.34 min; LC-MS (Method A): M+H=873.3

### Preparation of 4-[(2S,4R)-4-[2-(2-Isopropylamino-thiazol-4-yl)-7-methoxy-quinolin-4-yloxy]-2-((Z)-(5R,7S)-2,2,4-trioxo-2A⁶-thia-3,15-diaza-tricyclo[14.4.0.0^{5,7}]icosa-1(16),8,17,19-tetraen-5-ylcarbamoyl)-pyrrolidin-1-yl]-4-oxo-butyric acid

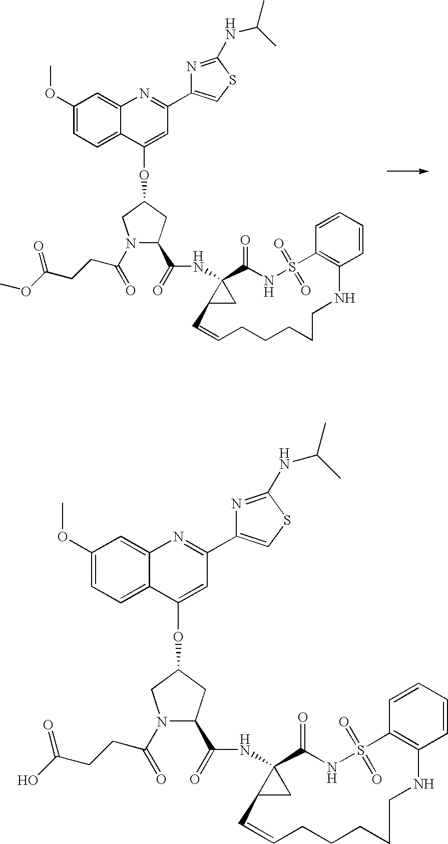

To a solution of 30 mg (0.034 mmol) (4-[(2S,4R)-4-[2-(2-Isopropylamino-thiazol-4-yl)-7-methoxy-quinolin-4-yloxy]-2-((Z)-(5R,7S)-2,2,4-trioxo-2A⁶-thia-3,15-diaza-tricyclo[14.4.0.0^{5,7}]icosa-1(16),8,17,19-tetraen-5-ylcarbamoyl)-pyrrolidin-1-yl]-4-oxo-butyric acid methyl ester in 0.2 mL of THF, 0.1 mL of MeOH and 0.1 mL of water are added 5.8 mg (0.137 mmol) lithium hydroxide hydrate. The reaction mixture is stirred at RT for 12 hours. Another 5.8 mg (0.137 mmol) of lithium hydroxide hydrate are added and the reaction is stirred for another 24 hours. It is then concentrated in vacuo and taken up in ethyl acetate and 1N HCl. The organic phase is dried and concentrated in vacuo to afford 4-{(2S,4R)-4-[2-(2-isopropylamino-thiazol-4-yl)-7-methoxy-quinolin-4-yloxy]-2-((Z)-(5R,7S)-2,2,4-trioxo-2A⁶-thia-3,15-diaza-tricyclo[14.4.0.0^{5,7}]icosa-1(16),8,17,19-tetraen-5-ylcarbamoyl)-pyrrolidin-1-yl]-4-oxo-butyric acid as a yellow solid. LC-MS (Method A): Rt 3.197 min; M+H=860.2.

### Example 47

The following compound is prepared according to the same procedure described in Example 22 using (R)-2-isobutylsuccinic acid-1-methyl ester instead of (S)-3-methyl-2-methylcarbamoylmethyl-butyric acid.

### (R)-2-{2-[(2S,4R)-4-[2-(2-Isopropylamino-thiazol-4-yl)-7-methoxy-quinolin-4-yloxy]-2-((Z)-(5R,7S)-2,2,4-trioxo-2A⁶-thia-3,15-diaza-tricyclo[14.4.0.0^{5,7}]icosa-1(16),8,17,19-tetraen-5-ylcarbamoyl)-pyrrolidin-1-yl]-2-oxo-ethyl}-4-methyl-pentanoic acid methyl ester

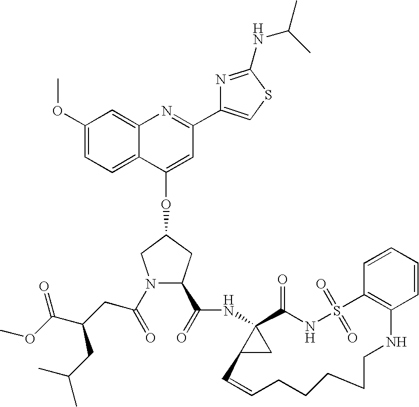

HPLC (Method C): Rt 3.84 min; LC-MS (Method A): M+H=930.4

### Example 48

The following compound is prepared according to the same procedure described in Example 46 using (R)-2-{2-[(2S,4R)-4-[2-(2-Isopropylamino-thiazol-4-yl)-7-methoxy-quinolin-4-yloxy]-2-((Z)-(5R,7S)-2,2,4-trioxo-2A⁶-thia-3,15-diaza-tricyclo[14.4.0.0^{5,7}]icosa-1(16),8,17,19-tetraen-5-ylcarbamoyl)-pyrrolidin-1-yl]-2-oxo-ethyl}-4-methyl-pentanoic acid methyl ester instead of 4-[(2S,4R)-4-[2-(2-Isopropylamino-thiazol-4-yl)-7-methoxy-quinolin-4-yloxy]-2-((Z)-(5R,7S)-2,2,4-trioxo-2A⁶-thia-3,15-diaza-tricyclo[14.4.0.0^{5,7}]icosa-1(16),8,17,19-tetraen-5-ylcarbamoyl)-pyrrolidin-1-yl]-4-oxo-butyric acid methyl ester.

### (2S,4R)-4-[2-(2-Isopropylamino-thiazol-4-yl)-7-methoxy-quinolin-4-yloxy]-1-((R)-5-methyl-3-methylcarbamoyl-hexanoyl)-pyrrolidine-2-carboxylic acid ((Z)-(5R,7S)-2,2,4-trioxo-2A⁶-thia-3,15-diaza-tricyclo[14.4.0.0^{5,7}]icosa-1(16),8,17,19-tetraen-5-yl)-amide

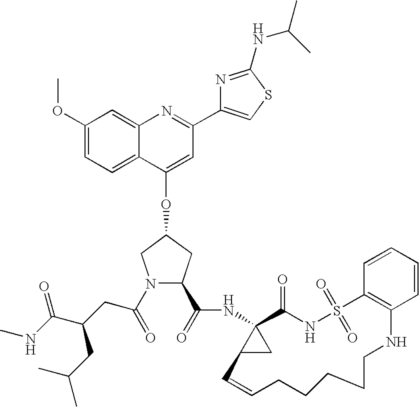

HPLC (Method C): Rt 3.61 min; LC-MS (Method A): M+H=929.3

### Example 49

The following compound is prepared according to the same procedures described in Examples 45 and 46 using (S)-2-isobutylsuccinic acid-1-methyl ester instead of monomethyl succinate.

### (2S,4R)-4-[2-(2-Isopropylamino-thiazol-4-yl)-7-methoxy-quinolin-4-yloxy]-1-((S)-5-methyl-3-methylcarbamoyl-hexanoyl)-pyrrolidine-2-carboxylic acid ((Z)-(5R,7S)-2,2,4-trioxo-2A⁶-thia-3,15-diaza-tricyclo[14.4.0.0^{5,7}]icosa-1(16),8,17,19-tetraen-5-yl)-amide

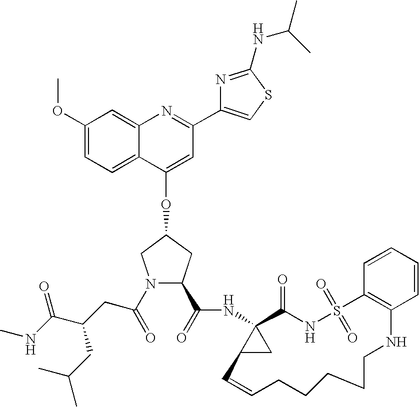

HPLC (Method C): Rt 3.68 min; LC-MS (Method A): M+H=929.3

### Example 50

The following compound is prepared according to the same procedure described in Example 22 using indole-2-carboxylic acid instead of (S)-3-Methyl-2-methylcarbamoylmethyl-butyric acid.

### (2S,4R)-1-(1H-Indole-2-carbonyl)-4-[2-(2-isopropylamino-thiazol-4-yl)-7-methoxy-quinolin-4-yloxy]-pyrrolidine-2-carboxylic acid ((Z)-(5R,7S)-2,2,4-trioxo-2A⁶-thia-3,15-diaza-tricyclo[14.4.0.0^{5,7}]icosa-1(16),8,17,19-tetraen-5-yl)-amide

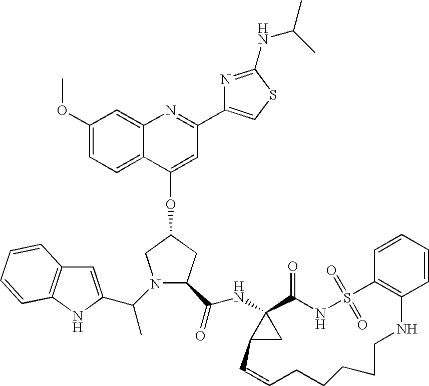

HPLC (Method C): Rt 3.78 min; LC-MS (Method A): M−H=902.3

### Example 51

The following compound is prepared according to the same, procedure as for the preparation of (2S,4R)-4-[2-(2-Isopropylamino-thiazol-4-yl)-7-methoxy-quinolin-4-yloxy]-2-((E)-(5R,7S)-2,2,4,14-tetraoxo-2A⁶-thia-3,15-diaza-tricyclo[14.4.0.0^{5,7}]icosa-1(16),8,17,19-tetraen-5-ylcarbamoyl)-pyrrolidine-1-carboxylic acid tert-butyl ester (described in Example 10) using (Z)-(5R,7S)-5-Amino-2,2-dioxo-2A⁶-thia-3,15-diaza-tricyclo[14.4.0.0^{5,7}]icosa-1(16),8,17,19-tetraene-4,14-dione hydrochloride described in example 1 instead of (Z)-(5R,7S)-5-Amino-2,2-dioxo-2A⁶-thia-3,15-diaza-tricyclo[14.4.0.05 7]icosa-1(16),8,17,19-tetraene-4,14-dione hydrochloride.

### (2S,4R)-4-(2-(2-Isopropylamino-thiazol-4-yl)-7-methoxy-quinolin-4-yloxy]-2-((Z)-(5R,7S)-2,2,4,14-tetraoxo-2A⁶-thia-3,15-diaza-tricyclo[14.4.0.0^{5,7}]icosa-1(16),8,17,19-tetraen-5-ylcarbamoyl)-pyrrolidine-1-carboxylic acid tert-butyl ester

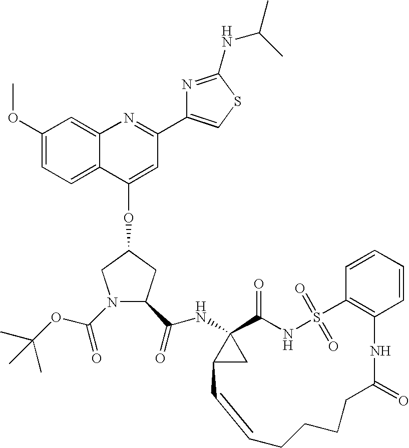

HPLC (Method C): Rt 3.59 min; LC-MS (Method A): M+H=874.3

### Example 52

### (2S,4R)-1-Acetyl-4-[2-(2-isopropylamino-thiazol-4-yl)-7-methoxy-quinolin-4-yloxy]-pyrrolidine-2-carboxylic acid ((Z)-(5R,7S)-2,2,4,14-tetraoxo-2A⁶-thia-3,15-diaza-tricyclo[14.4.0.0^{5,7}]icosa-1(16),8,17,19-tetraen-5-yl)-amide

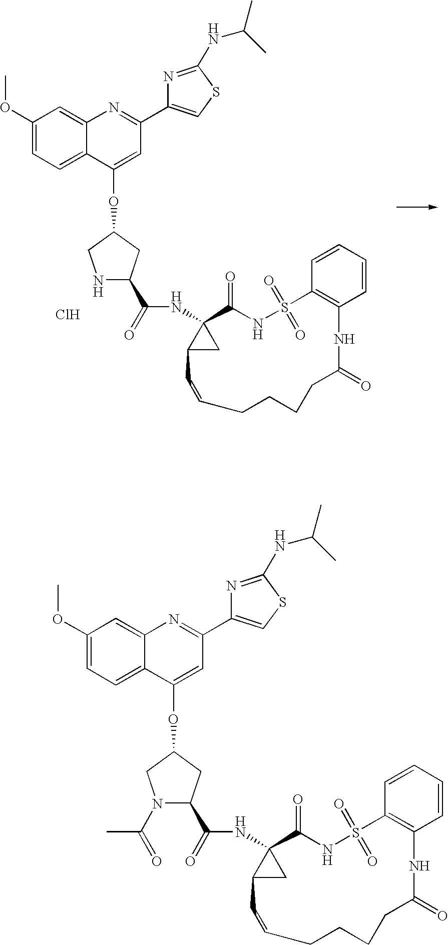

To a solution of 26 mg (0.028 mmol) (2S,4R)-4-[2-(2-isopropylamino-thiazol-4-yl)-7-methoxy-quinolin-4-yloxy]-pyrrolidine-2-carboxylic acid ((Z)-(5R,7S)-2,2,4,14-tetraoxo-2A⁶-thia-3,15-diaza-tricyclo[14.4.0.0^{5,7}]icosa-1(16),8,17,19-tetraen-5-yl)-amide hydrochloride, 1.92 μL (0.033 mmol) acetic acid and 14.6 μL (0.083 mmol) DIPEA in 0.5 mL of DMF are added 16.2 mg (0.041 mmol) HBTU at 0° C. The reaction mixture is allowed to warm to room temperature and is stirred for 12 hours. It is then diluted with ethyl acetate and 1N HCl. The organic phase is dried and concentrated in vacuo. The residue is purified by preparative reverse phase HPLC (Method E) to afford (2S,4R)-1-Acetyl-4-[2-(2-isopropylamino-thiazol-4-yl)-7-methoxy-quinolin-4-yloxy]-pyrrolidine-2-carboxylic acid ((Z)-(5R,7S)-2,2,4,14-tetraoxo-2A⁶-thia-3,15-diaza-tricylclo[14.4.0.0^{5,7}]icosa-1(16),8,17,19-tetraen-5-yl)-amide as a yellow solid. HPLC (Method C): Rt 3.24 min; LC-MS (Method A): M+H=816.3

### Preparation of (2S,4R)-4-[2-(2-Isopropylamino-thiazol-4-yl)-7-methoxy-quinolin-4-yloxy]-pyrrolidine-2-carboxylic acid ((Z)-(5R,7S)-2,2,4,14-tetraoxo-2A⁶-thia-3,15-diaza-tricyclo[14.4.0.0^{5,7}]icosa-1(16),8,17,19-tetraen-5-yl)-amide hydrochloride

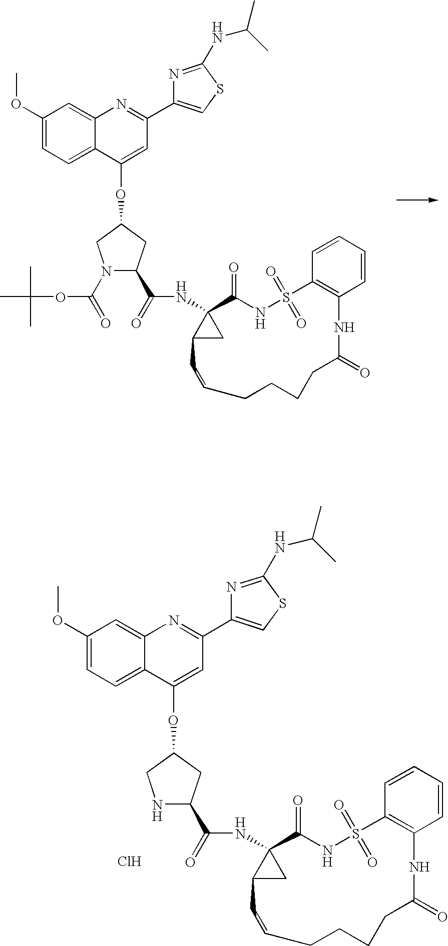

To a solution of 50 mg (0.057 mmol) (2S,4R)-4-[2-(2-Isopropylamino-thiazol-4-yl)-7-methoxy-quinolin-4-yloxy]-2-((Z)-(5R,7S)-2,2,4,14-tetraoxo-2 A⁶-thia-3,15-diaza-tricyclo[14.4.0.0^{5,7}]icosa-1(16),8,17,19-tetraen-5-ylcarbamoyl)-pyrrolidine-1-carboxylic acid tert-butyl ester in 0.2 mL of dioxane are added 0.140 mL HCl in dioxane (4N) and the mixture is stirred at RT for 1 hour. The reaction is concentrated in vacuo to afford (2S,4R)-4-[2-(2-Isopropylamino-thiazol-4-yl)-7-methoxy-quinolin-4-yloxy]-pyrrolidine-2-carboxylic acid ((Z)-(5R,7S)-2,2,4,14-tetraoxo-2A⁶-thia-3,15-diaza-tricyclo[14.4.0.0^{5,7}]icosa-1(16),8,17,19-tetraen-5-yl)-amide hydrochloride as a yellow solid. LC-MS (method A): Rt 2.398 min; M+H =774.2

### Example 53

The following compound is prepared according to the same procedure described in Example 51 using Boc-L-valine instead of acetic acid.

### {(S)-1-[(2S,4R)-4-[2-(2-Isopropylamino-thiazol-4-yl)-7-methoxy-quinolin-4-yloxy]-2-((Z)-(5R,7S)-2,2,4,14-tetraoxo-2A⁶-thia-3,15-diaza-tricyclo[14.4.0.0^{5,7}]icosa-1(16),8,17,19-tetraen-5-ylcarbamoyl)-pyrrolidine-1-carbonyl]-2-methyl-propyl}-carbamic acid tert-butyl ester

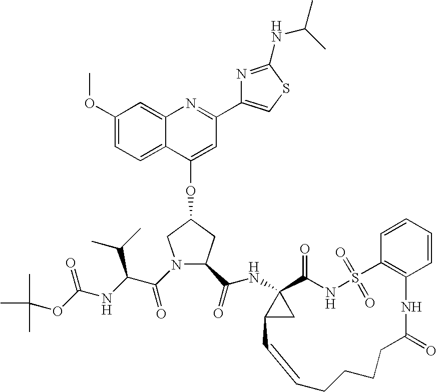

HPLC (Method C): Rt 3.67 min; LC-MS (Method A): M+H=974.3

### Example 54

The following compound is prepared according to the same procedure described in Example 51 using glycolic acid instead of acetic acid and using (2S,4R)-4-[2-(2-Isopropylamino-thiazol-4-yl)-7-methoxy-quinolin-4-yloxy]-2-((E)-(5R,7S)-2,2,4,14-tetraoxo-2A⁶-thia-3,15-diaza-tricyclo[14.4.0.0^{5,7}]icosa-1(16),8,17,19-tetraen-5-ylcarbamoyl)-pyrrolidine-1-carboxylic acid tert-butyl ester (prepared in Example 10) instead of (2S,4R)-4-[2-(2-Isopropylamino-thiazol-4-yl)-7-methoxy-quinolin-4-yloxy]-2-((Z)-(5R,7S)-2,2,4,14-tetraoxo-2A⁶-thia-3,15-diaza-tricyclo[14.4.0.0^{5,7}]icosa-1(16),8,17,19-tetraen-5-ylcarbamoyl)-pyrrolidine-1-carboxylic acid tert-butyl ester

### (2S,4R)-1-(2-Hydroxy-acetyl)-4-[2-(2-isopropylamino-thiazol-4-yl)-7-methoxy-quinolin-4-yloxy]-pyrrolidine-2-carboxylic acid ((E)-(5R,7S)-2,2,4,14-tetraoxo-2A⁶-thia-3,15-diaza-tricyclo14.4.0.0^{5,7}]icosa-1(16),8,17,19-tetraen-5-yl)-amide

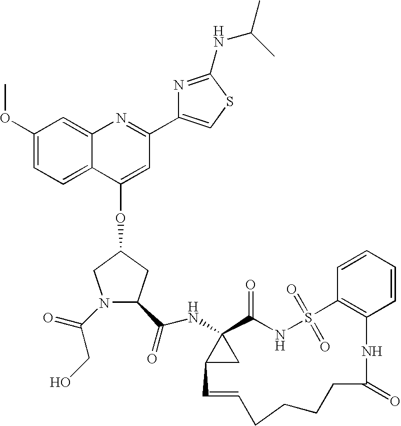

HPLC (Method C): Rt 3.83 min; LC-MS (Method A): M+H=832.3

The following compounds (Examples 55-57) are prepared according to the same procedures described in Examples 1 and 3:

### Example 55

### (2S,4R)-1-Acetyl-4-(7-methoxy-2-phenyl-quinolin-4-yloxy)-pyrrolidine-2-carboxylic acid ((5R,7R)-2,2,4,15-tetraoxo-2A⁶-thia-3,16-diaza-tricyclo[15.4.0.0^{5,7}]henicosa-1(17),18,20-trien-5-yl)-amide

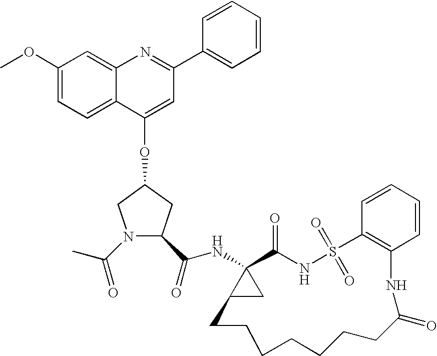

HPLC (Method C): Rt=3.16 min; LC-MS (Method A): M+H=767.9

### Example 56

### (2S,4R)-1-Acetyl-4-(7-methoxy-2-phenyl-quinolin-4-yloxy)-pyrrolidine-2-carboxylic acid ((E)-(5R,7S)-2,2,4,17-tetraoxo-2A⁶-thia-3,16-diaza-tricyclo[16.3.1.0^{5,7}]docosa-1(22),8,18,20-tetraen-5-yl)-amide

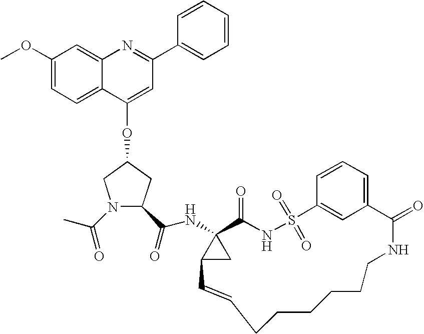

HPLC (Method C): Rt=3.00 min; LC-MS (Method A): M+H=781.0

### Example 57

(2S,4R)-1-Acetyl-4-(7-methoxy-2-phenyl-quinolin-4-yloxy)-pyrrolidine-2-carboxylic acid ((5R,7R)-2,2,4,17-tetraoxo-2A⁶-thia-3,16-diaza-tricyclo[16.3.1.0^{5,7}]docosa-1(22),18,20-trien-5-yl)-amide

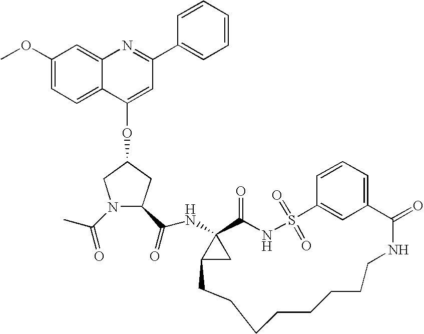

HPLC (Method C): Rt=3.08 min; LC-MS (Method A): M+H=782.2

The following compounds (Examples 58, 59) are prepared according to the same procedures described in Examples 15 and 16:

### Example 58

### (2S,4R)-1-Acetyl-4-(7-methoxy-2-phenyl-quinolin-4-yloxy)-pyrrolidine-2-carboxylic acid ((Z)-(5R,7S)-2,2,4-trioxo-16-oxa-2A⁶-thia-3-aza-tricyclo[5.4.0.0^{5,7}]henicosa-1(17),8,18,20-tetraen-5-yl)-amide

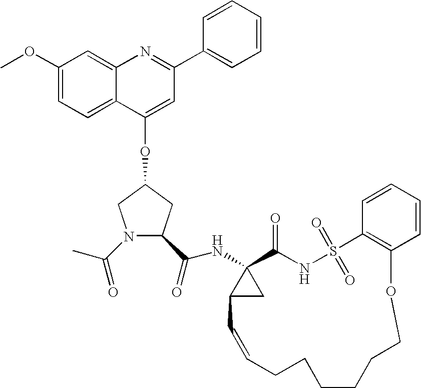

HPLC (Method C): Rt=3.25 min; LC-MS (Method A): M+H=754, M−H=751.8

### Example 59

### (2S,4R)-1-Acetyl-4-(7-methoxy-2-phenyl-quinolin-4-yloxy)-pyrrolidine-2-carboxylic acid ((5R,7R)-2,2,4-trioxo-15-oxa-2A⁶-thia-3-aza-tricyclo[14.4.0.0^{5,7}]icosa-1(16),17,19-trien-5-yl)-amide

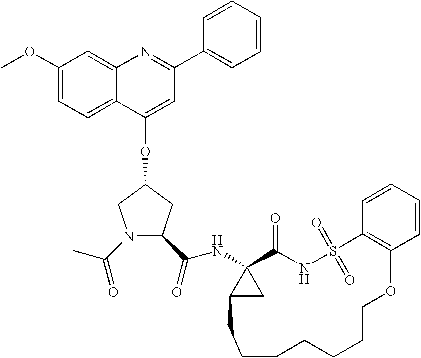

HPLC (Method C): Rt=3.17 min ; LC-MS (Method A): M+H=741.2

### Example 60

### 4-Fluoro-1,3-dihydro-isoindole-2-carboxylic acid (3R,5S)-1-acetyl-5-((Z)-(5R,7S)-2,2,4-trioxo-2A⁶-thia-3,15-diaza-tricyclo[14.4.0.0^{5,7}]icosa-1(16),8,17,19-tetraen-5-ylcarbamoyl)-pyrrolidin-3-yl ester

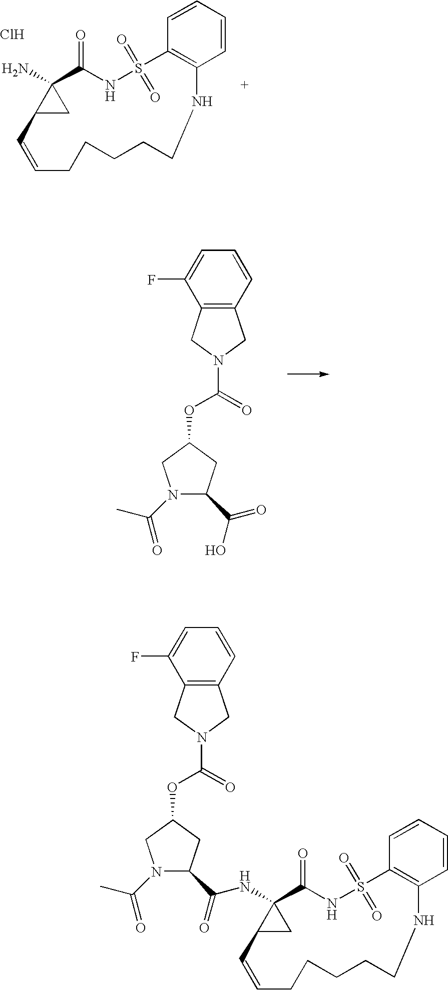

To a solution of 28 mg (0.073 mmol) (Z)-(5R,7S)-5-amino-2,2-dioxo-2A⁶-thia-3,15-diaza-tricyclo[14.4.0.0^{5,7}]icosa-1(16),8,17,19-tetraen-4-one hydrochloride (see Example 4), 24.4 mg (0.073 mmol) 4-fluoro-1,3-dihydro-isoindole-2-carboxylic acid (3R,5S)-1-acetyl-5-carboxy-pyrrolidin-3-yl ester and 51 μL (0.29 mmol) DIPEA in 0.7 mL of DMF are added 35.7 mg (0.094 mmol) HBTU at 0° C. The reaction mixture is allowed to warm to room temperature and is stirred for 12 hours. It is then diluted with ethyl acetate and 1N HCl. The organic phase is dried and concentrated in vacuo. The residue is purified by preparative reverse phase HPLC (Method E) to afford 4-Fluoro-1,3-dihydro-isoindole-2-carboxylic acid (3R,5S)-1-acetyl-5-((Z)-(5R,7S)-2,2,4-trioxo-2A⁶-thia-3,15-diaza-tricyclo[14.4.0.0^{5,7}]icosa-1(16),8,17,19-tetraen-5-ylcarbamoyl)-pyrrolidin-3-yl ester as a white solid. HPLC (Method C): Rt=3.68 min; LC-MS (Method A): M+H=668.2, M+Na=690.2, M−H=666.3

### Example 61

### 4-Fluoro-1,3-dihydro-isoindole-2-carboxylic acid (3R,5S)-1-tert-butoxycarbonyl-5-((Z)-(5R,7S)-2,2,4-trioxo-2A⁶-thia-3,15-diaza-tricyclo[14.4.0.0^{5,7}]icosa-1(16),8,17,19-tetraen-5-ylcarbamoyl)-pyrrolidin-3-yl ester

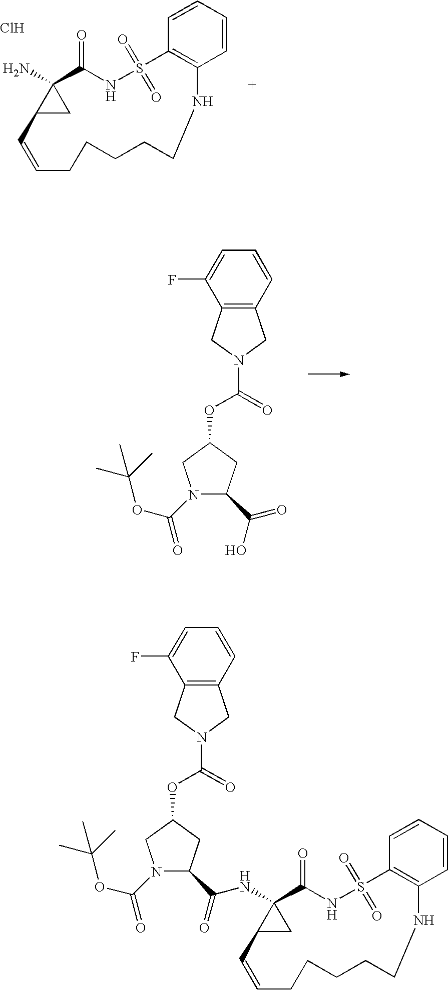

To a solution of 115 mg (0.298 mmol) (Z)-(5R,7S)-5-amino-2,2-dioxo-2A⁶-thia-3,15-diaza-tricyclo[14.4.0.0^{5,7}]icosa-1(16),8,17,19-tetraen-4-one hydrochloride (see Example 4), 118 mg (0.298 mmol) (2S,4R)-4-(4-fluoro-1,3-dihydro-isoindole-2-carbonyloxy)-pyrrolidine-1,2-dicarboxylic acid 1-tert-butyl ester and 208 μL (1.19 mmol) DIPEA in 2.3 mL of DMF are added 147 mg (0.387 mmol) HBTU at 0° C. The reaction mixture is allowed to warm to room temperature and is stirred for 12 hours. It is then diluted with ethyl acetate and 1N HCl. The organic phase is dried and concentrated in vacuo. The residue is purified by preparative reverse phase HPLC (Method E) to afford 4-Fluoro-1,3-dihydro-isoindole-2-carboxylic acid (3R,5S)-1-tert-butoxycarbonyl-5-((Z)-(5R,7S)-2,2,4-trioxo-2A⁶-thia-3,15-diaza-tricyclo[14.4.0.0^{5,7}]icosa-1(16),8,17,19-tetraen-5-ylcarbamoyl)-pyrrolidin-3-yl ester as a white solid. LC-MS (Method A): Rt=4.48 min; M+H=726.2, M−H=724.2

### Example 62

### (3R,5S-1-(tert-butoxycarbonyl)-5-{[(1R,13E,15S)-4,4-dioxido-2-oxo-4-thia-3-azaspiro[bicyclo[13.1.0]hexadecane-5,1-cyclopropane]-13-en-1-yl]carbamoyl}pyrrolidin-3-yl 4-fluoro-1,3-dihydro-2H-isoindole-2-carboxylate

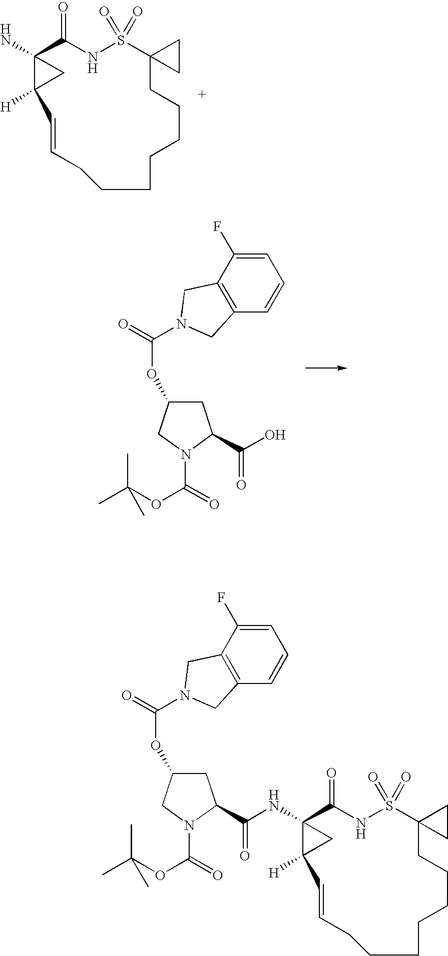

A mixture of 48 mg (0.1 mmol) (2S,4R)-4-(4-fluoro-1,3-dihydro-isoindole-2-carbonyloxy)-pyrrolidine-1,2-dicarboxylic acid 1-tert-butyl ester, 0.06 mL (0.1 mmol) DIPEA and 51 mg (0.1 mmol) HBTU in 2 mL DMF is stirred at rt for 30 min. 40 mg (0.1 mmol) (1R,13E,15S)-1-amino-4-thia-3-azaspiro[bicyclo[13.1.0]hexadecane-5,1′-cyclopropane]-13-en-2-one 4,4-dioxide is added and the mixture is stirred at rt for 5 h before it is partitioned between DCM and aq. K₂CO₃ solution. The aq. layer is extracted with DCM and the combined organic layers are washed with 10% aq. KHSO₄ solution and brine, dried over Na₂SO₄ and concentrated under reduced pressure. The crude is purified by prep. HPLC (method E) to yield the desired product. LC-MS (method A): Rt=4.67 min, M+H=703.2

### Step 1

### Cyclopropanesulfonic Acid Amide

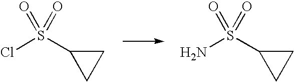

A mixture of 2.9 g (20 mmol) cyclopropanesulfonyl chloride and 100 mL ammonia in dioxane (0.5 M) is stirred at rt overnight. The formed solids are filtered of and the filtrate is concentrated in vacuo. The residue is triturated with DCM to give the desired product as a solid.

### Step 2

### tert-butyl(cyclopropylsulfonyl)carbamate

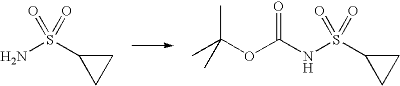

A mixture of 1.3 g (11 mmol) cyclopropanesulfonic acid amide, 2.8 g (13 mmol) Boc2O, 2.2 mL (16 mmol) triethylamine and 66 mg (0.5 mmol) DMAP in 15 mL DCM is stirred, at rt for 72 h. The mixture is concentrated in vacuo and the residue is taken up in EtOAc and washed with 1N HCl and brine. The organic layer is dried over Na₂SO₄ and concentrated to give the product.

### Step 3

### tert-butyl [(1-non-8-en-1-ylcyclopropyl)sulfonyl]carbamate

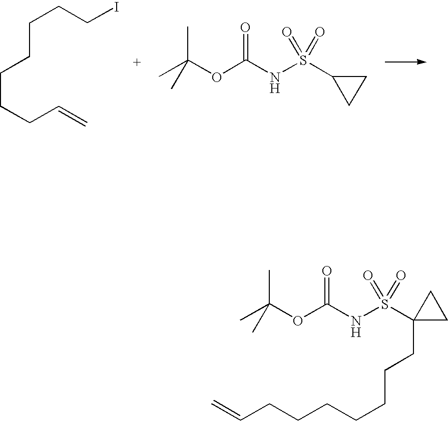

A solution of 3 mL (22 mmol) DIPEA in 30 mL THF is cooled to 0° C and 13 mL (21 mmol) nBuLi in hexanes (1.6 M) is added. After 1 h the mixture is cooled to −78° C and a solution of 1.8 g (8 mmol) tert-butyl (cyclopropylsulfonyl)carbamate in 5 mL THF is added. After 1 h 2.3 g (9 mmol) 9-iodo-non-1-ene is added and the mixture is allowed to reach rt overnight. Sat. aq. NH₄Cl is added and the aq. layer is extracted with EtOAc. The combined organic layers are dried over Na₂SO₄ and concentrated under reduced pressure. Silica gel chromatography (hexanes/EtOAc 7/3) yields the desired product.

### Step 4

### 1-Non-8-enyl-cyclopropanesulfonic acid amide

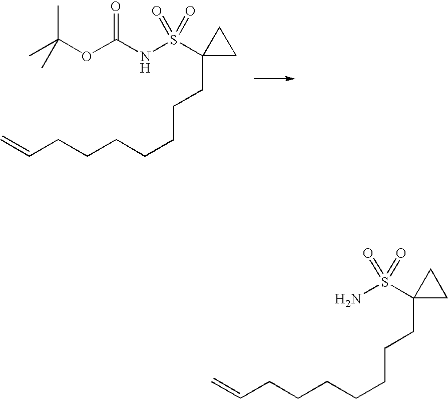

A mixture of 1.7 g (5 mmol) tert-butyl [(1-non-8-en-1-ylcyclopropyl)sulfonyl]carbamate and 9 mL HCl in dioxane (4 M) in 92 mL dioxane is stirred at rt for 12 h. The mixture is concentrated and co-evaporated with DCM to give the desired product. LC-MS (method A): Rt=3.70 min, M+H=246.3

### Step 5

### tert-butyl [(1R,2S)-1-{[(1-non-8-en-1-ylcyclopropyl)sulfonyl]carbamoyl}-2-vinylcyclopropyl]carbamate

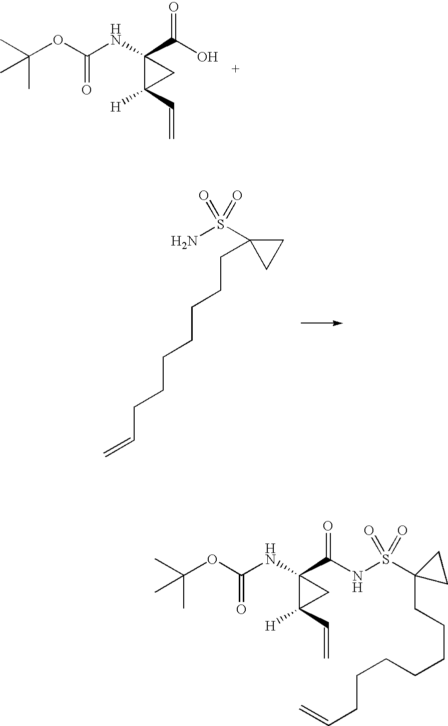

A mixture of 1.3 g (5.6 mmol) (1R,2S)-1-tert-Butoxycarbonylamino-2-vinyl-cyclopropane-carboxylic acid and 1.4 g (8.4 mmol) CDI in 40 mL THF is heated under reflux for 1 h. After cooling to rt, 1.3 mL (8.4 mmol) DBU and a solution of 1.4 g (5.8 mmol) 1-non-8-enyl-cyclopropanesulfonic acid amide in 5 mL THF is added and the mixture is stirred at rt overnight. After concentration under reduced pressure, the residue is partitioned between EtOAc and 1 N HCl. The aq. layer is extracted with EtOAc and the combined organic layers are dried over Na₂SO₄ and concentrated. The crude is purified by flash chromatography (silica gel, DCM/MeOH 98:2) to give the title compound. LC-MS (method A): Rt=4.99 min, M+H=455.2

### Step 6

### (1R,2S)-1-(1-Non-8-enyl-cyclopropahesulfonylaminocarbonyl)-2-vinyl-cyclopropyl]-carbamic acid tert-butyl ester

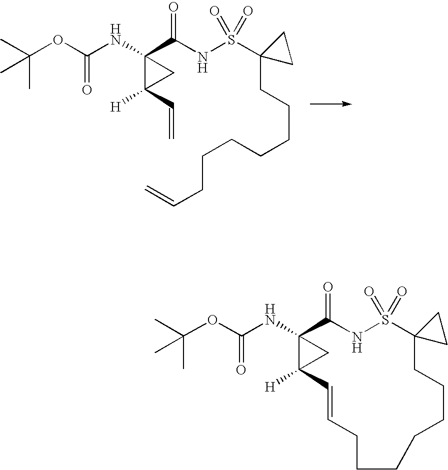

A mixture of 510 mg (1.1 mmol) tert-butyl [(1R,2S)-1-{[(1-non-8-en-1-ylcyclopropyl)-sulfonyl]carbamoyl}-2-vinylcyclopropyl]carbamate and 141 mg (0.2 mmol) Hoveyda-Grubbs II catalyst in 370 mL DCM is heated to 40° C overnight. The mixture is concentrated under reduced pressure and the crude is purified by prep. HPLC (method E) to give the desired product. LC-MS (method A): Rt=4.16 min, M+H=427.1

### Step 7

### (1R,13E,15S)-1-amino-4-thia-3-azaspiro[bicyclo[13.1.0]hexadecane-5,1′-cyclopropane]-13-en-2-one 4,4-dioxide

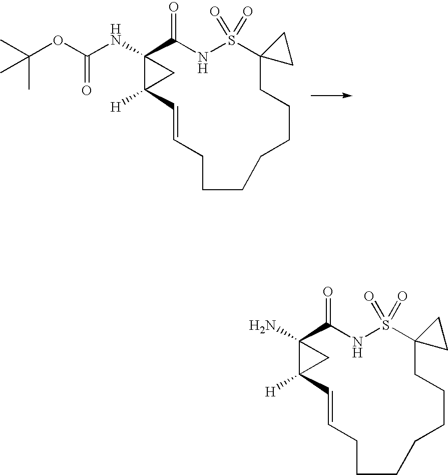

A mixture of 155 mg (0.4 mmol) [(1R,2S)-1-(1-Non-8-enyl-cyclopropanesulfonylamino-carbonyl)-vinyl-cyclopropyl]-carbamate acid tert-butyl ester, 2 mL HCl in dioxane (4 M) and 2 mL dioxane is stirred at rt for 2 h. The mixture is concentrated under reduced pressure and co-evaporated with DCM to give the desired product as a hydrochloride salt. LC-MS (method A): Rt=2.71 min, M+H=363.3

### Example 63

### N-(tert-butoxycarbonyl)-L-valyl-(4R)-N-[(1R,13E,15S)-4,4-dioxido-2-oxo-4-thia-3-azaspiro(bicyclo[13.1.0]hexadecane-5,1′-cyclopropane]-13-en-1-yl)-4-{[(4-fluoro-1,3-dihydro-2H-isoindol-2-yl)carbonyl]oxy}-L-prolinamide

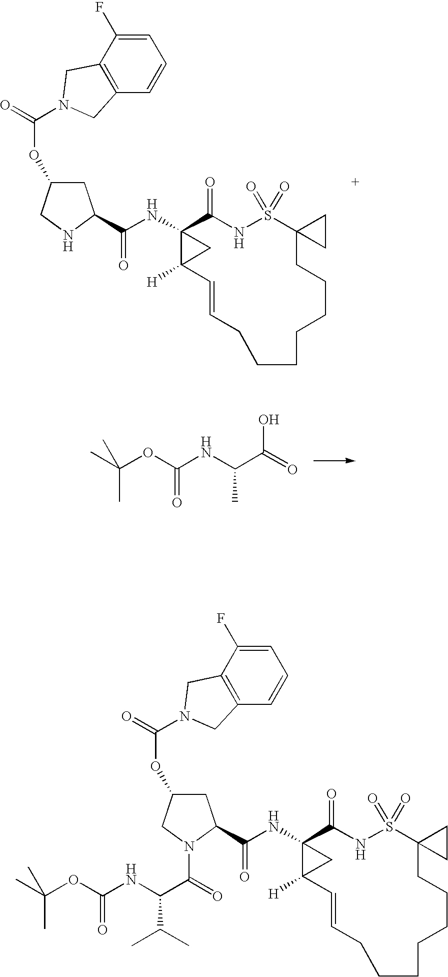

A mixture of 13 mg (0.06 mmol) (S)-2-tert-Butoxycarbonylamino-3-methyl-butyric acid, 0.02 mL (0.06 mmol) DIPEA and 22 mg (0.06 mmol) HBTU in 2 mL DMF is stirred at rt for 30 min, then 33 mg (0.05 mmol) (3R,5S)-5-{[(1R,13E,15S)-4,4-dioxido-2-oxo-4-thia-3-azaspiro[bicyclo[13.1.0]hexadecane-5,1′-cyclopropane]-13-en-1-yl]carbamoyl}pyrrolidin-3-yl 4-fluoro-1,3-dihydro-2H-isoindole-2-carboxylate is added. After 5 h the mixture is partitioned between DGM and aq. K₂CO₃ and the aq. layer is extracted with DCM. The combined organic layers are washed with aq. 10% KHSO₄ solution and brine, dried over Na₂SO₄ and concentrated under reduced pressure. The crude is purified by prep. HPLC (method E) to give the desired product. LC-MS (method A): Rt=4.36 min, M+H=802.2

### Preparation of (3R,5S)-5-{[(1R,13E,15S)-4,4-dioxido-2-oxo-4-thia-3-azaspiro[bicyclo[13.1.0]hexadecane-5,1′-cyclopropane]-13-en-1-yl]carbamoyl}pyrrolidin-3-yl 4-fluoro-1,3-dihydro-2H-isoindole-2-carboxylate

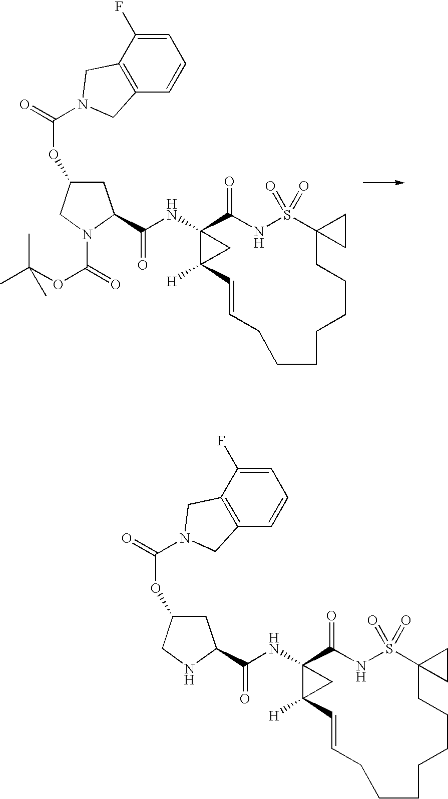

A mixture of 38 mg (0.05 mmol)) (3R,5S)-1-(tert-butoxycarbonyl)-5-{[(1R,13E,15S)-4,4-dioxido-2-oxo-4-thia-3-azaspiro[bicyclo[13.1.0]hexadecane-5,1′-cyclopropane]-13-en-1-yl]carbamoyl}pyrrolidin-3-yl 4-fluoro-1,3-dihydro-2H-isoindole-2-carboxylate and 2 mL HCl in dioxane (4 M) in 2 mL dioxane is stirred at rt for 2 h. The mixture is concentrated and co-evaporated with DCM to give the desired product as a hydrochloride salt. LC-MS (method A): Rt=2.88 min, M+H=693.2

### Example 64

N-[(cyclopentyloxy)carbonyl]-L-valyl-(4R)-N-[(1R,13E,15S)-4,4-dioxido-2-oxo-4-thia-3-azaspiro(bicyclo[13.1.0]hexadecane-5,1′-cyclopropane]-13-en-1-yl]-4-{[(4-fluoro-1,3-dihydro-2H-isoindol-2-yl)carbonyl]oxy}-L-prolinamide

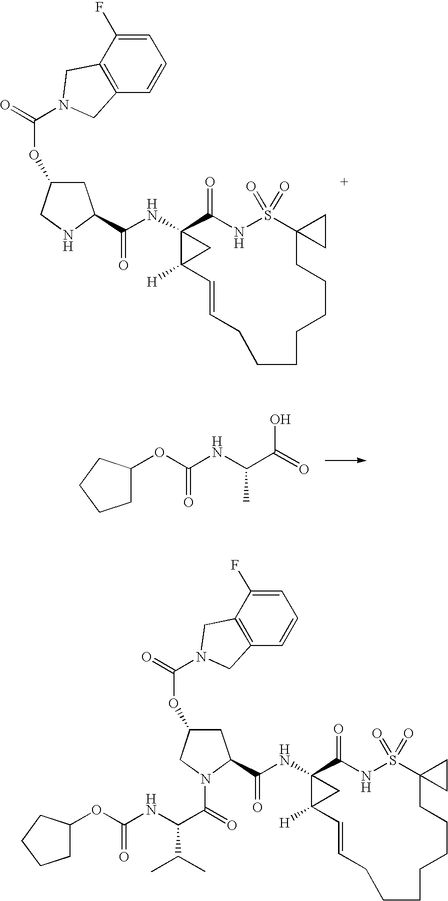

A mixture of 73 mg (0.27 mmol) (S)-2-Cyclopentyloxycarbonylamino-3-methyl-butyric acid, 0.14 mL (0.32 mmol) DIPEA and 122 mg (0.32 mmol) HBTU in 6 mL DMF is stirred at rt for 30 min, then 73 mg (0.32 mmol) (3R,5S)-5-{[(1R,13E,15S)-4,4-dioxido-2-oxo-4-thia-3-azaspiro[bicyclo[13.1.0]hexadecane-5,1′-cyclopropane]-13-en-1-yl]carbamoyl}pyrrolidin-3-yl 4-fluoro-1,3-dihydro-2H-isoindole-2-carboxylate is added. After 12 h the mixture is partitioned between DCM and aq. K₂CO₃ and the aq. layer is extracted with DCM. The combined organic layers are washed with aq. 10% KHSO₄ solution and brine, dried over Na₂SO₄ and concentrated under reduced pressure. The crude is purified by prep. HPLC (method E) to give the desired product. LC-MS (method A): Rt=4.32 min, M+H=836.7

### Example 65

### tert-butyl (2S,4R)-2-{[(1R,13E,15S)-4,4-dioxido-2-oxo-4-thia-3-azaspiro(bicyclo[13.1.0]hexadecane-5,1′-cyclopropane]-13-en-1-yl]carbamoyl}-4-({2-[2-(isopropylamino)-1,3-thiazol-4-yl]-7-methoxyquinolin-4-yl}oxy)pyrrolidine-1-carboxylate

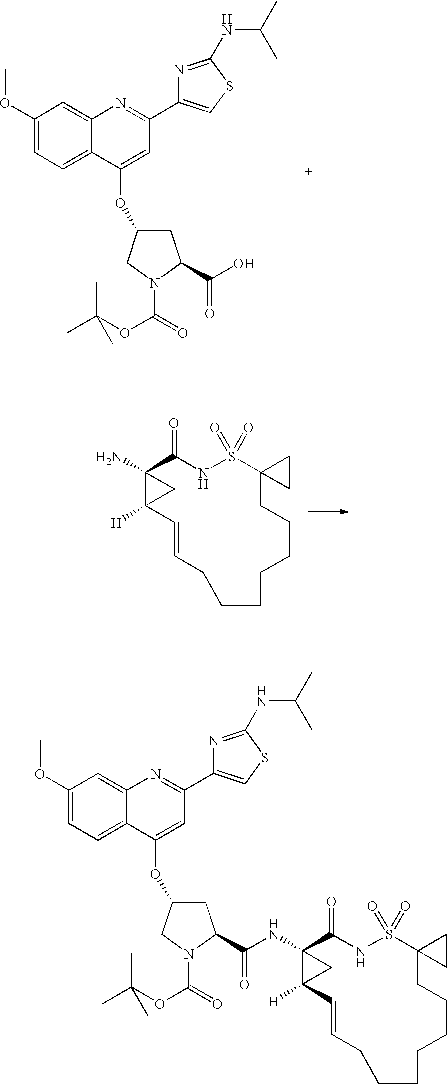

A mixture of 64 mg (0.1 mmol), (2S,4R)-4-[2-(2-Isopropylamino-thiazol-4-yl)-7-methoxy-quinolin-4-yloxy]-pyrrolidine-1,2-dicarboxylic acid 1-tert-butyl ester, 0.06 mL (0.1 mmol) DIPEA and 51 mg (0.1 mmol) HBTU in 2 mL DMF is stirred at rt for 30 min, then 40 mg (0.1 mmol) (1R,13E,15S)-1-amino-4-thia-3-azaspiro[bicyclo[13.1.0]hexadecane-5,1′-cyclopropane]-13-en-2-one 4,4-dioxide is added. After 12 h the mixture is partitioned between DGM and aq. K₂CO₃ and the aq. layer is extracted with DCM. The combined organic layers are washed with aq. 10% KHSO₄ solution and brine, dried over Na₂SO₄ and concentrated under reduced pressure. The crude is purified by prep. HPLC.(method E) to give the desired product. LC-MS (method A): Rt=3.91 min, M+H=838.2

### Example 66

### N-(tert-butoxycarbonyl)-L-valyl-(4R)-N-[(1R,13E,15S)-4,4-dioxido-2-oxo-4-thia-3-azaspiro[bicyclo[13.1.0]hexadecane-5,1′-cyclopropane]-13-en-1-yl)-4-({2-[2-(isopropylamino)-1,3-thiazol-4-yl]-7-methoxyquinolin-4-yl}oxy)-L-prolinamide

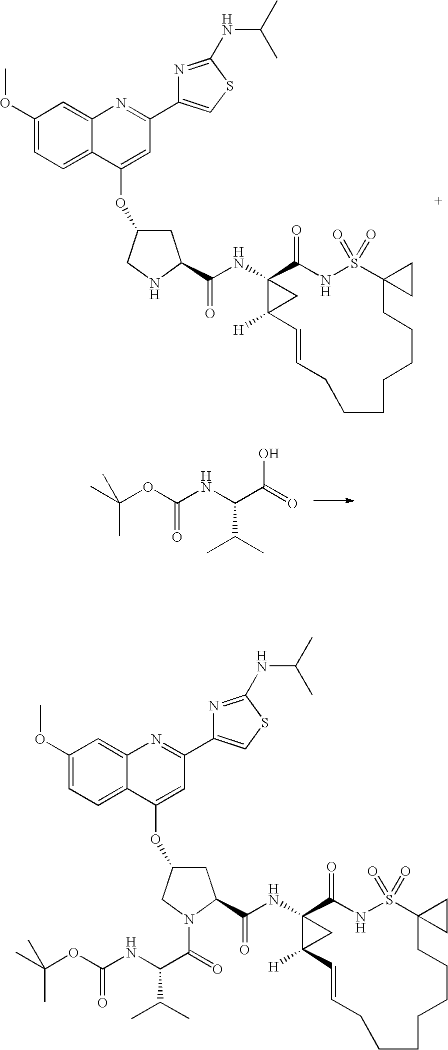

A mixture of 14 mg (0.07 mmol) (S)-2-tert-Butoxycarbonylamino-3-methyl-butyric acid, 0.03 mL (0.07 mmol) DIPEA and 25 mg (0.07 mmol) HBTU in 2 mL DMF is stirred at rt for 30 min, then 43 mg (0.06 mmol) (4R)-N-[(1R,13E,15S)-4,4-dioxido-2-oxo-4-thia-3-azaspiro[bicyclo[13.1.0]hexadecane-5,1′-cyclopropane]-13-en-1-yl]-4-({2-[2-(isopropylamino)-1,3-thiazol-4-yl]-7-methoxyquinolin-4-yl}oxy)-L-prolinamide is added. After 12 h the mixture is partitioned between DCM and aq. K₂CO₃ and the aq. layer is extracted with DCM. The combined organic layers are washed with aq. 10% KHSO₄ solution and brine, dried over Na₂SO₄ and concentrated under reduced pressure. The crude is purified by prep. HPLC (method E) to give the desired product. LC-MS (method A): Rt=3.89 min, M−H=935.2

### Preparation of (4R)-N-[(1R,13E,15S)-4,4-dioxido-2-oxo-4-thia-3-azaspiro[bicyclo[13.1.0]hexadecane-5,1′-cyclopropane]-13-en-1-yl]-4-({2-[2-(isopropylamino)-1,3-thiazol-4-yl]-7-methoxyquinolin-4-yl}oxy)-L-prolinamide

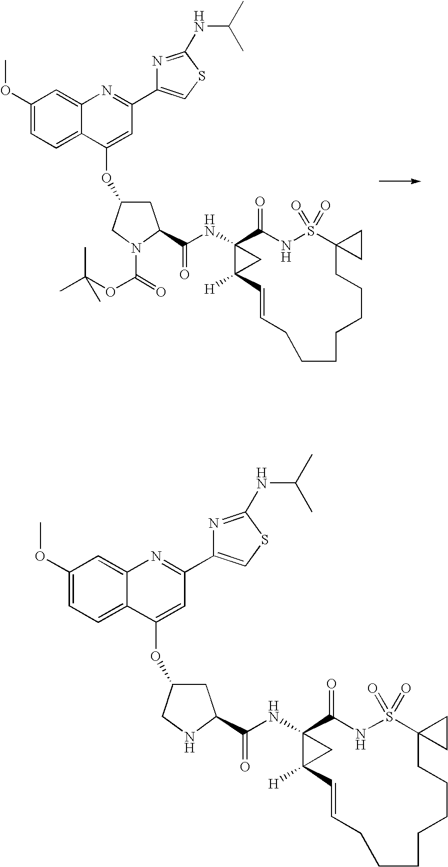

A mixture of 47 mg (0.06 mmol) tert-butyl (2S,4R)-2-{[(1R,13E,15S)-4,4-dioxido-2-oxo-4-thia-3-azaspiro[bicyclo[13.1.0]hexadecane-5,1′-cyclopropane]-13-en-1-yl]carbamoyl}-4-({2-[2-(isopropylamino)-1,3-thiazol-4-yl]-7-methoxyquinolin-4-yl}oxy)pyrrolidine-1-carboxylate and 2 mL HCl in dioxane (4 M) in 2 mL dioxane is stirred at rt for 3 h. The mixture is concentrated and co-evaporated with DCM to give the desired product as a hydrochloride salt. LC-MS (method A): Rt=2.65 min, M+H=773.3

### Example 67

### tert-butyl(2R,4R)-2-{[(1R,15R)-4,4-dioxido-2-oxo-4-thia-3-azaspiro[bicyclo[13.1.0]hexadecane-5,1′-cyclopropan]-1-yl]carbamoyl}-4-({2-[2-(isopropylamino)-1,3-thiazol-4-yl]-7-methoxyquinolin-4-yl}oxy)pyrrolidine-1-carboxylate

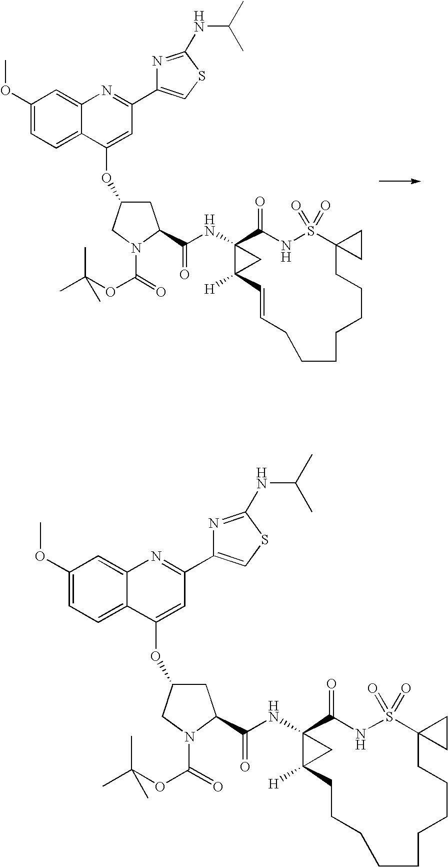

A mixture of 82 mg (0.10 mmol) tert-butyl (2S,4R)-2-{[(1R,13E,15S)-4,4-dioxido-2-oxo-4-thia-3-azaspiro[bicyclo[13.1.0]hexadecane-5,1′-cyclopropane]-13-en-1-yl]carbamoyl}-4-({2-[2-(isopropylamino)-1,3-thiazol-4-yl]-7-methoxyquinolin-4-yl}oxy)pyrrolidine-1-carboxylate, 456 mg (2.4 mmol) potassium diazodicarboxylate and 2.4 mL acetic acid (0.5 M in DCM) in 10 mL DCM is heated at 45° C for 72 h. Additional 456 mg potassium diazodicarboxylate and 1.5 mL acetic acid (0.5 M in DCM) are added and the mixture is heated for 72 h. The mixture washed with 1 N HCl and the organic layer is dried over Na₂SO₄ and concentrated. The crude is triturated with CH3CN and the solid product is i filtered and dried. LC-MS (method A): Rt=4.00 min, M+H=838.3

### Example 68

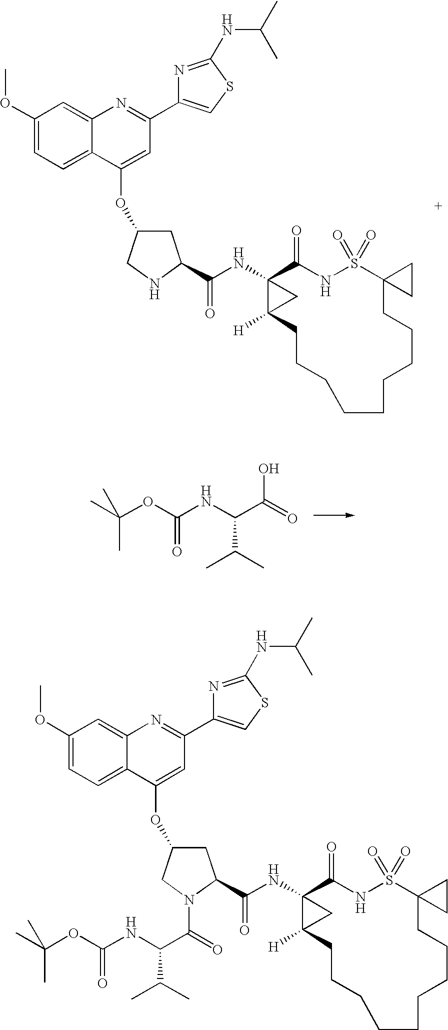

A mixture of 4 mg (0.02 mmol) (S)-2-tert-Butoxycarbonylamino-3-methyl-butyric acid, 0.008 mL (0.02 mmol) DIPEA and 7 mg (0.02 mmol) HBTU in 2 mL DMF is stirred at rt for 30 min, then 12 mg (0.02 mmol) (4R)-N-[(1R,15R)-4,4-dioxido-2-oxo-4-thia-3-azaspiro[bicyclo[13.1.0]hexadecane-5,1′-cyclopropan]-1-yl]-4-({2-[2-(isopropylamino)-1,3-thiazol-4-yl]-7-methoxyquinolin-4-yl}oxy)-L-prolinamide is added. After 12 h the mixture is partitioned between DCM and aq. K₂CO₃ and the aq. layer is extracted with DCM. The combined organic layers are washed with aq. 10% KHSO₄ solution and brine, dried over Na₂SO₄ and concentrated under reduced pressure. The crude is purified by prep. HPLC (method E) to give the desired product. LC-MS (method A): Rt=4.00 min, M+H=939.4

### Preparation of (4R)-N-[(1R,15R)-4,4-dioxido-2-oxo-4-thia-3-azaspiro[bicyclo[13.1.0]hexadecane-5,1′-cyclopropan)-1-yl]-4-({2-[2-(isopropylamino)-1,3-thiazol-4-yl]-7-methoxyquinolin-4-yl}oxy)-L-prolinamide

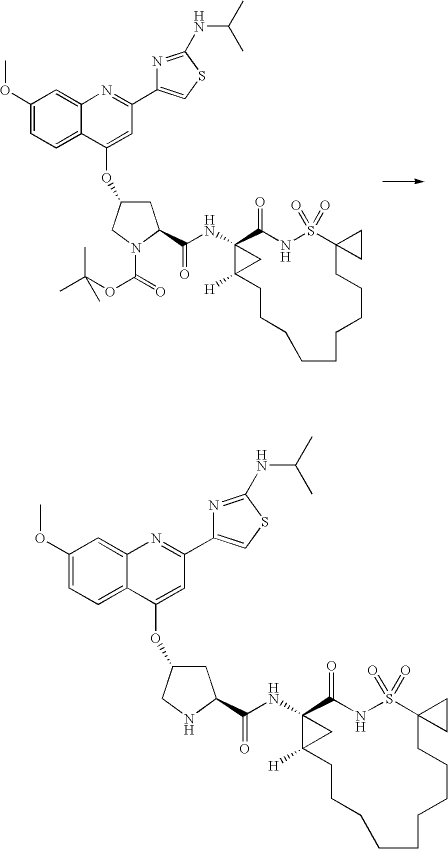

A mixture of 88 mg (0.11 mmol) tert-butyl (2S,4R)-2-{[(1R,15S)-4,4-dioxido-2-oxo-4-thia-3-azaspiro[bicyclo[13.1.0]hexadecane-5,1′-cyclopropan]-1-yl]carbamoyl}-4-({2-[2-(isopropylamino)-1,3-thiazol-4-yl]-7-methoxyquinolin-4-yl}oxy)pyrrolidine-1-carboxylate and 1 mL HCl in dioxane (4 M) in 1 mL dioxane is stirred at rt for 12 h. The mixture is concentrated and co-evaporated with DCM to give the desired product as a hydrochloride salt. LC-MS (method A): Rt=2.84 min, M+H=739.3

Table E provides additional compounds (Examples 69-109) of the invention which may be prepared by routine modification of the synthetic procedures as described supra in Examples 1-68.

TABLE E

| | | | HPLC | |
|---|---|---|---|---|
| | | | Method C | LC-MS |
| Ex | Name | Structure | Rt in min | Method A |
| 69 | (2S,4R)-4-(7-Methoxy- 2-phenyl-quinolin-4- yloxy)-pyrrolidine-1,2- dicarboxylic acid 1- benzylamide 2-[((Z)- (5R,7S)-2,2,4-trioxo- 2Λ⁶-thia-3,15-diaza- tricyclo[14.4.0.0^{5,7}]icosa- 1(16),8,17,19-tetraen- 5-yl)-amide] | 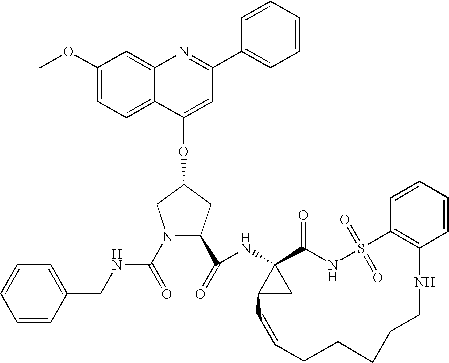 | 3.62 | 828.0 (M − H) |
| 70 | (2S,4R)-1-Acetyl-4-[2- (2-Isopropylamino- thiazol-4-yl)-7- methoxy-quinolin-4- yloxy]-pyrrolidine-2- carboxylic acid ((Z)- (5R,7S)-2,2,4-trioxo- 2Λ⁶-thia-3,15-diaza- tricyclo[14.4.0.0^{5,7}]icosa- 1(16),8,17,19-tetraen- 5-yl)-amide | 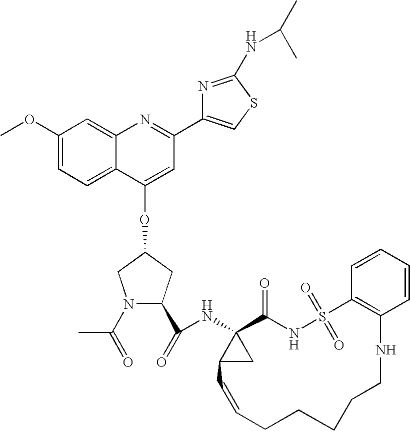 | 3.47 | 802.3 (M + H) |
| 71 | (2S,4R)-1-(2-Hydroxy- 2-methyl-propionyl)-4- [2-(2-isopropylamino- thiazol-4-yl)-7- methoxy-quinolin-4- yloxy]-pyrrolidine-2- carboxylic acid ((Z)- (5R,7S)-2,2,4-trioxo- 2Λ⁶-thia-3,15-diaza- tricyclo[14.4.0.0^{5,7}]icosa- 1(16),8,17,19-tetraen- 5-yl)-amide | 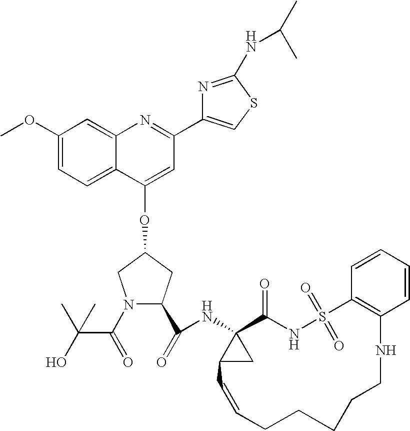 | 3.52 | 847.3 (M + H) |
| 72 | (2S,4R)-1-(2,2- Dimethyl-propionyl)-4- [2-(2-isopropylamino- thiazol-4-yl)-7- methoxy-quinolin-4- yloxy]-pyrrolidine-2- carboxylic acid ((Z)- (5R,7S)-2,2,4-trioxo- 2Λ⁶-thia-3,15-diaza- tricyclo[14.4.0.0^{5,7}]icosa- 1(16),8,17,19-tetraen- 5-yl)-amide | 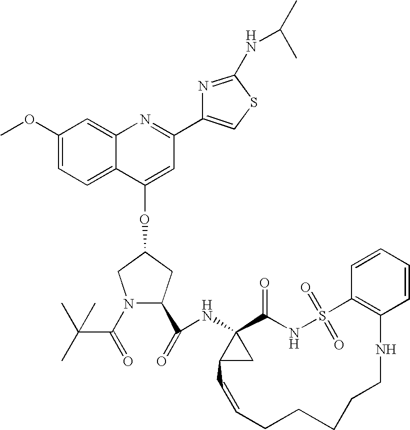 | 3.74 | 845.3 (M + H) |
| 73 | (2S,4R)-1-(3,3- Dimethyl-butyryl)-4- [2-(2-isopropylamino- thiazol-4-yl)-7- methoxy-quinolin-4- yloxy]-pyrrolidine-2- carboxylic acid ((Z)- (5R,7S)-2,2,4-trioxo- 2Λ⁶-thia-3,15-diaza- tricyclo[14.4.0.0^{5,7}]icosa- 1(16),8,17,19-tetraen- 5-yl)-amide | 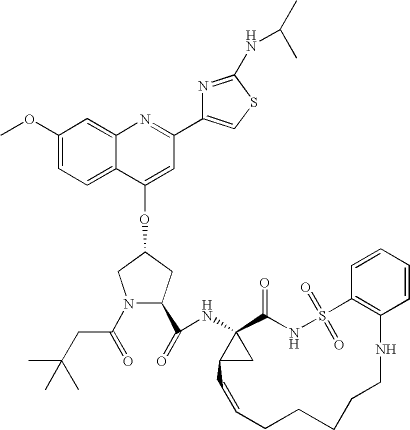 | 3.82 | 859.3 (M + H) |
| 74 | (2S,4R)-4-[2-(2- Isopropylamino- thiazol-4-yl)-7- methoxy-quinolin-4- yloxy]-1-((S)-5-oxo- pyrrolidine-2- carbonyl)-pyrrolidine- 2-carboxylic acid ((Z)- (5R,7S)-2,2,4-trioxo- 2Λ⁶-thia-3,15-diaza- tricyclo[14.4.0.0^{5,7}]icosa- 1(16),8,17,19-tetraen- 5-yl)-amide | 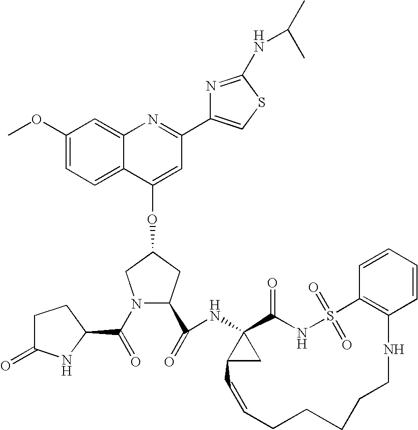 | 3.27 | 872.3 (M + H) |
| 75 | (2S,4R)-1-((R)-2- Hydroxy-3-methyl- butyryl)-4-[2-(2- isopropylamino- thiazol-4-yl)-7- methoxy-quinolin-4- yloxy]-pyrrolidine-2- carboxylic acid ((Z)- (5R,7S)-2,2,4-trioxo- 2Λ⁶-thia-3,15-diaza- tricyclo[14.4.0.0^{5,7}]icosa- 1(16),8,17,19-tetraen- 5-yl)-amide | 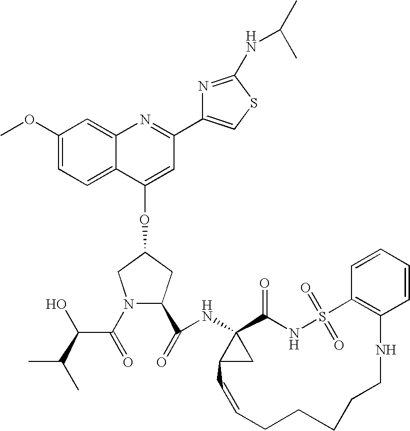 | 3.53 | 859.3 (M − H) |
| 76 | (2S,4R)-4-[2-(2- Isopropylamino- thiazol-4-yl)-7- methoxy-quinolin-4- yloxy]-1-(2H-pyrazole- 3-carbonyl)- pyrrolidine-2- carboxylic acid ((Z)- (5R,7S)-2,2,4-trioxo- 2Λ⁶-thia-3,15-diaza- tricyclo[14.4.0.0^{5,7}]icosa- 1(16),8,17,19-tetraen- 5-yl)-amide | 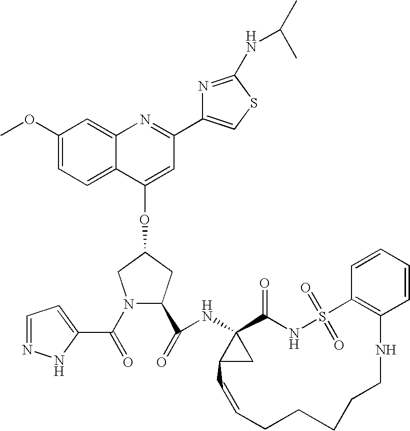 | 3.45 | 855.3 (M + H) |
| 77 | (2S,4R)-4-[2-(2- Isopropylamino- thiazol-4-yl)-7- methoxy-quinolin-4- yloxy]-1-(pyridine-2- carbonyl)-pyrrolidine- 2-carboxylic acid ((Z)- (5R,7S)-2,2,4-trioxo- 2Λ⁶-thia-3,15-diaza- tricyclo[14.4.0.0^{5,7}]icosa- 1(16),8,17,19-tetraen- 5-yl)-amide | 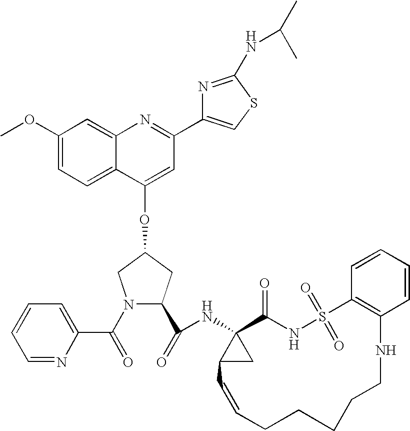 | 3.52 | 864.3 (M − H) |
| 78 | (2S,4R)-1-(1H-Indole- 2-carbonyl)-4-[2-(2- isopropylamino- thiazol-4-yl)-7- methoxy-quinolin-4- yloxy]-pyrrolidine-2- carboxylic acid ((Z)- (5R,7S)-2,2,4-trioxo- 2Λ⁶-thia-3,15-diaza- tricyclo[14.4.0.0^{5,7}]icosa- 1(16),8,17,19-tetraen- 5-yl)-amide | 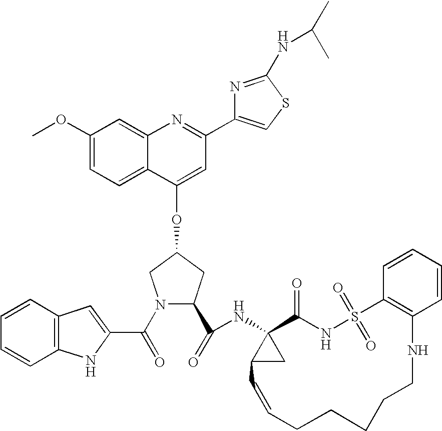 | 3.78 | 902.3 (M − H) |
| 79 | (2S,4R)-1-Benzoyl-4- [2-(2-isopropylamino- thiazol-4-yl)-7- methoxy-quinolin-4- yloxy]-pyrrolidine-2- carboxylic acid ((Z)- (5R,7S)-2,2,4-trioxo- 2Λ⁶-thia-3,15-diaza- tricyclo[14.4.0.0^{5,7}]icosa- 1(16),8,17,19-tetraen- 5-yl)-amide | 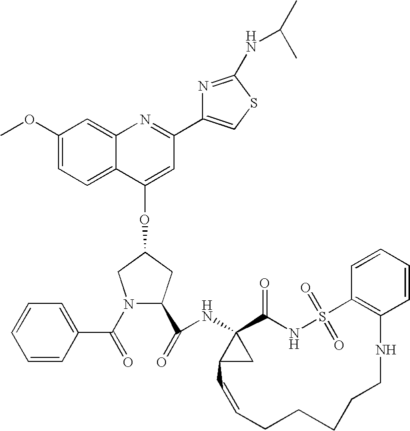 | 3.64 | 863.2 (M − H) |
| 80 | (2S,4R)-1-(3- Acetylamino-benzoyl)- 4-[2-(2- isopropylamino- thiazol-4-yl)-7- methoxy-quinolin-4- yloxy]-pyrrolidine-2- carboxylic acid ((Z)- (5R,7S)-2,2,4-trioxo- 2Λ⁶-thia-3,15-diaza- tricyclo[14.4.0.0^{5,7}]icosa- 1(16),8,17,19-tetraen- 5-yl)-amide | 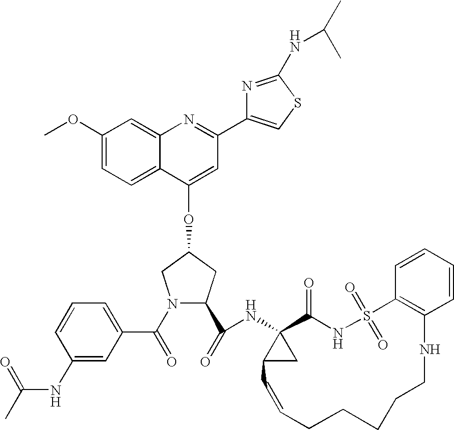 | 3.44 | 922.3 (M + H) |
| 81 | (2S,4R)-1-(2- Acetylamino-benzoyl)- 4-[2-(2- isopropylamino- thiazol-4-yl)-7- methoxy-quinolin-4- yloxy]-pyrrolidine-2- carboxylic acid ((Z)- (5R,7S)-2,2,4-trioxo- 2Λ⁶-thia-3,15-diaza- tricyclo[14.4.0.0^{5,7}]icosa- 1(16),8,17,19-tetraen- 5-yl)-amide | 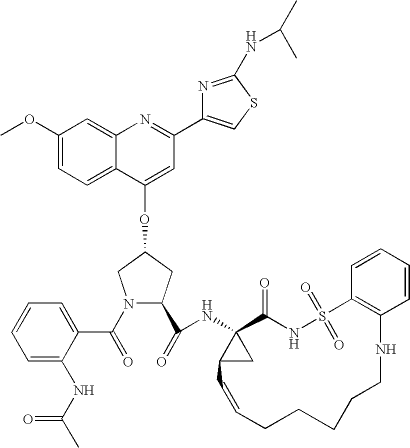 | 3.57 | 921.3 (M + H) |
| 82 | {3-[(2S,4R)-4-[2-(2- Isopropylamino- thiazol-4-yl)-7- methoxy-quinolin-4- yloxy]-2-((Z)-(5R,7S)- 2,2,4-trioxo-2Λ⁶-thia- 3,15-diaza- tricyclo[14.4.0.0^{5,7}]icosa- 1(16),8,17,19-tetraen- 5-ylcarbamoyl)- pyrrolidin-1-yl]-3-oxo- propyl}-carbamic acid tert-butyl ester | 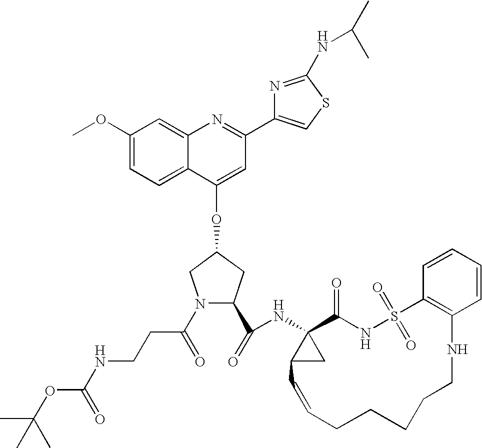 | 3.60 | 930.3 (M − H) |
| 83 | (2S,4R)-1-(3- Acetylamino- propionyl)-4-[2-(2- isopropylamino- thiazol-4-yl)-7- methoxy-quinolin-4- yloxy]-pyrrolidine-2- carboxylic acid ((Z)- (5R,7S)-2,2,4-trioxo- 2Λ⁶-thia-3,15-diaza- tricyclo[14.4.0.0^{5,7}]icosa- 1(16),8,17,19-tetraen- 5-yl)-amide | 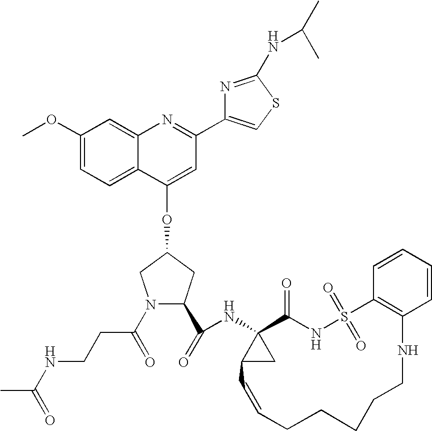 | 3.53 | 871.3 (M − H) |
| 84 | ((R)-1-{2-[(2S,4R)-4- [2-(2-isopropylamino- thiazol-4-yl)-7- methoxy-quinolin-4- yloxy]-2-((Z)-(5R,7S)- 2,2,4-trioxo-2Λ⁶-thia- 3,15-diaza- tricyclo[14.4.0.0^{5,7}]icosa- 1(16),8,17,19-tetraen- 5-ylcarbamoyl)- pyrrolidin-1-yl]-2-oxo- ethyl}-2-methyl- propyl)-carbamic acid tert-butyl ester | 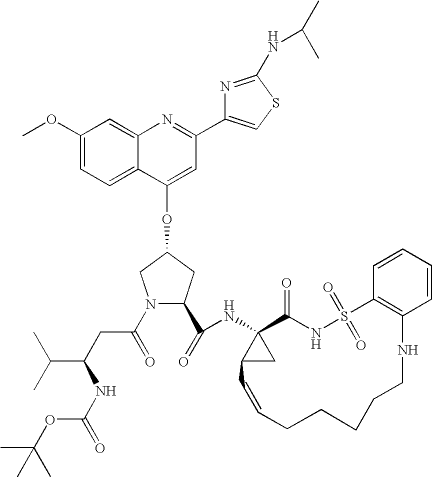 | 3.91 | 972.4 (M − H) |
| 85 | (2S,4R)-1-((R)-3- Acetylamino-4-methyl- pentanoyl)-4-[2-(2- isopropylamino- thiazol-4-yl)-7- methoxy-quinolin-4- yloxy]-pyrrolidine-2- carboxylic acid ((Z)- (5R,7S)-2,2,4-trioxo- 2Λ⁶-thia-3,15-diaza- tricyclo[14.4.0.0^{5,7}]icosa- 1(16),8,17,19-tetraen- 5-yl)-amide | 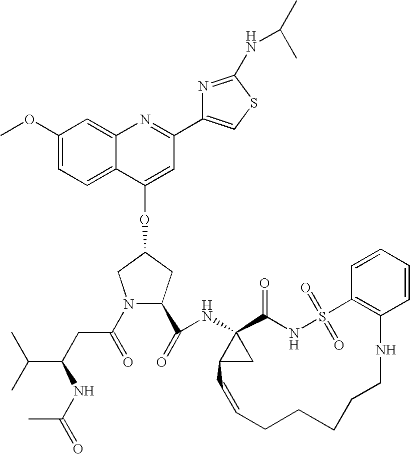 | 3.51 | 914.3 (M − H) |
| 86 | ((S)-1-{2-[(2S,4R)-4- [2-(2-isopropylamino- thiazol-4-yl)-7- methoxy-quinolin-4- yloxy]-2-((Z)-(5R,7S)- 2,2,4-trioxo-2Λ⁶-thia- 3,15-diaza- tricyclo[14.4.0.0^{5,7}]icosa- 1(16),8,17,19-tetraen- 5-ylcarbamoyl)- pyrrolidin-1-yl]-2-oxo- ethyl}-3-methyl-butyl)- carbamic acid tert-butyl ester | 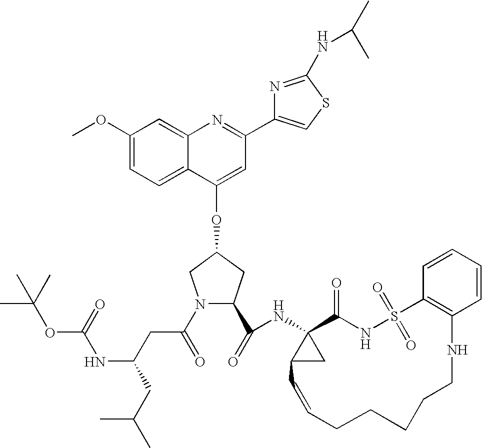 | 3.95 | 987.3 (M + H) |
| 87 | (2S,4R)-1-((S)-3- Acetylamino-5-methyl- hexanoyl)-4-[2-(2- isopropylamino- thiazol-4-yl)-7- methoxy-quinolin-4- yloxy]-pyrrolidine-2- carboxylic acid ((Z)- (5R,7S)-2,2,4-trioxo- 2Λ⁶-thia-3,15-diaza- tricyclo[14.4.0.0^{5,7}]icosa- 1(16),8,17,19-tetraen- 5-yl)-amide | 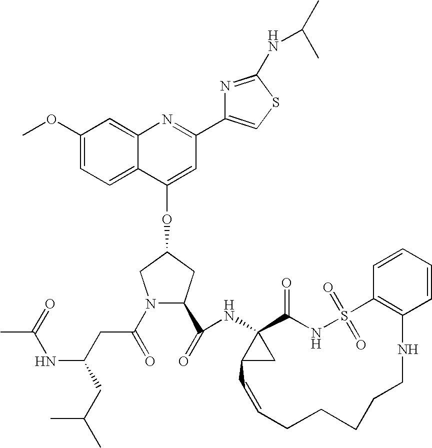 | 3.58 | 930.3 (M + H) |
| 88 | 4-[(2S,4R)-4-[2-(2- Isopropylamino- thiazol-4-yl)-7- methoxy-quinolin-4- yloxy]-2-((Z)-(5R,7S)- 2,2,4-trioxo-2Λ⁶-thia- 3,15-diaza- tricyclo[14.4.0.0^{5,7}]icosa- 1(16),8,17,19-tetraen- 5-ylcarbamoyl)- pyrrolidin-1-yl]-4-oxo- butyric acid methyl ester | 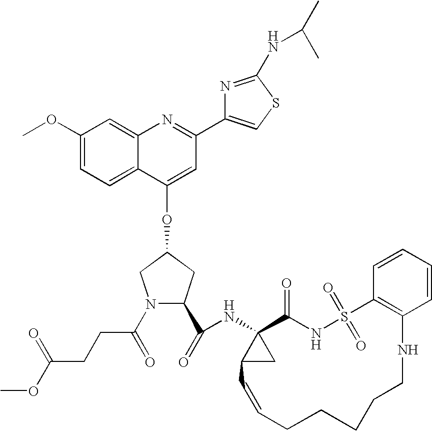 | 3.50 | 874.3 (M + H) |
| 89 | (2S,4R)-4-[2-(2- Isopropylamino- thiazol-4-yl)-7- methoxy-quinolin-4- yloxy]-1-(3- methylcarbamoyl- propionyl)-pyrrolidine- 2-carboxylic acid ((Z)- (5R,7S)-2,2,4-trioxo- 2Λ⁶-thia-3,15-diaza- tricyclo[14.4.0.0^{5,7}]icosa- 1(16),8,17,19-tetraen- 5-yl)-amide | 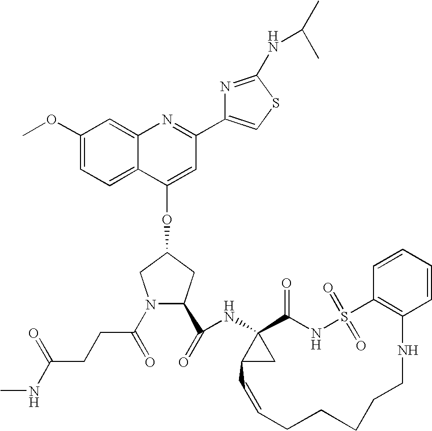 | 3.34 | 873.3 (M + H) |
| 90 | (2S,4R)-4-[2-(2- isopropylamino- thiazol-4-yl)-7- methoxy-quinolin-4- yloxy]-1-((S)-3-methyl- 2- methycarbamoylmethyl- butyryl)-pyrrolidine- 2-carboxylic acid ((Z)- (5R,7S)-2,2,4-trioxo- 2Λ⁶-thia-3,15-diaza- tricyclo[14.4.0.0^{5,7}]icosa- 1(16),8,17,19-tetraen- 5-yl)-amide | 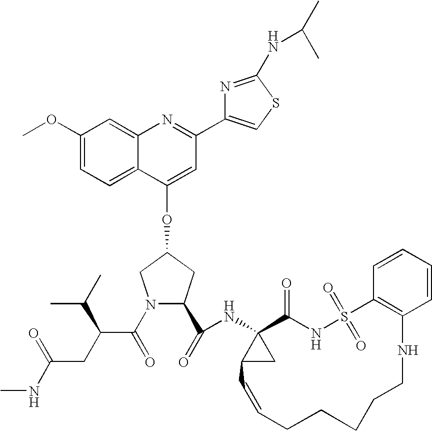 | 3.51 | 915.3 (M + H) |
| 91 | (2S,4R)-4-[2-(2- isopropylamino- thiazol-4-yl)-7- methoxy-quinolin-4- yloxy]-1-((S)-5-methyl- 3-methylcarbamoyl- hexanoyl)-pyrrolidine- 2-carboxylic acid ((Z)- (5R,7S)-2,2,4-trioxo- 2Λ⁶-thia-3,15-diaza- tricyclo[14.4.0.0^{5,7}]icosa- 1(16),8,17,19-tetraen- 5-yl)-amide | 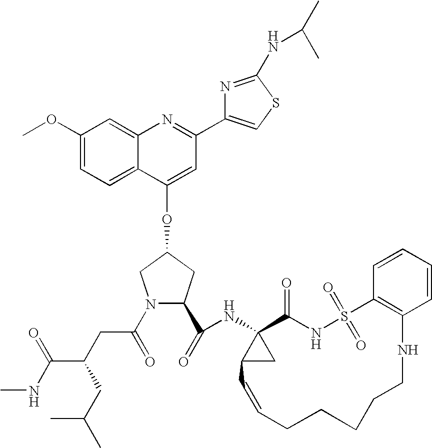 | 3.68 | 929.3 (M + H) |
| 92 | (R)-2-{2-[(2S,4R)-4-[2- (2-Isopropylamino- thiazol-4-yl)-7- methoxy-quinolin-4- yloxy]-2-((Z)-(5R,7S)- 2,2,4-trioxo-2Λ⁶-thia- 3,15-diaza- tricyclo[14.4.0.0^{5,7}]icosa- 1(16),8,17,19-tetraen- 5-ylcarbamoyl)- pyrrolidin-1-yl]-2-oxo- ethyl}-4-methyl- pentanoic acid methyl ester | 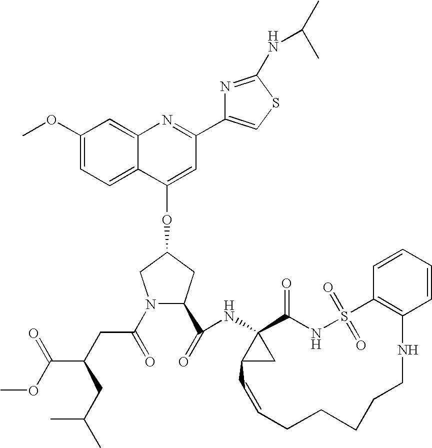 | 3.84 | 930.4 (M + H) |
| 93 | (2S,4R)-4-[2-(2- Isopropylamino- thiazol-4-yl)-7- methoxy-quinolin-4- yloxy]-1-((R)-5- methyl-3- methylcarbamoyl- hexanoyl)-pyrrolidine- 2-carboxylic acid ((Z)- (5R,7S)-2,2,4-trioxo- 2Λ⁶-thia-3,15-diaza- tricyclo[14.4.0.0^{5,7}]icosa- 1(16),8,17,19-tetraen- 5-yl)-amide | 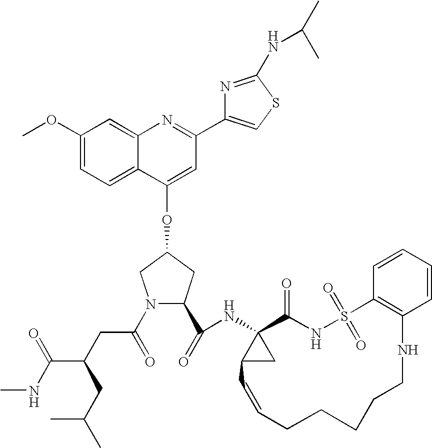 | 3.61 | 929.3 (M + H) |
| 94 | (2S,4R)-4-[2-(2- Isopropylamino- thiazol-4-yl)-7- methoxy-quinolin-4- yloxy]-pyrrolidine-1,2- dicarboxylic acid 1- benzylamide 2-[((Z)- (5R,7S)-2,2,4-trioxo- 2Λ⁶-thia-3,15-diaza- tricyclo[14.4.0.0^{5,7}]icosa- 1(16),8,17,19-tetraen- 5-yl)-amide] | 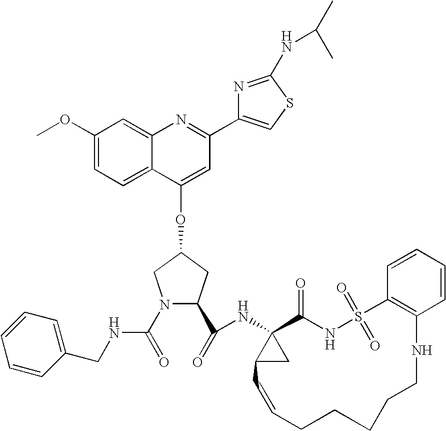 | 3.73 | 894.3 (M + H) |
| 95 | (2S,4R)-4-[2-(2- Isopropylamino- thiazol-4-yl)-7- methoxy-quinolin-4- yloxy]-pyrrolidine-1,2- dicarboxylic acid 1- phenylamide 2-[((Z)- (5R,7S)-2,2,4-trioxo- 2Λ⁶-thia-3,15-diaza- tricyclo[14.4.0.0^{5,7}]icosa- 1(16),8,17,19-tetraen- 5-yl)-amide] | 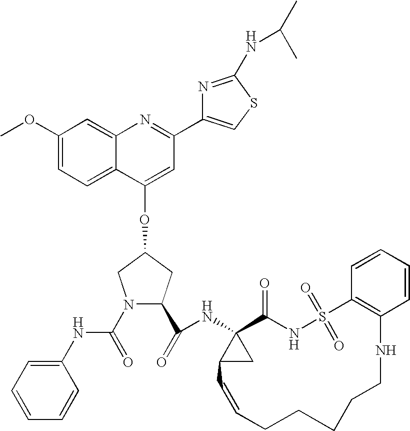 | 3.73 | 879.3 (M + H) |
| 96 | (2S,4R)-4-[2-(2- Isopropylamino- thiazol-4-yl)-7- methoxy-quinolin-4- yloxy]-pyrrolidine-1,2- dicarboxylic acid 1- tert-butylamide 2-[((Z)- (5R,7S)-2,2,4-trioxo- 2Λ⁶-thia-3,15-diaza- tricyclo[14.4.0.0^{5,7}]icosa- 1(16),8,17,19-tetraen- 5-yl)-amide] | 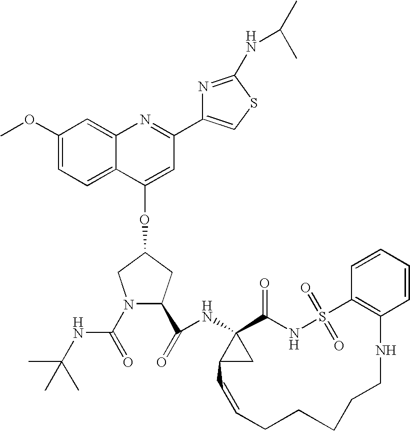 | 3.77 | 860.3 (M + H) |
| 97 | (2S,4R)-4-[2-(2- Isopropylamino- thiazol-4-yl)-7- methoxy-quinolin-4- yloxy]-pyrrolidine-1,2- dicarboxylic acid 1- amide 2-[((Z)-(5R,7S)- 2,2,4-trioxo-2Λ⁶-thia- 3,15-diaza- tricyclo[14.4.0.0^{5,7}]icosa- 1(16),8,17,19-tetraen- 5-yl)-amide] | 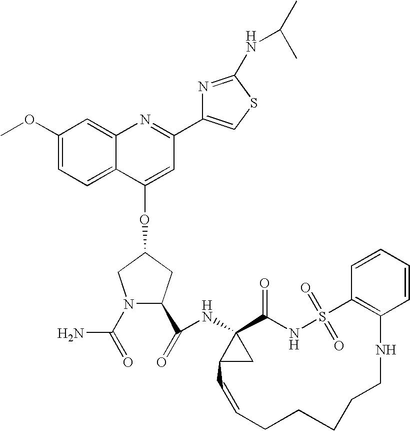 | 3.37 | 803.2 (M + H) |
| 98 | (2S,4R)-1-(N′-Acetyl- N-isopropyl- hydrazinocarbonyl)-4- [2-(2-isopropylamino- thiazol-4-yl)-7- methoxy-quinolin-4- yloxy]-pyrrolidine-2- carboxylic acid ((Z)- (5R,7S)-2,2,4-trioxo- 2Λ⁶-thia-3,15-diaza- tricyclo[14.4.0.0^{5,7}]icosa- 1(16),8,17,19-tetraen- 5-yl)-amide | 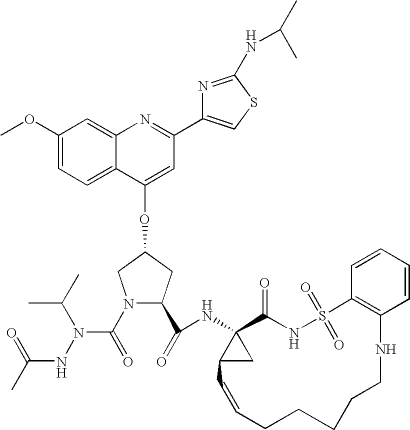 | 3.66 | 902.3 (M + H) |
| 99 | 4-Fluoro-1,3-dihydro- isoindole-2-carboxylic acid (3R,5S)-1-acetyl- 5-((Z)-(5R,7S)-2,2,4- trioxo-2Λ⁶-thia-3,15- diaza- tricyclo[14.4.0.0^{5,7}]icosa- 1(16),8,17,19-tetraen- 5-ylcarbamoyl)- pyrrolidin-3-yl ester | 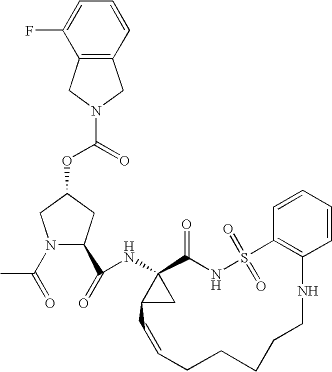 | 3.68 | 668.2 (M + H) 666.3 (M − H) 690.2 (M + Na) |
| 100 | 4-Fluoro-1,3-dihydro- isoindole-2-carboxylic acid (3R,5S)-1-tert- butoxycarbonyl-5-((Z)- (5R,7S)-2,2,4-trioxo- 2Λ⁶-thia-3,15-diaza- tricyclo[14.4.0.0^{5,7}]icosa- 1(16),8,17,19-tetraen- 5-ylcarbamoyl)- pyrrolidin-3-yl ester | 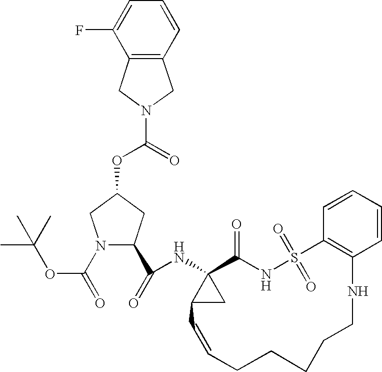 | 4.48 | 726.2 (M + H) 724.2 (M − H) |
| 101 | (2S,4R)-4-[2-(2- Isopropylamino- thiazol-4-yl)-7- methoxy-quinolin-4- yloxy]-2-((Z)-(5R,7S)- 2,2,4,14-tetraoxo-2Λ⁶- thia-3,15-diaza- tricyclo[14.4.0.0^{5,7}]icosa- 1(16),8,17,19-tetraen- 5-ylcarbamoyl)- pyrrolidine-1- carboxylic acid tert- butyl ester | 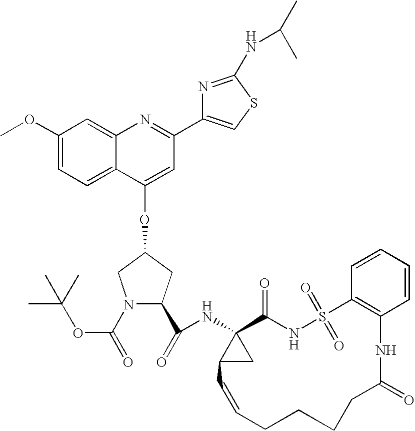 | 3.59 | 874.3 (M + H) |
| 102 | (2S,4R)-1-Acetyl-4-[2- (2-isopropylamino- thiazol-4-yl)-7- methoxy-quinolin-4- yloxy]-pyrrolidine-2- carboxylic acid ((Z)- (5R,7S)-2,2,4,14- tetraoxo-2Λ⁶-thia-3,15- diaza- tricyclo[14.4.0.0^{5,7}]icosa- 1(16),8,17,19-tetraen- 5-yl)-amide | 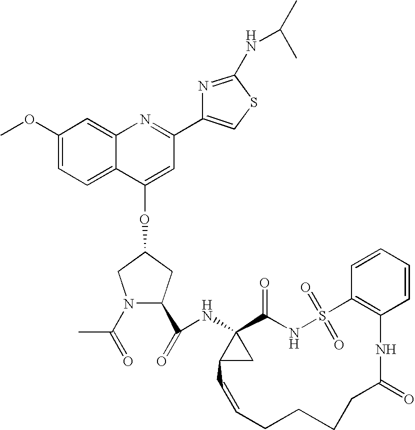 | 3.24 | 816.3 (M + H) |
| 103 | {(S)-1-[(2S,4R)-4- Hydroxy-2-((Z)- (5R,7S)-2,2,4,14- tetraoxo-2Λ⁶-thia-3,15- diaza- tricyclo[14.4.0.0^{5,7}]icosa- 1(16),8,17,19-tetraen- 5-ylcarbamoyl)- pyrrolidine-1- carbonyl]-2-methyl- propyl}-carbamic acid tert-butyl ester | 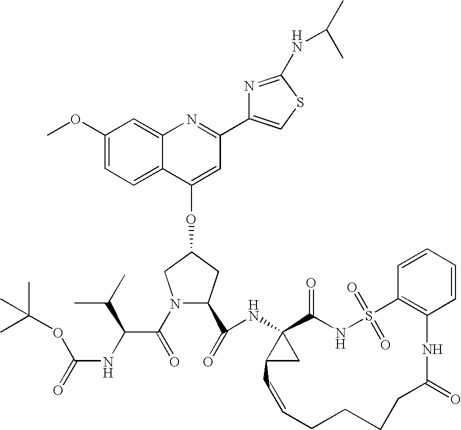 | 3.67 | 974.3 (M + H) |
| 104 | (2S,4R)-1-(2-Hydroxy- acetyl)-4-[2-(2- isopropylamino- thiazol-4-yl)-7- methoxy-quinolin-4- yloxy]-pyrrolidine-2- carboxylic acid ((E)- (5R,7S)-2,2,4,14- tetraoxo-2Λ⁶-thia-3,15- diaza- tricyclo[14.4.0.0^{5,7}]icosa- 1(16),8,17,19-tetraen- 5-yl)-amide | 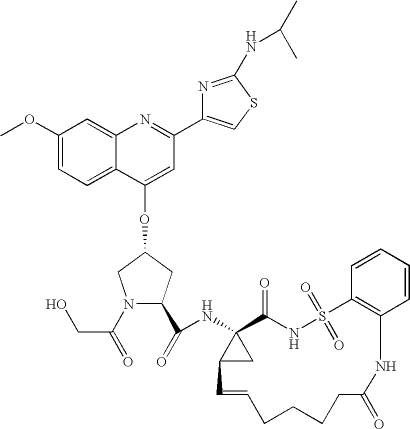 | 3.83 | 832.3 (M + H) |
| 105 | (2S,4R)-1-Acetyl-4-(7- methoxy-2-phenyl- quinolin-4-yloxy)- pyrrolidine-2- carboxylic acid ((5R,7R)-2,2,4,15- tetraoxo-2Λ⁶-thia-3,16- diaza- tricyclo[15.4.0.0^{5,7}]heni- cosa-1(17),18,20-trien- 5-yl)-amide | 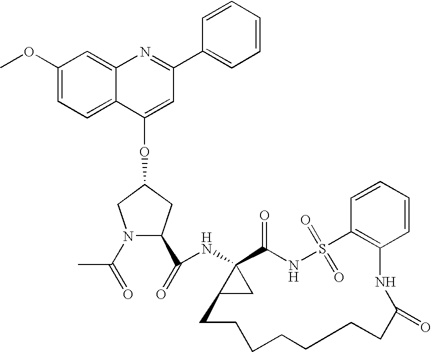 | 3.16 | 767.9 (M + H) |
| 106 | (2S,4R)-1-Acetyl-4-(7- methoxy-2-phenyl- quinolin-4-yloxy)- pyrrolidine-2- carboxylic acid ((Z)- (5R,7S)-2,2,4-trioxo- 16-oxa-2Λ⁶-thia-3-aza- tricyclo[15.4.0.0^{5,7}]heni- cosa-1(17),8,18,20- tetraen-5-yl)-amide | 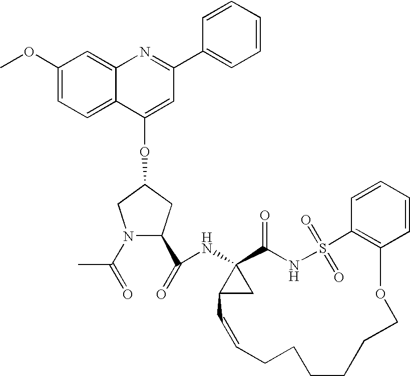 | 3.25 | 754 (M + H) 751.8 (M − H) |
| 107 | (2S,4R)-1-Acetyl-4-(7- methoxy-2-phenyl- quinolin-4-yloxy)- pyrrolidine-2- carboxylic acid ((5R,7R)-2,2,4-trioxo- 15-oxa-2Λ⁶-thia-3-aza- tricyclo[14.4.0.0^{5,7}]icosa- 1(16),17,19-trien-5- yl)-amide | 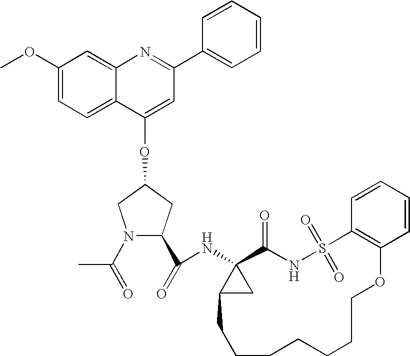 | 3.17 | 741.2 (M + H) |
| 108 | (2S,4R)-1-Acetyl-4-(7- methoxy-2-phenyl- quinolin-4-yloxy)- pyrrolidine-2- carboxylic acid ((E)- (5R,7S)-2,2,4,17- tetraoxo-2Λ⁶-thia-3,16- diaza- tricyclo[16.3.1.0^{5,7}] docosa-1(22),8,18,20- tetraen-5-yl)-amide | 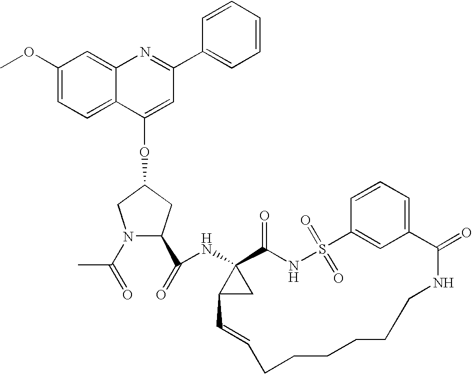 | 3.00 | 781.0 (M + H) |
| 109 | (2S,4R)-1-Acetyl-4-(7- methoxy-2-phenyl- quinolin-4-yloxy)- pyrrolidine-2- carboxylic acid ((5R,7R)-2,2,4,17- tetraoxo-2Λ⁶-thia-3,16- diaza- tricyclo[16.3.1.0^{5,7}] docosa-1(22),18,20-trien- 5-yl)-amide | 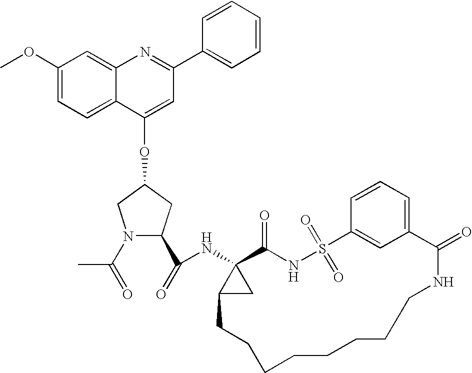 | 3.08 | 782.2 (M + H) |

### Biological Activity

### Example 110

### HCV NS3-4A Protease Assay

The inhibitory activity of certain compounds of Table A against HCV NS3-4A serine protease is determined in a homogenous assay using the full-length NS3-4A protein (genotype 1a, strain HCV-1) and a commercially available internally-quenched fluorogenic peptide substrate as described by taliani, M., et al. 1996 Anal. Biochem. 240:60-67, which is incorporated by reference in its entirety.

### Example 111

### Luciferase-Based HCV Replicon Assay

The antiviral activity and cytotoxicity of certain compounds of Table A is determined using a subgenomic genotype 1b HCV replicon cell line (Huh-Luc/neo-ET) containing a luciferase reporter gene, the expression of which is under the control of HCV RNA replication and translation. Briefly, 5,000 replicon cells are seeded in each well of 96-well tissue culture plates and are allowed to attach in complete culture media without G418 overnight. On the next day, the culture media are replaced with media containing a serially diluted compound of Table A in the presence of 10% FBS and 0.5% DMSO. After a 48-h treatment with the compound of Table A, the remaining luciferase activities in the cells are determined using BriteLite reagent (Perkin Elmer, Wellesley, Mass.) with a LMaxII plate reader (Molecular Probe, Invitrogen). Each data point represents the average of four replicates in cell culture. IC₅₀ is the concentration of the at which the luciferase activity in the replicon cells is reduced by 50%: The cytotoxicity of the compound of Table A is evaluated using an MTS-based cell viability assay.

Compounds of Table A supra have been tested in at least one of the protease assay of Example 110 or the replicon assay of Example 111 and exhibit an IC₅₀ of less than about 10 μM or less in at least one of the assays recited in Examples 110 and 111. Equivalents

Those skilled in the art will recognize, or be able to ascertain using no more than routine experimentation, many equivalents to the specific embodiments and methods described herein. Such equivalents are intended to be encompassed by the scope of the following claims.

### Incorporation by Reference

The entire contents of all patents, published patent applications and other references cited herein are hereby expressly incorporated herein in their entireties by reference. The entire contents of copending applications U.S. Ser. No. 60/791,318, U.S. Ser. No. 60/791,320, U.S. Ser. No. 60/791,578, and U.S. Ser. No. 60/791,611, each of which was filed on Apr. 11, 2006and each of which is expressly incorporated herein, in their entirety, as applied to the compounds of the present invention.

1. A compound of the formula: 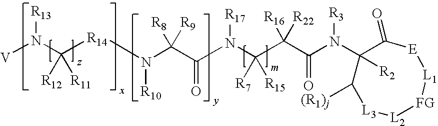
and pharmaceutically acceptable salts, enantiomers, stereoisomers, retainers, tautomers, diastereomers, or racemates thereof;
wherein
the macrocyde: 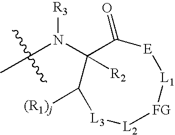
comprises between 10 to 25 ring atoms;
m, x and z are each independently selected from 0 or 1;
j, p and y are independently selected at each occurrence from the group consisting of 0, 1 and 2;
R₁ and R₂ are independently selected, at each occurrence, from hydrogen or from the group consisting of alkyl, alkenyl, alkynyl, cycloalkyl, cyano, alkoxy, and cycloalkyloxy, each of which is unsubstituted or substituted with 1-6 moieties which can be the same or different and are independently selected from the group consisting of hydroxy, oxo, alkyl, aryl, alkoxy, aryloxy, thio, alkylthio, arylthio, amino, alkylamino, arylamino, alkylsulfonyl, arylsulfonyl, alkylsulfonamino, arylsulfonamido, heterarylsulfonamido, arylaminosulfonyl, heteroarylaminosulfonyl, mono and dialkylaminosulfonyl, carboxy, carbalkoxy, amido, carboxamido, alkoxycarbonylamino, aminocarbonyloxy, alkoxycarbonyloxy, alkylureido, arylureido, halogen, cyano, or nitro; wherein each of said alkyl, alkoxy, and aryl can be unsubstituted or optionally independently substituted with one or more moieties which can be the same or different and are independently selected from alkyl alkenyl, alkynyl, cycloalkyl, cycloalkyl-alkyl, heterocycyl, heterocyclylalkyl aryl, alkylaryl, aralkyl, arylheteroaryl, heteroaryl, heterocycylamino, alkylheteroaryl and heteroaralkyl;
R₃ is selected from the group consisting of H and C₁₋₄-alkyl;
E is a divalent residue selected from the group consisting of NR₂₃, C(O)NR₂₃ and NR₂₃S(O)ₚNR₂₃;
L₁ and L₂ are divalent residues independently selected from the group consisting of alkylene, (CH₂)₁—FG-(CH₂)ₓ, alkanylene, alkynylene, arylsne, heteroarylene, cycloalkylene and heterocyeloalkylene, each of which is substituted with 0 to 4 independently selected X₁ or X₂ groups;
i and k are independently selected integers of from 0 to 7;
L₃ is absent or a divalent ethylene or acetylene residue, wherein the divalent ethylene is substituted by 0-2 substituents selected from alkyl, aryl, heteroaryl mono- or di-alkylamino-C₀-C₆alkyl, hydroxyl alkyl or alkoxyalkyl;
FG is absent or a divalent residue selected from the group consisting of O, S(O)ₚ, NR₂₃, C(O), C(O)NR₂₃, NR₂₃C(O), OC(O)NR₂₃, NR₂₃C(O)O, NR₂₃C(O)NR₂₃, S(O)ₚNR₂₃, NR₂₃S(O)ₚ, and NR₂₃S(O)NR₂₃;
R₂₃ is independently selected at each occurrence from hydrogen or the group consisting of alkyl, alkenyl, alkynyl, cycloalkyl, cycloalkylalkyl, aryl, heteroaryl, heteroaralkyl and aralkyl, each of which is substituted with 0-2 substituents independently selected from halogen, alkyl, and alkoxy;
R₇, R₈, R₉, R₁₀, R₁₁, R₁₂, R₁₃, R₁₆, R₁₅, R₁₇, R₂₂, and V are each, independently, selected from the group consisting of H, alkyl, alkenyl, alkynyl, aryl, alkyl-aryl, heteroalkyl, heterocycyl, heteroaryl, aryl-heteroaryl, alkyl-heteroaryl, cycloalkyl, alkyloxy, alkyl-aryloxy, aryloxy, heteroaryloxy, heterocyclyloxy, cycloalkyloxy, amino, alkylamino, arylamino, alkyl-arylamino, arylamino, heteroarylamino, cycloalkylamino, carboxyalkylamino, aralkyloxy and heterocycylamino; each of which may be further substituted 0 to 5 limes with substituents independently selected from X₁ and X₂;
X₁ is alkyl, alkenyl, alkynyl, cycloalkyl, spirocycloalkyl cycloalkyl-alkyl, heterocyclyl, heterocyclylalkyl, aryl, alkylaryl, aralkyl, arylheteroaryl, heteroaryl, heterocyclylamino, alkylheteroaryl, or heteroaralkyl; wherein X₁ can be independently substituted with one or more of X₂ moieties which can be the same or different and are independently selected;
X₂ is hydroxy, oxo, alkyl aryl, heteroaryl, alkoxy, aryloxy, heteroaryloxy, thio, alkylthio, arylthio, heteroarylthio, amino, alkylamino, arylamino, heteroarylamino, alkylsulfonyl, arylsulfonyl, heteroarylsulfonyl, alkylsulfonamido, arylsulfonamido, heterarylsulfonamido, arylaminosulfonyl, heteroarylaminosulfonyl, mono and dialkylaminosulfonyl, carboxy, carbalkoxy, amido, carboxamido, alkoxycarbonylamino, aminocarbonyloxy, alkoxycarbonyloxy, carbamoyl, ureido, alkylureido, arylureido, halogen, cyano, or nitro; wherein each of said alkyl, alkoxy, and aryl can be unsubstituted or optionally independently substituted with one or more moieties which can be the same or different and are independently selected from alkyl, alkenyl, alkynyl, cycloalkyl, cycloalkyl-alkyl, heterocyclyl, heterocyclylalkyl, aryl, alkylaryl, aralkyl, arylheteroaryl, heteroaryl, heterocyclylamino, alkylheteroaryl and heteroaralkyl;
R₁₄ is C(O) or S(O)ₚ;
V is selected from the group consisting of -Q¹-Q², wherein Q¹ is absent, C(O), S(O)ₚ, N(H), N(C₁₋₄alkyl), O═N(CN), C═N(SO₂CH₃), or C═N—COH, and Q² is H, C₁₋₄-alkyl, C═N—COH—C₁₋₄-alkyl, O—C₁₋₄-alkyl, NH₂, N(H)—C₁₋₄alkyl, N(C₁₋₄-alkyl)₂, SO₂-aryl, SO₂—C₁₋₄alkyl, C₃₋₆cycloalkyl-C₀₋₄alkyl, aryl, heteroaryl and heterocycle, each of which may be independently substituted one or more times with a halogen atom. C₁₋₄-alkyl, C₂₋₄-alkenyl C₂₋₄-alkynyl, C₁₋₄-alkoxyl, C₂₋₄-alkenyloxy. C₂₋₄-alkynyloxy, C₁₋₄-alkyl substituted by one or more halogen atoms, C₃₋₆-cycloalkyl, carboxylate, carboxamido, mono- and di-alkylamine, or mono- and di-alkylcarboxamido;
or R₂₂ and R₁₆ may together form a 3, 4, 5, 8 or 7-membered ring and may contain one or more heteroatoms, wherein the ring may be further substituted one or more times;
or R₇ and R₁₅ may together form a 3, 4, 5, 6 or 7-membered ring and may contain one or more heteroatoms, wherein the ring may be further substituted one or more times;
or R₁₅ and R₁₇ may together form a 3,4, 5, 8 or 7-membered ring and may contain one or more heteroatoms, wherein the ring may be further substituted one or more times;
or R₁₅ and R₁₆ may together form a 4, 5, 6 or 7-membered ring and may contain one or more heteroatoms, wherein the ring may be further substituted one or more times;
or R₁₅ and R₁₆ may together form an arylene or heteroarylene ring and R₇ and R₂₂ are absent, wherein the ring may be further substituted one or more times;
or R₁ and R₂ may together form a 3, 4, 5, 8 or 7-membered ring that is saturated or partially unsaturated and may contain one or more heteroatoms, wherein the ring may be further substituted one or more times;
or R₁₇ and R₁₆ may together form a 4, 5, 8, 7 or 8-membered ring of the formula: 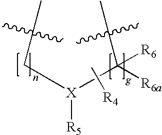
wherein
n and g are each, independently, 0, 1 or 2;
X is O, S, N, NR₅, CR₅ or CR₅R₅ₐ;
R₄ is selected from the group consisting of H, C₁₋₆-alkyl, C_{3,7}-cycloalkyl, aryl, heterocycle and heteroaryl, each of which may be independently substituted one or more times with a halogen atom or C₁₋₄-alkyl;
R₅ is selected from the group consisting of H, hydroxyl, oxo, C₁₋₈-alkyl, C₂₋₈-alkenyl, C₂₋₈-alkynyl, C₃₋₈cycloalkyl-C₀₋₄-alkyl aryl-C₀₋₄-alkyl heterocycle-C₀₋₄-alkyl, heteroaryl-C₀₋₄-alkyl; C₃₋₈-cycloalkyloxy, aryloxy, NR₂₃COR₂₃, CONR₂₃R₂₃, NR₂₃CONHR₂₃, OCONR₂₃R₂₃, NR₂₃COOR₂₃, OCOR₂₃, COOR₂₃, aryl-C(O)O, aryl-C(O)NR₂₃, heteroaryloxy, heteroaryl-C(O)O, heteroaryl-C(O)NR₂₃, each of which may be independently substituted one or more times with a halogen atom, aryl, heteroaryl, trihalomethyl, C₁₋₄-alkyl, or C₁₋₄-alkoxy;
R₅ₐ is selected from the group consisting of H, hydroxyl, C₁₋₈-alkyl, C₂₋₈-alkenyl, C₂₋₈alkynyl, C₃₋₈-cycloalkyl-C₀₋₄-alkyl, aryl-C₀₋₄-alkyl and heteroaryl-C₀₋₄-alkyl,
or R₄ and R₅ may together form a fused dimethyl cyclopropyl ring, a fused cyclopentane ring, a fused phenyl ring or a fused pyridyl ring, each of which may he substituted with a halogen atom, aryl, heteroaryl, trihalomethyl, C₁₋₄-alkoxy or C₁₋₄-alkyl;
or R₅ and R₅ₐ may together form a spirocyclic ring having between 3 and 7 ring atoms which is optionally substituted by 0-4 substituents selected from cyano, halogen, hydroxyl, amino, thiol, C₁₋₈-alkyl, C₂₋₈-alkenyl. C₂₋₈-alkynyl: C₁₋₈-alkoxide, C₁₋₈-haloalkyl; C₂₋₈-haloalkenyl, C₂₋₈-haloalkynyl, C₁₋₈-haloalkoxide, C₁₋₈-alkylthio, C₁₋₈-alkylsulfonyl, C₁₋₈-alkylsulfoxide, C₁₋₈-alkanoyl, C₁₋₈-alkoxycarbonyl, C₃₋₇-cycloalkyl-C₀₋₄-alkyl, aryl-C₀₋₄alkyl, heterocyclyl-C₀₋₄-alkyl, heteroaryl-C₀₋₄-alkyl, COOH, C(O)NH₂, mono- and di-C₁₋₄-alkyl-carboxamide, SO₃H; SO₂NH₂, and mono-and di-C₁₋₄-alkylsulfonamide, or two substituents taken together form a fused or spirocyclic 3 to 7 membered ring having 0, 1 or 2 ring heteroatoms selected from N, O and S, which fused or spirocyclic ring has 0 to 2 independently selected substituents selected from halogen, C₁₋₄-alkyl, C₁₋₄-alkoxy, C₁₋₄-alkanoyl, mono- and di-C₁₋₄-alkylamino, mono- and di-C₁₋₄-alkyl-carboxamide, C₁₋₄-alkoxycarbonyl, and phenyl; and
R₆ and R₈ₐ are independently selected at each occurrence from the group consisting of H, C₁₋₄-alkyl and (CH₂)₀₋₄—C₃₋₈-cycloalkyl;
or R₆ and R₆ₐ may together form a spirocyclic ring having between 3 and 7 ring atoms which is optionally substituted by 0-4 substituents selected from cyano, halogen, hydroxyl, amino, thiol, C₁₋₈-alkyl, C₂₋₈-alkenyl, C₂₋₈-alkynyl, C₁₋₈-alkoxide, C₁₋₈-haloalkyl, C₂₋₈-haloalkenyl, C₂₋₈-haloalkynyl, C₁₋₈-haloalkoxide, C₁₋₈-alkylthio, C₁₋₈-alkylsulfonyl, C₁₋₈-alkylsulfoxide; C₁₋₈-alkanoyl, C₁₋₈-alkoxycarbonyl, C₃₋₇-cycloalkyl-C₀₋₄-alkyl, aryl-C₀₋₄-alkyl, heterocyclyl-C₀₋₄-alkyl, heteroaryl-C₀₋₄-alkyl, COOH, C(O)NH₂, mono- and di-C₁₋₄-alkyl-carboxamide, SO₃H, SO₂NH₂ and mono-and di-C-₁₋₄-alkylsulfonamide, or two substituents taken together form a fused or spirocyclic 3 to 7 membered ring having 0, 1 or 2 ring heteroatoms selected from N, O and S, which fused or spirocyclic ring has 0 to 2 independently selected substituents selected from halogen, C-₁₋₄alkyl, C₁₋₄alkoxy, C₁₋₄alkanoyl, mono-and di-C-₁₋₄-alkylamino, mono- and di-C₁₋₄-alkyl-carboxamide, C₁₋₄-alkoxycarbonyl, and phenyl.

2-3. (canceled)

8. The compound of claim 1, wherein
L₁ is C₁-C₈alkylene, C₃-C₇cycloalkylene, arylene or heteroarylene each of which is substituted by 0-4 residues independently selected from C₁-C₄alkyl, C₁-C₄alkoxy, hydroxyl, amino, mono- and di-C₁-C₄alkyl amino, halogen, cyano, C₁-C₄-fluoroalkyl, C₁-C₄fluoroalkoxy, COOH, carboxamide (CONH₂), mono- and di-C₁-C₄alkylcarboxamide, aryl, heteroaryl and 5 or 6 membered saturated heterocycles:
L₂ is selected from C₁-C₆alkylene and C₂-C₈alkenylene, each of which is substituted by 0-4 residues independently selected from C₁-C₄-alkyl, C₁-C₄alkoxy, hydroxyl, amino, mono- and di- di-C₁-C₄alkylamino, halogen, cyano, C₁-C₄fluoroalkyl, C₁-C₄fluoroalkoxy, COOH, carboxamide (CONH₂), mono- and di-C₁-C₄alkylcarboxamide, aryl, heteroaryl, and 5 or 6 membered saturated heterocycles; and
L₃ is absent or a divalent ethylene residue which is substituted by 0 to 2 independently selected methyl or ethyl residues.

7-8. (canceled)

10. A compound of claim 1 wherein the compound is a compound of formula II: 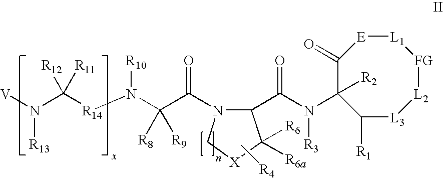
and pharmaceutically acceptable salts, enantiomers, stereoisomers, rotamers, tautomers, diastereomers, or racemates thereof.

11. The compound of claim 10, wherein
x is 0 or 1;
n is 0 or 1;
R₁₄ is C(O) or S(O)ₚ
R₁ is selected from the group consisting of H and C₁₋₄-alkyl;
R₂ is selected from the group consisting of C₁₋₄alkyl, C(O)C₁₋₄-alkyl, C(O)OC₁₋₄-alkyl, and (CH₂)₀₋₄—C₃₋₆-cycloalkyl;
or R₁ and R₂ together form a cyclopropane ring;
R₃ is selected from the group consisting of H and C₁₋₄-alkyl;
X is O, NR₅ or CR₆R₅ₐ;
R₄ is selected from the group consisting of H, C₁₋₄-alkyl, C₃₋₆cycloalkyl, aryl, heterocycle and heteroaryl, each of which may be independently substituted one or more times with a halogen atom or C₁₋₄-alkyl;
R₅ is selected from the group consisting of H, hydroxyl, oxo, C₁₋₆-alkyl, C₂₋₈-alkenyl, C₂₋₈-alkynyl, C₃₋₈-cycloalkyl-C₀₋₄-alkyl, aryl-C₀₋₄-alkyl, aryloxy, heteroaryloxy, heterocycle-C₀₋₄-alkyl and heteroaryl-C₀₋₄-alkyl, each of which may be independently substituted one or more times with a halogen atom, aryl, heteroaryl, trihalomethyl, C₁₋₄-alkoxy or C₁₋₄-alkyl;
R₅ₐ is selected from the group consisting of H, hydroxyl, C₁₋₈-alkyl, C₂₋₆-alkenyl, C₂₋₈-alkynyl, C₃₋₈-cycloalkyl-C₀₋₈-alkyl, aryl-C₀₋₄-alkyl and heteroaryl-C₀₋₄-alkyl,
or R₄ and R₅ may together form a fused dimethyl cyclopropyl ring, a fused cyclopentane ring, a fused phenyl ring or a fused pyridyl ring, each of which may be substituted with a halogen atom, aryl, heteroaryl, trihalomethyl, C₁₋₄-alkoxy or C₁₋₄-alkyl;
or R₅ and R₅ₐ may together form a spirocarbocyclic saturated ring having between 3 and 6 carbon ring atoms which is optionally substituted by 0-2 substituents selected from halogen, C₁₋₆-alkyl, C₂₋₆-alkenyl, C₂₋₆-alkynyl, C₁₋₆-alkoxide, C₃₋₇-cycloalkyl-C₀₋₄-alkyl, phenyl-C₀₋₄-alkyl, naphthyl-C₀₋₄-alkyl, heteroaryl-C₀₋₄-alkyl, or two substituents taken together form a fused or spirocyclic 3 to 7 membered carbocyclic ring, each of which is substituted with 0-3 independently selected halogen atoms or C₁₋₄-alkyl groups;
R₈, R₁₀ and R₁₁ are each, independently, selected from the group consisting of H and C₁₋₄-alkyl;
R₆ and R₁₃ is H;
R₉ and R₁₂ are each, independently, selected from the group consisting of H, C₁₋₄-alkyl and C₃₋₆-cycloalkyl; and
V is selected from the group consisting of -Q¹-Q², wherein Q¹ is absent, C(O), N(H), N(C₁₋₄-alkyl), C═N(CN), C═N(SO₂CH₃), or C═N—COH, and Q² is H, C₁₋₄-alkyl, C═N—COH—C₁₋₄-alkyl, O—C₁₋₄-alkyl NH₂, N(H)—C₁₋₄-alkyl, N(C₁₋₄-alkyl)₂, SO₂-aryl SO₂—C₁₋₄-alkyl, C₃₋₈cycloalkyl-C₀₋₄-alkyl, aryl, heteroaryl and heterocycle, each of which may be independently substituted one or more times with a halogen atom, C₁₋₄-alkyl, C₁₋₄-alkoxy, C₂-C₄alkenyloxy, C₂-C₄alkynyloxy, C₁₋₄-alkyl substituted by one or more halogen atoms, or C₃₋₆-cycloalkyl;
or when x is 0, R₁₀ and V can form a cyclopropyl ring that may be further substituted by an amide group.

12-13. (canceled)

14. The compound of claim 11, wherein X is CR⁵R^{5a}, R₄ is hydrogen, and R⁵ and R₅ₐ taken in combination form a 3 to 6 member spirocyclic carbocyclic substituted with 0-2 substituents selected from halogen, C₁₋₆-alkyl, C₂₋₈-alkenyl, C₂₋₈-alkynyl, C₁₋₈alkoxy, C₃₋₇cycloalkyl-C₀₋₄-alkyl, phenyl-C₀₋₄-alkyl, naphthyl-C₀₋₄-alkyl, heteroaryl-C₀₋₄-alkyl, or two substituents taken together form a fused or spirocyclic 3 to 7 membered carbocyclic ring, each of which is substituted with 0-3 independently selected halogen atoms or C₁₋₄-alkyl groups,

15. The compound of claim 11, wherein the divalent residue: 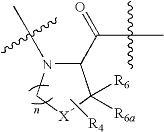
is selected from the group consisting of: 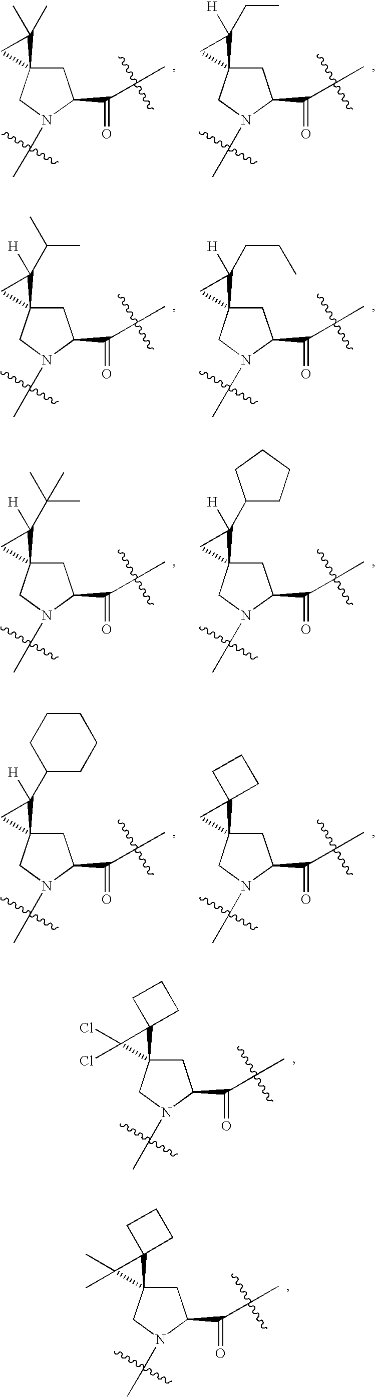 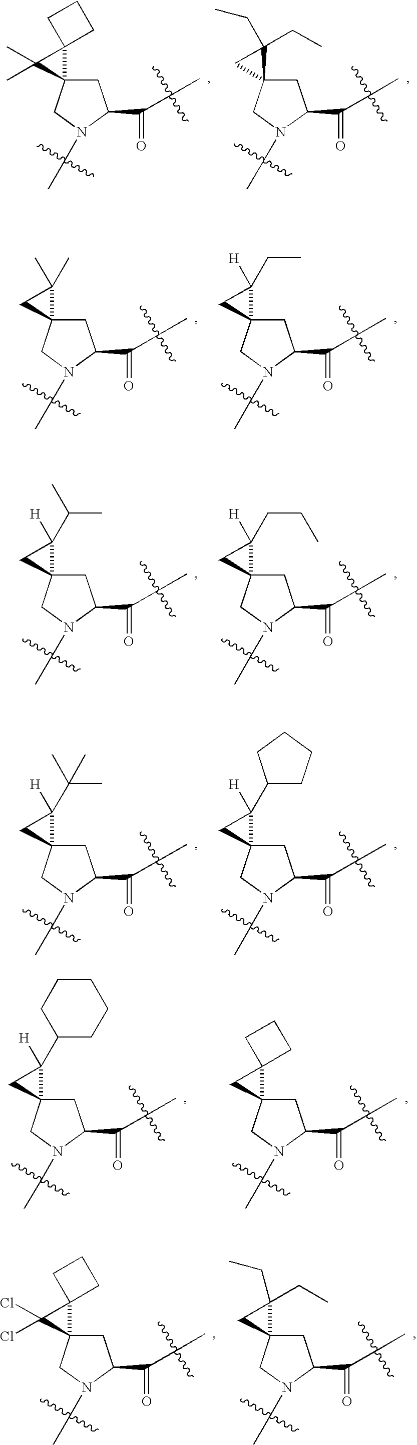 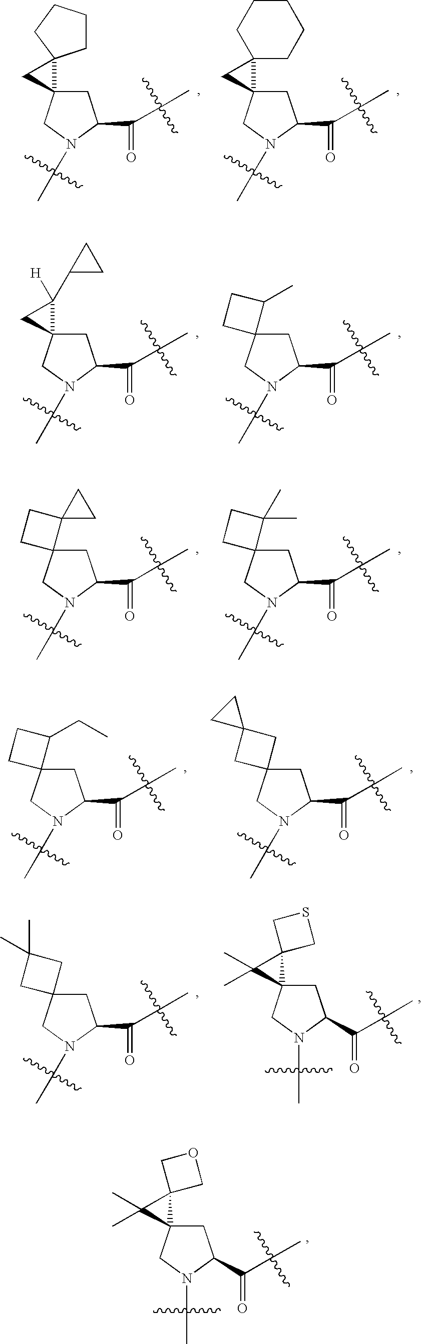 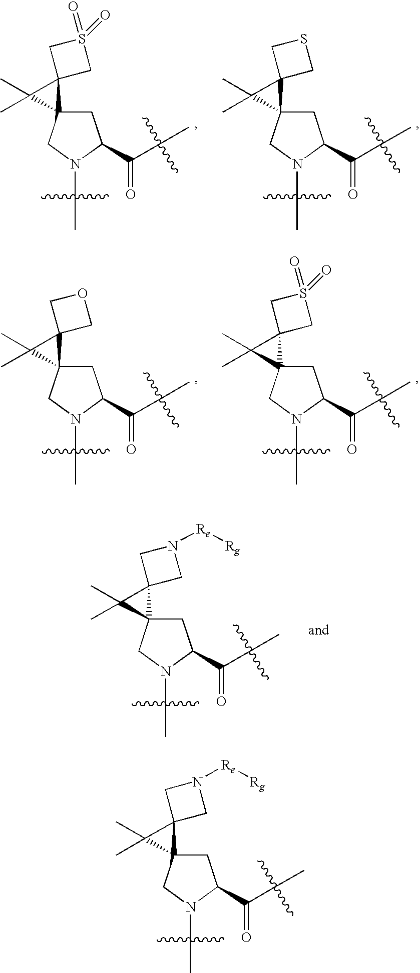
wherein Rₑ is absent, C(O), or S(O)₂; and R_{g} is selected hydrogen or selected from the group consisting of C₁₋₆alkyl, arylC₀₋₄alkyl, heteroarylC₀₋₄alkyl, heterocyclylC₀₋₄alkyl, and C₃₋₇cycloalkylC₀₋₄alkyl each of which is substituted with 0 to 4 independently selected substituents selected from the group consisting of cyano, halogen, hydroxyl, amino, thiol, C₁₋₈-alkyl, C₂₋₈-alkenyl, C₂₋₈-alkynyl C₁₋₈-alkoxy-C₀₋₄alkyl, C₁₋₈-haloalkyl, C₂₋₈-haloalkenyl, C₂₋₈-haloalkynyl, C₁₋₈-haloalkoxy, C₁₋₈-alkylthio, C₁₋₈-alkylsulfonyl, C₁₋₈-alkylsulfoxy, C₁₋₈-alkanoyl; C₁₋₈-alkoxycarbonyl, C₃₋₇-cycloalkyl-C₀₋₄alkyl, aryl-C₀₋₄-alkyl heteroaryl-C₀₋₄-alkyl, COOH, C(O)NH₂, mono- and di-C₁₋₄-alkyl-carboxamide, mono- and di-C₁₋₄-alkyl-amino-C₀₋₄alkyl, SO₃H, SO₂NH₂, and mono- and di-C₁₋₄-alkylsulfonamide.

16. (canceled)

17. A compound of claim 1, wherein the compound is a compound of formula III: 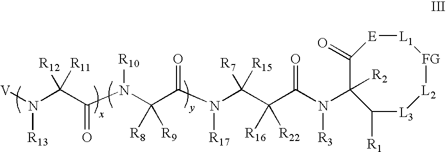
and pharmaceutical acceptable salts, enantiomers, stereoisomers, rotamers, tautomers, diastereomers, or racemates thereofs.

18-20. (canceled)

21. A compound of claim 1, wherein the compound is a compound of formula IX: 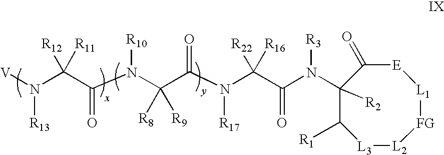
and pharmaceutical acceptable salts, enantiomers, stereoisomers, rotamers, tautomers, diastereomers, or racemates thereof.

22-24. (canceled)

25. A compound of the formula: 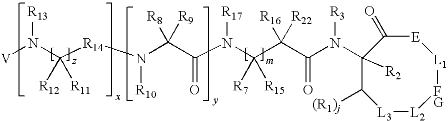
and pharmaceutically acceptable salts, enantiomers, stereoisomers, rotamers, tautomers, diastereomers, or racemates thereof;
wherein
the macrocycle: 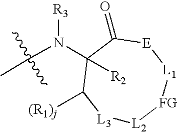
comprises between 10 to 25 ring atoms;
m, x and z are each independently selected from 0 or 1;
j, p and y are independently selected at each occurrence from the group consisting of 0, 1 and 2;
R₁ and R₂ are independently selected, at each occurrence, from hydrogen or from the group consisting of alkyl, alkenyl, alkynyl, cycloalkyl, cyano, alkoxy, and cycloalkyloxy, each of which is unsubstituted or substituted with 1-8 moieties which can be the same or different and are independently selected; wherein X₂ is hydroxy, oxo, alkyl, aryl, alkoxy, aryloxy, thio, alkylthio, arylthio, amino, alkylamino, arylamino, alkylsulfonyl, arylsulfonyl, alkylsulfonamido, arylsulfonamido, heteroarylsulfonamido, arylaminosulfonyl, heteroarylaminosulfonyl, mono and dialkylaminosulfonyl, carboxy, carbalkoxy, amido, carboxamido, alkoxycarbonylamino, aminocarbonyloxy, alkoxycarbonyloxy, alkylureido, arylureido, halogen, cyano, or nitro; wherein each of said alkyl, alkoxy, and aryl can be unsubstituted or optionally independently substituted with one or more moieties which can be the same or different and are independently selected from alkyl, alkenyl, alkynyl, cycloalkyl, cycloalkyl-alkyl, heterocyclyl heterocyclylalkyl, aryl, alkylaryl, aralkyl, arylheteroaryl, heteroaryl, heterocyclylamino, alkylheteroaryl and heteroaralkyl;
R₃ is selected from the group consisting of H and C₁₋₄-alkyl;
E is a divalent residue selected from the group consisting of NR₂₃, C(O)NR₂₃, NR₂₃S(O)ₚ, NR₂₃S(O)ₚNR₂₃;
L₁ and L₂ are divalent residue independently selected from the group consisting of alkylene, (CH₂)₁—FG-(CH₂)ₖ, arylene, heteroarylene, cycloalkylene, and heterocycloalkylene, each of which is substituted with 0 to 4 independently selected X₁ or X₂ groups;
i and k are independently selected integers of from 0 to 7;
L₃ is absent or a divalent ethylene or acetylene residue, wherein the divalent ethylene is substituted by 0-2 substituents selected from alkyl, aryl, heteroaryl, mono- or di-alkylamino-C₀-C₆alkyl, hydroxyl alkyl or alkoxyalkyl;
FG is a divalent residue selected from the group consisting of O, S(O)ₚ, NR₂₃, C(O), C(O)NR₂₃, NR₂₃C(O), OC(O)NR₂₃, NR₂₃C(O)NR₂₃, S(O)NR₂₃, NR₂₃S(O)ₚ, and NR₂₃S(O)ₚNR₂₃;
R₂₃ is independently selected at each occurrence from hydrogen or the group consisting of alkyl, alkenyl, alkynyl, cycloalkyl, cycloalkylalkyl, aryl, heteroaryl heteroaralkyl and aralkyl, each of which is substituted with 0-2 substituents independently selected from halogen, alkyl, and alkoxy;
R₇, R₈, R₉, R₁₀, R₁₁, R₁₂, R₁₃, R₁₆, R₁₅, R₁₇, R₂₂, and V are each, independently, selected from the group consisting of H, alkyl, alkenyl, alkynyl, aryl, alkyl-aryl, heteroalkyl, heterocyclyl, heteroaryl, aryl-heteroaryl, alkyl-heteroaryl, cycloalkyl, alkyloxy, alkyl-aryloxy, aryloxy, heteroaryloxy, heterocyclyloxy, cycloalkyloxy, amino, alkylamino, arylamino, alkyl-arylamino, arylamino, heteroarylamino, cycloalkylamino, carboxyalkylamino, aralkyloxy and heterocyclylamino; each of which may be further independently substituted one or more times with X₁ and X₂;
X₁ is alkyl alkenyl, alkynyl, cycloalkyl, spirocycloalkyl, cycloalkyl-alkyl, heterocyclyl, heterocyclylalkyl, aryl, alkylaryl, aralkyl, arylheteroaryl, heteroaryl, heterocyclylamino, alkylheteroaryl, or heteroaralkyl; wherein X₁ can be independently substituted with one or more of X₂ moieties which can be the same or different and are independently selected;
X₂ is hydroxy, oxo, alkyl, aryl, heteroaryl, alkoxy, aryloxy, heteroaryloxy, thio, alkylthio, arylthio, heteroarylthio, amino, alkylamino, arylamino, heteroarylamino, alkylsulfonyl, arylsulfonyl, heteroarylsulfonyl, alkylsulfonamido, arylsulfonamido, heteroarylsulfonamido, arylaminosulfonyl, heteroarylaminosulfonyl, mono- and di-alkylamino, carboxy, carbalkoxy, amido, carboxamido, alkoxycarbonylamino, aminocarbonyloxy, alkoxycarbonyloxy, carbamoyl, ureido, alkylureido, arylureido, halogen, cyano, or nitro; wherein each of said alkyl, alkoxy, and aryl can be unsubstituted or optionally independently substituted with one or more moieties which can be the same or different and are independently selected from alkyl, alkenyl, alkynyl, cycloalkyl, cycloalkyl-alkyl, heterocycyl, heterocyclylalkyl, aryl, alkylaryl, aralkyl, arylheteroaryl, heteroaryl, heterocyclylamino, alkylheteroaryl and heteroaralkyl;
R₁₄ is C(O) or S(O)ₚ;
V is selected from the group consisting of -Q¹-Q², wherein Q¹ is absent, C(O), S(O)ₚ, N(H), N(C₁₋₄alkyl), C═N(CN), C═N(SO₂CH₃), or C═N—COH, and Q² is H, C₁₋₄-alkyl, C═N—COH—CH₁₋₄-alkyl, O—C₁₋₄-alkyl, NH₂, N(H)—C₁₋₄-alkyl, N(C₁₋₄-alkyl)₂, SO₂-aryl, SO₂—C₁₋₄alkyl, C₃₋₈-cycloalkyl-C₀₋₄-alkyl, aryl, heteroaryl and heterocycle, each of which may be independently substituted one or more times with a halogen atom, C₁₋₄-alkyl, C₂₋₄-alkenyl, C₂₋₄-alkynyl, C₁₋₄-alkoxy, C₂₋₄-alkenyloxy, C₂₋₄-alkynyloxy, C₁₋₄-alkyl substituted by one or more halogen atoms, C₃₋₆-cycloalkyl, carboxylate, carboxamido, mono-and di-alkylamino, or mono-and di-alkylcarboxamido;
or R₂₂ and R₁₆ may together form a 3, 4, 5, 8 or 7-membered ring and may contain one or more heteroatoms, wherein the ring may be further substituted one or more times;
or R₇ and R₁₅ may together form a 3, 4, 5, 8 or 7-membered ring and may contain one or more heteroatoms, wherein the ring may be further substituted one or more times;
or R₁₅ and R₁₇ may together form a 3, 4, 5, 8 or 7-membered ring and may contain one or more heteroatoms, wherein the ring may be further substituted one or more times;
or R₁₅ and R₁₆ may together form a 4, 5, 6 or 7-membered ring and may contain one or more heteroatoms, wherein the ring may be further substituted one or more times;
or R₁₅ and R₁₆ may together form an arylene or heteroarylene ring and R₇ and R₂₂ are absent, wherein the ring may be further substituted one or more times;
or R₁ and R₂ may together form a 3, 4, 5, 8 or 7-membered ring that is saturated or partially unsaturated and may contain one or more heteroatoms, wherein the ring may be further substituted one or more times;
or R₁₇ and R₁₆ may together form a 4, 5, 6, 7 or 8-membered ring of the formula: 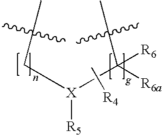
wherein
n and g are each, independently, 0, 1 or 2;
X is O, S, N, NR₅, CR₅ or CR₅R₅ₐ;
R₄ is selected from the group consisting of H, C₁₋₆-alkyl, C₃₋₇cycloalkyl, aryl, heterocycle and heteroaryl, each of which may be independently substituted one or more times with a halogen atom or C₁₋₄-alkyl;
R₅ is selected from the group consisting of H, hydroxyl, oxo, C₁₋₈-alkyl, C₂₋₈-alkenyl, C₂₋₈-alkynyl, C₃₋₈cycloalkyl-C₀₋₄-alkyl, aryl-C₀₋₄-alkyl, heterocycle-C₀₋₄-alkyl, heteroaryl-C₀₋₄alkyl, C₃₋₈-cycloalkyloxy, aryloxy, NR₂₃COR₂₃, CONR₂₃R₂₃, NR₂₃CONHR₂₃, OCONR₂₃R₂₃, NR₂₃COOR₂₃, OCOR₂₃, COOR₂₃, aryl-C(O)O, aryl-C(O)NR₂₃, heteroaryloxy, heteroaryl-C(O)O, heteroaryl-C(O)NR₂₃, each of which may he independently substituted one or more times with a halogen atom, aryl, heteroaryl, trihalomethyl, C₁₋₄-alkyl, or C₁₋₄-alkoxy;
R₅ₐ is selected from the group consisting of H, hydroxyl, C₁₋₈-alkyl, C₂₋₈-alkenyl, C₂₋₈-alkynyl, C₃₋₈-cycloalkyl-C₀₋₄-alkyl, aryl-C₀₋₄-alkyl and heteroaryl-C₀₋₄-alkyl,
or R₄ and R₅ may together form a fused dimethyl cyclopropyl ring, a fused cyclopentane ring, a fused phenyl ring or a fused pyridyl ring, each of which may be substituted with a halogen atom, aryl, heteroaryl, trihalomethyl, C₁₋₄-alkoxy or C₁₋₄-alkyl;
or R₅ and R₅ₐ may together form a spirocyclic ring having between 3 and 7 ring atoms which is optionally substituted by 0-4 substituents selected from cyano, halogen, hydroxyl, amino, thiol, C₁₋₈-alkyl, C₂₋₈alkenyl, C₂₋₈-alkynyl, C₁₋₈-alkoxide, C₁₋₈-haloalkyl, C₂₋₈-haloalkenyl, C₂₋₈-haloalkynyl, C₁₋₆-haloalkoxide, C₁₋₈alkylthio, C₁₋₈-alkylsulfonyl, C₁₋₈-alkylsulfoxide, C₁₋₈-alkanoyl, C₁₋₈-alkoxycarbonyl, C₃₋₇-cycloalkyl-C₀₋₄-alkyl, aryl-C₀₋₄-alkyl, heterocyclyl-C₀₋₄-alkyl, heteroaryl-C₀₋₄-alkyl, COOH, C(O)NH₂, mono- and di-C₁₋₄-alkyl-carboxamide, SO₃H, SO₂NH₂, and mono-and di-C₁₋₄-alkylsulfonamide, or two substituents taken together form a fused or spirocycilc 3 to 7 membered ring having 0, 1 or 2 ring heteroatoms selected from N, O and S, which fused or spirocycilc ring has 0 to 2 independently selected substituents selected from halogen, C₁₋₄alkyl, C₁₋₄alkoxy, C₁₋₄alkanoyl, mono- and di-C₁₋₄-alkylamino, mono- and di-C₁₋₄-alkyl-carboxamide, C₁₋₄-alkoxycarbonyl, and phenyl; and
R₆ and R₆ₐ are independently selected at each occurrence from the group consisting of H, C₁₋₄-alkyl and (CH₂)₀₋₄—C₃₋₆-cycloalkyl;
or R₆ and R₆ₐ may together form a spirocyclic ring having between 3 and 7 ring atoms which is optionally substituted by 0-4 substituents selected from cyano, halogen, hydroxyl, amino, thiol, C₁₋₈-alkyl, C₂₋₈-alkenyl, C₂₋₈-alkynyl, C₁₋₈-alkoxide, C₁₋₈-haloalkyl, C₂₋₈-haloalkenyl, C₂₋₈-haloalkynyl, C₁₋₈-haloalkoxide, C₁₋₈-alkylthio, C₁₋₈-alkylsulfonyl, C₁₋₈-alkylsulfoxide, C₁₋₈-alkanoyl, C₁₋₈-alkoxycarbonyl, C₃₋₇-cycloalkyl-C₀₋₄-alkyl, aryl-C₀₋₄-alkyl, heterocyclyl-C₀₋₄-alkyl, heteroaryl-C₀₋₄-alkyl, COOH, C(O)NH₂, mono- and di-C₁₋₄-alkyl-carboxamide, SO₃H, SO₂NH₂, and mono- and di-C₁₋₄-alkylsulfonamide, or two substituents taken together form a fused or spirocyclic 3 to 7 membered ring having 0, 1 or 2 ring heteroatoms selected from N, O and S, which fused or spirocyclic ring has 0 to 2 independently selected substituents selected from halogen, C₁₋₄alkyl, C₁₋₄alkoxy, C₁₋₄alkanoyl, mono- and di-C₁₋₄-alkylamino, mono- and di-C₁₋₄-alkyl-carboxamide, C₁₋₄-alkoxycarbonyl, and phenyl.

28-29. (canceled)

30. The compound of claim 25, wherein
L₁ is C₁-C₆alkylene, C₃-C₇cycloalkylene, arylene or heteroarylene, each of which is substituted by 0-4 residues independently selected from C₁-C₄alkyl, C₁-C₄alkoxy, hydroxyl, amino, mono-and di-C₁-C₄alkylamino, halogen, cyano, C₁-C₄-fluoroalkyl, C₁-C₄fluoroalkoxy, COOH, carboxamide (CONH₃), mono- and di-C₁-C₄alkylcarboxamide, aryl, heteroaryl and 5 or 6 membered saturated heterocycles;
L₂ is selected from C₁-C₆alkylene and C₂-C₆alkenylene, each of which is substituted by 0-4 residues independently selected from C₁-C₄alkyl, C₁-C₄alkoxy, hydroxyl, amino, mono- and di-C₁-C₄alkylamino, halogen, cyano, C₁-C₄fluoroalkyl, C₁-C₄fluoroalkoxy, COOH, carboxamide (CONH₂), mono- and di-C₁-C₄alkylcarboxamide, aryl, heteroaryl and 5 or 8 membered saturated heterocycles; and
L₃ is absent or a divalent ethylene residue which is substituted by 0 to 2 independently selected methyl or ethyl residues.

31-33. (canceled)

34. A compound of claim 25, wherein the compound is a compound of formula II: 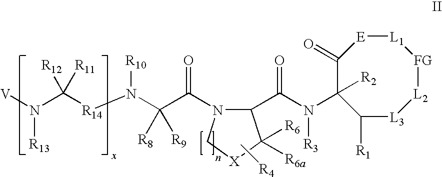
and pharmaceutically acceptable salts, enantiomers, stereoisomers, rotamers, tautomers, diastereomers, or racemates thereof.

35-38. (canceled)

39. The compound of claim 34, wherein the divalent residue: 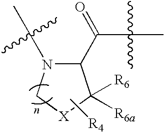
is selected from the group consisting of: 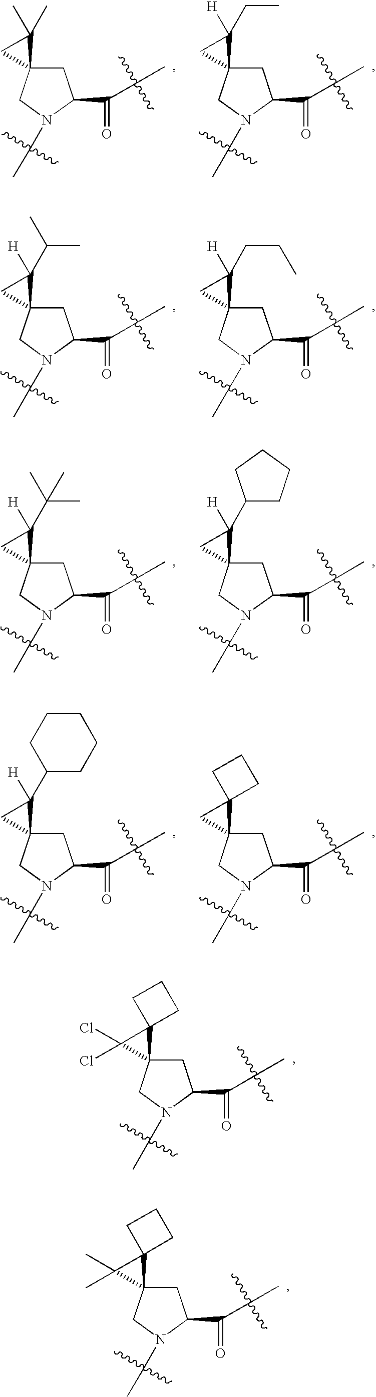 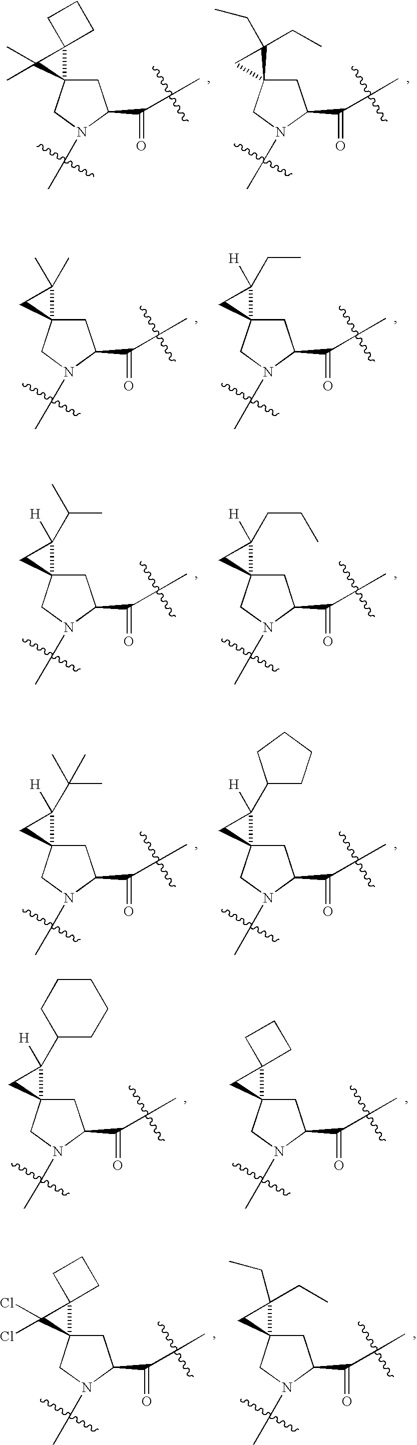 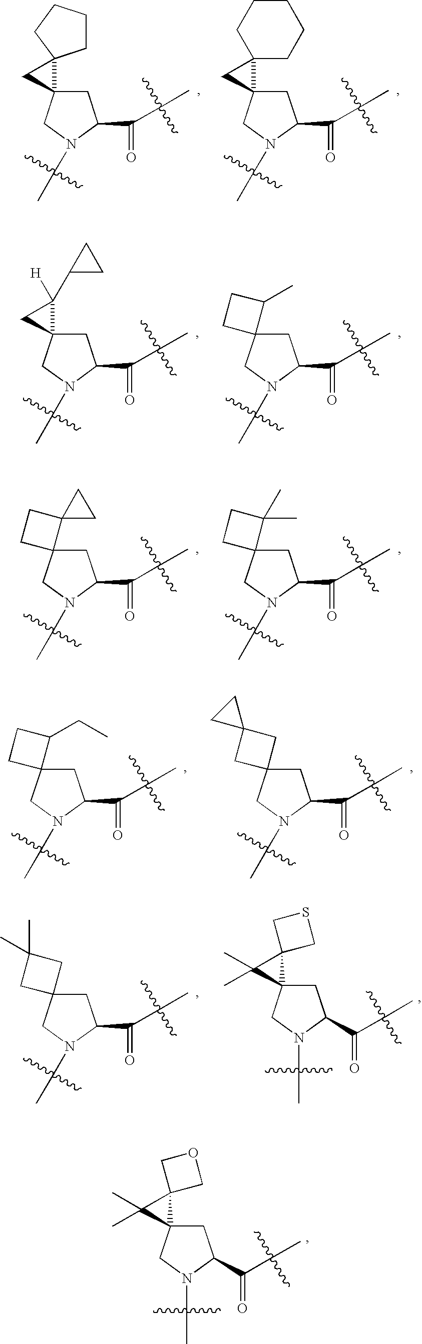 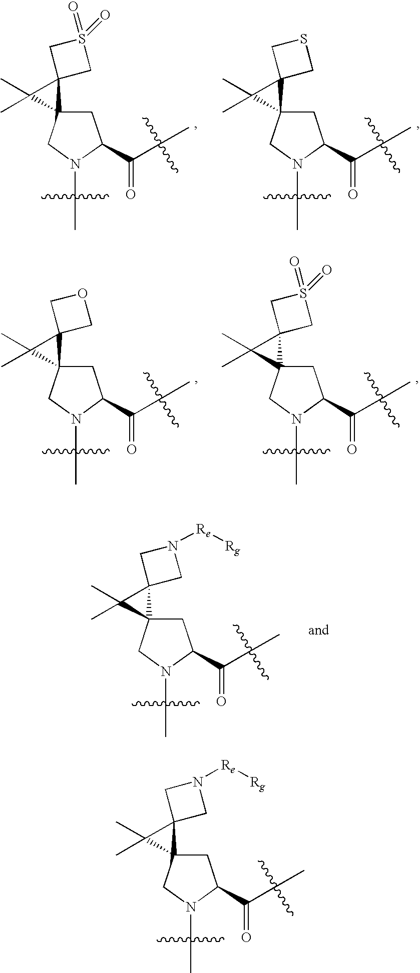
wherein Rₑ is absent, C(O), or S(O)₂; and R_{g} is selected hydrogen or selected from the group consisting of C₁₋₆alkyl, arylC₀₋₄alkyl, heteroarylC₀₋₄alkyl, heterocyclylC₀₋₄alkyl and C₃₋₇cyclcalkylC₀₋₄alkyl, each of which is substituted with 0 to 4 independently selected substituents selected from the group consisting of cyano, halogen, hydroxyl, amino, thiol C₁₋₆-alkyl, C₂₋₈-alkenyl, C₂₋₈alkynyl, C₁₋₈-alkoxy-C₀₋₄alkyl, C₁₋₈-haloalkyl, C₂₋₈-haloalkenyl, C₂₋₈-haloalkynyl, C₁₋₈haloalkoxy, C₁₋₈alkylthio, C₁₋₈-alkylsulfonyl, C₁₋₈-alkylsulfoxy, C₁₋₈-alkanoyl, C₁₋₈-alkoxycarbonyl, C₃₋₇cycloalkyl-C₀₋₄-alkyl, aryl-C₀₋₄alkyl, heteroaryl-C₀₋₄alkyl, COOH, C(O)NH₂, mono- and di-C₁₋₄-alkyl-carboxamide, mono- and di-C₁₋₄-alkyl-amino-C₀₋₄alkyl, SO₃H, SO₂NH₂, and mono-and di-C₁₋₄-alkylsulfonamide.

40-48. (canceled)

49. A compound of the formula: 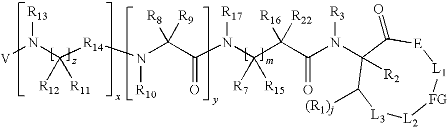
and pharmaceutical acceptable salts, enantiomers, stereoisomers, rotamers, tautomers, diastereomers, or racemates thereof;
wherein
the macrocycle: 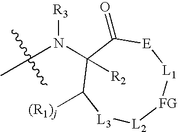
comprises between 10 to 25 ring atoms;
m, x and z are each independently selected from 0 or 1;
j, p and y are independently selected at each occurrence from the group consisting of 0, 1 and 2;
R₁ and R₂ are independently selected from hydrogen or from the group consisting of alkyl, alkenyl, alkynyl, cycloalkyl, cyano, alkoxy, and cycloalkyloxy, each of which is unsubstituted or substituted with 1-6 moieties which can be the same or different and are independently selected; wherein X₂ is hydroxy, oxo, alkyl, aryl, alkoxy, aryloxy, thio, alkylthio, arylthio, amino, alkylamino, arylamino, alkylsulfonyl, arylsulfonyl, alkylsulfonamido, arylsulfonamido, heteroarylsulfonamido, arylaminosulfonyl, heteroarylaminosulfonyl, mono and dialkylaminosulfonyl, carboxy, carbalkoxy, amide, carboxamido, alkoxycarbonylamino, aminocarbonyloxy, alkoxycarbonyloxy, alkylureido, arylureido, halogen, cyano, or nitro; wherein each of said alkyl, alkoxy, and aryl can be unsubstituted or optionally independently substituted with one or more moieties which can be the same or different and are independently selected from alkyl, alkenyl, alkynyl, cycloalkyl, cycloalkyl-alkyl, heterocyclyl, heterocyclylalkyl, aryl, alkylaryl, aralkyl, arylheteroaryl, heteroaryl, heterocyclylamino, alkylheteroaryl and heteroaralkyl;
R₃ is selected from the group consisting of H and C₁₋₄-alkyl;
E is a divalent residue selected from the group consisting of NR₂₃, C(O)NR₂₃, NR₂₃S(O)ₚ, NR₂₃S(O)NR₂₃;
L₁ is a divalent residue selected from the group consisting of arylene, heteroarylene, and cycloalkylene, which is substituted with 0 to 4 independently selected X₁ or X₂ groups;
L₂ is a divalent residue selected from the group consisting of alkylene, (CH₂)₁—FG-(CH₂)ₖ, arylene, heteroarylene, cycloalkylene and heterocycloalkylene, which is substituted with 0 to 4 independently selected X₁ or X₂ groups;
i and k are independently selected integers of from 0 to 7;
L₃ is absent or a divalent ethylene or acetylene residue, wherein the divalent ethylene is substituted by 0-2 substituents selected from alkyl aryl, heteroaryl, mono- or di-alkylamino-C₃-C₆alkyl, hydroxyl alkyl or alkoxyalkyl;
FG is absent or a divalent residue selected from the group consisting of O, S(O)ₚ, NR₂₃, C(O), C(O)NR₂₃, NR₂₃C(O), OC(O)NR₂₃, NR₂₃C(O)O, NR₂₃C(O)NR₂₃, S(O)ₚNR₂₃, NR₂₃S(O)ₚ, and NR₂₃S(O)ₚNR₂₃;
R₂₃ is independently selected at each occurrence from hydrogen or the group consisting of alkyl, alkenyl, alkynyl, cycloalkyl, cycloalkylalkyl, aryl, heteroaryl, heteroaralkyl and aralkyl, each of which is substituted with 0-2 substituents independently selected from halogen, alkyl, and alkoxy;
R₇, R₆, R₉, R₁₀, R₁₁, R₁₂, R₁₃, R₁₆, R₁₅, R₁₇, R₂₂, and V are each, independently, selected from the group consisting of H, alkyl, alkenyl, alkynyl, aryl, alkyl-aryl, heteroalkyl, heterocycyl, heteroaryl, aryl-heteroaryl, alkyl-heteroaryl, cycloalkyl, alkyloxy, alkyl-aryloxy, aryloxy, heteroaryloxy, heterocyclyloxy, cycloalkyloxy, amino, alkylamino, arylamino, alkyl-arylamino, arylamino, heteroarylamino, cycloalkylamino, carboxyalkylamino, aralkyloxy and heterocyclylamino; each of which may be further independently substituted one or more times with X₁ and X₂;
X₁ is alkyl, alkenyl, alkynyl, cycloalkyl, spirocycloalkyl, cycloalkyl-alkyl, heterocycyl, heterocyclylalkyl, aryl, alkylaryl, aralkyl, arylheteroaryl heteroaryl, heterocyclylamino, alkylheteroaryl, or heteroaralkyl; wherein X₁ can be independently substituted with one or more of X₂ moieties which can be the same or different and are independently selected;
X₂ is hydroxy, oxo, alkyl, aryl, heteroaryl, alkoxy, aryloxy, heteroaryloxy, thio, alkylthio, arylthio, heteroarylthio, amino, alkylamino, arylamino, heteroarylamino, alkylsulfonyl, arylsulfonyl, heteroarylsulfonyl, alkylsulfonamido, arylsulfonamido, heteroarylsulfonamido, arylaminosulfonyl, heteroarylaminosulfonyl, mono and dialkylamino sulfonyl, carboxy, carbalkoxy, amido, carboxamido, alkoxycarbonylamino, aminocarbonyloxy, alkoxycarbonyloxy, carbamoyl, ureido, alkylureido, arylureido, halogen, cyano, or nitro; wherein each of said alkyl, alkoxy, and aryl can be unsubstituted or optionally independently substituted with one or more moieties which can be the same or different and are independently selected from alkyl, alkenyl, alkynyl, cycloalkyl, cycloalkyl-alkyl, heterocycyl, heterocyclylalkyl, aryl, alkylaryl, aralkyl, arylheteroaryl, heteroaryl, heterocyclylamino, alkylheteroaryl and heteroaralkyl;
R₁₄ is C(O) or S(O)ₚ;
V is selected from the group consisting of -Q¹-Q², wherein Q¹ is absent C(O), S(O)ₚ, N(H), N(C₁₋₄-alkyl), C═N(CN), C═N(SO₂CH₃), or C═N—COH, and Q² is H, C₁₋₄-alkyl, C═N—COH—C₁₋₄-alkyl, O—C₁₋₄-alkyl, NH₂, N(H)—C₁₋₄-alkyl, N(C₁₋₄-alkyl)₂, SO₂-aryl, SO₂—C₁₋₄alkyl, C₃₋₆-cycloalkyl-C₀₋₄-alkyl, aryl heteroaryl and heterocycle, each of which may be independently substituted one or more times with a halogen atom, C₁₋₄-alkyl, C₂₋₄-alkenyl, C₂₋₄-alkynyl, C₁₋₄-alkoxy, C₂₋₄-alkenyloxy, C₂₋₄-alkynyloxy, C₁₋₄-alkyl substituted by one or more halogen atoms, C₃₋₆-cycloalkyl, carboxylate, carboxamido, mono- and di-alkylamino, or mono- and di-alkylcarboxamido;
or R₂₂ and R₁₈ may together form a 3, 4, 5, 8 or 7-membered ring and may contain one or more heteroatoms, wherein the ring may be further substituted one or more times;
or R₇ and R₁₅ may together form, a 3, 4, 5, 8 or 7-membered ring and may contain one or more heteroatoms, wherein the ring may be further substituted one or more times;
or R₁₅ and R₁₇ may together form a 3, 4, 5, 8 or 7-membered ring and may contain one or more heteroatoms, wherein the ring may be further substituted one or more times;
or R₁₅ and R₁₆ may together form a 4, 5, 8 or 7-membered ring and may contain one or more heteroatoms, wherein the ring may be further substituted one or more times;
or R₁₅ and R₁₆ may together form an arylene or heteroarylene ring and R₇ and R₂₂ are absent, wherein the ring may be further substituted one or more times;
or R₁ and R₂ may together form a 3, 4, 5, 8 or 7-membered ring that is saturated or partially unsaturated and may contain one or more heteroatoms, wherein the ring may be further substituted one or more times;
or R₁₇ and R₁₆ may together form a 4, 5, 6, 7 or 8-membered ring of the formula: 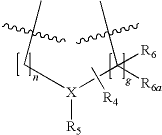
wherein
n and g are each, independently, 0, 1 or 2;
X is O, S, N, NR₅; CR₅ or CR₅R₅ₐ;
R₄ is selected from the group consisting of H, C₁₋₆-alkyl, C₃₋₇-cycloalkyl, aryl, heterocycle and heteroaryl, each of which may be independently substituted one or more times with a halogen atom or C₁₋₄-alkyl;
R₅ is selected from the group consisting of H, hydroxyl oxo, C₁₋₈-alkyl, C₂₋₈alkenyl, C₂₋₈-alkynyl, C₃₋₈-cycloalkyl-C₀₋₄-alkyl, aryl-C₀₋₄-alkyl, heterocycle-C₀₋₄-alkyl, heteroaryl-C₀₋₄-alkyl, C₃₋₈-cycloalkyloxy, aryloxy, NR₂₃COR₂₃, CONR₂₃R₂₃, NR₂₃CONHR₂₃, OCONR₂₃R₂₃, NR₂₃COOR₂₃, OCOR₂₃, COOR₂₃, aryl-C(O)O, aryl-C(O)NR₂₃, heteroaryloxy, heteroaryl-C(O)O, heteroaryl-C(O)NR₂₃, each of which may be independently substituted one or more times with a halogen atom, aryl, heteroaryl, trihalomethyl, C₁₋₄-alkyl, or C₁₋₄-alkoxy;
R₅ₐ is selected from the group consisting of H, hydroxyl, C₁₋₈-alkyl, C₂₋₈-alkenyl, C₂₋₈-alkynyl, C₃₋₈-cycloalkyl-C₀₋₄-alkyl, aryl-C₀₋₄-alkyl and heteroaryl-C₀₋₄-alkyl,
or R₄ and R₅ may together form a fused dimethyl cyclopropyl ring, a fused cyclopentane ring, a fused phenyl ring or a fused pyridyl ring, each of which may be substituted with a halogen atom, aryl, heteroaryl, trihalomethyl, C₁₋₄-alkoxy or C₁₋₄-alkyl;
or R₆ and R₆ₐ may together form a spirocyclic ring having between 3 and 7 ring atoms which is optionally substituted by 0-4 substituents selected from cyano, halogen, hydroxyl, amino, thiol, C₁₋₈-alkyl, C₂₋₈-alkenyl C₂₋₈-alkynyl, C₁₋₈-alkoxide, C₁₋₈-haloalkyl, C₂₋₈haloalkenyl, C₂₋₈-haloalkynyl, C₁₋₈-haloalkoxide, C₁₋₈-alkylthio, C₁₋₈-alkylsulfonyl, C₁₋₈-alkylsulfoxide, C₁₋₈-alkanoyl, C₁₋₈-alkoxycarbonyl, C₃₋₇-cycloalkyl-C₀₋₄-alkyl, aryl-C₀₋₄-alkyl, heterocyclyl-C₀₋₄-alkyl, heteroaryl-C₀₋₄-alkyl, COOH, C(O)NH₂, mono- and di-C₁₋₄-alkyl-carboxamide, SO₃H, SO₂NH₂, and mono- and di-C₁₋₄-alkylsulfonamide, or two substituents taken together form a fused or spirocyclic 3 to 7 membered ring having 0, 1 or 2 ring heteroatoms selected from N, O and S, which fused or spirocyclic ring has 0 to 2 independently selected substituents selected from halogen, C₁₋₄alkyl, C₁₋₄alkoxy, C₁₋₄alkanoyl, mono- and di-C₁₋₄-alkylamino, mono- and di-C₁₋₄-alkyl-carboxamide, C₁₋₄-alkoxycarbonyl, and phenyl; and
R₆ and R₆ₐ are independently selected at each occurrence from the group consisting of H, C₁₋₄-alkyl and (CH₂)₀₋₄—C₃₋₆-cycloalkyl;
or R₆ and R₆ₐ may together form a spirocyclic ring having between 3 and 7 ring atoms which is optionally substituted by 0-4 substituents selected from cyano, halogen, hydroxyl amino, thiol, C₁₋₈-alkyl, C₂₋₈-alkenyl, C₂₋₈-alkynyl, C₁₋₈-alkoxide, C₁₋₈-haloalkyl, C₂₋₈haloalkenyl, C₂₋₈-haloalkynyl, C₁₋₈-haloalkoxide, C₁₋₈-alkylthio, C₁₋₈-alkylsulfonyl, C₁₋₈-alkylsulfoxide, C₁₋₈-alkanoyl, C₁₋₈-alkoxycarbonyl, C₃₋₇-cycloalkyl-C₀₋₄-alkyl, aryl-C₀₋₄-alkyl, heterocyclyl-C₀₋₄-alkyl, heteroaryl-C₀₋₄-alkyl, COOH, C(O)NH₂, mono- and di-C₁₋₄-alkyl-carboxamide, SO₃H, SO_{2N}H₂, and mono-and di-C₁₋₄-alkylsulfonamide, or two substituents taken together form a fused or spirocyclic 3 to 7 membered ring having 0, 1 or 2 ring heteroatoms selected from N, O and S, which fused or spirocyclic ring has 0 to 2 independently selected substituents selected from halogen, C₁₋₄alkyl, C₁₋₄alkoxy, C₁₋₄alkanoyl, mono- and di-C₁₋₄-alkylamino, mono- and di-C₁₋₄-alkyl-carboxamide, C₁₋₄-alkoxycarbonyl, and phenyl.

50-53. (canceled)

54. The compound of claim 49, wherein
L₁ is C₃-C₇cycloalkylene, arylene or heteroarylene which is substituted by 0-4 residues independently selected from C₁-C₄alkyl, C₁-C₄alkoxy, hydroxyl, amino, mono- and di-C₁-C₄alkylamino, halogen, cyano, C₁-C₄fluoroalkyl, C₁-C₄fluoroalkoxy, COOH, carboxamide (CONH₂), mono- and di-C₁-C₄alkylcarboxamide, aryl, heteroaryl and 5 or 6 membered saturated heterocycles;
L₂ is selected from C₁-C₆alkylene and C₂-C₆alkenylene, each of which is substituted by 0-4 residues independently selected from C₁-C₄alkyl, C₁-C₄alkoxy, hydroxyl, amino, mono- and di-C₁-C₄alkylamino, halogen, cyano, d-C₁-C₄fluoroalkyl, C₁-C₄fluoroalkoxy, COOH, carboxamide (CONH₂), mono- and di-C₁-C₄alkylcarboxamide, aryl, heteroaryl and 5 or 8 membered saturated heterocycles; and
L₃ is absent or a divalent ethylene residue which is substituted by 0 to 2 independently selected methyl or ethyl residues.

55-57. (canceled)

58. A compound of claim 49, wherein the compound is a compound of formula II: 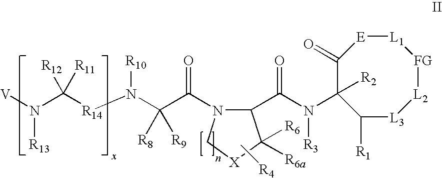
and pharmaceutically acceptable salts, enantiomers, stereoisomers, rotamers, tautomers, diastereomers, or racemates thereof.

59. The compound of claim 58, wherein
x is 0 or 1;
n is 0 or 1;
R₁₄ is C(O) or S(O)ₚ
R₁ is selected from the group consisting of H and C₁₋₄-alkyl;
R₂ is selected from the group consisting of C₁₋₄-alkyl, C(O)C₁₋₄-alkyl, C(O)OC₁₋₄-alkyl, and (CH₂)₀₋₄-C₃₋₆-cycloalkyl;
or R₁ and R₂ together form a cyclopropane ring;
R₃ is selected from the group consisting of H and C₁₋₄-alkyl;
X is O, NR₅ or CR₅R₅ₐ;
R₄ is selected from the group consisting of H, C₁₋₄-alkyl, C₃₋₆-cycloalkyl, aryl, heterocycle and heteroaryl each of which may be independently substituted one or more times with a halogen atom or C₁₋₄alkyl;
R₅ is selected from the group consisting of H, hydroxyl, oxo, C₁₋₈-alkyl C₂₋₈-alkenyl, C₂₋₈-alkynyl, C₃₋₈-cycloalkyl-C₀₋₄-alkyl aryl-C₀₋₄-alkyl aryloxy, heteroaryloxy, heterocycle-C₀₋₄-alkyl and heteroaryl-C₀₋₄-alkyl, each of which may be independently substituted one or more times with a halogen atom, aryl, heteroaryl, trihalomethyl, C₁₋₄-alkoxy or C₁₋₄-alkyl;
R₅ₐ is selected from the group consisting of H, hydroxyl, C₁₋₈-alkyl, C₂₋₈-alkenyl, C₂₋₈-alkynyl, C₃₋₈-cycloalkyl-C₀₋₄-alkyl, aryl-C₀₋₄-alkyl and heteroaryl-C₀₋₄-alkyl,
or R₄ and R₅ may together form a fused dimethyl cyclopropyl ring, a fused cyclopentane ring, a fused phenyl ring or a fused pyridyl ring, each of which may be substituted with a halogen atom, aryl, heteroaryl, trihalomethyl, C₁₋₄-alkoxy or C₁₋₄-alkyl;
or R₅ and R₅ₐ may together form a spirocarbocyclic saturated ring having between 3 and 6 carbon ring atoms which is optionally substituted by 0-2 substituents selected from halogen, C₁₋₆-alkyl, C₂₋₆-alkenyl, C₂₋₆-alkynyl, C₁₋₆-alkoxide, C₃₋₇-cycloalkyl-C₀₋₄-alkyl, phenyl-C₀₋₄-alkyl, naphthyl-C₀₋₄-alkyl, heteroaryl-C₀₋₄-alkyl, or two substituents taken together form a fused or spirocyclic 3 to 7 membered carbocyclic ring, each of which is substituted with 0-3 independently selected halogen atoms or C₁₋₄-alkyl groups;
R₈, R₁₀ and R₁₁ are each, independently, selected from the group consisting of H and C₁₋₄-alkyl;
R₆ and R₁₃ is H;
R₉ and R₁₂ are each, independently, selected from the group consisting of H, C₁₋₄-alkyl and C₃₋₆cycloalkyl; and
V is selected from the group consisting of -Q¹-Q², wherein Q¹ is absent, C(O), N(H), H(C₁₋₄-alkyl), C═N(CN), C═N(SO₂CH₃), or C═N—COH, and Q² is H, C₁₋₄-alkyl, C—N—COH—C₁₋₄-alkyl, O—C₁₋₄-alkyl, NH₂, N(H)-C₁₋₄-alkyl, N(C₁₋₄-alkyl)₂, SO₂-aryl, SO₂-C₁₋₄-alkyl, C₃₋₆-cycloalkyl-C₀₋₄-alkyl, aryl, heteroaryl and heterocycle, each of which may be independently substituted one or more times with a halogen atom, C₁₋₄-alkyl, C₁₋₄alkoxy, C₂-C₄alkenyloxy, C₂-C₄alkynyloxy, C₁₄-alkyl substituted by one or more halogen atoms, or C₃₋₆-cycloalkyl;
or when y is 0, R₁₀ and V can form a cyclopropyl ring that may be further substituted by an amide group.

60-62. (canceled)

63. The compound of claim 58, wherein the divalent residue: 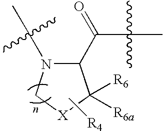
is selected from the group consisting of: 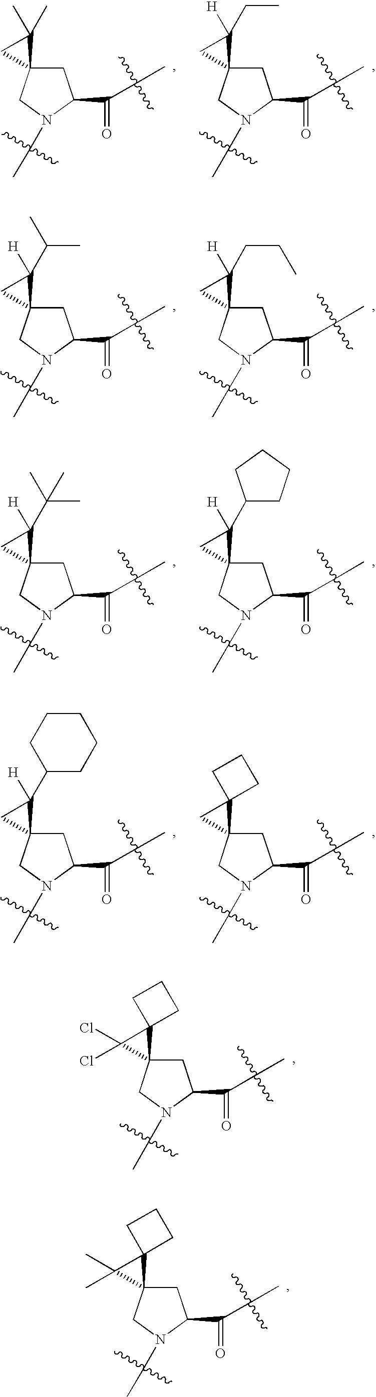 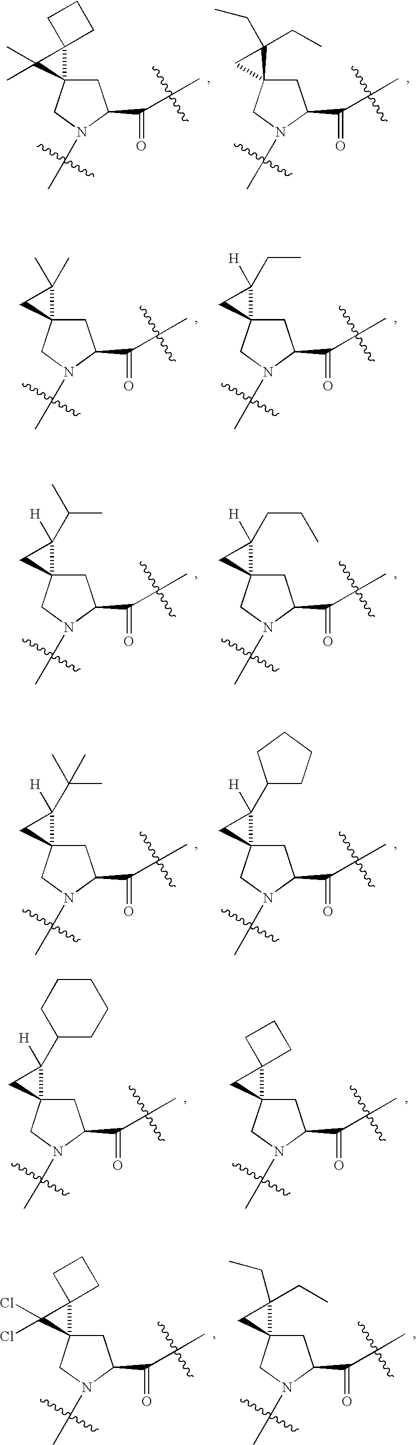 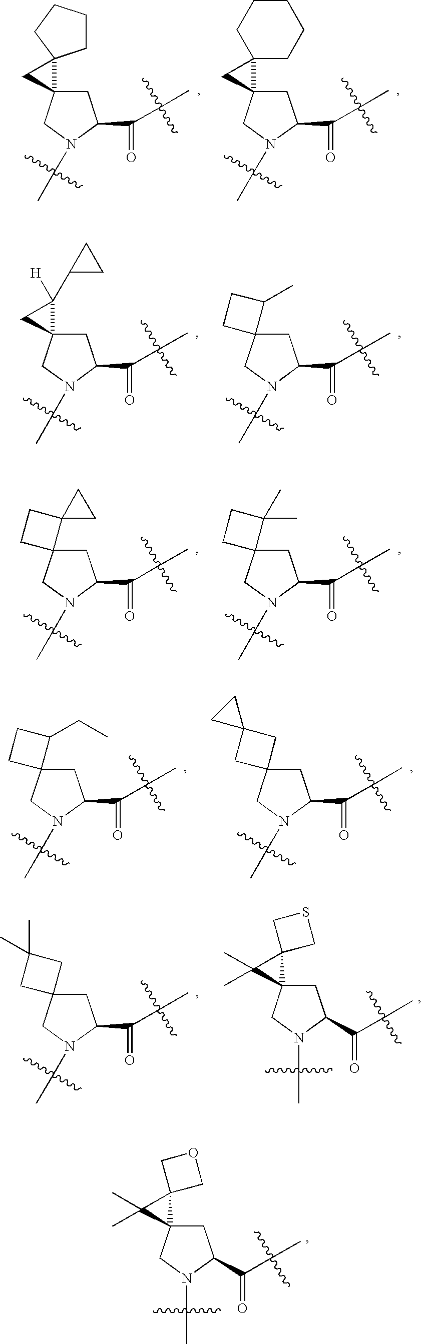 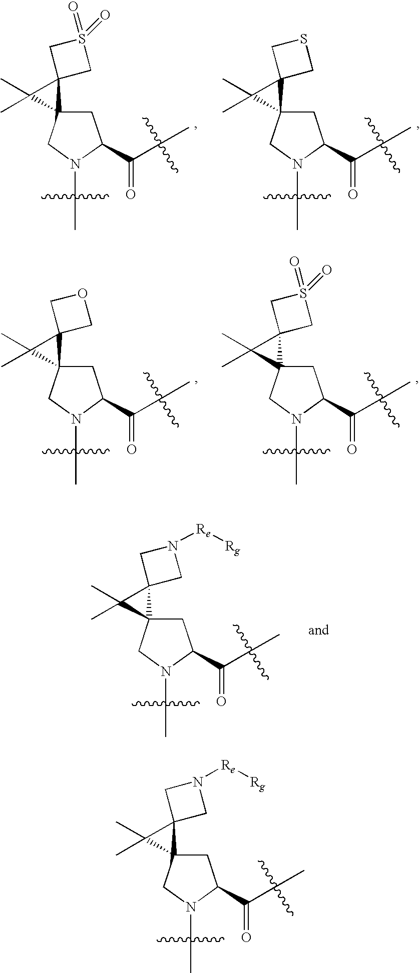
wherein R₈ is absent, C(O), or S(O)₂: and R₈ is selected hydrogen or selected from the group consisting of C₁₋₈alkyl, arylC₀₋₄alkyl, heteroarylC₀₋₄alkyl, heterocyelylC₀₋₄alkyl, and C₃₋₇-cycloalkylC₀₋₄alkyl, each of which is substituted with 0 to 4 independently selected substituents selected from the group consisting of cyano, halogen, hydroxyl, amino, thiol, C₁₋₈-alkyl, C₂₋₈-alkenyl, C₂₋₈-alkynyl, C₁₋₈-alkoxy-C₀₋₄alkyl C₁₋₈-haloalkyl, C₂₋₈-haloalkenyl, C₂₋₈-haloalkynyl, C₁₋₈-haloalkoxy, C₁₋₈-alkylthio, C₁₋₈-alkylsulfonyl, C₁₋₈-alkylsulfoxy, C₁₋₈-alkanoyl, C₁₋₈-alkoxycarbonyl, C₃₋₇-cycloalkyl-C₀₋₄alkyl, aryl-C₀₋₄alkyl, heteroaryl-C₀₋₄-alkyl, COOH, C(O)NH₂, mono- and di-C₁₋₄-alkyl-carboxamide, mono- and di-C₁₋₄-alkyl-amino-C₀₋₄alkyl, SO₃H, SO₂NH₂, and mono-and di-C₁₋₄alkylsulfonamide.

84-72. (canceled)

73. A pharmaceutical composition comprising at least one compound according to any one of the preceding claims and a pharmaceutically acceptable carrier.

74-75. (canceled)

76. The pharmaceutical composition of claim 73, wherein the additional HCV-modulating compound is interferon or derivatized interferon.

77-79. (canceled)

80. A method of treating an HCV-associated disorder comprising administering to a subject in need thereof a pharmaceutically acceptable amount of a compound according to any one of the preceding claims.

81-82. (canceled)

83. A method of treating, inhibiting or preventing the activity of HCV in a subject in need thereof, comprising administering to the subject a pharmaceutically acceptable amount of a compound according to any one of the preceding claims.

84-110. (canceled)
